(19) United States
(12) Patent Application Publication
(10) Pub. No.: US 2023/0265405 A1
(43) Pub. Date: Aug. 24, 2023

(54) ENGINEERED NUCLEASES AND METHODS OF USE THEREOF

(71) Applicant: Massachusetts Institute of Technology, Cambridge, MA (US)

(72) Inventors: VIKASH PAL SINGH CHAUHAN, CAMBRIDGE, MA (US); PHILLIP A. SHARP, NEWTON, MA (US); ROBERT LANGER, NEWTON, MA (US)

(21) Appl. No.: 18/069,387

(22) Filed: Dec. 21, 2022

Related U.S. Application Data
(60) Provisional application No. 63/268,340, filed on Feb. 22, 2022.

(51) Int. Cl. C12N 9/22

(52) U.S. Cl. CPC C12N 9/22; C12N 2310/20

(57) ABSTRACT

The present disclosure provides a method of editing a genome in a cell including exposing the cell to an engineered Cas nuclease comprising one or more mutations within the DNA binding cleft of the Cas nuclease, wherein exposure to the engineered Cas nuclease decreases, inhibits, or prevents non-homologous end joining (NHEJ) in the cell, and wherein exposure to the engineered Cas nuclease increases one or more homology-driven repair pathways within the cell. The mutant Cas nuclease is also disclosed herein.

### RELATED APPLICATION

This application claims the benefit of U.S. Provisional Patent Application No. 63/268,340, filed on Feb. 22, 2022, the entire disclosure of which is hereby incorporated herein by reference.

### STATEMENT AS TO FEDERALLY FUNDED RESEARCH

This invention was made with government support under R01 CA208205 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML file, created on Dec. 20, 2022, is named 083474-033PC_ST2650177518.1 and is 387 kilobytes in size.

### BACKGROUND

Genome editing with CRISPR-Cas nucleases harnesses cellular double-strand break (DSB) repair pathways, such as non-homologous end-joining (NHEJ), microhomology-mediated end-joining (MMEJ), and homology-directed repair (HDR), to mutate targeted loci. The Cas nuclease is programmed to create a DNA break, such as a DSB, at a specific target DNA sequence using an associated guide RNA (gRNA). NHEJ produces semi-random insertion/deletion mutations (indels) that are generally small. In contrast, MMEJ makes sequence-specific indels that can be small or large by using small homologous sequences flanking the DSB. Meanwhile, HDR utilizes a homologous DNA repair template that recombines with the DSB site to create specific mutations called precise edits. These repair pathways compete to repair each DSB due to multi-faceted regulation, though NHEJ is the dominant mechanism. The relative frequencies of these repair pathways and the mutations they produce follow a predictable distribution that is thought to be defined by the sequence of the targeted locus and the cell state. A central goal of the field is to develop means for controlling the relative frequencies of particular repair pathways, primarily with the aim of making precise editing by HDR or specific indels by MMEJ more frequent. This requires understanding what underlies competition between repair pathways and accordingly designing strategies to redirect DNA repair.

CRISPR-Cas nucleases can also be utilized in complex genome editing tools where one or more proteins are fused to a Cas, such as prime editing systems. Prime editors are Cas nucleases paired with extended gRNAs featuring homologous DNA synthesis templates (pegRNAs) whose sequences can be copied into broken DNA ends by a fused polymerase. The sequence of the DNA synthesis template is therefore directly written into the target DNA by extension of the broken target DNA end; thus, it does not require repair mechanisms such as HDR. Yet prime editing systems frequently produce indels when the polymerase fails to extend the target DNA ends and/or DNA repair pathways reject the newly extended target DNA strand, through pathways such as NHEJ and mismatch repair (MMR), and single-strand break repair (SSBR). These indels produce somewhat unpredictable mutations that need to be addressed for many applications of genome editing.

Existing CRISPR-Cas technologies tend to create indel mutations by NHEJ, MMR, SSBR, and related pathways, and there is little room to control this outcome without directly inhibiting these indel-producing pathways. There remains a need for a more precise editing mechanism, including an editing composition and system that can reliably function in dividing and non-dividing cells.

### BRIEF SUMMARY

In one aspect, this disclosure is directed to a method of editing a genome in a cell. This method includes exposing the cell to an engineered Cas nuclease comprising one or more mutations within the DNA binding cleft of the Cas nuclease, wherein exposure to the engineered Cas nuclease decreases, inhibits, or prevents an indel-producing DNA repair pathway, related non-homologous DNA repair pathways, or other means of indel generation in the cell, and wherein exposure to the engineered Cas nuclease increases one or more precise editing repair pathways within the cell.

In one embodiment, the engineered Cas nuclease is an engineered Cas9 nuclease.

In another embodiment, the homology-driven repair pathway is homology directed repair (HDR), non-homologous end joining (NHEJ), or microhomology mediated end-joining (MMEJ).

In another embodiment, the precise editing repair pathway is a combination of micro-homology end joining (MMEJ) and homology directed repair (HDR).

In another embodiment, the Cas nuclease decreases indel production. In some embodiments, the Cas nuclease decreases indel production through a particular mechanism. In some embodiments, the Cas nuclease decreases indel production through multiple pathways. In some embodiments, the Cas nuclease decreases indel production generally.

In another embodiment, the level of NHEJ and the level of HDR are measured by sequencing.

In another embodiment, the ratio of NHEJ to HDR is decreased as compared to that of a cell exposed to a reference Cas nuclease lacking the same mutations in the DNA binding cleft.

In another embodiment, the homology-driven repair pathway is microhomology mediated end-joining (MMEJ).

In another embodiment, the level of NHEJ and the level of MMEJ are measured by sequencing.

In another embodiment, the ratio of NHEJ to MMEJ is decreased as compared to that of a cell exposed to a reference Cas nuclease lacking the same mutations in the DNA binding cleft.

In another embodiment, the level of NHEJ in the cell exposed to the engineered Cas nuclease is decreased by at least 10% compared to that of a cell exposed to a reference Cas nuclease lacking the same mutations in the DNA binding cleft.

In another embodiment, the level of NHEJ in the cell exposed to the engineered Cas nuclease is decreased by at least 25% compared to that of a cell exposed to a reference Cas nuclease lacking the same mutations in the DNA binding cleft.

In another embodiment, the level of NHEJ in the cell exposed to the engineered Cas nuclease is decreased by at least 40% compared to that of a cell exposed to a reference Cas nuclease lacking the same mutations in the DNA binding cleft.

In another embodiment, the level of NHEJ in the cell exposed to the engineered Cas nuclease is decreased by at least 50% compared to that of a cell exposed to a reference Cas nuclease lacking the same mutations in the DNA binding cleft.

In another embodiment, the level of HDR in the cell exposed to the engineered Cas nuclease is increased by at least 10% compared to that of a cell exposed to a reference Cas nuclease lacking the same mutations in the DNA binding cleft.

In another embodiment, the level of HDR in the cell exposed to the engineered Cas nuclease is increased by at least 25% compared to that of a cell exposed to a reference Cas nuclease lacking the same mutations in the DNA binding cleft.

In another embodiment, the level of HDR in the cell exposed to the engineered Cas nuclease is increased by at least 40% compared to that of a cell exposed to a reference Cas nuclease lacking the same mutations in the DNA binding cleft.

In another embodiment, the level of HDR in the cell exposed to the engineered Cas nuclease is increased by at least 50% compared to that of a cell exposed to a reference Cas nuclease lacking the same mutations in the DNA binding cleft.

In another embodiment, the level of MMEJ in the cell exposed to the engineered Cas nuclease is increased by at least 10% compared to that of a cell exposed to a reference Cas nuclease lacking the same mutations in the DNA binding cleft.

In another embodiment the level of MMEJ in the cell exposed to the engineered Cas nuclease is increased by at least 25% compared to that of a cell exposed to a reference Cas nuclease lacking the same mutations in the DNA binding cleft.

In another embodiment, the level of MMEJ in the cell exposed to the engineered Cas nuclease is increased by at least 40% compared to that of a cell exposed to a reference Cas nuclease lacking the same mutations in the DNA binding cleft.

In another embodiment, the level of MMEJ in the cell exposed to the engineered Cas nuclease is increased by at least 50% compared to that of a cell exposed to a reference Cas nuclease lacking the same mutations in the DNA binding cleft.

In another embodiment, the genome is in a non-dividing cell.

In another embodiment, the non-dividing cell is a quiescent cell, a senescent cell, or a fully differentiated cell.

In another embodiment, the one or more mutations comprise mutations of an amino acid residue at a position corresponding to D54, S55, K848, R976, N980, H982, K1003, T1314, N1317, or A1322 of SEQ ID NO: 2.

In another embodiment, the one or more mutations comprise mutations of one or more amino acid residues that occupy the same position in the three-dimensional structure of the DNA binding cleft as amino acids S55, R976, K1003, or T1314 from a Streptococcus pyogenes Cas9 protein.

In another embodiment, the engineered Cas9 nuclease comprises one or more mutations in the DNA binding cleft.

In another embodiment, the engineered Cas9 nuclease comprises a replacement of a sequence in the DNA binding cleft, wherein two or more sequential amino acids in the DNA binding cleft are replaced.

In another embodiment, the replacement sequence comprises the same number of amino acids, fewer amino acids, or more amino acids than the original sequence.

In another embodiment, the Cas9 nuclease further comprises a mutation outside of the DNA binding cleft.

In another embodiment, the engineered Cas9 nuclease comprises one mutation in the DNA binding cleft.

In another embodiment, the engineered Cas9 nuclease comprises two mutations in the DNA binding cleft.

In another embodiment, the engineered Cas9 nuclease comprises three mutations in the DNA binding cleft.

In another embodiment, the engineered Cas9 nuclease comprises four mutations in the DNA binding cleft.

In another embodiment, the engineered Cas9 nuclease comprises S55R, R976A, K1003A, and T1314A mutations.

In another embodiment, the engineered Cas9 nuclease comprises K848A and H982A mutations.

In some embodiments, the engineered Cas9 nuclease system is part of a prime editing system.

In some embodiments, the engineered Cas9 comprises amino acids R221 and N394.

In some embodiments, the engineered Cas9 comprises amino acids R221, N394, A848, and A982.

In another embodiment, wherein the engineered Cas nuclease decreases, inhibits, or prevents non-homologous end joining when compared to a reference Cas nuclease lacking said mutations in the DNA binding cleft.

In another embodiment, the Cas nuclease is a Cas9 nuclease.

In another embodiment, the reference Cas9 comprises mutations, insertions, or deletions of amino acids outside of the DNA binding cleft.

In another embodiment, the engineered Cas nuclease retains at least 85%, at least 95% or at least 99% of the activity of a reference Cas nuclease without the corresponding mutations in the DNA binding cleft.

In another embodiment, the engineered Cas nuclease has the same or greater activity than a reference Cas nuclease without the corresponding mutations in the DNA binding cleft.

In another embodiment, the Cas nuclease is a fusion protein.

In another embodiment, the fusion protein is a fusion of a Cas nuclease fused to a reverse transcriptase.

In another embodiments, the Cas nuclease is a fusion protein, which is further fused to a reverse transcriptase.

In another aspect, this disclosure is directed to a method of precisely editing the genome of a non-dividing cell, the method comprising administering to the cell an agent capable of inhibiting or preventing non-homologous end joining (NHEJ) and increasing homology-driven repair (HDR).

In another embodiment, the agent is a modified Cas nuclease.

In another embodiment, the agent is a modified Cas9 nuclease.

In another embodiment, the engineered Cas9 nuclease comprises one mutation in the DNA binding cleft.

In another embodiment, the engineered Cas9 nuclease comprises two mutations in the DNA binding cleft.

In another embodiment, the engineered Cas9 nuclease comprises three mutations in the DNA binding cleft.

In another embodiment, the engineered Cas9 nuclease comprises four mutations in the DNA binding cleft.

In another embodiment, the modified Cas9 nuclease comprises mutations at one or more amino acid residues in the DNA binding cleft.

In another aspect, this disclosure is directed to an engineered Cas nuclease variant comprising two or more amino acid substitutions, mutations, or deletions in the DNA binding cleft such that the engineered Cas nuclease variant predominantly engages a homology-driven DNA repair pathway.

In another embodiment, the Cas nuclease is a Cas9 nuclease.

In another embodiment, the engineered Cas9 nuclease inhibits or prevents non-homologous end joining.

In another embodiment, the inhibition or prevention of NHEJ is determined by sequencing.

In another embodiment, the homology-driven DNA repair pathway is homology directed repair (HDR).

In another embodiment, the homology-driven DNA repair pathway is micro-homology end joining (MMEJ).

In another embodiment, the homology-driven DNA repair pathway is a combination of micro-homology end joining (MMEJ) and homology directed repair (HDR).

In another embodiment, the engineered Cas9 nuclease decreases the number of semi-random insertion/deletion (indel) mutations when compared to a reference Cas9 nuclease in a non-dividing cell.

In another embodiment, the two or more substitutions, mutations, or deletions within the DNA binding cleft are located at an amino acid residue corresponding to D54, S55, R221, N394, K848, R976, N980, H982, K1003, T1314, N1317, or A1322 of SEQ ID NO: 2, or any combination thereof.

In another embodiment, the one or more mutations within the DNA binding cleft comprise mutations S55R, R976A, K1003A, and T1314A corresponding to SEQ ID NO: 2.

In another embodiment, the one or more mutations within the DNA binding cleft comprise mutations at amino acid residues R221, N394, K848 and H982 corresponding to SEQ ID NO: 2.

In another aspect, this disclosure is directed to a method of switching a cell from a predominantly non-homologous DNA repair pathway to a homology-driven DNA repair pathway, the method comprising exposing the cell to a modified or engineered Cas nuclease.

In another aspect, this disclosure is directed to a method of decreasing or preventing indel generation in a cell during DNA repair pathways, the method comprising exposing the cell to a modified or engineered Cas nuclease.

In another embodiment, the Cas nuclease is a Cas9 nuclease.

In another embodiment, the modified or engineered Cas9 nuclease comprises one or mutations in the DNA binding cleft.

In another embodiment, the engineered Cas9 nuclease comprises one mutation in the DNA binding cleft.

In another embodiment, the engineered Cas9 nuclease comprises two mutations in the DNA binding cleft.

In another embodiment, the engineered Cas9 nuclease comprises three mutations in the DNA binding cleft.

In another embodiment, the engineered Cas9 nuclease comprises four mutations in the DNA binding cleft.

In another embodiment, the modified or engineered Cas9 nuclease comprises one or more mutations located at an amino acid residue corresponding to D54, S55, R976, N980, K1003, T1314, N1317, or A1322 of SEQ ID NO: 2, or any combination thereof.

In another embodiment, the modified or engineered Cas 9 nucleases comprise the amino acids R221, N394, A848, and A982.

In another aspect, this disclosure is directed to a method of editing a genome in a cell. This method includes exposing the cell to an engineered Cas nuclease comprising one or more mutations within the DNA binding cleft of the Cas nuclease, wherein the engineered Cas nuclease is fused or otherwise associated with a polymerase to form a prime editor, wherein exposure to the engineered Cas nuclease increases precise genome editing initiated by action of the associated polymerase, and wherein exposure to the engineered Cas nuclease decreases, inhibits, or prevents byproduct indel formation in the cell.

In one embodiment, the engineered Cas nuclease is an engineered Cas9 nuclease within a prime editing system.

In another embodiment, the ratio of byproduct indels to precise genome edits is decreased compared to that of a cell exposed to a reference Cas nuclease within a prime editing system lacking the same mutations in the DNA binding cleft.

In another embodiment, the engineered Cas9 nuclease comprises mutations at one or more amino acid residues in the DNA binding cleft.

In another embodiment, the one or more mutations comprise mutations of an amino acid residue at a position corresponding to R780, K810, K848, K855, R976, H982, or T1314 of SEQ ID NO: 2.

In another embodiment, the one or more mutations comprise mutations of one or more amino acid residues that occupy the same position in the three-dimensional structure of the DNA binding cleft as amino acids R780, K810, K848, K855, R976, H982, or T1314 from a Streptococcus pyogenes Cas9 protein.

In another embodiment, the engineered Cas nuclease comprises the amino acids R221, N394, or both R221 and N394.

In another embodiment, the engineered Cas nuclease is vPE.

In another aspect, this disclosure is directed to an engineered Cas nuclease variant comprising one or more amino acid substitutions, mutations, or deletions in the DNA binding cleft, wherein the engineered Cas nuclease is fused or otherwise associated with a polymerase to form a prime editor, such that the engineered Cas nuclease variant suppresses indel formation.

### BRIEF DESCRIPTION OF THE DRAWINGS

Aspects, features, benefits, and advantages of the embodiments described herein will be apparent with regard to the following description, appended claims, and accompanying drawings.

FIG. 1A shows sequence alignments illustrating the design of gRNA and HDR templates to introduce a precise edit for the EMX1 locus. FIG. 1B is a diagram showing the distribution of indel sizes induced without (noT) or with (T) a repair template for the EMX1 locus, wherein data are analyzed by deep next-generation sequencing and represent means of n=3 independent replicates with standard errors.

FIG. 2A shows sequence alignments illustrating the design of gRNA and HDR templates to introduce a precise edit at the AAVS1. FIG. 2B is a diagram showing the distribution of indel sizes induced without (noT) or with (T) a repair template at the AAVS1 locus, wherein data are analyzed by deep next-generation sequencing and represent means of n=3 independent replicates with standard errors.

FIG. 3A shows sequence alignments illustrating the design of gRNA and HDR templates to introduce a precise edit at the CXCR4. FIG. 3B is a diagram showing the distribution of indel sizes induced without (noT) or with (T) a repair template at the CXCR4 locus, wherein data are analyzed by deep next-generation sequencing and represent means of n=3 independent replicates with standard errors.

FIG. 4A shows sequence alignments illustrating the design of gRNA and HDR templates to introduce a precise edit at the VEGFA. FIG. 4B is a diagram showing the distribution of indel sizes induced without (noT) or with (T) a repair template at the VEGFA locus, wherein data are analyzed by deep next-generation sequencing and represent means of n=3 independent replicates with standard errors.

FIG. 5A is a graph showing the degree of depletion by a repair template for indels of different sizes at the AAVS1 locus, wherein the data are analyzed by deep next-generation sequencing and represent means of n=3 independent replicates with standard errors. FIG. 5B is a graph showing the degree of depletion by a repair template for indels of different sizes at the CXCR4 locus, wherein the data are analyzed by deep next-generation sequencing and represent means of n=3 independent replicates with standard errors. FIG. 5C is a graph showing the degree of depletion by a repair template for indels of different sizes at the EMX1 locus, wherein the data are analyzed by deep next-generation sequencing and represent means of n=3 independent replicates with standard errors. FIG. 5D is a graph showing the degree of depletion by a repair template for indels of different sizes at the VEGFA locus, wherein the data are analyzed by deep next-generation sequencing and represent means of n=3 independent replicates with standard errors.

FIG. 6 is a graph showing the degree of depletion by a repair template for indels of different sizes average over several loci, wherein the longer the indel size the greater the depletion, and wherein data are analyzed by deep next-generation sequencing and represent means of n=3 independent replicates with standard errors.

FIG. 7 is a diagram showing the frequencies of repair pathways engaged without (noT) or with (T) a repair template at several loci, wherein NHEJ mutations are subset into insertions or deletions, while MMEJ mutations are subset by microhomology length (MH1 to MH>5), wherein * indicates p<0.05 for frequency compared between noT and T, and wherein data are analyzed by deep next-generation sequencing and represent means of n=3 independent replicates with standard errors.

FIG. 8 is a schematic representation illustrating a model of the balance between NHEJ and HDR or MMEJ repair pathways for Cas9.

FIG. 9 is a schematic representation of Cas9 residues at the interface with the substrate DNA strands that selected for Alanine substitution and altered repair pathway screening, wherein the mutated residues (red) are located in either the mobile HNH domain (green) or the immobile RuvC domain (blue), which are connected by linker segments (yellow).

FIG. 10A is a schematic of a wide view of Cas9 residues at the interface with the substrate DNA strands that are selected for mutation and screening, wherein the mutated residues (red) are located in either the mobile HNH domain (green) or the immobile RuvC domain (blue), which are connected by linkers (yellow). FIG. 10B is a focused view of the residues from FIG. 10A in the target cleft. FIG. 10C is a focused view of the residues from FIG. 10A in the nontarget cleft.

FIG. 11A is a schematic showing a location of the residues from FIG. 10A in a structure of Cas9 without the DNA substrate bound. FIG. 11B is focus view of the residues from FIG. 11A in the nontarget cleft.

FIG. 12 is diagram showing a screen of precise editing and indel frequencies for engineered Cas9 variants using an HDR template targeting the EMX1 locus, wherein * indicates p<0.05 for precise editing frequency compared to Cas9 from Streptococcus pyogenes (SEQ ID NO: 1), and wherein the data are analyzed by Sanger sequencing and represent means of n=2-3 independent replicates with standard errors.

FIG. 13 is a diagram showing a screen of precise editing and indel frequencies for engineered Cas9 variants using an HDR template targeting the CXCR4 locus, wherein * indicates p<0.05 for precise editing frequency compared to Cas9 from Streptococcus pyogenes (SEQ ID NO: 1), and wherein data are analyzed by Sanger sequencing and represent means of n=2-3 independent replicates with standard errors.

FIG. 14A shows sequence alignments illustrating the design of a gRNA and HDR template to introduce a GFP to BFP conversion. FIG. 14B is a diagram showing the frequency of precise gene conversion from GFP to BFP, wherein * indicates p<0.05 for precise editing frequency compared to Cas9 from Streptococcus pyogenes (SEQ ID NO: 1), and wherein data are analyzed by flow cytometry and represent means of n=3 independent replicates with standard errors.

FIG. 15A is a flow cytometry plot showing BFP-positive (indicating precise edits), nonfluorescent (indicating indels), and GFP-positive (indicating unedited cells) cell population fractions for parental GFP+ cells; FIG. 15B is a flow cytometry plot showing BFP-positive (indicating precise edits), nonfluorescent (indicating indels), and GFP-positive (indicating unedited cells) cell population fractions of cells exposed to Cas9 from Streptococcus pyogenes (SEQ ID NO: 1). FIG. 15C is a flow cytometry plot showing BFP-positive (indicating precise edits), nonfluorescent (indicating indels), and GFP-positive (indicating unedited cells) cell population fractions of cells exposed to a R780A-H982A Cas9 mutant. FIG. 15D is a flow cytometry plot showing BFP-positive (indicating precise edits), nonfluorescent (indicating indels), and GFP-positive (indicating unedited cells) cell population fractions of cells exposed to a K855A-R976A Cas9 mutant. FIG. 15E is a flow cytometry plot showing BFP-positive (indicating precise edits), nonfluorescent (indicating indels), and GFP-positive (indicating unedited cells) cell population fractions of cells exposed to Cas9 from Streptococcus pyogenes (SEQ ID NO: 1) without an HDR template. FIG. 15F is a flow cytometry plot showing BFP-positive (indicating precise edits), nonfluorescent (indicating indels), and GFP-positive (indicating unedited cells) cell population fractions of cells exposed to a R976-H982A Cas9 mutant. FIG. 15G is a flow cytometry plot showing BFP-positive (indicating precise edits), nonfluorescent (indicating indels), and GFP-positive (indicating unedited cells) cell population fractions of cells exposed to a R976-K1003A Cas9 mutant. FIG. 15H is a flow cytometry plot showing BFP-positive (indicating precise edits), nonfluorescent (indicating indels), and GFP-positive (indicating unedited cells) cell population fractions of cells exposed to a R780A-H982A-K1003A Cas9 mutant. In each plot, data represent means of n=3 independent replicates with standard errors.

FIG. 16 is a schematic representation of Cas9 residues proximal to R976 that are selected for Arginine substitution and activity rescue.

FIG. 17 is a diagram showing a screen of precise editing and indel frequencies for engineered Cas9 variants using an HDR template targeting the VEGFA locus, wherein * indicates p<0.05 for total editing frequency compared to Cas9 from Streptococcus pyogenes (SEQ ID NO: 1), and wherein the data are analyzed by Sanger sequencing and represent means of n=2-3 independent replicates with standard errors.

FIG. 18 is a diagram showing a screen of precise editing and indel frequencies for engineered Cas9 variants using an HDR template targeting the EMX1 locus, wherein * indicates p<0.05 for precise editing frequency compared to Cas9 from Streptococcus pyogenes (SEQ ID NO: 1), and wherein data are analyzed by Sanger sequencing and represent means of n=2-3 independent replicates with standard errors.

FIG. 19 is a diagram showing the precise editing and indel frequencies using HDR templates at several loci, wherein * indicates p<0.05 for precise editing frequency compared to Cas9 from Streptococcus pyogenes (SEQ ID NO: 1), and wherein the data are analyzed by deep next-generation sequencing and represent means of n=2-3 independent replicates with standard errors.

FIG. 20A is a diagram showing indel frequencies at on-target (T) and off-target (OT1-OT3) loci for EMX1 locus, wherein * indicates p<0.05 for indel frequency compared to Cas9 from Streptococcus pyogenes (SEQ ID NO: 1); FIG. 20B is a diagram showing indel frequencies at on-target (T) and off-target (OT1-OT3) loci for VEGFA locus, wherein * indicates p<0.05 for indel frequency compared to Cas9 from Streptococcus pyogenes (SEQ ID NO: 1); wherein data are analyzed by Sanger sequencing and represent means of n=2-3 independent replicates with standard errors.

FIG. 21 is a diagram showing the frequency of precise gene conversion from GFP to BFP, wherein * indicates p<0.05 for precise editing frequency compared to Cas9 from Streptococcus pyogenes (SEQ ID NO: 1), and data are analyzed by flow cytometry and represent means of n=3 independent replicates with standard errors.

FIG. 22A is a flow cytometry plot showing BFP-positive (indicating precise edits), nonfluorescent (indicating indels), and GFP-positive (indicating unedited cells) cell population fractions for parental GFP+ cells. FIG. 22B is a flow cytometry plot showing BFP-positive (indicating precise edits), nonfluorescent (indicating indels), and GFP-positive (indicating unedited cells) cell population fractions of cells exposed to Cas9 from Streptococcus pyogenes (SEQ ID NO: 1). FIG. 22C is a flow cytometry plot showing BFP-positive (indicating precise edits), nonfluorescent (indicating indels), and GFP-positive (indicating unedited cells) cell population fractions of cells exposed to vCas9. Data represent means of n=3 independent replicates with standard errors.

FIG. 23A is a graph showing the correlation between mean indel size (for remaining indels after precise editing) and precise editing frequency using HDR templates across Cas9 variants from the Alanine-substitution screen for the EMX1 locus; FIG. 23B is a graph showing the correlation between mean indel size (for remaining indels after precise editing) and precise editing frequency using HDR templates across Cas9 variants from the Alanine-substitution screen for the CXCR4 locus. Data are analyzed by Sanger sequencing and represent means of n=2-3 independent replicates with standard errors.

FIG. 24 is a diagram showing distributions of indel sizes induced at AAVS1 locus, wherein * indicates p<0.05 for precise editing frequency compared to Cas9 from Streptococcus pyogenes (SEQ ID NO: 1), and wherein data are analyzed by deep next-generation sequencing and represent means of n=3 independent replicates with standard errors.

FIG. 25 is a diagram showing the distributions of indel sizes induced for CXCR4 locus, wherein data are analyzed by deep next-generation sequencing and represent means of n=3 independent replicates with standard errors.

FIG. 26 is a diagram showing the distributions of indel sizes induced for EMX1 locus, wherein data are analyzed by deep next-generation sequencing and represent means of n=3 independent replicates with standard errors.

FIG. 27 is a diagram showing the distributions of indel sizes induced for VEGFA locus, wherein data are analyzed by deep next-generation sequencing and represent means of n=3 independent replicates with standard errors.

FIG. 28 is a diagram showing mean indel sizes induced at several loci, wherein * indicates p<0.05 for precise editing frequency compared to Cas9 from Streptococcus pyogenes (SEQ ID NO: 1), and wherein data are analyzed by deep next-generation sequencing and represent means of n=3 independent replicates with standard errors.

FIG. 29 is a diagram showing frequencies of repair pathways engaged without (noT) a repair template at several loci, wherein NHEJ mutations are subset into insertions or deletions, while MMEJ mutations are subset by microhomology length (MH1 to MH>5), wherein * indicates p<0.05 for precise editing frequency compared to Cas9 from Streptococcus pyogenes (SEQ ID NO: 1), and wherein data are analyzed by deep next-generation sequencing and represent means of n=3 independent replicates with standard errors.

FIG. 30 is a diagram showing frequencies of repair pathways engaged with a repair template (T) at several loci, wherein NHEJ mutations are subset into insertions or deletions, while MMEJ mutations are subset by microhomology length (MH1 to MH>5), wherein * indicates p<0.05 for precise editing frequency compared to Cas9 from Streptococcus pyogenes (SEQ ID NO: 1), and wherein data are analyzed by deep next-generation sequencing and represent means of n=3 independent replicates with standard errors.

FIG. 31 is a diagram showing precise editing and indel frequencies using HDR templates without or with an MMEJ inhibitor (Rucaparib) or NHEJ inhibitor (NU7026), wherein * indicates p<0.05 for precise editing frequency compared to Cas9 from Streptococcus pyogenes (SEQ ID NO: 1), wherein ** indicates p<0.05 for precise editing frequency compared to vCas9, and wherein data are analyzed by deep next-generation sequencing and represent means of n=3 independent replicates with standard errors.

FIG. 32 is a diagram showing the precise editing and indel frequencies using HDR templates targeted to the MYC locus without or with an MMEJ inhibitor (Rucaparib) or NHEJ inhibitor (NU7026), wherein * indicates p<0.05 for precise editing frequency compared to Cas9 from Streptococcus pyogenes (SEQ ID NO: 1), wherein ** indicates p<0.05 for precise editing frequency compared to vCas9, and wherein data are analyzed by deep next-generation sequencing and represent means of n=3 independent replicates with standard errors.

FIG. 33 is a graph showing the degree of depletion by a repair template for indels of different sizes averaged over several loci, wherein data are analyzed by deep next-generation sequencing and represent means of n=3 independent replicates with standard errors.

FIG. 34A is a graph showing the degree of depletion by a repair template for indels of different sizes for AAVS1 locus, wherein vCas9 increases competitiveness of templated precise editing; FIG. 34B is a graph showing the degree of depletion by a repair template for indels of different sizes for CXCR4 locus, wherein vCas9 increases competitiveness of templated precise editing; FIG. 34C is a graph showing the degree of depletion by a repair template for indels of different sizes for EMX1 locus, wherein vCas9 increases competitiveness of templated precise editing; FIG. 34D is a graph showing the degree of depletion by a repair template for indels of different sizes for VEGFA locus, wherein vCas9 increases competitiveness of templated precise editing; data are analyzed by deep next-generation sequencing and represent means of n=3 independent replicates with standard errors.

FIG. 35A shows sequence alignments illustrating the design of gRNAs and MDR templates to introduce precise edits for AAVS1 locus; FIG. 35B shows sequence alignments illustrating the design of gRNAs and MDR templates to introduce precise edits for CD274 locus; FIG. 35C shows sequence alignments illustrating the design of gRNAs and MDR templates to introduce precise edits for CXCR4 locus; FIG. 35D shows sequence alignments illustrating the design of gRNAs and MDR templates to introduce precise edits for MYC locus; FIG. 35E shows sequence alignments illustrating the design of gRNAs and MDR templates to introduce precise edits for TGFB1 locus; FIG. 35F shows sequence alignments illustrating the design of a gRNA and MDR template to introduce a precise edit at the KRAS locus.

FIG. 36A shows sequence alignments illustrating the design of gRNAs and MDR templates to introduce a GFP to BFP conversion for TGFB1 locus; FIG. 36B is a diagram showing the precise editing and indel frequencies using MDR templates with varied microhomology arm lengths (0-10 bp at the 5′ ends, 20 bp at the 3′ ends); FIG. 36C is a diagram showing the frequency of precise gene conversion from GFP to BFP; wherein all data are analyzed by flow cytometry and represent means of n=3 independent replicates with standard errors.

FIG. 37A is a diagram showing the precise editing and indel frequencies using MDR templates in dividing HEK293T cells, wherein * indicates p<0.05 for precise editing frequency compared to Cas9 from Streptococcus pyogenes (SEQ ID NO: 1), FIG. 37B is a diagram showing the precise editing and indel frequencies using MDR templates in non-dividing (quiescent) primary human dermal fibroblasts, wherein * indicates p<0.05 for precise editing frequency compared to Cas9 from Streptococcus pyogenes (SEQ ID NO: 1); wherein all data are analyzed by deep next-generation sequencing and represent means of n=3 independent replicates with standard errors.

FIG. 38A is diagram showing the cell cycle profiling of dividing and quiescent primary human dermal fibroblasts for dividing and quiescent cells, wherein data are analyzed by flow cytometry and represent means of n=3 independent replicates with standard errors, according to embodiments of the present teachings; FIG. 38B is a flow cytometry plot showing EdU-high (indicating S phase), Propidium Iodide-high (indicating G2 phase), and EdU-low Propidium Iodide-low (indicating G1 or G0 phase) cell population fractions for dividing cells; FIG. 38C is a flow cytometry plot showing EdU-high (indicating S phase), Propidium Iodide-high (indicating G2 phase), and EdU-low Propidium Iodide-low (indicating G1 or G0 phase) cell population fractions for quiescent cells, wherein data represent means of n=3 independent replicates with standard errors.

FIG. 39 is a diagram showing the distributions of frequencies of repair pathways engaged without (noT) or with (T) a repair template at several loci. * Indicates p<0.05 for MMEJ frequency compared between noT and T in c. Data were analyzed by deep sequencing and represent means of n=3 independent replicates with standard errors.

FIG. 40A is a diagram showing the results of a screen of precise editing and indel frequencies for engineered Cas9 single-mutant variants using an HDR template targeting the EMX1 locus. FIG. 40B is a diagram showing the results of a screen of precise editing and indel frequencies for engineered Cas9 double-mutant variants using an HDR template targeting the EMX1 locus. Except where noted, the annotation used in FIG. 40A is used throughout the application, such that “−5 sub 3 bp” indicates a 3 base pair substitution at the −5 position.

FIG. 41A is a diagram showing the results of a screen of precise editing and indel frequencies for engineered Cas9 single-mutant variants using an HDR template targeting the CXCR4 locus. FIG. 41B is a diagram showing the results of a screen of precise editing and indel frequencies for engineered Cas9 double-mutant variants using an HDR template targeting the CXCR4 locus.

FIG. 42A is a diagram showing the results of a screen of precise editing and indel frequencies for engineered Cas9 triple-mutant variants using an HDR template targeting the VEGFA locus. FIG. 42B is a diagram showing the results of a screen of precise editing and indel frequencies for engineered Cas9 quadruple-mutant variants using an HDR template targeting the VEGFA locus.

FIG. 43A is a diagram showing the results of a screen of precise editing and indel frequencies for Cas9 and vCas9 using a small (1-10 bp) HDR template targeting the AAVS1, CXCR4, or VEGFA loci in HeLa cells. FIG. 43B is a diagram showing the results of a screen of precise editing and indel frequencies for Cas9 and vCas9 using a small (1-10 bp) HDR template targeting the CD274, KRAS, or VEGFA loci in A549 cells. FIG. 43C is a diagram showing the results of a screen of precise editing and indel frequencies for Cas9 and vCas9 using a small (1-10 bp) HDR template targeting the KRAS, MYC, or TGFB1 loci in Panc1 cells.

FIG. 44A is a design of a gRNA and HDR template to introduce a large insertion into the THROLNC locus. FIG. 44B is a diagram showing the results of a screen examining precise editing and indel frequencies using large insertion (˜50 bp) templates at several loci.

FIG. 45A is a design of a gRNA to induce untemplated collapse of duplications. FIG. 45B is a diagram showing the results of a screen examining precise editing and indel frequencies for untemplated collapse of duplications (10-20 bp) at several loci. * indicates p<0.05 for precise editing frequency compared to wild-type Cas9.

FIG. 46A is a diagram showing the results of a screen examining precise editing at the CXCR4 locus by vCas9 compared to other Cas9 variants and fusions. FIG. 46B is a diagram showing the results of a screen examining precise editing and indel frequencies at the CXCR4 locus between various Cas9 fusions and variants using an HDR template. * indicates p<0.05 for precise editing frequency compared to wild-type Cas9.

FIG. 47 is a schematic representation of an assay for DNA break structure, where paired DSBs lead to perfect deletion junctions for blunt cuts and insertions in the deletion junctions for staggered cuts. The sequences of the insertions indicate cut positions in each strand.

FIG. 48 is a heatmap of DNA break positions in the target strand (TS) and non-target strand (NTS) for engineered Cas9 variants. Deeper blue represents a higher frequency at a particular position.

FIG. 49A is a scatter plot showing the correlation between staggered cut frequency and precise editing frequency across Cas9 variants from the Alanine-substitution screen. FIG. 49B is a series of heatmaps showing the frequency of DNA break positions at the EMX1 locus and CXCR4 locus of Cas9 and vCas9.

FIG. 50A displays the rates of the top sequences resulting from editing with Cas9 variants at the EMX1 locus. Substitutions, insertions, and deletions are depicted. Deletions at microhomologies are labeled. The most frequent fifteen sequences are displayed along with percentages out of all sequencing reads. FIG. 50B displays the rates of the top sequences resulting from editing with vCas9 variants at the EMX1 locus. Substitutions, insertions, and deletions are depicted. Deletions at microhomologies are labeled. The most frequent fifteen sequences are displayed along with percentages out of all sequencing reads. Data were analyzed by deep sequencing and represent a single replicate. Substitutions are represented in bold, insertions are represented by a red box, deletions are represented by a single dash, microhomologies are represented by a blue box, and the predicted cleavage position is represented by a series of dashes.

FIG. 51A displays the rates of the top sequences resulting from editing with Cas9 variants at the AAVS1 locus. Substitutions, insertions, and deletions are depicted. Deletions at microhomologies are labeled. The most frequent fifteen sequences are displayed along with percentages out of all sequencing reads. FIG. 51B displays the rates of the top sequences resulting from editing with vCas9 variants at the AAVS1 locus. Substitutions, insertions, and deletions are depicted. Deletions at microhomologies are labeled. The most frequent fifteen sequences are displayed along with percentages out of all sequencing reads. Data were analyzed by deep sequencing and represent a single replicate. Substitutions are represented in bold, insertions are represented by a red box, deletions are represented by a single dash, microhomologies are represented by a blue box, and the predicted cleavage position is represented by a series of dashes.

FIG. 52A is a schematic model of the balance between indels and precise edits for prime editor variants. FIG. 52B is a schematic that depicts how a mutation in a DNA binding cleft might lead to suppressed indel frequency.

FIG. 53 is a diagram showing the results of an experiment examining precise editing and indel frequencies for PE variants using pegRNAs and nicking gRNAs at an EMX1 locus, a GFP locus, a KRAS locus, and a MYC locus.

FIG. 54. is a bar graph showing the results of an experiment examining of precise editing and indel frequencies for engineered prime editor single-mutant variants using a pegRNA and nicking gRNA.

FIG. 55A is a bar graph showing the results of an experiment examining the frequency of precise gene conversion from GFP to BFP for prime editing variants using a pegRNA and nicking gRNA. FIG. 55B is a bar graph showing the results of an experiment examining of precise editing and indel frequencies for engineered prime editor dual-mutant variants using a pegRNA and nicking gRNA. FIG. 55C is a bar graph showing the results of an experiment examining precise editing and indel frequencies for engineered PE triple-mutant variants using a pegRNA and nicking gRNA.

FIG. 56 is a scatter plot showing the Correlation between staggered cut frequency for Cas9 variants and precise editing frequency for corresponding prime editing variants from the single mutant Alanine-substitution screen.

FIG. 57A is a bar graph showing the results of an experiment examining the frequency of precise gene conversion from GFP to BFP using a pegRNA and nicking gRNA. FIG. 57B-D are flow cytometry plots showing BFP-positive (indicating precise edits), nonfluorescent (indicating indels), and GFP-positive (indicating unedited cells) cell population fractions in control GFP+ cell populations (FIG. 57B), in cells treated with a prime editor (PE) (FIG. 57C), or in cells treated with vPE (FIG. 57D). Data represent means of n=3 independent replicates with standard errors.

FIG. 58A is a bar graph showing the results of an experiment examining precise editing and indel frequencies using small edit pegRNAs and nicking gRNAs at several loci. FIG. 58B is a bar graph showing the results of an experiment examining precise editing and indel frequencies for PE variants using pegRNAs and nicking gRNAs at several loci.

FIG. 59A and FIG. 59B show the rates of the top sequences resulting from editing with PE (FIG. 59A) and vPE (FIG. 59B) variants at the STAT1 locus. The most frequent fifteen sequences are displayed along with percentages out of all sequencing reads. Data were analyzed by deep sequencing and represent a single replicate. Substitutions are represented in bold, insertions are represented by a red box, deletions are represented by a single dash, Precise edits are represented by a blue box, and the predicted cleavage position is represented by a series of dashes.

FIG. 60A and FIG. 60B show the rates of the top sequences resulting from editing with PE (FIG. 60A) and vPE (FIG. 60B) variants at the TGFB1 locus. The most frequent fifteen sequences are displayed along with percentages out of all sequencing reads. Data were analyzed by deep sequencing and represent a single replicate. Substitutions are represented in bold, insertions are represented by a red box, deletions are represented by a single dash, Precise edits are represented by a blue box, and the predicted cleavage position is represented by a series of dashes.

FIG. 61A is a bar graph showing the results of an experiment examining frequencies of repair outcomes using pegRNAs at several loci, comparing PE to vPE. FIG. 61B is a bar graph showing the results of an experiment examining precise editing and indel frequencies using small edit pegRNAs at several loci. * indicates p<0.05 for precise editing frequency compared to PE. Data were analyzed by deep sequencing represent means of n=3 independent replicates with standard errors.

FIG. 62 is a bar graph showing the results of an experiment examining frequencies of repair outcomes using pegRNAs at several loci, comparing PE to vPE. * indicates p<0.05 for precise editing frequency compared to PE. Data were analyzed by deep sequencing represent means of n=3 independent replicates with standard errors.

### DETAILED DESCRIPTION

It will be appreciated that for clarity, the following discussion will describe various aspects of embodiments of the applicants teachings. It should be noted that the specific embodiments are not intended as an exhaustive description or as a limitation to the broader aspects discussed herein.

### Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an,” and “the” include both singular and plural referents unless the context clearly dictates otherwise.

As used herein, the term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used here, the terms “about” or “approximately” when referring to a measurable value such as a parameter, an amount, a temporal duration, and the like, are meant to encompass variations of and from the specified value, such as variations of +/−10% or less, +/−5% or less, +/−1% or less, +/−0.5% or less, and +/−0.1% or less of and from the specified value, insofar such variations are appropriate to perform in the disclosed invention. It is to be understood that the value to which the modifier “about” or “approximately” refers is itself also specifically disclosed.

As used herein, the term “engineered” refers to molecules or cells that have an alteration from their natural state.

As used herein, the term “DNA” means a deoxyribonucleic acid comprising two polynucleotide chains that coil around each other to form a double helix carrying genetic instructions.

As used herein, the term “PAM” means a protospacer adjacent motif. A PAM is a region of between two and six nucleotides adjacent to, but not part of, a target sequence that is recognized by a Cas nuclease to identify a sequence of DNA for cleavage. The commonly accepted PAM sequence for Cas nucleases derived from Streptococcus pyogenes is nGG, wherein n is any nucleotide selected from cytosine, adenine, guanine, and thymine. However, the engineered Cas nucleases disclosed herein are not limited to identifying PAM sequences of the formula nGG.

As used herein, the term “guide RNA sequence,” or “gRNA sequence,” refers to a sequence of ribonucleic acid capable of targeting a specific complementary sequence. The present disclosure contemplates guide RNA sequences which target a complementary sequence within a genome. The present disclosure also contemplates guide RNA sequences which target a complementary sequence within a non-genomic vector. The present disclosure also contemplates guide RNA sequences which target a complementary sequence ex vivo.

As used herein, the term “non-homologous end joining,” or “NHEJ,” refers to the process of repairing double-strand breaks in DNA by directly joining both ends of the break.

As used herein, the term “microhomology-mediated end joining,” or “MMEJ,” refers to the process of repairing double-strand breaks in DNA by using a homologous sequences flanking the double-strand break.

As used herein, the terms “homology directed repair,” or “HDR,” refers to the process of repairing double-strand breaks in DNA by using a homologous DNA repair template.

As used herein, the terms “prime editing system” and “prime editors” refer to an editing system that is not classified as HDR or NHEJ. Primer editors use a fused polymerase, generally reverse transcriptase, to write new sequences directly into a nicked genomic DNA strand in a cell. They use a pegRNA as a template for writing these new sequences in. They also result in indels when that direct writing process fails. The precise edits are therefore homology-dependent, and the indels are non-homologous, meaning they are considered neither HDR or NHEJ, but nor are they non-homologous.

As used herein, the term “polypeptide” and the likes refer to an amino acid sequence including a plurality of consecutive polymerized amino acid residues (e.g., at least about 2 consecutive polymerized amino acid residues). “Polypeptide” refers to an amino acid sequence, oligopeptide, peptide, protein, enzyme, nuclease, or portions thereof, and the terms “polypeptide,” “oligopeptide,” “peptide,” “protein,” “enzyme,” and “nuclease,” are used interchangeably.

Polypeptides as described herein also include polypeptides having various amino acid additions, deletions, or substitutions relative to the native amino acid sequence of a polypeptide. In some embodiments, polypeptides that are homologs of a polypeptide contain non-conservative changes of certain amino acids relative to the native sequence of a polypeptide. In some embodiments, polypeptides that are homologs of a polypeptide contain conservative changes of certain amino acids relative to the native sequence of a polypeptide, and thus may be referred to as conservatively modified variants. A conservatively modified variant may include individual substitutions, deletions or additions to a polypeptide sequence which result in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles.

As used herein, the term “fusion protein” refers to at least a first polypeptide sequence linked either directly or indirectly to at least a second polypeptide sequence with which it is not normally directly linked in nature.

As used herein, the term “DNA binding cleft” may be used to refer to the amino acid residues of a Cas protein or variant, or amino acid residues in a region of a Cas protein or variant, which come into contact with DNA, either stably or transiently. “DNA binding cleft” may also be used to refer to residues that may act to stabilize these residues and/or regions.

As used herein, the term “indel” refers to a molecular edit to a nucleic acid sequence, including but not limited to genomic sequences, that can be either an insertion or a deletion of a number of nucleotides within a nucleic acid sequence. The present disclosure contemplates both small and large indels.

As used herein, the terms “mutation,” “variant,” and the like refer to a polypeptide or nucleotide sequence that differs from a given polypeptide or nucleotide sequence in amino acid or nucleic acid sequence by the addition (e.g., insertion), deletion, or conservative substitution of amino acids or nucleotides, but that retains some or all the biological activity of the given polypeptide (e.g., a variant nucleic acid could still encode the same or a similar amino acid sequence). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al., J. Mol. Biol., 157: 105-132 (1982)). The hydropathic index of an amino acid is based on a consideration of its hydrophobicity and charge. It is known in the art that amino acids of similar hydropathic indexes can be substituted and still retain protein function. The present disclosure provides amino acids having hydropathic indexes of ±2 that can be substituted. The hydrophilicity of amino acids also can be used to reveal substitutions that would result in proteins retaining some or all biological functions. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). The term “variant” also can be used to describe a polypeptide or fragment thereof that has been differentially processed, such as by proteolysis, phosphorylation, or other post-translational modification, yet retains some or all its biological and/or antigen reactivities. Use of “variant” herein is intended to encompass fragments of a variant unless otherwise contradicted by context.

Alternatively, or additionally, a “variant” is to be understood as a polynucleotide or protein which differs in comparison to the polynucleotide or protein from which it is derived by one or more changes in its length or sequence. The polypeptide or polynucleotide from which a protein variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence in comparison to the parent molecule. Also encompassed modified molecules such as but not limited to post-translationally modified proteins (e.g. glycosylated, biotinylated, phosphorylated, ubiquitinated, palmitoylated, or proteolytically cleaved proteins). Also, mixtures of different molecules such as but not limited to RNA-DNA hybrids, are encompassed by the term “variant.” Typically, a variant is constructed artificially by gene-technological means whilst the parent polypeptide is a wild-type protein. However, naturally occurring variants are to be understood to be encompassed by the term “variant” as used herein. Further, the variants usable in the present disclosure may also be derived from homologs, orthologs, or paralogs of the parent molecule or from artificially constructed variant, provided that the variant exhibits at least one or more biological activity of the parent molecule, i.e. is functionally active.

Alternatively, or additionally, a “variant” as used herein can be characterized by a certain degree of sequence identity to the parent polypeptide or parent polynucleotide from which it is derived. A protein variant in the context of the present disclosure can exhibit at least 90% sequence identity to its parent polypeptide. A polynucleotide variant in the context of the present disclosure can exhibit at least 80% sequence identity to its parent polynucleotide. A polynucleotide variant in the context of the present disclosure can exhibit at least 70% sequence identity to its parent polynucleotide. A polynucleotide variant in the context of the present disclosure can exhibit at least 60% sequence identity to its parent polynucleotide. A polynucleotide variant in the context of the present disclosure can exhibit at least 50% sequence identity to its parent polynucleotide. The term “at least 50% sequence identity” refers to a sequence identity of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

The similarity of nucleotide and amino acid sequences, i.e. the percentage of sequence identity, can be determined via sequence alignments. Such alignments can be carried out with several art-known algorithms with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on http://www.ebi.ac.uk/Tools/clustalw/or on http://www.ebi.ac.uk/Tools/clustalw2/index.html or on http://npsa-pbil.ibcp.fr/cgi-bin/npsa_automat.pl?page=/NPSA/npsa_clustalw.html. Parameters used are the default parameters as they are set on http://www.ebi.ac.uk/Tools/clustalw/or http://www.ebi.ac.uk/Tools/clustalw2/index.html. The grade of sequence identity (sequence matching) may be calculated using e.g. BLAST, BLAT or BlastZ (or BlastX). A similar algorithm is incorporated into the BLASTN and BLASTP programs of Altschul et al. (1990) J. Mol. Biol. 215: 403-410. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs can be used. Sequence matching analysis may be supplemented by established homology mapping techniques like Shuffle-LAGAN (Brudno M., Bioinformatics 2003b, 19 Suppl 1:154-162) or Markov random fields. When percentages of sequence identity are referred to in the present application, these percentages are calculated in relation to the full length of the longer sequence, if not specifically indicated otherwise.

### DNA Repair Pathways

Some embodiments disclosed herein are directed to non-naturally occurring or engineered systems, methods, and compositions for the repair of DNA targets. DNA repair pathways are discussed in more detail below.

Cells have developed a number of pathways for repairing single (SSB) and double-strand breaks (DSB) in DNA. One pathway normally active in non-dividing cells is non-homologous end joining (NHEJ). NHEJ repairs double stranded DNA breaks by directly ligating the two ends of nucleic acid together. The result of NHEJ is a semi-random insertion or deletion of a DNA sequence, called an indel, that are generally small. NHEJ does not employ a homologous sequence as a template for these indels and may result in losing sequence information in the process. In other words, this repair mechanism can be mutagenic. NHEJ relies on chance pairings, or microhomologies, between the single-stranded tails of the two DNA fragments to be joined.

Another DSB repair pathway is microhomology-mediated end-joining (MMEJ). MMEJ process generally involves the following steps: resection of the DSB ends, annealing of microhomologous region, removal of heterologous flaps, fill-in synthesis and ligation. A certain degree of end resection may also be needed for MMEJ. Following end resection, the exposed microhomologous sequence is annealed to form an intermediate with 3′-flap and gaps on both sides of the DSB. The microhomologous sequences then move closer and perform annealing, which may start in a thermodynamically-driven fashion and be regulated with proteins factors or enzymes. After microhomologous annealing, the non-homologous 3′ tail (3′-heterologous flaps) is removed to allow DNA polymerase to fill-in the gap and stabilize the annealed intermediate. This step can be executed by substrate structure specific endonuclease, such as XPF/ERCC1 in mammals. The final step of MMEJ is DNA ligase mediated break end ligation.

Another DSB repair pathway is homology-directed repair (HDR). HDR process is one of the most accurate for DSB repair due to the requirement of higher sequence homology between the damaged and intact donor strands of DNA. The process can be error-free as long as the DNA template used for repair is identical to the original DNA sequence at the DSB. If not, specific mutations could be introduced into the damaged DNA.

The HDR process generally involves the following steps: the 5′-ended DNA strand is resected at the break to create a 3′ overhang. This can serve as both a substrate for proteins required for strand invasion and a primer for DNA repair synthesis. The invasive strand can then displace one strand of the homologous DNA duplex and pair with the other, which results in the formation of the hybrid DNA (a displacement loop or D loop). The recombination intermediates can then be resolved to complete the DNA repair process.

Prime editors are Cas9 nickases (Cas9n) paired with extended gRNAs featuring homologous templates (pegRNAs) whose sequences can be copied into nicked DNA ends by a fused reverse transcriptase (RT). Unlike the DSB pathways described above, nicked DNA is a single strand break.

Another DNA repair pathway is mismatch repair (MMR). MMR repairs mismatched DNA sequences between complementary strands. The MMR process generally involves the following steps: the mismatch is identified by the bulged DNA structure it creates. One strand of mismatched DNA, generally the newly synthesized and nicked strand, is partially excised. The other strand is used as a template to resynthesize the excised strand. The resynthesized strand is then ligated to complete the repair process.

An additional DNA repair pathway is single-strand break repair (SSBR). SSBR is comprised of several related pathways that repair single-strand breaks, or nicks. The nicks can arise through many processes. The SSBR process generally involves the following steps: the single-strand break is identified. The DNA ends at the break are processed, which may lead to large or small gaps in the nicked strand. The other strand is used as a template to resynthesize the DNA and fill in the gap. The resynthesized strand is then ligated to complete the repair process.

### CRISPR-Cas Protein

Some embodiments disclosed herein are directed to non-naturally occurring or engineered systems, methods, and compositions comprising a Cas protein. Cas proteins are discussed in more details below.

In the conflict between bacterial hosts and their associated viruses, CRISPR-Cas systems provide an adaptive defense mechanism that utilizes programmed immune memory. CRISPR-Cas systems provide their defense through three stages: adaptation, the integration of short nucleic acid sequences into the CRISPR array that serves as memory of past infections; expression, the transcription of the CRISPR array into a pre-crRNA (CRISPR RNA) transcript and processing of the pre-crRNA into functional crRNA species targeting foreign nucleic acids; and interference, the programming of CRISPR effectors by crRNA to cleave nucleic acid of foreign threats. Across all CRISPR-Cas systems, these fundamental stages display enormous variation, including the identity of the target nucleic acid (either RNA, DNA, or both) and the diverse domains and proteins involved in the effector ribonucleoprotein complex of the system.

CRISPR-Cas systems can be broadly split into two classes based on the architecture of the effector modules involved in pre-crRNA processing and interference. Class 1 systems have multi-subunit effector complexes composed of many proteins, whereas Class 2 systems rely on single-effector proteins with multi-domain capabilities for crRNA binding and interference; Class 2 effectors often provide pre-crRNA processing activity as well. Class 1 systems contain 3 types (type I, III, and IV) and 33 subtypes, including the RNA and DNA targeting type III-systems. Class 2 CRISPR families encompass 3 types (type II, V, and VI) and 17 subtypes of systems, including the RNA-guided DNases Cas9 and Cas12 and the RNA-guided RNase Cas13. Continual sequencing of novel bacterial genomes and metagenomes uncovers new diversity of CRISPR-Cas systems and their evolutionary relationships, necessitating experimental work that reveals the function of these systems and develops them into new tools.

### Prime Editing Systems

Some embodiments disclosed herein are directed to non-naturally occurring or engineered systems, methods, and compositions comprising a Cas protein variant fused to a polymerase in a prime editing system. The Cas variants contemplated herein include variants of Cas9, Cas12, or Cas13. The polymerases contemplated herein include variants of DNA polymerases including reverse transcriptases. Prime editing systems are discussed in more detail below.

Prime editing systems are made of a Cas nuclease fused to a DNA polymerase, or ‘prime editor,’ coupled with extended guide RNAs which include a DNA synthesis template, or ‘pegRNA.’ The Cas enzyme in the fusion protein may produce a DSB, or may be engineered to instead produce a single-strand break, or ‘nick.’ The polymerase may be a reverse transcriptase or other DNA polymerase that can utilize the DNA synthesis template in the pegRNA as a primer for incorporating genome edits into a target DNA sequence.

The prime editing system components work in concert to introduce a programmed genome edit in the target DNA. In this process, the Cas enzyme in the prime editor is guided to a target DNA sequence, such as a genomic DNA sequence, using the guide RNA sequence in the pegRNA. The Cas enzyme makes a DNA break, such as a DSB or nick, in the target DNA sequence. The DNA synthesis template extension of the pegRNA then anneals to complementary sequences in the cut target DNA. The polymerase in the prime editor then extends the cut target DNA, copying the DNA synthesis template extension sequence in the pegRNA. The newly extended target DNA strand now contains a replacement sequence for the original target DNA sequence that includes the programmed genome edit. Through DNA repair, such as HDR, MMR, SSBR, and related mechanisms, the endogenous sequence is replaced by the newly synthesized target DNA strand extension. Thus, the prime editing system directly writes replacement DNA sequences into target DNA and utilizes DNA repair to resolve the resulting mismatches between the original and replacement sequences.

Prime editing systems may fail to incorporate the programmed genome edit, and may instead introduce a byproduct mutation referred to as an indel as described above. These indels may result from failure of the cut target DNA to bind, failure of the polymerase to extend the target DNA sequence with the template extension sequence, failure of DNA repair mechanisms in replacing the endogenous target DNA sequence with the replacement sequence, errors in DNA repair, or other processes. The DNA repair mechanisms introducing indels may be NHEJ, MMEJ, MMR, SSBR, and related mechanisms.

### CRISPR-Cas Protein Variants

Some embodiments disclosed herein are directed to non-naturally occurring or engineered systems, methods, and compositions comprising a Cas protein variant. The Cas variants contemplated herein include variants of Cas9, Cas12, or Cas13. Cas protein variants are discussed in more detail below.

The Cas protein variants of the disclosure can be variants of a Cas nuclease, such as a Cas9 nuclease variant. The Cas protein of the disclosure can be a fusion protein. The Cas protein variant of the disclosure can be an engineered Cas nuclease, such as an engineered Cas9 nuclease. In one embodiment, the fusion protein can be Cas9 fused to Reverse Transcriptase. Such engineered Cas9-nickase fusions may be referred to as prime editors. Further disclosed herein are Cas protein variants linked either directly or indirectly to a Cas nickase, which is a Cas nuclease with one or more mutations (including, but not limited to, modifications to amino acids D10, D54, S55, E762 H840, K848, D839, N863, R976, N980, H982, H983, D986 K1003, T1314, N1317, or A1322 of SEQ ID NO: 2, that inactive one nuclease domain.

The engineered Cas nuclease variant can comprise one or more mutations at one or more amino acids residues, such as one or more mutations at one or more amino acid residues of a DNA binding cleft of the nuclease, such that the engineered Cas nuclease variant engages a different repair pathway as compared to the natural nuclease under normal circumstances. The DNA-binding cleft includes those amino acids in contact with DNA, either stably (e.g., in a particular conformation) or transiently (e.g., during conformational shifts) and can also include amino acid residues in positions that could possibly stabilize these regions. In one embodiment, the engineered Cas nuclease variant predominantly engages a homology-driven DNA repair pathway. In another embodiment, the engineered Cas9 nuclease variant inhibits or prevents non-homologous end joining (NHEJ). The inhibition or prevention of NHEJ can be determined by sequencing. In another embodiment, the homology-driven DNA repair pathway is homology directed repair (HDR). In another embodiment, the homology-driven DNA repair pathway is micro-homology end joining (MMEJ). In another embodiment, the homology-driven DNA repair pathway is a combination of micro-homology end joining (MMEJ) and homology directed repair (HDR). In another embodiment, the engineered Cas9 nuclease variant decreases the number of semi-random insertion/deletion (indel) mutations when compared to a reference Cas9 nuclease in a non-dividing cell.

The engineered Cas nuclease can comprise the same number of amino acids, fewer amino acids, or more amino acids than the original sequence. In some embodiments, the amino acid sequence of the engineered Cas nuclease has at least about 1 mutation, about 2 mutations, about 3 mutations, about 4 mutations, about 5 mutations, about 6 mutations, about 7 mutations, about 8 mutations, about 9 mutations, about 10 mutations, about 11 mutations, about 12 mutations, about 13 mutations, about 14 mutations, about 15 mutations, about 16 mutations, about 17 mutations, about 18 mutations, about 19 mutations, about 20 mutations, about 21 mutations, about 22 mutations, about 23 mutations, about 24 mutations, about 25 mutations, or any ranges that are made of any two or more points in the above list.

The engineered Cas nuclease can comprise one or more mutations of an amino acid residue inside, outside, or a mixture of both inside and outside of the DNA binding cleft of the nuclease. In another embodiment, the mutations correspond to one or more of to D54, S55, R976, N980, K1003, T1314, N1317, or A1322 of SEQ ID NO: 2. The engineered Cas nuclease can comprise one or more mutations of one or more amino acid residues that occupy the same position in the three-dimensional structure of the DNA binding cleft as amino acids S55, R976, K1003, or T1314 from a Streptococcus pyogenes Cas9 protein. The engineered Cas nuclease can comprise one or more of the D54, S55, R976, N980, K1003, T1314, N1317, or T1314 mutations of SEQ ID NO: 2. The engineered Cas nuclease can comprise all of the S55R, R976A, K1003A, and T1314A mutations of SEQ ID NO: 2.

The engineered Cas nuclease of the disclosure can comprise one or more replacements of a sequence, such as one or more replacement of a sequence in a DNA binding cleft of the nuclease and/or one or more replacement of a sequence outside a DNA binding cleft of the nuclease. The engineered Cas nuclease can have at least about 1 non-sequential or sequential amino acids, about 2 non-sequential or sequential amino acids, about 3 non-sequential or sequential amino acids, about 4 non-sequential or sequential amino acids, about 5 non-sequential or sequential amino acids, about 6 non-sequential or sequential amino acids, about 7 non-sequential or sequential amino acids, about 8 non-sequential or sequential amino acids, about 9 non-sequential or sequential amino acids, or about 10 non-sequential or sequential amino acids that are replaced in the DNA binding cleft.

The engineered Cas nuclease of the disclosure can retain between about 10% and 100% nuclease activity when compared to a reference Cas nuclease without the corresponding one or more mutations as measured by sequencing. In some embodiments, nuclease activity can be measured by next generation sequencing. In some embodiments, nuclease activity can be measured by Sanger sequencing. In some embodiments, nuclease activity can be measured by long-read sequencing.

The engineered Cas nuclease of the disclosure can retain about 10% about 11%, about 12%, about 13%, about 14%, about 15%, about 16%, about 17%, about 18%, about 19%, about 20%, about 21%, about 22%, about 23%, about 24% about 25%, about 26%, about 27%, about 28%, about 29%, about 30% about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the nuclease activity of a reference Cas nuclease without the corresponding one or more mutations. The engineered Cas nuclease disclosed herein can have the same or greater activity than a reference Cas nuclease without mutation.

The engineered Cas nuclease disclosed herein can have the same or greater activity than a reference Cas nuclease without mutation. The engineered Cas nuclease can comprise an amino acid sequence selected from the group consisting of SEQ ID NOs: 3-13. For example, the engineered Cas nuclease can comprise an amino acid sequence at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOs: 3-13.

The engineered Cas nuclease can comprise an amino acid sequence selected from the group consisting of SEQ ID NOs: 3-13. For example, the engineered Cas nuclease can comprise an amino acid sequence at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOs: 3-13.

The engineered Cas nuclease of the disclosure can be used in a method of editing a genome in a cell. The method can comprise exposing the cell to an engineered Cas nuclease comprising one or more mutations within the DNA binding cleft of the Cas nuclease. The exposure to the engineered Cas nuclease can decrease, inhibit, or prevent non-homologous end joining (NHEJ) in the cell, and can increase one or more homology-driven repair pathways within the cell. The engineered Cas nuclease can be an engineered Cas9 nuclease. The homology-driven repair pathway can be homology directed repair (HDR) or microhomology mediated end-joining (MMEJ). The level of NHEJ, HDR and MMEJ can be measured by sequencing.

The ratio of NHEJ to HDR is decreased in some embodiments. In some embodiments, the level of NHEJ in the cell exposed to the engineered Cas nuclease is decreased by at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, or about 50% compared to a cell exposed to a reference Cas nuclease lacking the same mutations in the DNA binding cleft.

In some embodiments, the level of HDR in the cell exposed to the engineered Cas nuclease is increased by at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, or about 50% compared to a cell exposed to a reference Cas nuclease lacking the same mutations in the DNA binding cleft.

In some embodiments, the homology-driven repair pathway is microhomology mediated end-joining (MMEJ). The level of NHEJ and the level of MMEJ are measured by sequencing. In some embodiments, the ratio of NHEJ to MMEJ is decreased. In some embodiments, the level of MMEJ in the cell exposed to the engineered Cas nuclease is increased by at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, or about 50% compared to a cell exposed to a reference Cas nuclease lacking the same mutations in the DNA binding cleft. The genome can be in a non-dividing cell, such as a quiescent cell, a senescent cell, or a fully differentiated cell, or a dividing cell. In another embodiment, the method comprises precisely editing the genome of a non-dividing cell by administering to the cell an agent capable of inhibiting or preventing non-homologous end joining (NHEJ) and increasing homology-driven repair. In another embodiment, the method includes switching a cell from a predominantly non-homologous DNA repair pathway to a homology-driven DNA repair pathway by exposing the cell to a modified or engineered Cas nuclease.

In any of the methods described herein, the engineered Cas nuclease can contain any or all of the modifications and/or sequences described above.

The Cas protein of the disclosure can be a fusion protein. Fusion proteins may include fusions with heterologous domains or functional domains (e.g., localization signals, catalytic domains, etc.). In certain embodiments, various modifications may be combined (e.g., a mutated nuclease which is catalytically inactive, and which further is fused to a functional domain. As used herein, “altered functionality” includes without limitation an altered specificity (e.g., altered target recognition, increased (e.g., “enhanced” Cas proteins) or decreased specificity, or altered PAM recognition), altered activity (e.g., increased or decreased catalytic activity, including catalytically inactive), and/or altered stability (e.g., fusions with destabilization domains). Suitable heterologous domains include without limitation a nuclease, a ligase, a repair protein, a methyltransferase, (viral) integrase, a recombinase, a transposase, an argonaute, a cytidine deaminase, a retron, a group II intron, a phosphatase, a phosphorylase, a sulfurylase, a kinase, a polymerase, an exonuclease, etc. Examples of all these modifications are known in the art. It will be understood that a “modified” nuclease as referred to herein, and in particular a “modified” Cas or “modified” Cas system or complex has the capacity to interact with or bind to the polynucleic acid (e.g., in complex with the guide molecule).

### Delivery

In some embodiments, the nuclease is introduced into a cell as a nucleic acid encoding each protein. The nucleic acid introduced into the eukaryotic cell is a plasmid DNA or viral vector. In some embodiments, the target specific nuclease and blunting enzyme are introduced into a cell via a ribonucleoprotein (RNP).

Delivery is in the form of a vector which may be a viral vector, such as a lenti- or baculo- or adeno-viral/adeno-associated viral vectors, but other means of delivery are known (such as yeast systems, microvesicles, gene guns/means of attaching vectors to gold nanoparticles) and are provided. The viral vector may be selected from a variety of families/genera of viruses, including, but not limited to Myoviridae, Siphoviridae, Podoviridae, Corticoviridae, Lipothrixviridae, Poxviridae, Iridoviridae, Adenoviridae, Polyomaviridae, Papillomaviridae, Mimiviridae, Pandoravirusa, Salterprovirusa, Inoviridae, Microviridae, Parvoviridae, Circoviridae, Hepadnaviridae, Caulimoviridae, Retroviridae, Cystoviridae, Reoviridae, Birnaviridae, Totiviridae, Partitiviridae, Filoviridae, Orthomyxoviridae, Deltavirusa, Leviviridae, Picornaviridae, Marnaviridae, Secoviridae, Potyviridae, Caliciviridae, Hepeviridae, Astroviridae, Nodaviridae, Tetraviridae, Luteoviridae, Tombusviridae, Coronaviridae, Arteriviridae, Flaviviridae, Togaviridae, Virgaviridae, Bromoviridae, Tymoviridae, Alphaflexiviridae, Sobemovirusa, or Idaeovirusa.

A vector may mean not only a viral or yeast system (for instance, where the nucleic acids of interest may be operably linked to and under the control of (in terms of expression, such as to ultimately provide a processed RNA) a promoter), but also direct delivery of nucleic acids into a host cell. For example, baculoviruses may be used for expression in insect cells. These insect cells may, in turn be useful for producing large quantities of further vectors, such as AAV or lentivirus adapted for delivery of the present engineered nuclease. Also envisaged is a method of delivering the target specific nuclease and blunting enzyme comprising delivering to a cell mRNAs encoding each.

In some embodiments, expression of a nucleic acid sequence encoding the nuclease may be driven by a promoter. In some embodiments, the nuclease is a Cas. In some embodiments, the nuclease is a Cas9. In some embodiments, the nuclease is a mutant or variant Cas9. In some embodiments, a single promoter drives expression of a nucleic acid sequence encoding a Cas. In some embodiments, the Cas is operably linked to and expressed from the same promoter. In some embodiments, the CRISPR enzyme is expressed from different promoters. For example, the promoter(s) can be, but are not limited to, a UBC promoter, a PGK promoter, an EF1A promoter, a CMV promoter, an EFS promoter, a SV40 promoter, and a TRE promoter. The promoter may be a weak or a strong promoter. The promoter may be a constitutive promoter or an inducible promoter. In some embodiments, the promoter can also be an AAV ITR, and can be advantageous for eliminating the need for an additional promoter element, which can take up space in the vector. The additional space freed up by use of an AAV ITR can be used to drive the expression of additional elements, such as guide sequences. In some embodiments, the promoter may be a tissue specific promoter.

In some embodiments, an enzyme coding sequence encoding a nuclease is codon-optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a Cas protein correspond to the most frequently used codon for a particular amino acid.

In some embodiments, a vector encodes a nuclease comprising one or more nuclear localization sequences (NLSs), such as about or more than about 1, 2, 3, 4, 5, 6, 7, 8, 9, 10, or more NLSs. In some embodiments, the Cas protein comprises about or more than 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Typically, an NLS consists of one or more short sequences of positively charged lysines or arginines exposed on the protein surface, but other types of NLS are known. In some embodiments, the NLS is between two domains, for example between the Cas protein and the viral protein. The NLS may also be between two functional domains separated or flanked by a glycine-serine linker.

In general, the one or more NLSs are of sufficient strength to drive accumulation of the nuclease in a detectable amount in the nucleus of a eukaryotic cell. In general, strength of nuclear localization activity may derive from the number of NLSs in the nuclease, the particular NLS used, or a combination of these factors. Detection of accumulation in the nucleus may be performed by any suitable technique. For example, a detectable marker may be fused to the nuclease, such that location within a cell may be visualized, such as in combination with a means for detecting the location of the nucleus (e.g., a stain specific for the nucleus such as DAPI). Examples of detectable markers include fluorescent proteins (such as green fluorescent proteins, or GFP; RFP; CFP), and epitope tags (HA tag, FLAG tag, SNAP tag). Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In some aspects, the invention provides methods comprising delivering one or more polynucleotides, such as one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a Cas protein optionally in combination with (and optionally complexed) with a guide sequence is delivered to a cell. Conventional viral and non-viral based gene transfer methods can be used to introduce nucleic acids in mammalian cells or target tissues. Such methods can be used to administer nucleic acids encoding a target specific nuclease and/or a blunting enzyme to cells in culture, or in a host organism. Non-viral vector delivery systems include DNA plasmids, RNA (e.g., a transcript of a vector described herein), naked nucleic acid, nucleic acid complexed with a delivery vehicle, such as a liposome, and ribonucleoprotein. Viral vector delivery systems include DNA and RNA viruses, which have either episomal or integrated genomes after delivery to the cell. For a review of gene therapy procedures, see Anderson, Science 256:808-8313 (1992); Navel and Felgner, TIBTECH 11:211-217 (1993); Mitani and Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer and Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994).

The nuclease can be delivered using adeno-associated virus (AAV), lentivirus, adenovirus, or other viral vector types, or combinations thereof. In some embodiments, Cas protein(s) can be packaged into one or more viral vectors. In some embodiments, the targeted trans-splicing system is delivered via AAV as a split intein system, similar to Levy et al. (Nature Biomedical Engineering, 2020, DOI: https://doi.org/10.1038/s41551-019-0501-5). In other embodiments, the target specific nuclease and/or the blunting enzyme can be delivered via AAV as a trans-splicing system, similar to Lai et al. (Nature Biotechnology, 2005, DOI: 10.1038/nbt1153). In some embodiments, the viral vector is delivered to the tissue of interest by, for example, an intramuscular injection, while other times the viral delivery is via intravenous, transdermal, intranasal, oral, mucosal, intrathecal, intracranial or other delivery methods. Such delivery may be either via a single dose, or multiple doses. One skilled in the art understands that the actual dosage to be delivered herein may vary greatly depending upon a variety of factors, such as the vector chosen, the target cell, organism, or tissue, the general condition of the subject to be treated, the degree of transformation/modification sought, the administration route, the administration mode, the type of transformation/modification sought, etc.

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In certain embodiments, delivery of the nuclease and to a cell is non-viral. In certain embodiments, the non-viral delivery system is selected from a ribonucleoprotein, cationic lipid vehicle, electroporation, nucleofection, calcium phosphate transfection, transfection through membrane disruption using mechanical shear forces, mechanical transfection, and nanoparticle delivery.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences.

### Kit

The present disclosure provides kits for carrying out a method. The present disclosure provides kits containing any one or more of the elements disclosed in the above methods and compositions. In some embodiments, the kit comprises a vector system and instructions for using the kit. In some embodiments, the kit comprises a vector system comprising regulatory elements and polynucleotides encoding the target specific nuclease and/or the blunting enzyme. In some embodiments, the kit comprises a viral delivery system of the target specific nuclease and/or the blunting enzyme. In some embodiments, the kit comprises a non-viral delivery system of the target specific nuclease and/or the blunting enzyme. Elements may be provided individually or in combinations, and may be provided in any suitable container, such as a vial, a bottle, or a tube. In some embodiments, the kit includes instruction in one or more languages, for examples, in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element.

### Sequences

TABLE 1
Cas and Cas variant sequences

| Name | Sequence | |
|---|---|---|
| Modified | ATGGACAAGA AGTACAGCAT CGGCCTGGAC ATCGGCACCA ACTCTGTGGG CTGGGCCGTG | 60 |
| Nucleic acid | ATCACCGACG AGTACAAGGT GCCCAGCAAG AAATTCAAGG TGCTGGGCAA CACCGACCGG | 120 |
| sequence of | CACAGCATCA AGAAGAACCT GATCGGAGCC CTGCTGTTCG ACAGCGGCGA AACAGCCGAG | 180 |
| Cas9 from S. | GCCACCCGGC TGAAGAGAAC CGCCAGAAGA AGATACACCA GACGGAAGAA CCGGATCTGC | 240 |
| pyogenes | TATCTGCAAG AGATCTTCAG CAACGAGATG GCCAAGGTGG ACGACAGCTT CTTCCACAGA | 300 |
| SEQ ID NO: | CTGGAAGAGT CCTTCCTGGT GGAAGAGGAT AAGAAGCACG AGCGGCACCC CATCTTCGGC | 360 |
| 1 | AACATCGTGG ACGAGGTGGC CTACCACGAG AAGTACCCCA CCATCTACCA CCTGAGAAAG | 420 |
| | AAACTGGTGG ACAGCACCGA CAAGGCCGAC CTGCGGCTGA TCTATCTGGC CCTGGCCCAC | 480 |
| | ATGATCAAGT TCCGGGGCCA CTTCCTGATC GAGGGCGACC TGAACCCCGA CAACAGCGAC | 540 |
| | GTGGACAAGC TGTTCATCCA GCTGGTGCAG ACCTACAACC AGCTGTTCGA GGAAAACCCC | 600 |
| | ATCAACGCCA GCGGCGTGGA CGCCAAGGCC ATCCTGTCTG CCAGACTGAG CAAGAGCAGA | 660 |
| | CGGCTGGAAA ATCTGATCGC CCAGCTGCCC GGCGAGAAGA AGAATGGCCT GTTCGGAAAC | 720 |
| | CTGATTGCCC TGAGCCTGGG CCTGACCCCC AACTTCAAGA GCAACTTCGA CCTGGCCGAG | 780 |
| | GATGCCAAAC TGCAGCTGAG CAAGGACACC TACGACGACG ACCTGGACAA CCTGCTGGCC | 840 |
| | CAGATCGGCG ACCAGTACGC CGACCTGTTT CTGGCCGCCA AGAACCTGTC CGACGCCATC | 900 |
| | CTGCTGAGCG ACATCCTGAG AGTGAACACC GAGATCACCA AGGCCCCCCT GAGCGCCTCT | 960 |
| | ATGATCAAGA GATACGACGA GCACCACCAG GACCTGACCC TGCTGAAAGC TCTCGTGCGG | 1020 |
| | CAGCAGCTGC CTGAGAAGTA CAAAGAGATT TTCTTCGACC AGAGCAAGAA CGGCTACGCC | 1080 |
| | GGCTACATTG ACGGCGGAGC CAGCCAGGAA GAGTTCTACA AGTTCATCAA GCCCATCCTG | 1140 |
| | GAAAAGATGG ACGGCACCGA GGAACTGCTC GTGAAGCTGA ACAGAGAGGA CCTGCTGCGG | 1200 |
| | AAGCAGCGGA CCTTCGACAA CGGCAGCATC CCCCACCAGA TCCACCTGGG AGAGCTGCAC | 1260 |
| | GCCATTCTGC GGCGGCAGGA AGATTTTTAC CCATTCCTGA AGGACAACCG GGAAAAGATC | 1320 |
| | GAGAAGATCC TGACCTTCCG CATCCCCTAC TACGTGGGCC CTCTGGCCAG GGGAAACAGC | 1380 |
| | AGATTCGCCT GGATGACCAG AAAGAGCGAG GAAACCATCA CCCCCTGGAA CTTCGAGGAA | 1440 |
| | GTGGTGGACA AGGGCGCTTC CGCCCAGAGC TTCATCGAGC GGATGACCAA CTTCGATAAG | 1500 |
| | AACCTGCCCA ACGAGAAGGT GCTGCCCAAG CACAGCCTGC TGTACGAGTA CTTCACCGTG | 1560 |
| | TATAACGAGC TGACCAAAGT GAAATACGTG ACCGAGGGAA TGAGAAAGCC CGCCTTCCTG | 1620 |
| | AGCGGCGAGC AGAAAAAGGC CATCGTGGAC CTGCTGTTCA AGACCAACCG GAAAGTGACC | 1680 |
| | GTGAAGCAGC TGAAAGAGGA CTACTTCAAG AAAATCGAGT GCTTCGACTC CGTGGAAATC | 1740 |
| | TCCGGCGTGG AAGATCGGTT CAACGCCTCC CTGGGCACAT ACCACGATCT GCTGAAAATT | 1800 |
| | ATCAAGGACA AGGACTTCCT GGACAATGAG GAAAACGAGG ACATTCTGGA AGATATCGTG | 1860 |
| | CTGACCCTGA CACTGTTTGA GGACAGAGAG ATGATCGAGG AACGGCTGAA AACCTATGCC | 1920 |
| | CACCTGTTCG ACGACAAAGT GATGAAGCAG CTGAAGCGGC GGAGATACAC CGGCTGGGGC | 1980 |
| | AGGCTGAGCC GGAAGCTGAT CAACGGCATC CGGGACAAGC AGTCCGGCAA GACAATCCTG | 2040 |
| | GATTTCCTGA AGTCCGACGG CTTCGCCAAC AGAAACTTCA TGCAGCTGAT CCACGACGAC | 2100 |
| | AGCCTGACCT TTAAAGAGGA CATCCAGAAA GCCCAGGTGT CCGGCCAGGG CGATAGCCTG | 2160 |
| | CACGAGCACA TTGCCAATCT GGCCGGCAGC CCCGCCATTA AGAAGGGCAT CCTGCAGACA | 2220 |
| | GTGAAGGTGG TGGACGAGCT CGTGAAAGTG ATGGGCCGGC ACAAGCCCGA GAACATCGTG | 2280 |
| | ATCGAAATGG CCAGAGAGAA CCAGACCACC CAGAAGGGAC AGAAGAACAG CCGCGAGAGA | 2340 |
| | ATGAAGCGGA TCGAAGAGGG CATCAAAGAG CTGGGCAGCC AGATCCTGAA AGAACACCCC | 2400 |
| | GTGGAAAACA CCCAGCTGCA GAACGAGAAG CTGTACCTGT ACTACCTGCA GAATGGGCGG | 2460 |
| | GATATGTACG TGGACCAGGA ACTGGACATC AACCGGCTGT CCGACTACGA TGTGGACCAT | 2520 |
| | ATCGTGCCTC AGAGCTTTCT GAAGGACGAC TCCATCGACA ACAAGGTGCT GACCAGAAGC | 2580 |
| | GACAAGAACC GGGGCAAGAG CGACAACGTG CCCTCCGAAG AGGTCGTGAA GAAGATGAAG | 2640 |
| | AACTACTGGC GGCAGCTGCT GAACGCCAAG CTGATTACCC AGAGAAAGTT CGACAATCTG | 2700 |
| | ACCAAGGCCG AGAGAGGCGG CCTGAGCGAA CTGGATAAGG CCGGCTTCAT CAAGAGACAG | 2760 |
| | CTGGTGGAAA CCCGGCAGAT CACAAAGCAC GTGGCACAGA TCCTGGACTC CCGGATGAAC | 2820 |
| | ACTAAGTACG ACGAGAATGA CAAGCTGATC CGGGAAGTGA AAGTGATCAC CCTGAAGTCC | 2880 |
| | AAGCTGGTGT CCGATTTCCG GAAGGATTTC CAGTTTTACA AAGTGCGCGA GATCAACAAC | 2940 |
| | TACCACCACG CCCACGACGC CTACCTGAAC GCCGTCGTGG GAACCGCCCT GATCAAAAAG | 3000 |
| | TACCCTAAGC TGGAAAGCGA GTTCGTGTAC GGCGACTACA AGGTGTACGA CGTGCGGAAG | 3060 |
| | ATGATCGCCA AGAGCGAGCA GGAAATCGGC AAGGCTACCG CCAAGTACTT CTTCTACAGC | 3120 |
| | AACATCATGA ACTTTTTCAA GACCGAGATT ACCCTGGCCA ACGGCGAGAT CCGGAAGCGG | 3180 |
| | CCTCTGATCG AGACAAACGG CGAAACCGGG GAGATCGTGT GGGATAAGGG CCGGGATTTT | 3240 |
| | GCCACCGTGC GGAAAGTGCT GAGCATGCCC CAAGTGAATA TCGTGAAAAA GACCGAGGTG | 3300 |
| | CAGACAGGCG GCTTCAGCAA AGAGTCTATC CTGCCCAAGA GGAACAGCGA TAAGCTGATC | 3360 |
| | GCCAGAAAGA AGGACTGGGA CCCTAAGAAG TACGGCGGCT TCGACAGCCC CACCGTGGCC | 3420 |
| | TATTCTGTGC TGGTGGTGGC CAAAGTGGAA AAGGGCAAGT CCAAGAAACT GAAGAGTGTG | 3480 |
| | AAAGAGCTGC TGGGGATCAC CATCATGGAA AGAAGCAGCT TCGAGAAGAA TCCCATCGAC | 3540 |
| | TTTCTGGAAG CCAAGGGCTA CAAAGAAGTG AAAAAGGACC TGATCATCAA GCTGCCTAAG | 3600 |
| | TACTCCCTGT TCGAGCTGGA AAACGGCCGG AAGAGAATGC TGGCCTCTGC CGGCGAACTG | 3660 |
| | CAGAAGGGAA ACGAACTGGC CCTGCCCTCC AAATATGTGA ACTTCCTGTA CCTGGCCAGC | 3720 |
| | CACTATGAGA AGCTGAAGGG CTCCCCCGAG GATAATGAGC AGAAACAGCT GTTTGTGGAA | 3780 |
| | CAGCACAAGC ACTACCTGGA CGAGATCATC GAGCAGATCA GCGAGTTCTC CAAGAGAGTG | 3840 |
| | ATCCTGGCCG ACGCTAATCT GGACAAAGTG CTGTCCGCCT ACAACAAGCA CCGGGATAAG | 3900 |
| | CCCATCAGAG AGCAGGCCGA GAATATCATC CACCTGTTTA CCCTGACCAA TCTGGGAGCC | 3960 |
| | CCTGCCGCCT TCAAGTACTT TGACACCACC ATCGACCGGA AGAGGTACAC CAGCACCAAA | 4020 |
| | GAGGTGCTGG ACGCCACCCT GATCCACCAG AGCATCACCG GCCTGTACGA GACACGGATC | 4080 |
| | GACCTGTCTC AGCTGGGAGG CGACTAA | 4107 |
| Modified | MDKKYSIGLD IGTNSVGWAV ITDEYKVPSK KFKVLGNTDR HSIKKNLIGA LLFDSGETAE | 60 |
| Amino acid | ATRLKRTARR RYTRRKNRIC YLQEIFSNEM AKVDDSFFHR LEESFLVEED KKHERHPIFG | 120 |
| sequence of | NIVDEVAYHE KYPTIYHLRK KLVDSTDKAD LRLIYLALAH MIKFRGHFLI EGDLNPDNSD | 180 |
| Wild-type | VDKLFIQLVQ TYNQLFEENP INASGVDAKA ILSARLSKSR RLENLIAQLP GEKKNGLFGN | 240 |
| Cas9 from S. | LIALSLGLTP NFKSNFDLAE DAKLQLSKDT YDDDLDNLLA QIGDQYADLF LAAKNLSDAI | 300 |
| pyogenes | LLSDILRVNT EITKAPLSAS MIKRYDEHHQ DLTLLKALVR QQLPEKYKEI FFDQSKNGYA | 360 |
| SEQ ID NO: | GYIDGGASQE EFYKFIKPIL EKMDGTEELL VKLNREDLLR KQRTFDNGSI PHQIHLGELH | 420 |
| 2 | AILRRQEDFY PFLKDNREKI EKILTFRIPY YVGPLARGNS RFAWMTRKSE ETITPWNFEE | 480 |
| | VVDKGASAQS FIERMTNFDK NLPNEKVLPK HSLLYEYFTV YNELTKVKYV TEGMRKPAFL | 540 |
| | SGEQKKAIVD LLFKTNRKVT VKQLKEDYFK KIECFDSVEI SGVEDRFNAS LGTYHDLLKI | 600 |
| | IKDKDFLDNE ENEDILEDIV LTLTLFEDRE MIEERLKTYA HLFDDKVMKQ LKRRRYTGWG | 660 |
| | RLSRKLINGI RDKQSGKTIL DFLKSDGFAN RNFMQLIHDD SLTFKEDIQK AQVSGQGDSL | 720 |
| | HEHIANLAGS PAIKKGILQT VKVVDELVKV MGRHKPENIV IEMARENQTT QKGQKNSRER | 780 |
| | MKRIEEGIKE LGSQILKEHP VENTQLQNEK LYLYYLQNGR DMYVDQELDI NRLSDYDVDH | 840 |
| | IVPQSFLKDD SIDNKVLTRS DKNRGKSDNV PSEEVVKKMK NYWRQLLNAK LITQRKFDNL | 900 |
| | TKAERGGLSE LDKAGFIKRQ LVETRQITKH VAQILDSRMN TKYDENDKLI REVKVITLKS | 960 |
| | KLVSDFRKDF QFYKVREINN YHHAHDAYLN AVVGTALIKK YPKLESEFVY GDYKVYDVRK | 1020 |
| | MIAKSEQEIG KATAKYFFYS NIMNFFKTEI TLANGEIRKR PLIETNGETG EIVWDKGRDF | 1080 |
| | ATVRKVLSMP QVNIVKKTEV QTGGFSKESI LPKRNSDKLI ARKKDWDPKK YGGFDSPTVA | 1140 |
| | YSVLVVAKVE KGKSKKLKSV KELLGITIME RSSFEKNPID FLEAKGYKEV KKDLIIKLPK | 1200 |
| | YSLFELENGR KRMLASAGEL QKGNELALPS KYVNFLYLAS HYEKLKGSPE DNEQKQLFVE | 1260 |
| | QHKHYLDEII EQISEFSKRV ILADANLDKV LSAYNKHRDK PIREQAENII HLFTLTNLGA | 1320 |
| | PAAFKYFDTT IDRKRYTSTK EVLDATLIHQ SITGLYETRI DLSQLGGD | |
| vCas9 (SEQ | MDKKYSIGLD IGTNSVGWAV ITDEYKVPSK KFKVLGNTDR HSIKKNLIGA LLFDRGETAE | 60 |
| ID NO: 3) | ATRLKRTARR RYTRRKNRIC YLQEIFSNEM AKVDDSFFHR LEESFLVEED KKHERHPIFG | 120 |
| | NIVDEVAYHE KYPTIYHLRK KLVDSTDKAD LRLIYLALAH MIKFRGHFLI EGDLNPDNSD | 180 |
| | VDKLFIQLVQ TYNQLFEENP INASGVDAKA ILSARLSKSR RLENLIAQLP GEKKNGLFGN | 240 |
| | LIALSLGLTP NFKSNFDLAE DAKLQLSKDT YDDDLDNLLA QIGDQYADLF LAAKNLSDAI | 300 |
| | LLSDILRVNT EITKAPLSAS MIKRYDEHHQ DLTLLKALVR QQLPEKYKEI FFDQSKNGYA | 360 |
| | GYIDGGASQE EFYKFIKPIL EKMDGTEELL VKLNREDLLR KQRTFDNGSI PHQIHLGELH | 420 |
| | AILRRQEDFY PFLKDNREKI EKILTFRIPY YVGPLARGNS RFAWMTRKSE ETITPWNFEE | 480 |
| | VVDKGASAQS FIERMTNFDK NLPNEKVLPK HSLLYEYFTV YNELTKVKYV TEGMRKPAFL | 540 |
| | SGEQKKAIVD LLFKTNRKVT VKQLKEDYFK KIECFDSVEI SGVEDRFNAS LGTYHDLLKI | 600 |
| | IKDKDFLDNE ENEDILEDIV LTLTLFEDRE MIEERLKTYA HLFDDKVMKQ LKRRRYTGWG | 660 |
| | RLSRKLINGI RDKQSGKTIL DFLKSDGFAN RNFMQLIHDD SLTFKEDIQK AQVSGQGDSL | 720 |
| | HEHIANLAGS PAIKKGILQT VKVVDELVKV MGRHKPENIV IEMARENQTT QKGQKNSRER | 780 |
| | MKRIEEGIKE LGSQILKEHP VENTQLQNEK LYLYYLQNGR DMYVDQELDI NRLSDYDVDH | 840 |
| | IVPQSFLKDD SIDNKVLTRS DKNRGKSDNV PSEEVVKKMK NYWRQLLNAK LITQRKFDNL | 900 |
| | TKAERGGLSE LDKAGFIKRQ LVETRQITKH VAQILDSRMN TKYDENDKLI REVKVITLKS | 960 |
| | KLVSDFRKDF QFYKVAEINN YHHAHDAYLN AVVGTALIKK YPALESEFVY GDYKVYDVRK | 1020 |
| | MIAKSEQEIG KATAKYFFYS NIMNFFKTEI TLANGEIRKR PLIETNGETG EIVWDKGRDF | 1080 |
| | ATVRKVLSMP QVNIVKKTEV QTGGFSKESI LPKRNSDKLI ARKKDWDPKK YGGFDSPTVA | 1140 |
| | YSVLVVAKVE KGKSKKLKSV KELLGITIME RSSFEKNPID FLEAKGYKEV KKDLIIKLPK | 1200 |
| | YSLFELENGR KRMLASAGEL QKGNELALPS KYVNFLYLAS HYEKLKGSPE DNEQKQLFVE | 1260 |
| | QHKHYLDEII EQISEFSKRV ILADANLDKV LSAYNKHRDK PIREQAENII HLFRLTNLGA | 1320 |
| | PAAFKYFDTT IDRKRYTSTK EVLDATLIHQ SITGLYETRI DLSQLGGD | 1368 |
| R976A | MDKKYSIGLD IGTNSVGWAV ITDEYKVPSK KFKVLGNTDR HSIKKNLIGA LLFDSGETAE | 60 |
| K1003A | ATRLKRTARR RYTRRKNRIC YLQEIFSNEM AKVDDSFFHR LEESFLVEED KKHERHPIFG | 120 |
| SEQ ID NO: | NIVDEVAYHE KYPTIYHLRK KLVDSTDKAD LRLIYLALAH MIKFRGHFLI EGDLNPDNSD | 180 |
| 4 | VDKLFIQLVQ TYNQLFEENP INASGVDAKA ILSARLSKSR RLENLIAQLP GEKKNGLFGN | 240 |
| | LIALSLGLTP NFKSNFDLAE DAKLQLSKDT YDDDLDNLLA QIGDQYADLF LAAKNLSDAI | 300 |
| | LLSDILRVNT EITKAPLSAS MIKRYDEHHQ DLTLLKALVR QQLPEKYKEI FFDQSKNGYA | 360 |
| | GYIDGGASQE EFYKFIKPIL EKMDGTEELL VKLNREDLLR KQRTFDNGSI PHQIHLGELH | 420 |
| | AILRRQEDFY PFLKDNREKI EKILTFRIPY YVGPLARGNS RFAWMTRKSE ETITPWNFEE | 480 |
| | VVDKGASAQS FIERMTNFDK NLPNEKVLPK HSLLYEYFTV YNELTKVKYV TEGMRKPAFL | 540 |
| | SGEQKKAIVD LLFKTNRKVT VKQLKEDYFK KIECFDSVEI SGVEDRFNAS LGTYHDLLKI | 600 |
| | IKDKDFLDNE ENEDILEDIV LTLTLFEDRE MIEERLKTYA HLFDDKVMKQ LKRRRYTGWG | 660 |
| | RLSRKLINGI RDKQSGKTIL DFLKSDGFAN RNFMQLIHDD SLTFKEDIQK AQVSGQGDSL | 720 |
| | HEHIANLAGS PAIKKGILQT VKVVDELVKV MGRHKPENIV IEMARENQTT QKGQKNSRER | 780 |
| | MKRIEEGIKE LGSQILKEHP VENTQLQNEK LYLYYLQNGR DMYVDQELDI NRLSDYDVDH | 840 |
| | IVPQSFLKDD SIDNKVLTRS DKNRGKSDNV PSEEVVKKMK NYWRQLLNAK LITQRKFDNL | 900 |
| | TKAERGGLSE LDKAGFIKRQ LVETRQITKH VAQILDSRMN TKYDENDKLI REVKVITLKS | 960 |
| | KLVSDFRKDF QFYKVAEINN YHHAHDAYLN AVVGTALIKK YPALESEFVY GDYKVYDVRK | 1020 |
| | MIAKSEQEIG KATAKYFFYS NIMNFFKTEI TLANGEIRKR PLIETNGETG EIVWDKGRDF | 1080 |
| | ATVRKVLSMP QVNIVKKTEV QTGGFSKESI LPKRNSDKLI ARKKDWDPKK YGGFDSPTVA | 1140 |
| | YSVLVVAKVE KGKSKKLKSV KELLGITIME RSSFEKNPID FLEAKGYKEV KKDLIIKLPK | 1200 |
| | YSLFELENGR KRMLASAGEL QKGNELALPS KYVNFLYLAS HYEKLKGSPE DNEQKQLFVE | 1260 |
| | QHKHYLDEII EQISEFSKRV ILADANLDKV LSAYNKHRDK PIREQAENII HLFTLTNLGA | 1320 |
| | PAAFKYFDTT IDRKRYTSTK EVLDATLIHQ SITGLYETRI DLSQLGGD | 1368 |
| D54R | MDKKYSIGLD IGTNSVGWAV ITDEYKVPSK KFKVLGNTDR HSIKKNLIGA LLFRSGETAE | 60 |
| R976A | ATRLKRTARR RYTRRKNRIC YLQEIFSNEM AKVDDSFFHR LEESFLVEED KKHERHPIFG | 120 |
| K1003A | NIVDEVAYHE KYPTIYHLRK KLVDSTDKAD LRLIYLALAH MIKFRGHFLI EGDLNPDNSD | 180 |
| SEQ ID NO: | VDKLFIQLVQ TYNQLFEENP INASGVDAKA ILSARLSKSR RLENLIAQLP GEKKNGLFGN | 240 |
| 5 | LIALSLGLTP NFKSNFDLAE DAKLQLSKDT YDDDLDNLLA QIGDQYADLF LAAKNLSDAI | 300 |
| | LLSDILRVNT EITKAPLSAS MIKRYDEHHQ DLTLLKALVR QQLPEKYKEI FFDQSKNGYA | 360 |
| | GYIDGGASQE EFYKFIKPIL EKMDGTEELL VKLNREDLLR KQRTFDNGSI PHQIHLGELH | 420 |
| | AILRRQEDFY PFLKDNREKI EKILTFRIPY YVGPLARGNS RFAWMTRKSE ETITPWNFEE | 480 |
| | VVDKGASAQS FIERMTNFDK NLPNEKVLPK HSLLYEYFTV YNELTKVKYV TEGMRKPAFL | 540 |
| | SGEQKKAIVD LLFKTNRKVT VKQLKEDYFK KIECFDSVEI SGVEDRFNAS LGTYHDLLKI | 600 |
| | IKDKDFLDNE ENEDILEDIV LTLTLFEDRE MIEERLKTYA HLFDDKVMKQ LKRRRYTGWG | 660 |
| | RLSRKLINGI RDKQSGKTIL DFLKSDGFAN RNFMQLIHDD SLTFKEDIQK AQVSGQGDSL | 720 |
| | HEHIANLAGS PAIKKGILQT VKVVDELVKV MGRHKPENIV IEMARENQTT QKGQKNSRER | 780 |
| | MKRIEEGIKE LGSQILKEHP VENTQLQNEK LYLYYLQNGR DMYVDQELDI NRLSDYDVDH | 840 |
| | IVPQSFLKDD SIDNKVLTRS DKNRGKSDNV PSEEVVKKMK NYWRQLLNAK LITQRKFDNL | 900 |
| | TKAERGGLSE LDKAGFIKRQ LVETRQITKH VAQILDSRMN TKYDENDKLI REVKVITLKS | 960 |
| | KLVSDFRKDF QFYKVAEINN YHHAHDAYLN AVVGTALIKK YPALESEFVY GDYKVYDVRK | 1020 |
| | MIAKSEQEIG KATAKYFFYS NIMNFFKTEI TLANGEIRKR PLIETNGETG EIVWDKGRDF | 1080 |
| | ATVRKVLSMP QVNIVKKTEV QTGGFSKESI LPKRNSDKLI ARKKDWDPKK YGGFDSPTVA | 1140 |
| | YSVLVVAKVE KGKSKKLKSV KELLGITIME RSSFEKNPID FLEAKGYKEV KKDLIIKLPK | 1200 |
| | YSLFELENGR KRMLASAGEL QKGNELALPS KYVNFLYLAS HYEKLKGSPE DNEQKQLFVE | 1260 |
| | QHKHYLDEII EQISEFSKRV ILADANLDKV LSAYNKHRDK PIREQAENII HLFTLTNLGA | 1320 |
| | PAAFKYFDTT IDRKRYTSTK EVLDATLIHQ SITGLYETRI DLSQLGGD | 1368 |
| S55R | MDKKYSIGLD IGTNSVGWAV ITDEYKVPSK KFKVLGNTDR HSIKKNLIGA LLFDRGETAE | 60 |
| R976A | ATRLKRTARR RYTRRKNRIC YLQEIFSNEM AKVDDSFFHR LEESFLVEED KKHERHPIFG | 120 |
| K1003A | NIVDEVAYHE KYPTIYHLRK KLVDSTDKAD LRLIYLALAH MIKFRGHFLI EGDLNPDNSD | 180 |
| SEQ ID NO: | VDKLFIQLVQ TYNQLFEENP INASGVDAKA ILSARLSKSR RLENLIAQLP GEKKNGLFGN | 240 |
| 6 | LIALSLGLTP NFKSNFDLAE DAKLQLSKDT YDDDLDNLLA QIGDQYADLF LAAKNLSDAI | 300 |
| | LLSDILRVNT EITKAPLSAS MIKRYDEHHQ DLTLLKALVR QQLPEKYKEI FFDQSKNGYA | 360 |
| | GYIDGGASQE EFYKFIKPIL EKMDGTEELL VKLNREDLLR KQRTFDNGSI PHQIHLGELH | 420 |
| | AILRRQEDFY PFLKDNREKI EKILTFRIPY YVGPLARGNS RFAWMTRKSE ETITPWNFEE | 480 |
| | VVDKGASAQS FIERMTNFDK NLPNEKVLPK HSLLYEYFTV YNELTKVKYV TEGMRKPAFL | 540 |
| | SGEQKKAIVD LLFKTNRKVT VKQLKEDYFK KIECFDSVEI SGVEDRFNAS LGTYHDLLKI | 600 |
| | IKDKDFLDNE ENEDILEDIV LTLTLFEDRE MIEERLKTYA HLFDDKVMKQ LKRRRYTGWG | 660 |
| | RLSRKLINGI RDKQSGKTIL DFLKSDGFAN RNFMQLIHDD SLTFKEDIQK AQVSGQGDSL | 720 |
| | HEHIANLAGS PAIKKGILQT VKVVDELVKV MGRHKPENIV IEMARENQTT QKGQKNSRER | 780 |
| | MKRIEEGIKE LGSQILKEHP VENTQLQNEK LYLYYLQNGR DMYVDQELDI NRLSDYDVDH | 840 |
| | IVPQSFLKDD SIDNKVLTRS DKNRGKSDNV PSEEVVKKMK NYWRQLLNAK LITQRKFDNL | 900 |
| | TKAERGGLSE LDKAGFIKRQ LVETRQITKH VAQILDSRMN TKYDENDKLI REVKVITLKS | 960 |
| | KLVSDFRKDF QFYKVAEINN YHHAHDAYLN AVVGTALIKK YPALESEFVY GDYKVYDVRK | 1020 |
| | MIAKSEQEIG KATAKYFFYS NIMNFFKTEI TLANGEIRKR PLIETNGETG EIVWDKGRDF | 1080 |
| | ATVRKVLSMP QVNIVKKTEV QTGGFSKESI LPKRNSDKLI ARKKDWDPKK YGGFDSPTVA | 1140 |
| | YSVLVVAKVE KGKSKKLKSV KELLGITIME RSSFEKNPID FLEAKGYKEV KKDLIIKLPK | 1200 |
| | YSLFELENGR KRMLASAGEL QKGNELALPS KYVNFLYLAS HYEKLKGSPE DNEQKQLFVE | 1260 |
| | QHKHYLDEII EQISEFSKRV ILADANLDKV LSAYNKHRDK PIREQAENII HLFTLTNLGA | 1320 |
| | PAAFKYFDTT IDRKRYTSTK EVLDATLIHQ SITGLYETRI DLSQLGGD | 1368 |
| R976A | MDKKYSIGLD IGTNSVGWAV ITDEYKVPSK KFKVLGNTDR HSIKKNLIGA LLFDSGETAE | 60 |
| N980R | ATRLKRTARR RYTRRKNRIC YLQEIFSNEM AKVDDSFFHR LEESFLVEED KKHERHPIFG | 120 |
| K1003A | NIVDEVAYHE KYPTIYHLRK KLVDSTDKAD LRLIYLALAH MIKFRGHFLI EGDLNPDNSD | 180 |
| SEQ ID NO: | VDKLFIQLVQ TYNQLFEENP INASGVDAKA ILSARLSKSR RLENLIAQLP GEKKNGLFGN | 240 |
| 7 | LIALSLGLTP NFKSNFDLAE DAKLQLSKDT YDDDLDNLLA QIGDQYADLF LAAKNLSDAI | 300 |
| | LLSDILRVNT EITKAPLSAS MIKRYDEHHQ DLTLLKALVR QQLPEKYKEI FFDQSKNGYA | 360 |
| | GYIDGGASQE EFYKFIKPIL EKMDGTEELL VKLNREDLLR KQRTFDNGSI PHQIHLGELH | 420 |
| | AILRRQEDFY PFLKDNREKI EKILTFRIPY YVGPLARGNS RFAWMTRKSE ETITPWNFEE | 480 |
| | VVDKGASAQS FIERMTNFDK NLPNEKVLPK HSLLYEYFTV YNELTKVKYV TEGMRKPAFL | 540 |
| | SGEQKKAIVD LLFKTNRKVT VKQLKEDYFK KIECFDSVEI SGVEDRFNAS LGTYHDLLKI | 600 |
| | IKDKDFLDNE ENEDILEDIV LTLTLFEDRE MIEERLKTYA HLFDDKVMKQ LKRRRYTGWG | 660 |
| | RLSRKLINGI RDKQSGKTIL DFLKSDGFAN RNFMQLIHDD SLTFKEDIQK AQVSGQGDSL | 720 |
| | HEHIANLAGS PAIKKGILQT VKVVDELVKV MGRHKPENIV IEMARENQTT QKGQKNSRER | 780 |
| | MKRIEEGIKE LGSQILKEHP VENTQLQNEK LYLYYLQNGR DMYVDQELDI NRLSDYDVDH | 840 |
| | IVPQSFLKDD SIDNKVLTRS DKNRGKSDNV PSEEVVKKMK NYWRQLLNAK LITQRKFDNL | 900 |
| | TKAERGGLSE LDKAGFIKRQ LVETRQITKH VAQILDSRMN TKYDENDKLI REVKVITLKS | 960 |
| | KLVSDFRKDF QFYKVAEINR YHHAHDAYLN AVVGTALIKK YPALESEFVY GDYKVYDVRK | 1020 |
| | MIAKSEQEIG KATAKYFFYS NIMNFFKTEI TLANGEIRKR PLIETNGETG EIVWDKGRDF | 1080 |
| | ATVRKVLSMP QVNIVKKTEV QTGGFSKESI LPKRNSDKLI ARKKDWDPKK YGGFDSPTVA | 1140 |
| | YSVLVVAKVE KGKSKKLKSV KELLGITIME RSSFEKNPID FLEAKGYKEV KKDLIIKLPK | 1200 |
| | YSLFELENGR KRMLASAGEL QKGNELALPS KYVNFLYLAS HYEKLKGSPE DNEQKQLFVE | 1260 |
| | QHKHYLDEII EQISEFSKRV ILADANLDKV LSAYNKHRDK PIREQAENII HLFTLTNLGA | 1320 |
| | PAAFKYFDTT IDRKRYTSTK EVLDATLIHQ SITGLYETRI DLSQLGGD | 1368 |
| R976A | MDKKYSIGLD IGTNSVGWAV ITDEYKVPSK KFKVLGNTDR HSIKKNLIGA LLFDSGETAE | 60 |
| K1003A | ATRLKRTARR RYTRRKNRIC YLQEIFSNEM AKVDDSFFHR LEESFLVEED KKHERHPIFG | 120 |
| T1314R | NIVDEVAYHE KYPTIYHLRK KLVDSTDKAD LRLIYLALAH MIKFRGHFLI EGDLNPDNSD | 180 |
| SEQ ID NO: | VDKLFIQLVQ TYNQLFEENP INASGVDAKA ILSARLSKSR RLENLIAQLP GEKKNGLFGN | 240 |
| 8 | LIALSLGLTP NFKSNFDLAE DAKLQLSKDT YDDDLDNLLA QIGDQYADLF LAAKNLSDAI | 300 |
| | LLSDILRVNT EITKAPLSAS MIKRYDEHHQ DLTLLKALVR QQLPEKYKEI FFDQSKNGYA | 360 |
| | GYIDGGASQE EFYKFIKPIL EKMDGTEELL VKLNREDLLR KQRTFDNGSI PHQIHLGELH | 420 |
| | AILRRQEDFY PFLKDNREKI EKILTFRIPY YVGPLARGNS RFAWMTRKSE ETITPWNFEE | 480 |
| | VVDKGASAQS FIERMTNFDK NLPNEKVLPK HSLLYEYFTV YNELTKVKYV TEGMRKPAFL | 540 |
| | SGEQKKAIVD LLFKTNRKVT VKQLKEDYFK KIECFDSVEI SGVEDRFNAS LGTYHDLLKI | 600 |
| | IKDKDFLDNE ENEDILEDIV LTLTLFEDRE MIEERLKTYA HLFDDKVMKQ LKRRRYTGWG | 660 |
| | RLSRKLINGI RDKQSGKTIL DFLKSDGFAN RNFMQLIHDD SLTFKEDIQK AQVSGQGDSL | 720 |
| | HEHIANLAGS PAIKKGILQT VKVVDELVKV MGRHKPENIV IEMARENQTT QKGQKNSRER | 780 |
| | MKRIEEGIKE LGSQILKEHP VENTQLQNEK LYLYYLQNGR DMYVDQELDI NRLSDYDVDH | 840 |
| | IVPQSFLKDD SIDNKVLTRS DKNRGKSDNV PSEEVVKKMK NYWRQLLNAK LITQRKFDNL | 900 |
| | TKAERGGLSE LDKAGFIKRQ LVETRQITKH VAQILDSRMN TKYDENDKLI REVKVITLKS | 960 |
| | KLVSDFRKDF QFYKVAEINN YHHAHDAYLN AVVGTALIKK YPALESEFVY GDYKVYDVRK | 1020 |
| | MIAKSEQEIG KATAKYFFYS NIMNFFKTEI TLANGEIRKR PLIETNGETG EIVWDKGRDF | 1080 |
| | ATVRKVLSMP QVNIVKKTEV QTGGFSKESI LPKRNSDKLI ARKKDWDPKK YGGFDSPTVA | 1140 |
| | YSVLVVAKVE KGKSKKLKSV KELLGITIME RSSFEKNPID FLEAKGYKEV KKDLIIKLPK | 1200 |
| | YSLFELENGR KRMLASAGEL QKGNELALPS KYVNFLYLAS HYEKLKGSPE DNEQKQLFVE | 1260 |
| | QHKHYLDEII EQISEFSKRV ILADANLDKV LSAYNKHRDK PIREQAENII HLFRLTNLGA | 1320 |
| | PAAFKYFDTT IDRKRYTSTK EVLDATLIHQ SITGLYETRI DLSQLGGD | 1368 |
| R976A | MDKKYSIGLD IGTNSVGWAV ITDEYKVPSK KFKVLGNTDR HSIKKNLIGA LLFDSGETAE | 60 |
| K1003A | ATRLKRTARR RYTRRKNRIC YLQEIFSNEM AKVDDSFFHR LEESFLVEED KKHERHPIFG | 120 |
| N1317R | NIVDEVAYHE KYPTIYHLRK KLVDSTDKAD LRLIYLALAH MIKFRGHFLI EGDLNPDNSD | 180 |
| SEQ ID NO: | VDKLFIQLVQ TYNQLFEENP INASGVDAKA ILSARLSKSR RLENLIAQLP GEKKNGLFGN | 240 |
| 9 | LIALSLGLTP NFKSNFDLAE DAKLQLSKDT YDDDLDNLLA QIGDQYADLF LAAKNLSDAI | 300 |
| | LLSDILRVNT EITKAPLSAS MIKRYDEHHQ DLTLLKALVR QQLPEKYKEI FFDQSKNGYA | 360 |
| | GYIDGGASQE EFYKFIKPIL EKMDGTEELL VKLNREDLLR KQRTFDNGSI PHQIHLGELH | 420 |
| | AILRRQEDFY PFLKDNREKI EKILTFRIPY YVGPLARGNS RFAWMTRKSE ETITPWNFEE | 480 |
| | VVDKGASAQS FIERMTNFDK NLPNEKVLPK HSLLYEYFTV YNELTKVKYV TEGMRKPAFL | 540 |
| | SGEQKKAIVD LLFKTNRKVT VKQLKEDYFK KIECFDSVEI SGVEDRFNAS LGTYHDLLKI | 600 |
| | IKDKDFLDNE ENEDILEDIV LTLTLFEDRE MIEERLKTYA HLFDDKVMKQ LKRRRYTGWG | 660 |
| | RLSRKLINGI RDKQSGKTIL DFLKSDGFAN RNFMQLIHDD SLTFKEDIQK AQVSGQGDSL | 720 |
| | HEHIANLAGS PAIKKGILQT VKVVDELVKV MGRHKPENIV IEMARENQTT QKGQKNSRER | 780 |
| | MKRIEEGIKE LGSQILKEHP VENTQLQNEK LYLYYLQNGR DMYVDQELDI NRLSDYDVDH | 840 |
| | IVPQSFLKDD SIDNKVLTRS DKNRGKSDNV PSEEVVKKMK NYWRQLLNAK LITQRKFDNL | 900 |
| | TKAERGGLSE LDKAGFIKRQ LVETRQITKH VAQILDSRMN TKYDENDKLI REVKVITLKS | 960 |
| | KLVSDFRKDF QFYKVAEINN YHHAHDAYLN AVVGTALIKK YPALESEFVY GDYKVYDVRK | 1020 |
| | MIAKSEQEIG KATAKYFFYS NIMNFFKTEI TLANGEIRKR PLIETNGETG EIVWDKGRDF | 1080 |
| | ATVRKVLSMP QVNIVKKTEV QTGGFSKESI LPKRNSDKLI ARKKDWDPKK YGGFDSPTVA | 1140 |
| | YSVLVVAKVE KGKSKKLKSV KELLGITIME RSSFEKNPID FLEAKGYKEV KKDLIIKLPK | 1200 |
| | YSLFELENGR KRMLASAGEL QKGNELALPS KYVNFLYLAS HYEKLKGSPE DNEQKQLFVE | 1260 |
| | QHKHYLDEII EQISEFSKRV ILADANLDKV LSAYNKHRDK PIREQAENII HLFTLTRLGA | 1320 |
| | PAAFKYFDTT IDRKRYTSTK EVLDATLIHQ SITGLYETRI DLSQLGGD | 1368 |
| R976A | MDKKYSIGLD IGTNSVGWAV ITDEYKVPSK KFKVLGNTDR HSIKKNLIGA LLFDSGETAE | 60 |
| K1003A | ATRLKRTARR RYTRRKNRIC YLQEIFSNEM AKVDDSFFHR LEESFLVEED KKHERHPIFG | 120 |
| A1322R | NIVDEVAYHE KYPTIYHLRK KLVDSTDKAD LRLIYLALAH MIKFRGHFLI EGDLNPDNSD | 180 |
| SEQ ID NO: | VDKLFIQLVQ TYNQLFEENP INASGVDAKA ILSARLSKSR RLENLIAQLP GEKKNGLFGN | 240 |
| 10 | LIALSLGLTP NFKSNFDLAE DAKLQLSKDT YDDDLDNLLA QIGDQYADLF LAAKNLSDAI | 300 |
| | LLSDILRVNT EITKAPLSAS MIKRYDEHHQ DLTLLKALVR QQLPEKYKEI FFDQSKNGYA | 360 |
| | GYIDGGASQE EFYKFIKPIL EKMDGTEELL VKLNREDLLR KQRTFDNGSI PHQIHLGELH | 420 |
| | AILRRQEDFY PFLKDNREKI EKILTFRIPY YVGPLARGNS RFAWMTRKSE ETITPWNFEE | 480 |
| | VVDKGASAQS FIERMTNFDK NLPNEKVLPK HSLLYEYFTV YNELTKVKYV TEGMRKPAFL | 540 |
| | SGEQKKAIVD LLFKTNRKVT VKQLKEDYFK KIECFDSVEI SGVEDRFNAS LGTYHDLLKI | 600 |
| | IKDKDFLDNE ENEDILEDIV LTLTLFEDRE MIEERLKTYA HLFDDKVMKQ LKRRRYTGWG | 660 |
| | RLSRKLINGI RDKQSGKTIL DFLKSDGFAN RNFMQLIHDD SLTFKEDIQK AQVSGQGDSL | 720 |
| | HEHIANLAGS PAIKKGILQT VKVVDELVKV MGRHKPENIV IEMARENQTT QKGQKNSRER | 780 |
| | MKRIEEGIKE LGSQILKEHP VENTQLQNEK LYLYYLQNGR DMYVDQELDI NRLSDYDVDH | 840 |
| | IVPQSFLKDD SIDNKVLTRS DKNRGKSDNV PSEEVVKKMK NYWRQLLNAK LITQRKFDNL | 900 |
| | TKAERGGLSE LDKAGFIKRQ LVETRQITKH VAQILDSRMN TKYDENDKLI REVKVITLKS | 960 |
| | KLVSDFRKDF QFYKVAEINN YHHAHDAYLN AVVGTALIKK YPALESEFVY GDYKVYDVRK | 1020 |
| | MIAKSEQEIG KATAKYFFYS NIMNFFKTEI TLANGEIRKR PLIETNGETG EIVWDKGRDF | 1080 |
| | ATVRKVLSMP QVNIVKKTEV QTGGFSKESI LPKRNSDKLI ARKKDWDPKK YGGFDSPTVA | 1140 |
| | YSVLVVAKVE KGKSKKLKSV KELLGITIME RSSFEKNPID FLEAKGYKEV KKDLIIKLPK | 1200 |
| | YSLFELENGR KRMLASAGEL QKGNELALPS KYVNFLYLAS HYEKLKGSPE DNEQKQLFVE | 1260 |
| | QHKHYLDEII EQISEFSKRV ILADANLDKV LSAYNKHRDK PIREQAENII HLFTLTNLGA | 1320 |
| | PRAFKYFDTT IDRKRYTSTK EVLDATLIHQ SITGLYETRI DLSQLGGD | 1368 |
| S55R | MDKKYSIGLD IGTNSVGWAV ITDEYKVPSK KFKVLGNTDR HSIKKNLIGA LLFDRGETAE | 60 |
| R976A | ATRLKRTARR RYTRRKNRIC YLQEIFSNEM AKVDDSFFHR LEESFLVEED KKHERHPIFG | 120 |
| N980R | NIVDEVAYHE KYPTIYHLRK KLVDSTDKAD LRLIYLALAH MIKFRGHFLI EGDLNPDNSD | 180 |
| K1003A | VDKLFIQLVQ TYNQLFEENP INASGVDAKA ILSARLSKSR RLENLIAQLP GEKKNGLFGN | 240 |
| SEQ ID NO: | LIALSLGLTP NFKSNFDLAE DAKLQLSKDT YDDDLDNLLA QIGDQYADLF LAAKNLSDAI | 300 |
| 1 | LLSDILRVNT EITKAPLSAS MIKRYDEHHQ DLTLLKALVR QQLPEKYKEI FFDQSKNGYA | 360 |
| | GYIDGGASQE EFYKFIKPIL EKMDGTEELL VKLNREDLLR KQRTFDNGSI PHQIHLGELH | 420 |
| | AILRRQEDFY PFLKDNREKI EKILTFRIPY YVGPLARGNS RFAWMTRKSE ETITPWNFEE | 480 |
| | VVDKGASAQS FIERMTNFDK NLPNEKVLPK HSLLYEYFTV YNELTKVKYV TEGMRKPAFL | 540 |
| | SGEQKKAIVD LLFKTNRKVT VKQLKEDYFK KIECFDSVEI SGVEDRFNAS LGTYHDLLKI | 600 |
| | IKDKDFLDNE ENEDILEDIV LTLTLFEDRE MIEERLKTYA HLFDDKVMKQ LKRRRYTGWG | 660 |
| | RLSRKLINGI RDKQSGKTIL DFLKSDGFAN RNFMQLIHDD SLTFKEDIQK AQVSGQGDSL | 720 |
| | HEHIANLAGS PAIKKGILQT VKVVDELVKV MGRHKPENIV IEMARENQTT QKGQKNSRER | 780 |
| | MKRIEEGIKE LGSQILKEHP VENTQLQNEK LYLYYLQNGR DMYVDQELDI NRLSDYDVDH | 840 |
| | IVPQSFLKDD SIDNKVLTRS DKNRGKSDNV PSEEVVKKMK NYWRQLLNAK LITQRKFDNL | 900 |
| | TKAERGGLSE LDKAGFIKRQ LVETRQITKH VAQILDSRMN TKYDENDKLI REVKVITLKS | 960 |
| | KLVSDFRKDF QFYKVAEINR YHHAHDAYLN AVVGTALIKK YPALESEFVY GDYKVYDVRK | 1020 |
| | MIAKSEQEIG KATAKYFFYS NIMNFFKTEI TLANGEIRKR PLIETNGETG EIVWDKGRDF | 1080 |
| | ATVRKVLSMP QVNIVKKTEV QTGGFSKESI LPKRNSDKLI ARKKDWDPKK YGGFDSPTVA | 1140 |
| | YSVLVVAKVE KGKSKKLKSV KELLGITIME RSSFEKNPID FLEAKGYKEV KKDLIIKLPK | 1200 |
| | YSLFELENGR KRMLASAGEL QKGNELALPS KYVNFLYLAS HYEKLKGSPE DNEQKQLFVE | 1260 |
| | QHKHYLDEII EQISEFSKRV ILADANLDKV LSAYNKHRDK PIREQAENII HLFTLTNLGA | 1320 |
| | PAAFKYFDTT IDRKRYTSTK EVLDATLIHQ SITGLYETRI DLSQLGGD | 1368 |
| R976A | MDKKYSIGLD IGTNSVGWAV ITDEYKVPSK KFKVLGNTDR HSIKKNLIGA LLFDSGETAE | 60 |
| N980R | ATRLKRTARR RYTRRKNRIC YLQEIFSNEM AKVDDSFFHR LEESFLVEED KKHERHPIFG | 120 |
| K1003A | NIVDEVAYHE KYPTIYHLRK KLVDSTDKAD LRLIYLALAH MIKFRGHFLI EGDLNPDNSD | 180 |
| T1314R | VDKLFIQLVQ TYNQLFEENP INASGVDAKA ILSARLSKSR RLENLIAQLP GEKKNGLFGN | 240 |
| SEQ ID NO: | LIALSLGLTP NFKSNFDLAE DAKLQLSKDT YDDDLDNLLA QIGDQYADLF LAAKNLSDAI | 300 |
| 12 | LLSDILRVNT EITKAPLSAS MIKRYDEHHQ DLTLLKALVR QQLPEKYKEI FFDQSKNGYA | 360 |
| | GYIDGGASQE EFYKFIKPIL EKMDGTEELL VKLNREDLLR KQRTFDNGSI PHQIHLGELH | 420 |
| | AILRRQEDFY PFLKDNREKI EKILTFRIPY YVGPLARGNS RFAWMTRKSE ETITPWNFEE | 480 |
| | VVDKGASAQS FIERMTNFDK NLPNEKVLPK HSLLYEYFTV YNELTKVKYV TEGMRKPAFL | 540 |
| | SGEQKKAIVD LLFKTNRKVT VKQLKEDYFK KIECFDSVEI SGVEDRFNAS LGTYHDLLKI | 600 |
| | IKDKDFLDNE ENEDILEDIV LTLTLFEDRE MIEERLKTYA HLFDDKVMKQ LKRRRYTGWG | 660 |
| | RLSRKLINGI RDKQSGKTIL DFLKSDGFAN RNFMQLIHDD SLTFKEDIQK AQVSGQGDSL | 720 |
| | HEHIANLAGS PAIKKGILQT VKVVDELVKV MGRHKPENIV IEMARENQTT QKGQKNSRER | 780 |
| | MKRIEEGIKE LGSQILKEHP VENTQLQNEK LYLYYLQNGR DMYVDQELDI NRLSDYDVDH | 840 |
| | IVPQSFLKDD SIDNKVLTRS DKNRGKSDNV PSEEVVKKMK NYWRQLLNAK LITQRKFDNL | 900 |
| | TKAERGGLSE LDKAGFIKRQ LVETRQITKH VAQILDSRMN TKYDENDKLI REVKVITLKS | 960 |
| | KLVSDFRKDF QFYKVAEINR YHHAHDAYLN AVVGTALIKK YPALESEFVY GDYKVYDVRK | 1020 |
| | MIAKSEQEIG KATAKYFFYS NIMNFFKTEI TLANGEIRKR PLIETNGETG EIVWDKGRDF | 1080 |
| | ATVRKVLSMP QVNIVKKTEV QTGGFSKESI LPKRNSDKLI ARKKDWDPKK YGGFDSPTVA | 1140 |
| | YSVLVVAKVE KGKSKKLKSV KELLGITIME RSSFEKNPID FLEAKGYKEV KKDLIIKLPK | 1200 |
| | YSLFELENGR KRMLASAGEL QKGNELALPS KYVNFLYLAS HYEKLKGSPE DNEQKQLFVE | 1260 |
| | QHKHYLDEII EQISEFSKRV ILADANLDKV LSAYNKHRDK PIREQAENII HLFRLTNLGA | 1320 |
| | PAAFKYFDTT IDRKRYTSTK EVLDATLIHQ SITGLYETRI DLSQLGGD | 1368 |
| S55R | MDKKYSIGLD IGTNSVGWAV ITDEYKVPSK KFKVLGNTDR HSIKKNLIGA LLFDRGETAE | 60 |
| N976A | ATRLKRTARR RYTRRKNRIC YLQEIFSNEM AKVDDSFFHR LEESFLVEED KKHERHPIFG | 120 |
| N980R | NIVDEVAYHE KYPTIYHLRK KLVDSTDKAD LRLIYLALAH MIKFRGHFLI EGDLNPDNSD | 180 |
| K1003A | VDKLFIQLVQ TYNQLFEENP INASGVDAKA ILSARLSKSR RLENLIAQLP GEKKNGLFGN | 240 |
| T1314R | LIALSLGLTP NFKSNFDLAE DAKLQLSKDT YDDDLDNLLA QIGDQYADLF LAAKNLSDAI | 300 |
| SEQ ID NO: | LLSDILRVNT EITKAPLSAS MIKRYDEHHQ DLTLLKALVR QQLPEKYKEI FFDQSKNGYA | 360 |
| 13 | GYIDGGASQE EFYKFIKPIL EKMDGTEELL VKLNREDLLR KQRTFDNGSI PHQIHLGELH | 420 |
| | AILRRQEDFY PFLKDNREKI EKILTFRIPY YVGPLARGNS RFAWMTRKSE ETITPWNFEE | 480 |
| | VVDKGASAQS FIERMTNFDK NLPNEKVLPK HSLLYEYFTV YNELTKVKYV TEGMRKPAFL | 540 |
| | SGEQKKAIVD LLFKTNRKVT VKQLKEDYFK KIECFDSVEI SGVEDRFNAS LGTYHDLLKI | 600 |
| | IKDKDFLDNE ENEDILEDIV LTLTLFEDRE MIEERLKTYA HLFDDKVMKQ LKRRRYTGWG | 660 |
| | RLSRKLINGI RDKQSGKTIL DFLKSDGFAN RNFMQLIHDD SLTFKEDIQK AQVSGQGDSL | 720 |
| | HEHIANLAGS PAIKKGILQT VKVVDELVKV MGRHKPENIV IEMARENQTT QKGQKNSRER | 780 |
| | MKRIEEGIKE LGSQILKEHP VENTQLQNEK LYLYYLQNGR DMYVDQELDI NRLSDYDVDH | 840 |
| | IVPQSFLKDD SIDNKVLTRS DKNRGKSDNV PSEEVVKKMK NYWRQLLNAK LITQRKFDNL | 900 |
| | TKAERGGLSE LDKAGFIKRQ LVETRQITKH VAQILDSRMN TKYDENDKLI REVKVITLKS | 960 |
| | KLVSDFRKDF QFYKVAEINR YHHAHDAYLN AVVGTALIKK YPALESEFVY GDYKVYDVRK | 1020 |
| | MIAKSEQEIG KATAKYFFYS NIMNFFKTEI TLANGEIRKR PLIETNGETG EIVWDKGRDF | 1080 |
| | ATVRKVLSMP QVNIVKKTEV QTGGFSKESI LPKRNSDKLI ARKKDWDPKK YGGFDSPTVA | 1140 |
| | YSVLVVAKVE KGKSKKLKSV KELLGITIME RSSFEKNPID FLEAKGYKEV KKDLIIKLPK | 1200 |
| | YSLFELENGR KRMLASAGEL QKGNELALPS KYVNFLYLAS HYEKLKGSPE DNEQKQLFVE | 1260 |
| | QHKHYLDEII EQISEFSKRV ILADANLDKV LSAYNKHRDK PIREQAENII HLFRLTNLGA | 1320 |
| | PAAFKYFDTT IDRKRYTSTK EVLDATLIHQ SITGLYETRI DLSQLGGD | 1368 |
| Nucleic Acid | ATGGACAAGA AGTACAGCAT CGGCCTGGAC ATCGGCACCA ACTCTGTGGG CTGGGCCGTG | 60 |
| sequence of | ATCACCGACG AGTACAAGGT GCCCAGCAAG AAATTCAAGG TGCTGGGCAA CACCGACCGG | 120 |
| Cas9^{S55R-R976A-K1003A-} | CACAGCATCA AGAAGAACCT GATCGGAGCC CTGCTGTTCG ACCGGGGCGA AACAGCCGAG | 180 |
| ^{T1314R} (vCas9) | GCCACCCGGC TGAAGAGAAC CGCCAGAAGA AGATACACCA GACGGAAGAA CCGGATCTGC | 240 |
| SEQ ID NO: 285 | TATCTGCAAG AGATCTTCAG CAACGAGATG GCCAAGGTGG ACGACAGCTT CTTCCACAGA | 300 |
| | CTGGAAGAGT CCTTCCTGGT GGAAGAGGAT AAGAAGCACG AGCGGCACCC CATCTTCGGC | 360 |
| | AACATCGTGG ACGAGGTGGC CTACCACGAG AAGTACCCCA CCATCTACCA CCTGAGAAAG | 420 |
| | AAACTGGTGG ACAGCACCGA CAAGGCCGAC CTGCGGCTGA TCTATCTGGC CCTGGCCCAC | 480 |
| | ATGATCAAGT TCCGGGGCCA CTTCCTGATC GAGGGCGACC TGAACCCCGA CAACAGCGAC | 540 |
| | GTGGACAAGC TGTTCATCCA GCTGGTGCAG ACCTACAACC AGCTGTTCGA GGAAAACCCC | 600 |
| | ATCAACGCCA GCGGCGTGGA CGCCAAGGCC ATCCTGTCTG CCAGACTGAG CAAGAGCAGA | 660 |
| | CGGCTGGAAA ATCTGATCGC CCAGCTGCCC GGCGAGAAGA AGAATGGCCT GTTCGGAAAC | 720 |
| | CTGATTGCCC TGAGCCTGGG CCTGACCCCC AACTTCAAGA GCAACTTCGA CCTGGCCGAG | 780 |
| | GATGCCAAAC TGCAGCTGAG CAAGGACACC TACGACGACG ACCTGGACAA CCTGCTGGCC | 840 |
| | CAGATCGGCG ACCAGTACGC CGACCTGTTT CTGGCCGCCA AGAACCTGTC CGACGCCATC | 900 |
| | CTGCTGAGCG ACATCCTGAG AGTGAACACC GAGATCACCA AGGCCCCCCT GAGCGCCTCT | 960 |
| | ATGATCAAGA GATACGACGA GCACCACCAG GACCTGACCC TGCTGAAAGC TCTCGTGCGG | 1020 |
| | CAGCAGCTGC CTGAGAAGTA CAAAGAGATT TTCTTCGACC AGAGCAAGAA CGGCTACGCC | 1080 |
| | GGCTACATTG ACGGCGGAGC CAGCCAGGAA GAGTTCTACA AGTTCATCAA GCCCATCCTG | 1140 |
| | GAAAAGATGG ACGGCACCGA GGAACTGCTC GTGAAGCTGA ACAGAGAGGA CCTGCTGCGG | 1200 |
| | AAGCAGCGGA CCTTCGACAA CGGCAGCATC CCCCACCAGA TCCACCTGGG AGAGCTGCAC | 1260 |
| | GCCATTCTGC GGCGGCAGGA AGATTTTTAC CCATTCCTGA AGGACAACCG GGAAAAGATC | 1320 |
| | GAGAAGATCC TGACCTTCCG CATCCCCTAC TACGTGGGCC CTCTGGCCAG GGGAAACAGC | 1380 |
| | AGATTCGCCT GGATGACCAG AAAGAGCGAG GAAACCATCA CCCCCTGGAA CTTCGAGGAA | 1440 |
| | GTGGTGGACA AGGGCGCTTC CGCCCAGAGC TTCATCGAGC GGATGACCAA CTTCGATAAG | 1500 |
| | AACCTGCCCA ACGAGAAGGT GCTGCCCAAG CACAGCCTGC TGTACGAGTA CTTCACCGTG | 1560 |
| | TATAACGAGC TGACCAAAGT GAAATACGTG ACCGAGGGAA TGAGAAAGCC CGCCTTCCTG | 1620 |
| | AGCGGCGAGC AGAAAAAGGC CATCGTGGAC CTGCTGTTCA AGACCAACCG GAAAGTGACC | 1680 |
| | GTGAAGCAGC TGAAAGAGGA CTACTTCAAG AAAATCGAGT GCTTCGACTC CGTGGAAATC | 1740 |
| | TCCGGCGTGG AAGATCGGTT CAACGCCTCC CTGGGCACAT ACCACGATCT GCTGAAAATT | 1800 |
| | ATCAAGGACA AGGACTTCCT GGACAATGAG GAAAACGAGG ACATTCTGGA AGATATCGTG | 1860 |
| | CTGACCCTGA CACTGTTTGA GGACAGAGAG ATGATCGAGG AACGGCTGAA AACCTATGCC | 1920 |
| | CACCTGTTCG ACGACAAAGT GATGAAGCAG CTGAAGCGGC GGAGATACAC CGGCTGGGGC | 1980 |
| | AGGCTGAGCC GGAAGCTGAT CAACGGCATC CGGGACAAGC AGTCCGGCAA GACAATCCTG | 2040 |
| | GATTTCCTGA AGTCCGACGG CTTCGCCAAC AGAAACTTCA TGCAGCTGAT CCACGACGAC | 2100 |
| | AGCCTGACCT TTAAAGAGGA CATCCAGAAA GCCCAGGTGT CCGGCCAGGG CGATAGCCTG | 2160 |
| | CACGAGCACA TTGCCAATCT GGCCGGCAGC CCCGCCATTA AGAAGGGCAT CCTGCAGACA | 2220 |
| | GTGAAGGTGG TGGACGAGCT CGTGAAAGTG ATGGGCCGGC ACAAGCCCGA GAACATCGTG | 2280 |
| | ATCGAAATGG CCAGAGAGAA CCAGACCACC CAGAAGGGAC AGAAGAACAG CCGCGAGAGA | 2340 |
| | ATGAAGCGGA TCGAAGAGGG CATCAAAGAG CTGGGCAGCC AGATCCTGAA AGAACACCCC | 2400 |
| | GTGGAAAACA CCCAGCTGCA GAACGAGAAG CTGTACCTGT ACTACCTGCA GAATGGGCGG | 2460 |
| | GATATGTACG TGGACCAGGA ACTGGACATC AACCGGCTGT CCGACTACGA TGTGGACCAT | 2520 |
| | ATCGTGCCTC AGAGCTTTCT GAAGGACGAC TCCATCGACA ACAAGGTGCT GACCAGAAGC | 2580 |
| | GACAAGAACC GGGGCAAGAG CGACAACGTG CCCTCCGAAG AGGTCGTGAA GAAGATGAAG | 2640 |
| | AACTACTGGC GGCAGCTGCT GAACGCCAAG CTGATTACCC AGAGAAAGTT CGACAATCTG | 2700 |
| | ACCAAGGCCG AGAGAGGCGG CCTGAGCGAA CTGGATAAGG CCGGCTTCAT CAAGAGACAG | 2760 |
| | CTGGTGGAAA CCCGGCAGAT CACAAAGCAC GTGGCACAGA TCCTGGACTC CCGGATGAAC | 2820 |
| | ACTAAGTACG ACGAGAATGA CAAGCTGATC CGGGAAGTGA AAGTGATCAC CCTGAAGTCC | 2880 |
| | AAGCTGGTGT CCGATTTCCG GAAGGATTTC CAGTTTTACA AAGTGGCCGA GATCAACAAC | 2940 |
| | TACCACCACG CCCACGACGC CTACCTGAAC GCCGTCGTGG GAACCGCCCT GATCAAAAAG | 3000 |
| | TACCCTGCCC TGGAAAGCGA GTTCGTGTAC GGCGACTACA AGGTGTACGA CGTGCGGAAG | 3060 |
| | ATGATCGCCA AGAGCGAGCA GGAAATCGGC AAGGCTACCG CCAAGTACTT CTTCTACAGC | 3120 |
| | AACATCATGA ACTTTTTCAA GACCGAGATT ACCCTGGCCA ACGGCGAGAT CCGGAAGCGG | 3180 |
| | CCTCTGATCG AGACAAACGG CGAAACCGGG GAGATCGTGT GGGATAAGGG CCGGGATTTT | 3240 |
| | GCCACCGTGC GGAAAGTGCT GAGCATGCCC CAAGTGAATA TCGTGAAAAA GACCGAGGTG | 3300 |
| | CAGACAGGCG GCTTCAGCAA AGAGTCTATC CTGCCCAAGA GGAACAGCGA TAAGCTGATC | 3360 |
| | GCCAGAAAGA AGGACTGGGA CCCTAAGAAG TACGGCGGCT TCGACAGCCC CACCGTGGCC | 3420 |
| | TATTCTGTGC TGGTGGTGGC CAAAGTGGAA AAGGGCAAGT CCAAGAAACT GAAGAGTGTG | 3480 |
| | AAAGAGCTGC TGGGGATCAC CATCATGGAA AGAAGCAGCT TCGAGAAGAA TCCCATCGAC | 3540 |
| | TTTCTGGAAG CCAAGGGCTA CAAAGAAGTG AAAAAGGACC TGATCATCAA GCTGCCTAAG | 3600 |
| | TACTCCCTGT TCGAGCTGGA AAACGGCCGG AAGAGAATGC TGGCCTCTGC CGGCGAACTG | 3660 |
| | CAGAAGGGAA ACGAACTGGC CCTGCCCTCC AAATATGTGA ACTTCCTGTA CCTGGCCAGC | 3720 |
| | CACTATGAGA AGCTGAAGGG CTCCCCCGAG GATAATGAGC AGAAACAGCT GTTTGTGGAA | 3780 |
| | CAGCACAAGC ACTACCTGGA CGAGATCATC GAGCAGATCA GCGAGTTCTC CAAGAGAGTG | 3840 |
| | ATCCTGGCCG ACGCTAATCT GGACAAAGTG CTGTCCGCCT ACAACAAGCA CCGGGATAAG | 3900 |
| | CCCATCAGAG AGCAGGCCGA GAATATCATC CACCTGTTTC GGCTGACCAA TCTGGGAGCC | 3960 |
| | CCTGCCGCCT TCAAGTACTT TGACACCACC ATCGACCGGA AGAGGTACAC CAGCACCAAA | 4020 |
| | GAGGTGCTGG ACGCCACCCT GATCCACCAG AGCATCACCG GCCTGTACGA GACACGGATC | 4080 |
| | GACCTGTCTC AGCTGGGAGG CGAC | 4104 |
| PE SEQ ID | ATGAAACGGA CAGCCGACGG AAGCGAGTTC GAGTCACCAA AGAAGAAGCG GAAAGTCGAC | 60 |
| NO: 286 | AAGAAGTACA GCATCGGCCT GGACATCGGC ACCAACTCTG TGGGCTGGGC CGTGATCACC | 120 |
| | GACGAGTACA AGGTGCCCAG CAAGAAATTC AAGGTGCTGG GCAACACCGA CCGGCACAGC | 180 |
| | ATCAAGAAGA ACCTGATCGG AGCCCTGCTG TTCGACAGCG GCGAAACAGC CGAGGCCACC | 240 |
| | CGGCTGAAGA GAACCGCCAG AAGAAGATAC ACCAGACGGA AGAACCGGAT CTGCTATCTG | 300 |
| | CAAGAGATCT TCAGCAACGA GATGGCCAAG GTGGACGACA GCTTCTTCCA CAGACTGGAA | 360 |
| | GAGTCCTTCC TGGTGGAAGA GGATAAGAAG CACGAGCGGC ACCCCATCTT CGGCAACATC | 420 |
| | GTGGACGAGG TGGCCTACCA CGAGAAGTAC CCCACCATCT ACCACCTGAG AAAGAAACTG | 480 |
| | GTGGACAGCA CCGACAAGGC CGACCTGCGG CTGATCTATC TGGCCCTGGC CCACATGATC | 540 |
| | AAGTTCCGGG GCCACTTCCT GATCGAGGGC GACCTGAACC CCGACAACAG CGACGTGGAC | 600 |
| | AAGCTGTTCA TCCAGCTGGT GCAGACCTAC AACCAGCTGT TCGAGGAAAA CCCCATCAAC | 660 |
| | GCCAGCGGCG TGGACGCCAA GGCCATCCTG TCTGCCAGAC TGAGCAAGAG CAGACGGCTG | 720 |
| | GAAAATCTGA TCGCCCAGCT GCCCGGCGAG AAGAAGAATG GCCTGTTCGG AAACCTGATT | 780 |
| | GCCCTGAGCC TGGGCCTGAC CCCCAACTTC AAGAGCAACT TCGACCTGGC CGAGGATGCC | 840 |
| | AAACTGCAGC TGAGCAAGGA CACCTACGAC GACGACCTGG ACAACCTGCT GGCCCAGATC | 900 |
| | GGCGACCAGT ACGCCGACCT GITTCTGGCC GCCAAGAACC TGTCCGACGC CATCCTGCTG | 960 |
| | AGCGACATCC TGAGAGTGAA CACCGAGATC ACCAAGGCCC CCCTGAGCGC CTCTATGATC | 1020 |
| | AAGAGATACG ACGAGCACCA CCAGGACCTG ACCCTGCTGA AAGCTCTCGT GCGGCAGCAG | 1080 |
| | CTGCCTGAGA AGTACAAAGA GATTTTCTTC GACCAGAGCA AGAACGGCTA CGCCGGCTAC | 1140 |
| | ATTGACGGCG GAGCCAGCCA GGAAGAGTTC TACAAGTTCA TCAAGCCCAT CCTGGAAAAG | 1200 |
| | ATGGACGGCA CCGAGGAACT GCTCGTGAAG CTGAACAGAG AGGACCTGCT GCGGAAGCAG | 1260 |
| | CGGACCTTCG ACAACGGCAG CATCCCCCAC CAGATCCACC TGGGAGAGCT GCACGCCATT | 1320 |
| | CTGCGGCGGC AGGAAGATTT TTACCCATTC CTGAAGGACA ACCGGGAAAA GATCGAGAAG | 1380 |
| | ATCCTGACCT TCCGCATCCC CTACTACGTG GGCCCTCTGG CCAGGGGAAA CAGCAGATTC | 1440 |
| | GCCTGGATGA CCAGAAAGAG CGAGGAAACC ATCACCCCCT GGAACTTCGA GGAAGTGGTG | 1500 |
| | GACAAGGGCG CTTCCGCCCA GAGCTTCATC GAGCGGATGA CCAACTTCGA TAAGAACCTG | 1560 |
| | CCCAACGAGA AGGTGCTGCC CAAGCACAGC CTGCTGTACG AGTACTTCAC CGTGTATAAC | 1620 |
| | GAGCTGACCA AAGTGAAATA CGTGACCGAG GGAATGAGAA AGCCCGCCTT CCTGAGCGGC | 1680 |
| | GAGCAGAAAA AGGCCATCGT GGACCTGCTG TTCAAGACCA ACCGGAAAGT GACCGTGAAG | 1740 |
| | CAGCTGAAAG AGGACTACTT CAAGAAAATC GAGTGCTTCG ACTCCGTGGA AATCTCCGGC | 1800 |
| | GTGGAAGATC GGTTCAACGC CTCCCTGGGC ACATACCACG ATCTGCTGAA AATTATCAAG | 1860 |
| | GACAAGGACT TCCTGGACAA TGAGGAAAAC GAGGACATTC TGGAAGATAT CGTGCTGACC | 1920 |
| | CTGACACTGT TTGAGGACAG AGAGATGATC GAGGAACGGC TGAAAACCTA TGCCCACCTG | 1980 |
| | TTCGACGACA AAGTGATGAA GCAGCTGAAG CGGCGGAGAT ACACCGGCTG GGGCAGGCTG | 2040 |
| | AGCCGGAAGC TGATCAACGG CATCCGGGAC AAGCAGTCCG GCAAGACAAT CCTGGATTTC | 2100 |
| | CTGAAGTCCG ACGGCTTCGC CAACAGAAAC TTCATGCAGC TGATCCACGA CGACAGCCTG | 2160 |
| | ACCTTTAAAG AGGACATCCA GAAAGCCCAG GTGTCCGGCC AGGGCGATAG CCTGCACGAG | 2220 |
| | CACATTGCCA ATCTGGCCGG CAGCCCCGCC ATTAAGAAGG GCATCCTGCA GACAGTGAAG | 2280 |
| | GTGGTGGACG AGCTCGTGAA AGTGATGGGC CGGCACAAGC CCGAGAACAT CGTGATCGAA | 2340 |
| | ATGGCCAGAG AGAACCAGAC CACCCAGAAG GGACAGAAGA ACAGCCGCGA GAGAATGAAG | 2400 |
| | CGGATCGAAG AGGGCATCAA AGAGCTGGGC AGCCAGATCC TGAAAGAACA CCCCGTGGAA | 2460 |
| | AACACCCAGC TGCAGAACGA GAAGCTGTAC CTGTACTACC TGCAGAATGG GCGGGATATG | 2520 |
| | TACGTGGACC AGGAACTGGA CATCAACCGG CTGTCCGACT ACGATGTGGA CGCTATCGTG | 2580 |
| | CCTCAGAGCT TTCTGAAGGA CGACTCCATC GACAACAAGG TGCTGACCAG AAGCGACAAG | 2640 |
| | AACCGGGGCA AGAGCGACAA CGTGCCCTCC GAAGAGGTCG TGAAGAAGAT GAAGAACTAC | 2700 |
| | TGGCGGCAGC TGCTGAACGC CAAGCTGATT ACCCAGAGAA AGTTCGACAA TCTGACCAAG | 2760 |
| | GCCGAGAGAG GCGGCCTGAG CGAACTGGAT AAGGCCGGCT TCATCAAGAG ACAGCTGGTG | 2820 |
| | GAAACCCGGC AGATCACAAA GCACGTGGCA CAGATCCTGG ACTCCCGGAT GAACACTAAG | 2880 |
| | TACGACGAGA ATGACAAGCT GATCCGGGAA GTGAAAGTGA TCACCCTGAA GTCCAAGCTG | 2940 |
| | GTGTCCGATT TCCGGAAGGA TTTCCAGTTT TACAAAGTGC GCGAGATCAA CAACTACCAC | 3000 |
| | CACGCCCACG ACGCCTACCT GAACGCCGTC GTGGGAACCG CCCTGATCAA AAAGTACCCT | 3060 |
| | AAGCTGGAAA GCGAGTTCGT GTACGGCGAC TACAAGGTGT ACGACGTGCG GAAGATGATC | 3120 |
| | GCCAAGAGCG AGCAGGAAAT CGGCAAGGCT ACCGCCAAGT ACTTCTTCTA CAGCAACATC | 3180 |
| | ATGAACTTTT TCAAGACCGA GATTACCCTG GCCAACGGCG AGATCCGGAA GCGGCCTCTG | 3240 |
| | ATCGAGACAA ACGGCGAAAC CGGGGAGATC GTGTGGGATA AGGGCCGGGA TTTTGCCACC | 3300 |
| | GTGCGGAAAG TGCTGAGCAT GCCCCAAGTG AATATCGTGA AAAAGACCGA GGTGCAGACA | 3360 |
| | GGCGGCTTCA GCAAAGAGTC TATCCTGCCC AAGAGGAACA GCGATAAGCT GATCGCCAGA | 3420 |
| | AAGAAGGACT GGGACCCTAA GAAGTACGGC GGCTTCGACA GCCCCACCGT GGCCTATTCT | 3480 |
| | GTGCTGGTGG TGGCCAAAGT GGAAAAGGGC AAGTCCAAGA AACTGAAGAG TGTGAAAGAG | 3540 |
| | CTGCTGGGGA TCACCATCAT GGAAAGAAGC AGCTTCGAGA AGAATCCCAT CGACTTTCTG | 3600 |
| | GAAGCCAAGG GCTACAAAGA AGTGAAAAAG GACCTGATCA TCAAGCTGCC TAAGTACTCC | 3660 |
| | CTGTTCGAGC TGGAAAACGG CCGGAAGAGA ATGCTGGCCT CTGCCGGCGA ACTGCAGAAG | 3720 |
| | GGAAACGAAC TGGCCCTGCC CTCCAAATAT GTGAACTTCC TGTACCTGGC CAGCCACTAT | 3780 |
| | GAGAAGCTGA AGGGCTCCCC CGAGGATAAT GAGCAGAAAC AGCTGTTTGT GGAACAGCAC | 3840 |
| | AAGCACTACC TGGACGAGAT CATCGAGCAG ATCAGCGAGT TCTCCAAGAG AGTGATCCTG | 3900 |
| | GCCGACGCTA ATCTGGACAA AGTGCTGTCC GCCTACAACA AGCACCGGGA TAAGCCCATC | 3960 |
| | AGAGAGCAGG CCGAGAATAT CATCCACCTG TTTACCCTGA CCAATCTGGG AGCCCCTGCC | 4020 |
| | GCCTTCAAGT ACTTTGACAC CACCATCGAC CGGAAGAGGT ACACCAGCAC CAAAGAGGTG | 4080 |
| | CTGGACGCCA CCCTGATCCA CCAGAGCATC ACCGGCCTGT ACGAGACACG GATCGACCTG | 4140 |
| | TCTCAGCTGG GAGGTGACTC CGGCGGAAGC TCTGGTGGCA GCAAGCGGAC CGCCGACGGC | 4200 |
| | TCTGAATTCG AGAGCCCTAA GAAGAAAAGA AAGGTGAGCG GAGGCTCTAG CGGCGGAAGC | 4260 |
| | ACCCTGAACA TTGAAGACGA GTATAGACTG CATGAAACAA GCAAGGAACC CGACGTGTCC | 4320 |
| | CTGGGCTCCA CCTGGCTGTC CGACTTTCCC CAGGCCTGGG CCGAGACAGG AGGAATGGGC | 4380 |
| | CTGGCCGTGC GGCAGGCACC CCTGATCATC CCTCTGAAGG CCACCTCTAC ACCCGTGAGC | 4440 |
| | ATCAAGCAGT ACCCTATGTC TCAGGAGGCC AGACTGGGCA TCAAGCCTCA CATCCAGAGG | 4500 |
| | CTGCTGGACC AGGGCATCCT GGTGCCATGC CAGAGCCCCT GGAACACACC ACTGCTGCCC | 4560 |
| | GTGAAGAAGC CAGGCACCAA TGACTATAGA CCCGTGCAGG ATCTGAGAGA GGTGAACAAG | 4620 |
| | AGGGTGGAGG ATATCCACCC CACCGTGCCC AACCCTTACA ATCTGCTGTC CGGCCTGCCC | 4680 |
| | CCTTCTCACC AGTGGTATAC AGTGCTGGAC CTGAAGGATG CCTTCTTTTG TCTGAGACTG | 4740 |
| | CACCCTACCA GCCAGCCACT GTTCGCCTTT GAGTGGAGGG ACCCTGAGAT GGGCATCTCT | 4800 |
| | GGCCAGCTGA CCTGGACACG CCTGCCTCAG GGCTTCAAGA ATAGCCCAAC ACTGTTTAAC | 4860 |
| | GAGGCCCTGC ACCGCGACCT GGCAGATTTC CGGATCCAGC ACCCAGATCT GATCCTGCTG | 4920 |
| | CAGTACGTGG ACGATCTGCT GCTGGCCGCC ACCAGCGAGC TGGATTGCCA GCAGGGAACA | 4980 |
| | CGCGCCCTGC TGCAGACCCT GGGAAACCTG GGATATAGGG CATCCGCCAA GAAGGCCCAG | 5040 |
| | ATCTGTCAGA AGCAGGTGAA GTACCTGGGC TATCTGCTGA AGGAGGGCCA GAGATGGCTG | 5100 |
| | ACAGAGGCCA GGAAGGAGAC AGTGATGGGC CAGCCAACAC CCAAGACCCC AAGACAGCTG | 5160 |
| | AGGGAGTTCC TGGGCAAAGC AGGATTTTGC AGGCTGTTCA TCCCAGGATT CGCAGAGATG | 5220 |
| | GCAGCACCTC TGTACCCACT GACCAAGCCG GGCACCCTGT TTAATTGGGG CCCTGACCAG | 5280 |
| | CAGAAGGCCT ATCAGGAGAT CAAGCAGGCC CTGCTGACAG CACCAGCCCT GGGCCTGCCA | 5340 |
| | GACCTGACCA AGCCTTTCGA GCTGTTTGTG GATGAGAAGC AGGGCTACGC CAAGGGCGTG | 5400 |
| | CTGACCCAGA AGCTGGGACC ATGGAGACGG CCCGTGGCCT ATCTGTCCAA GAAGCTGGAC | 5460 |
| | CCAGTGGCAG CAGGATGGCC ACCATGCCTG AGGATGGTGG CAGCAATCGC CGTGCTGACA | 5520 |
| | AAGGATGCCG GCAAGCTGAC CATGGGACAG CCACTGGTCA TCCTGGCACC ACACGCAGTG | 5580 |
| | GAGGCCCTGG TGAAGCAGCC TCCAGATCGC TGGCTGTCTA ACGCCCGGAT GACACACTAC | 5640 |
| | CAGGCCCTGC TGCTGGACAC CGATCGCGTG CAGTTTGGCC CTGTGGTGGC CCTGAATCCA | 5700 |
| | GCCACCCTGC TGCCTCTGCC AGAGGAGGGC CTGCAGCACA ACTGTCTGGA CATCCTGGCA | 5760 |
| | GAGGCACACG GAACAAGGCC AGACCTGACC GATCAGCCCC TGCCTGACGC CGATCACACA | 5820 |
| | TGGTATACCG ATGGAAGCTC CCTGCTGCAG GAGGGCCAGA GGAAGGCAGG AGCAGCAGTG | 5880 |
| | ACCACAGAGA CAGAAGTGAT CTGGGCCAAG GCCCTGCCAG CAGGCACATC CGCCCAGCGG | 5940 |
| | GCCGAGCTGA TCGCCCTGAC CCAGGCCCTG AAGATGGCCG AGGGCAAGAA GCTGAACGTG | 6000 |
| | TACACAGACT CCAGATATGC CTTCGCCACC GCACACATCC ACGGAGAGAT CTACAGGCGC | 6060 |
| | CGGGGCTGGC TGACCTCTGA GGGCAAGGAG ATCAAGAACA AGGATGAGAT CCTGGCCCTG | 6120 |
| | CTGAAGGCCC TGTTTCTGCC CAAGCGGCTG AGCATCATCC ACTGTCCTGG ACACCAGAAG | 6180 |
| | GGACACTCCG CCGAGGCAAG GGGCAATCGG ATGGCCGACC AGGCCGCCAG AAAGGCTGCT | 6240 |
| | ATTACTGAAA CTCCCGACAC TTCCACTCTG CTGATTGAAA ACTCCTCCCC TTCTGGCGGC | 6300 |
| | TCAAAAAGAA CCGCCGACGG CAGCGAATTC GAGTCTCCCA AGAAGAAGAG GAAAGTCGGC | 6360 |
| | TCTGGCCCTG CCGCTAAGAG AGTGAAGCTG GAC | 6393 |
| PF^{K848A-H982A} | ATGAAACGGA CAGCCGACGG AAGCGAGTTC GAGTCACCAA AGAAGAAGCG GAAAGTCGAC | 60 |
| (vPE), | AAGAAGTACA GCATCGGCCT GGACATCGGC ACCAACTCTG TGGGCTGGGC CGTGATCACC | 120 |
| | GACGAGTACA AGGTGCCCAG CAAGAAATTC AAGGTGCTGG GCAACACCGA CCGGCACAGC | 180 |
| | ATCAAGAAGA ACCTGATCGG AGCCCTGCTG TTCGACAGCG GCGAAACAGC CGAGGCCACC | 240 |
| | CGGCTGAAGA GAACCGCCAG AAGAAGATAC ACCAGACGGA AGAACCGGAT CTGCTATCTG | 300 |
| | CAAGAGATCT TCAGCAACGA GATGGCCAAG GTGGACGACA GCTTCTTCCA CAGACTGGAA | 360 |
| | GAGTCCTTCC TGGTGGAAGA GGATAAGAAG CACGAGCGGC ACCCCATCTT CGGCAACATC | 420 |
| | GTGGACGAGG TGGCCTACCA CGAGAAGTAC CCCACCATCT ACCACCTGAG AAAGAAACTG | 480 |
| | GTGGACAGCA CCGACAAGGC CGACCTGCGG CTGATCTATC TGGCCCTGGC CCACATGATC | 540 |
| | AAGTTCCGGG GCCACTTCCT GATCGAGGGC GACCTGAACC CCGACAACAG CGACGTGGAC | 600 |
| | AAGCTGTTCA TCCAGCTGGT GCAGACCTAC AACCAGCTGT TCGAGGAAAA CCCCATCAAC | 660 |
| | GCCAGCGGCG TGGACGCCAA GGCCATCCTG TCTGCCAGAC TGAGCAAGAG CAGACGGCTG | 720 |
| | GAAAATCTGA TCGCCCAGCT GCCCGGCGAG AAGAAGAATG GCCTGTTCGG AAACCTGATT | 780 |
| | GCCCTGAGCC TGGGCCTGAC CCCCAACTTC AAGAGCAACT TCGACCTGGC CGAGGATGCC | 840 |
| | AAACTGCAGC TGAGCAAGGA CACCTACGAC GACGACCTGG ACAACCTGCT GGCCCAGATC | 900 |
| | GGCGACCAGT ACGCCGACCT GTTTCTGGCC GCCAAGAACC TGTCCGACGC CATCCTGCTG | 960 |
| | AGCGACATCC TGAGAGTGAA CACCGAGATC ACCAAGGCCC CCCTGAGCGC CTCTATGATC | 1020 |
| | AAGAGATACG ACGAGCACCA CCAGGACCTG ACCCTGCTGA AAGCTCTCGT GCGGCAGCAG | 1080 |
| | CTGCCTGAGA AGTACAAAGA GATTTTCTTC GACCAGAGCA AGAACGGCTA CGCCGGCTAC | 1140 |
| | ATTGACGGCG GAGCCAGCCA GGAAGAGTTC TACAAGTTCA TCAAGCCCAT CCTGGAAAAG | 1200 |
| | ATGGACGGCA CCGAGGAACT GCTCGTGAAG CTGAACAGAG AGGACCTGCT GCGGAAGCAG | 1260 |
| | CGGACCTTCG ACAACGGCAG CATCCCCCAC CAGATCCACC TGGGAGAGCT GCACGCCATT | 1320 |
| | CTGCGGCGGC AGGAAGATTT TTACCCATTC CTGAAGGACA ACCGGGAAAA GATCGAGAAG | 1380 |
| | ATCCTGACCT TCCGCATCCC CTACTACGTG GGCCCTCTGG CCAGGGGAAA CAGCAGATTC | 1440 |
| | GCCTGGATGA CCAGAAAGAG CGAGGAAACC ATCACCCCCT GGAACTTCGA GGAAGTGGTG | 1500 |
| | GACAAGGGCG CTTCCGCCCA GAGCTTCATC GAGCGGATGA CCAACTTCGA TAAGAACCTG | 1560 |
| | CCCAACGAGA AGGTGCTGCC CAAGCACAGC CTGCTGTACG AGTACTTCAC CGTGTATAAC | 1620 |
| | GAGCTGACCA AAGTGAAATA CGTGACCGAG GGAATGAGAA AGCCCGCCTT CCTGAGCGGC | 1680 |
| | GAGCAGAAAA AGGCCATCGT GGACCTGCTG TTCAAGACCA ACCGGAAAGT GACCGTGAAG | 1740 |
| | CAGCTGAAAG AGGACTACTT CAAGAAAATC GAGTGCTTCG ACTCCGTGGA AATCTCCGGC | 1800 |
| | GTGGAAGATC GGTTCAACGC CTCCCTGGGC ACATACCACG ATCTGCTGAA AATTATCAAG | 1860 |
| | GACAAGGACT TCCTGGACAA TGAGGAAAAC GAGGACATTC TGGAAGATAT CGTGCTGACC | 1920 |
| | CTGACACTGT TTGAGGACAG AGAGATGATC GAGGAACGGC TGAAAACCTA TGCCCACCTG | 1980 |
| | TTCGACGACA AAGTGATGAA GCAGCTGAAG CGGCGGAGAT ACACCGGCTG GGGCAGGCTG | 2040 |
| | AGCCGGAAGC TGATCAACGG CATCCGGGAC AAGCAGTCCG GCAAGACAAT CCTGGATTTC | 2100 |
| | CTGAAGTCCG ACGGCTTCGC CAACAGAAAC TTCATGCAGC TGATCCACGA CGACAGCCTG | 2160 |
| | ACCTTTAAAG AGGACATCCA GAAAGCCCAG GTGTCCGGCC AGGGCGATAG CCTGCACGAG | 2220 |
| | CACATTGCCA ATCTGGCCGG CAGCCCCGCC ATTAAGAAGG GCATCCTGCA GACAGTGAAG | 2280 |
| | GTGGTGGACG AGCTCGTGAA AGTGATGGGC CGGCACAAGC CCGAGAACAT CGTGATCGAA | 2340 |
| | ATGGCCAGAG AGAACCAGAC CACCCAGAAG GGACAGAAGA ACAGCCGCGA GAGAATGAAG | 2400 |
| | CGGATCGAAG AGGGCATCAA AGAGCIGGGC AGCCAGATCC TGAAAGAACA CCCCGTGGAA | 2460 |
| | AACACCCAGC TGCAGAACGA GAAGCTGTAC CTGTACTACC TGCAGAATGG GCGGGATATG | 2520 |
| | TACGTGGACC AGGAACTGGA CATCAACCGG CTGTCCGACT ACGATGTGGA CGCTATCGTG | 2580 |
| | CCTCAGAGCT TTCTGGCCGA CGACTCCATC GACAACAAGG TGCTGACCAG AAGCGACAAG | 2640 |
| | AACCGGGGCA AGAGCGACAA CGTGCCCTCC GAAGAGGTCG TGAAGAAGAT GAAGAACTAC | 2700 |
| | TGGCGGCAGC TGCTGAACGC CAAGCTGATT ACCCAGAGAA AGTTCGACAA TCTGACCAAG | 2760 |
| | GCCGAGAGAG GCGGCCTGAG CGAACTGGAT AAGGCCGGCT TCATCAAGAG ACAGCTGGTG | 2820 |
| | GAAACCCGGC AGATCACAAA GCACGTGGCA CAGATCCTGG ACTCCCGGAT GAACACTAAG | 2880 |
| | TACGACGAGA ATGACAAGCT GATCCGGGAA GTGAAAGTGA TCACCCTGAA GTCCAAGCTG | 2940 |
| | GTGTCCGATT TCCGGAAGGA TTTCCAGTTT TACAAAGTGC GCGAGATCAA CAACTACGCC | 3000 |
| | CACGCCCACG ACGCCTACCT GAACGCCGTC GTGGGAACCG CCCTGATCAA AAAGTACCCT | 3060 |
| | AAGCTGGAAA GCGAGTTCGT GTACGGCGAC TACAAGGTGT ACGACGTGCG GAAGATGATC | 3120 |
| | GCCAAGAGCG AGCAGGAAAT CGGCAAGGCT ACCGCCAAGT ACTTCTTCTA CAGCAACATC | 3180 |
| | ATGAACTTTT TCAAGACCGA GATTACCCTG GCCAACGGCG AGATCCGGAA GCGGCCTCTG | 3240 |
| | ATCGAGACAA ACGGCGAAAC CGGGGAGATC GTGTGGGATA AGGGCCGGGA TTTTGCCACC | 3300 |
| | GTGCGGAAAG TGCTGAGCAT GCCCCAAGTG AATATCGTGA AAAAGACCGA GGTGCAGACA | 3360 |
| | GGCGGCTTCA GCAAAGAGTC TATCCTGCCC AAGAGGAACA GCGATAAGCT GATCGCCAGA | 3420 |
| | AAGAAGGACT GGGACCCTAA GAAGTACGGC GGCTTCGACA GCCCCACCGT GGCCTATTCT | 3480 |
| | GTGCTGGTGG TGGCCAAAGT GGAAAAGGGC AAGTCCAAGA AACTGAAGAG TGTGAAAGAG | 3540 |
| | CTGCTGGGGA TCACCATCAT GGAAAGAAGC AGCTTCGAGA AGAATCCCAT CGACTTTCTG | 3600 |
| | GAAGCCAAGG GCTACAAAGA AGTGAAAAAG GACCTGATCA TCAAGCTGCC TAAGTACTCC | 3660 |
| | CTGTTCGAGC TGGAAAACGG CCGGAAGAGA ATGCTGGCCT CTGCCGGCGA ACTGCAGAAG | 3720 |
| | GGAAACGAAC TGGCCCTGCC CTCCAAATAT GTGAACTTCC TGTACCTGGC CAGCCACTAT | 3780 |
| | GAGAAGCTGA AGGGCTCCCC CGAGGATAAT GAGCAGAAAC AGCTGTTTGT GGAACAGCAC | 3840 |
| | AAGCACTACC TGGACGAGAT CATCGAGCAG ATCAGCGAGT TCTCCAAGAG AGTGATCCTG | 3900 |
| | GCCGACGCTA ATCTGGACAA AGTGCTGTCC GCCTACAACA AGCACCGGGA TAAGCCCATC | 3960 |
| | AGAGAGCAGG CCGAGAATAT CATCCACCTG TTTACCCTGA CCAATCTGGG AGCCCCTGCC | 4020 |
| | GCCTTCAAGT ACTTTGACAC CACCATCGAC CGGAAGAGGT ACACCAGCAC CAAAGAGGTG | 4080 |
| | CTGGACGCCA CCCTGATCCA CCAGAGCATC ACCGGCCTGT ACGAGACACG GATCGACCTG | 4140 |
| | TCTCAGCTGG GAGGTGACTC CGGCGGAAGC TCTGGTGGCA GCAAGCGGAC CGCCGACGGC | 4200 |
| | TCTGAATTCG AGAGCCCTAA GAAGAAAAGA AAGGTGAGCG GAGGCTCTAG CGGCGGAAGC | 4260 |
| | ACCCTGAACA TTGAAGACGA GTATAGACTG CATGAAACAA GCAAGGAACC CGACGTGTCC | 4320 |
| | CTGGGCTCCA CCTGGCTGTC CGACTTTCCC CAGGCCTGGG CCGAGACAGG AGGAATGGGC | 4380 |
| | CTGGCCGTGC GGCAGGCACC CCTGATCATC CCTCTGAAGG CCACCTCTAC ACCCGTGAGC | 4440 |
| | ATCAAGCAGT ACCCTATGTC TCAGGAGGCC AGACTGGGCA TCAAGCCTCA CATCCAGAGG | 4500 |
| | CTGCTGGACC AGGGCATCCT GGTGCCATGC CAGAGCCCCT GGAACACACC ACTGCTGCCC | 4560 |
| | GTGAAGAAGC CAGGCACCAA TGACTATAGA CCCGTGCAGG ATCTGAGAGA GGTGAACAAG | 4620 |
| | AGGGTGGAGG ATATCCACCC CACCGTGCCC AACCCTTACA ATCTGCTGTC CGGCCTGCCC | 4680 |
| | CCTTCTCACC AGTGGTATAC AGTGCTGGAC CTGAAGGATG CCTTCTTTTG TCTGAGACTG | 4740 |
| | CACCCTACCA GCCAGCCACT GTTCGCCTTT GAGTGGAGGG ACCCTGAGAT GGGCATCTCT | 4800 |
| | GGCCAGCTGA CCTGGACACG CCTGCCTCAG GGCTTCAAGA ATAGCCCAAC ACTGTTTAAC | 4860 |
| | GAGGCCCTGC ACCGCGACCT GGCAGATTTC CGGATCCAGC ACCCAGATCT GATCCTGCTG | 4920 |
| | CAGTACGTGG ACGATCTGCT GCTGGCCGCC ACCAGCGAGC TGGATTGCCA GCAGGGAACA | 4980 |
| | CGCGCCCTGC TGCAGACCCT GGGAAACCTG GGATATAGGG CATCCGCCAA GAAGGCCCAG | 5040 |
| | ATCTGTCAGA AGCAGGTGAA GTACCTGGGC TATCTGCTGA AGGAGGGCCA GAGATGGCTG | 5100 |
| | ACAGAGGCCA GGAAGGAGAC AGTGATGGGC CAGCCAACAC CCAAGACCCC AAGACAGCTG | 5160 |
| | AGGGAGTTCC TGGGCAAAGC AGGATTTTGC AGGCTGTTCA TCCCAGGATT CGCAGAGATG | 5220 |
| | GCAGCACCTC TGTACCCACT GACCAAGCCG GGCACCCTGT TTAATTGGGG CCCTGACCAG | 5280 |
| | CAGAAGGCCT ATCAGGAGAT CAAGCAGGCC CTGCTGACAG CACCAGCCCT GGGCCTGCCA | 5340 |
| | GACCTGACCA AGCCTTTCGA GCTGTTTGTG GATGAGAAGC AGGGCTACGC CAAGGGCGTG | 5400 |
| | CTGACCCAGA AGCTGGGACC ATGGAGACGG CCCGTGGCCT ATCTGTCCAA GAAGCTGGAC | 5460 |
| | CCAGTGGCAG CAGGATGGCC ACCATGCCTG AGGATGGTGG CAGCAATCGC CGTGCTGACA | 5520 |
| | AAGGATGCCG GCAAGCTGAC CATGGGACAG CCACTGGTCA TCCTGGCACC ACACGCAGTG | 5580 |
| | GAGGCCCTGG TGAAGCAGCC TCCAGATCGC TGGCTGTCTA ACGCCCGGAT GACACACTAC | 5640 |
| | CAGGCCCTGC TGCTGGACAC CGATCGCGTG CAGTTTGGCC CTGTGGTGGC CCTGAATCCA | 5700 |
| | GCCACCCTGC TGCCTCTGCC AGAGGAGGGC CTGCAGCACA ACTGTCTGGA CATCCTGGCA | 5760 |
| | GAGGCACACG GAACAAGGCC AGACCTGACC GATCAGCCCC TGCCTGACGC CGATCACACA | 5820 |
| | TGGTATACCG ATGGAAGCTC CCTGCTGCAG GAGGGCCAGA GGAAGGCAGG AGCAGCAGTG | 5880 |
| | ACCACAGAGA CAGAAGTGAT CTGGGCCAAG GCCCTGCCAG CAGGCACATC CGCCCAGCGG | 5940 |
| | GCCGAGCTGA TCGCCCTGAC CCAGGCCCTG AAGATGGCCG AGGGCAAGAA GCTGAACGTG | 6000 |
| | TACACAGACT CCAGATATGC CTTCGCCACC GCACACATCC ACGGAGAGAT CTACAGGCGC | 6060 |
| | CGGGGCTGGC TGACCTCTGA GGGCAAGGAG ATCAAGAACA AGGATGAGAT CCTGGCCCTG | 6120 |
| | CTGAAGGCCC TGTTTCTGCC CAAGCGGCTG AGCATCATCC ACTGTCCTGG ACACCAGAAG | 6180 |
| | GGACACTCCG CCGAGGCAAG GGGCAATCGG ATGGCCGACC AGGCCGCCAG AAAGGCTGCT | 6240 |
| | ATTACTGAAA CTCCCGACAC TTCCACTCTG CTGATTGAAA ACTCCTCCCC TTCTGGCGGC | 6300 |
| | TCAAAAAGAA CCGCCGACGG CAGCGAATTC GAGTCTCCCA AGAAGAAGAG GAAAGTCGGC | 6360 |
| | TCTGGCCCTG CCGCTAAGAG AGTGAAGCTG GAC | 6393 |
| gRNA | GAGGGCCTAT TTCCCATGAT TCCTTCATAT TTGCATATAC GATACAAGGC TGTTAGAGAG | 60 |
| cloning | ATAATTGGAA TTAATTTGAC TGTAAACACA AAGATATTAG TACAAAATAC GTGACGTAGA | 120 |
| backbone | AAGTAATAAT TTCTTGGGTA GTTTGCAGTT TTAAAATTAT GTTTTAAAAT GGACTATCAT | 180 |
| | ATGCTTACCG TAACTTGAAA GTATTTCGAT TTCTTGGCTT TATATATCTT GTGGAAAGGA | 240 |
| | CGAAACACCG GGTCTTCGAG AAGACCTGTT TTAGAGCTAG AAATAGCAAG TTAAAATAAG | 300 |
| | GCTAGTCCGT TATCAACTTG AAAAAGTGGC ACCGAGTCGG TGCTTTTTTT | 350 |
| PE | MKRTADGSEF ESPKKKRKVD KKYSIGLDIG TNSVGWAVIT DEYKVPSKKF KVLGNTDRHS | 60 |
| SEQ ID | IKKNLIGALL FDSGETAEAT RLKRTARRRY TRRKNRICYL QEIFSNEMAK VDDSFFHRLE | 120 |
| NO: 293 | ESFLVEEDKK HERHPIFGNI VDEVAYHEKY PTIYHLRKKL VDSTDKADLR LIYLALAHMI | 180 |
| | KFRGHFLIEG DLNPDNSDVD KLFIQLVQTY NQLFEENPIN ASGVDAKAIL SARLSKSRRL | 240 |
| | ENLIAQLPGE KKNGLFGNLI ALSLGLTPNF KSNFDLAEDA KLQLSKDTYD DDLDNLLAQI | 300 |
| | GDQYADLFLA AKNLSDAILL SDILRVNTEI TKAPLSASMI KRYDEHHQDL TLLKALVRQQ | 360 |
| | LPEKYKEIFF DQSKNGYAGY IDGGASQEEF YKFIKPILEK MDGTEELLVK LNREDLLRKQ | 420 |
| | RTFDNGSIPH QIHLGELHAI LRRQEDFYPF LKDNREKIEK ILTFRIPYYV GPLARGNSRF | 480 |
| | AWMTRKSEET ITPWNFEEVV DKGASAQSFI ERMTNFDKNL PNEKVLPKHS LLYEYFTVYN | 540 |
| | ELTKVKYVTE GMRKPAFLSG EQKKAIVDLL FKTNRKVTVK QLKEDYFKKI ECFDSVEISG | 600 |
| | VEDRFNASLG TYHDLLKIIK DKDFLDNEEN EDILEDIVLT LTLFEDREMI EERLKTYAHL | 660 |
| | FDDKVMKQLK RRRYTGWGRL SRKLINGIRD KQSGKTILDF LKSDGFANRN FMQLIHDDSL | 720 |
| | TFKEDIQKAQ VSGQGDSLHE HIANLAGSPA IKKGILQTVK VVDELVKVMG RHKPENIVIE | 780 |
| | MARENQTTQK GQKNSRERMK RIEEGIKELG SQILKEHPVE NTQLQNEKLY LYYLQNGRDM | 840 |
| | YVDQELDINR LSDYDVDAIV PQSFLKDDSI DNKVLTRSDK NRGKSDNVPS EEVVKKMKNY | 900 |
| | WRQLLNAKLI TQRKFDNLTK AERGGLSELD KAGFIKRQLV ETRQITKHVA QILDSRMNTK | 960 |
| | YDENDKLIRE VKVITLKSKL VSDFRKDFQF YKVREINNYH HAHDAYLNAV VGTALIKKYP | 1020 |
| | KLESEFVYGD YKVYDVRKMI AKSEQEIGKA TAKYFFYSNI MNFFKTEITL ANGEIRKRPL | 1080 |
| | IETNGETGEI VWDKGRDFAT VRKVLSMPQV NIVKKTEVQT GGFSKESILP KRNSDKLIAR | 1140 |
| | KKDWDPKKYG GFDSPTVAYS VLVVAKVEKG KSKKLKSVKE LLGITIMERS SFEKNPIDFL | 1200 |
| | EAKGYKEVKK DLIIKLPKYS LFELENGRKR MLASAGELQK GNELALPSKY VNFLYLASHY | 1260 |
| | EKLKGSPEDN EQKQLFVEQH KHYLDEIIEQ ISEFSKRVIL ADANLDKVLS AYNKHRDKPI | 1320 |
| | REQAENIIHL FTLTNLGAPA AFKYFDTTID RKRYTSTKEV LDATLIHQSI TGLYETRIDL | 1380 |
| | SQLGGDSGGS SGGSKRTADG SEFESPKKKR KVSGGSSGGS TLNIEDEYRL HETSKEPDVS | 1440 |
| | LGSTWLSDFP QAWAETGGMG LAVRQAPLII PLKATSTPVS IKQYPMSQEA RLGIKPHIQR | 1500 |
| | LLDQGILVPC QSPWNTPLLP VKKPGTNDYR PVQDLREVNK RVEDIHPTVP NPYNLLSGLP | 1560 |
| | PSHQWYTVLD LKDAFFCLRL HPTSQPLFAF EWRDPEMGIS GQLTWTRLPQ GFKNSPTLFN | 1620 |
| | EALHRDLADF RIQHPDLILL QYVDDLLLAA TSELDCQQGT RALLQTLGNL GYRASAKKAQ | 1680 |
| | ICQKQVKYLG YLLKEGQRWL TEARKETVMG QPTPKTPRQL REFLGKAGFC RLFIPGFAEM | 1740 |
| | AAPLYPLTKP GTLFNWGPDQ QKAYQEIKQA LLTAPALGLP DLTKPFELFV DEKQGYAKGV | 1800 |
| | LTQKLGPWRR PVAYLSKKLD PVAAGWPPCL RMVAAIAVLT KDAGKLTMGQ PLVILAPHAV | 1860 |
| | EALVKQPPDR WLSNARMTHY QALLLDTDRV QFGPVVALNP ATLLPLPEEG LQHNCLDILA | 1920 |
| | EAHGTRPDLT DQPLPDADHT WYTDGSSLLQ EGQRKAGAAV TTETEVIWAK ALPAGTSAQR | 1980 |
| | AELIALTQAL KMAEGKKLNV YTDSRYAFAT AHIHGEIYRR RGWLTSEGKE IKNKDEILAL | 2040 |
| | LKALFLPKRL SIIHCPGHQK GHSAEARGNR MADQAARKAA ITETPDTSTL LIENSSPSGG | 2100 |
| | SKRTADGSEF ESPKKKRKVG SGPAAKRVKL D | 2131 |
| vPE | MKRTADGSEF ESPKKKRKVD KKYSIGLDIG TNSVGWAVIT DEYKVPSKKF KVLGNTDRHS | 60 |
| SEQ ID | IKKNLIGALL FDSGETAEAT RLKRTARRRY TRRKNRICYL QEIFSNEMAK VDDSFFHRLE | 120 |
| NO: 294 | ESFLVEEDKK HERHPIFGNI VDEVAYHEKY PTIYHLRKKL VDSTDKADLR LIYLALAHMI | 180 |
| | KFRGHFLIEG DLNPDNSDVD KLFIQLVQTY NQLFEENPIN ASGVDAKAIL SARLSKSRRL | 240 |
| | ENLIAQLPGE KKNGLFGNLI ALSLGLTPNF KSNFDLAEDA KLQLSKDTYD DDLDNLLAQI | 300 |
| | GDQYADLFLA AKNLSDAILL SDILRVNTEI TKAPLSASMI KRYDEHHQDL TLLKALVRQQ | 360 |
| | LPEKYKEIFF DQSKNGYAGY IDGGASQEEF YKFIKPILEK MDGTEELLVK LNREDLLRKQ | 420 |
| | RTFDNGSIPH QIHLGELHAI LRRQEDFYPF LKDNREKIEK ILTFRIPYYV GPLARGNSRF | 480 |
| | AWMTRKSEET ITPWNFEEVV DKGASAQSFI ERMTNFDKNL PNEKVLPKHS LLYEYFTVYN | 540 |
| | ELTKVKYVTE GMRKPAFLSG EQKKAIVDLL FKTNRKVTVK QLKEDYFKKI ECFDSVEISG | 600 |
| | VEDRFNASLG TYHDLLKIIK DKDFLDNEEN EDILEDIVLT LTLFEDREMI EERLKTYAHL | 660 |
| | FDDKVMKQLK RRRYTGWGRL SRKLINGIRD KQSGKTILDF LKSDGFANRN FMQLIHDDSL | 720 |
| | TFKEDIQKAQ VSGQGDSLHE HIANLAGSPA IKKGILQTVK VVDELVKVMG RHKPENIVIE | 780 |
| | MARENQTTQK GQKNSRERMK RIEEGIKELG SQILKEHPVE NTQLQNEKLY LYYLQNGRDM | 840 |
| | YVDQELDINR LSDYDVDAIV PQSFLADDSI DNKVLTRSDK NRGKSDNVPS EEVVKKMKNY | 900 |
| | WRQLLNAKLI TQRKFDNLTK AERGGLSELD KAGFIKRQLV ETRQITKHVA QILDSRMNTK | 960 |
| | YDENDKLIRE VKVITLKSKL VSDFRKDFQF YKVREINNYA HAHDAYLNAV VGTALIKKYP | 1020 |
| | KLESEFVYGD YKVYDVRKMI AKSEQEIGKA TAKYFFYSNI MNFFKTEITL ANGEIRKRPL | 1080 |
| | IETNGETGEI VWDKGRDFAT VRKVLSMPQV NIVKKTEVQT GGFSKESILP KRNSDKLIAR | 1140 |
| | KKDWDPKKYG GFDSPTVAYS VLVVAKVEKG KSKKLKSVKE LLGITIMERS SFEKNPIDFL | 1200 |
| | EAKGYKEVKK DLIIKLPKYS LFELENGRKR MLASAGELQK GNELALPSKY VNFLYLASHY | 1260 |
| | EKLKGSPEDN EQKQLFVEQH KHYLDEIIEQ ISEFSKRVIL ADANLDKVLS AYNKHRDKPI | 1320 |
| | REQAENIIHL FTLTNLGAPA AFKYFDTTID RKRYTSTKEV LDATLIHQSI TGLYETRIDL | 1380 |
| | SQLGGDSGGS SGGSKRTADG SEFESPKKKR KVSGGSSGGS TLNIEDEYRL HETSKEPDVS | 1440 |
| | LGSTWLSDFP QAWAETGGMG LAVRQAPLII PLKATSTPVS IKQYPMSQEA RLGIKPHIQR | 1500 |
| | LLDQGILVPC QSPWNTPLLP VKKPGTNDYR PVQDLREVNK RVEDIHPTVP NPYNLLSGLP | 1560 |
| | PSHQWYTVLD LKDAFFCLRL HPTSQPLFAF EWRDPEMGIS GQLTWTRLPQ GFKNSPTLFN | 1620 |
| | EALHRDLADF RIQHPDLILL QYVDDLLLAA TSELDCQQGT RALLQTLGNL GYRASAKKAQ | 1680 |
| | ICQKQVKYLG YLLKEGQRWL TEARKETVMG QPTPKTPRQL REFLGKAGFC RLFIPGFAEM | 1740 |
| | AAPLYPLTKP GTLFNWGPDQ QKAYQEIKQA LLTAPALGLP DLTKPFELFV DEKQGYAKGV | 1800 |
| | LTQKLGPWRR PVAYLSKKLD PVAAGWPPCL RMVAAIAVLT KDAGKLTMGQ PLVILAPHAV | 1860 |
| | EALVKQPPDR WLSNARMTHY QALLLDTDRV QFGPVVALNP ATLLPLPEEG LQHNCLDILA | 1920 |
| | EAHGTRPDLT DQPLPDADHT WYTDGSSLLQ EGQRKAGAAV TTETEVIWAK ALPAGTSAQR | 1980 |
| | AELIALTQAL KMAEGKKLNV YTDSRYAFAT AHIHGEIYRR RGWLTSEGKE IKNKDEILAL | 2040 |
| | LKALFLPKRL SIIHCPGHQK GHSAEARGNR MADQAARKAA ITETPDTSTL LIENSSPSGG | 2100 |
| | SKRTADGSEF ESPKKKRKVG SGPAAKRVKL D | 2131 |

TABLE 2
Oligodeoxynucleotide sequences used for Cas9 mutagenesis and cloning.

| Name | Sequence (5′-3′) |
|---|---|
| cas9-mut-FWD | GGTTGGACCGGTGCCACC |
| SEQ ID NO: 14 | |
| cas9-mid-REV | GGCCAGAGGGCCCACGTAGTAGG |
| SEQ ID NO: 15 | |
| cas9-mid-FWD | CCTACTACGTGGGCCCTCTGGCC |
| SEQ ID NO: 16 | |
| cas9-mut-REV | CTCTAGGAATTCTTACTTTTTCTTTTTTGCCTGGCC |
| SEQ ID NO: 17 | |
| R780A-BOT | CCCTCTTCGATCCGCTTCATGGCCTCGCGGCTGTTCTTCTGTC |
| SEQ ID NO: 18 | C |
| R780A-TOP | GGACAGAAGAACAGCCGCGAGGCCATGAAGCGGATCGAAGA |
| SEQ ID NO: 19 | GGG |
| R783A-BOT | GCTCTTTGATGCCCTCTTCGATGGCCTTCATTCTCTCGCGGCT |
| SEQ ID NO: 20 | GTTC |
| R783A-TOP | GAACAGCCGCGAGAGAATGAAGGCCATCGAAGAGGGCATCA |
| SEQ ID NO: 21 | AAGAGC |
| K810A-BOT | TCTGCAGGTAGTACAGGTACAGGGCCTCGTTCTGCAGCTGGG |
| SEQ ID NO: 22 | TGTTT |
| K810A-TOP | AAACACCCAGCTGCAGAACGAGGCCCTGTACCTGTACTACCT |
| SEQ ID NO: 23 | GCAGA |
| R832A-BOT | GGTCCACATCGTAGTCGGACAGGGCGTTGATGTCCAGTTCCT |
| SEQ ID NO: 24 | GGTCC |
| R832A-TOP | GGACCAGGAACTGGACATCAACGCCCTGTCCGACTACGATGT |
| SEQ ID NO: 25 | GGACC |
| K848A-BOT | CCTTGTTGTCGATGGAGTCGTCGGCCAGAAAGCTCTGAGGCA |
| SEQ ID NO: 26 | CGATA |
| K848A-TOP | TATCGTGCCTCAGAGCTTTCTGGCCGACGACTCCATCGACAA |
| SEQ ID NO: 27 | CAAGG |
| K855A-BOT | TTGTCGCTTCTGGTCAGCACGGCGTTGTCGATGGAGTCGTCCT |
| SEQ ID NO: 28 | |
| K855A-TOP | AGGACGACTCCATCGACAACGCCGTGCTGACCAGAAGCGAC |
| SEQ ID NO: 29 | AA |
| S964A-BOT | AAATCCTTCCGGAAATCGGCCACCAGCTTGGACTTCAG |
| SEQ ID NO: 30 | |
| S964A-TOP | CTGAAGTCCAAGCTGGTGGCCGATTTCCGGAAGGATTT |
| SEQ ID NO: 31 | |
| K968A-BOT | CGCACTTTGTAAAACTGGAAATCGGCCCGGAAATCGGACACC |
| SEQ ID NO: 32 | AGCTTGG |
| K968A-TOP | CCAAGCTGGTGTCCGATTTCCGGGCCGATTTCCAGTTTTACAA |
| SEQ ID NO: 33 | AGTGCG |
| R976A-BOT | GCGTGGTGGTAGTTGTTGATCTCGGCCACTTTGTAAAACTGG |
| SEQ ID NO: 34 | AAATCCT |
| R976A-TOP | AGGATTTCCAGTTTTACAAAGTGGCCGAGATCAACAACTACC |
| SEQ ID NO: 35 | ACCACGC |
| H982A-BOT | GTAGGCGTCGTGGGCGTGGGCGTAGTTGTTGATCTCGCG |
| SEQ ID NO: 36 | |
| H982A-TOP | CGCGAGATCAACAACTACGCCCACGCCCACGACGCCTAC |
| SEQ ID NO: 37 | |
| K1003A-BOT | CGTACACGAACTCGCTTTCCAGGGCAGGGTACTTTTTGATCA |
| SEQ ID NO: 38 | GGGCG |
| K1003A-TOP | CGCCCTGATCAAAAAGTACCCTGCCCTGGAAAGCGAGTTCGT |
| SEQ ID NO: 39 | GTACG |
| K1047A-BOT | GCCGTTGGCCAGGGTAATCTCGGTGGCGAAAAAGTTCATGAT |
| SEQ ID NO: 40 | GTTGCTG |
| K1047A-TOP | CAGCAACATCATGAACTTTTTCGCCACCGAGATTACCCTGGC |
| SEQ ID NO: 41 | CAACGGC |
| R1060A-BOT | CCGTTTGTCTCGATCAGAGGGGCCTTCCGGATCTCGCCGTTGG |
| SEQ ID NO: 42 | |
| R1060A-TOP | CCAACGGCGAGATCCGGAAGGCCCCTCTGATCGAGACAAAC |
| SEQ ID NO: 43 | GG |
| R976A-H982A-BOT | GTAGGCGTCGTGGGCGTGGGCGTAGTTGTTGATCTCGGC |
| SEQ ID NO: 44 | |
| R976A-H982A-TOP | GCCGAGATCAACAACTACGCCCACGCCCACGACGCCTAC |
| SEQ ID NO: 45 | |
| D54R-TOP | CGGAGCCCTGCTGTTCCGGAGCGGCGAAACAGCCG |
| SEQ ID NO: 46 | |
| D54R-BOT | CGGCTGTTTCGCCGCTCCGGAACAGCAGGGCTCCG |
| SEQ ID NO: 47 | |
| S55R-TOP | GCCCTGCTGTTCGACCGGGGCGAAACAGCCGAG |
| SEQ ID NO: 48 | |
| S55R-BOT | CTCGGCTGTTTCGCCCCGGTCGAACAGCAGGGC |
| SEQ ID NO: 49 | |
| N980R-TOP | CAAAGTGGCCGAGATCAACCGGTACCACCACGCCCACGACG |
| SEQ ID NO: 50 | |
| N980R-BOT | CGTCGTGGGCGTGGTGGTACCGGTTGATCTCGGCCACTTTG |
| SEQ ID NO: 51 | |
| T1314R-TOP | CGAGAATATCATCCACCTGTTTCGGCTGACCAATCTGGGAGC |
| SEQ ID NO: 52 | CCCTG |
| T1314R-BOT | CAGGGGCTCCCAGATTGGTCAGCCGAAACAGGTGGATGATAT |
| SEQ ID NO: 53 | TCTCG |
| N1317R-TOP | CCACCTGTTTACCCTGACCCGGCTGGGAGCCCCTGCCGCCT |
| SEQ ID NO: 54 | |
| N1317R-BOT | AGGCGGCAGGGGCTCCCAGCCGGGTCAGGGTAAACAGGTGG |
| SEQ ID NO: 55 | |
| A1322R-TOP | CCCTGACCAATCTGGGAGCCCCTCGGGCCTTCAAGTACTTTG |
| SEQ ID NO: 56 | ACACCAC |
| A1322R-BOT | GTGGTGTCAAAGTACTTGAAGGCCCGAGGGGCTCCCAGATTG |
| SEQ ID NO: 57 | GTCAGGG |
| pe-FWD | GCTAGAGATCCGCGGCCGCTAATAC |
| SEQ ID NO: 194 | |
| pe-mid-REV | CACTTTCACGAGCTCGTCCACCAC |
| SEQ ID NO: 195 | |
| pe-mid-FWD | GTGGTGGACGAGCTCGTGAAAGTG |
| SEQ ID NO: 196 | |
| pe-rt-REV | CTGGGTGCTGGATCCGGAAATCTG |
| SEQ ID NO: 197 | |
| N980R-H982A-BOT | CGTCGTGGGCGTGGGCGTACCGGTTGATCTCGCGCACTTTG |
| SEQ ID NO: 198 | |
| N980R-H982A-TOP | CAAAGTGCGCGAGATCAACCGGTACGCCCACGCCCACGACG |
| SEQ ID NO: 199 | |

TABLE 3
Oligodeoxynucleotide sequences used for gRNA cloning.

| Name | Sequence (5′-3′) |
|---|---|
| gRNA-scaffold-NheI- | CTCAGCTAGCGAGGGCCTATTTCCCATGATTCCTTCATAT |
| FWDE | TTGC |
| SEQ ID NO: 58 | |
| gRNA-scaffold-EcoRI- | ATCAGAATTCAAAAAAAGCACCGACTCGGTGCCACTT |
| REV | |
| SEQ ID NO: 59 | |
| AAVS1-gRNA1-BOT | AAACCTAGGGACAGGATTGGTGAC |
| SEQ ID NO: 60 | |
| AAVS1-gRNA1-TOP | CACCGTCACCAATCCTGTCCCTAG |
| SEQ ID NO: 61 | |
| AAVS1-gRNA2-BOT | AAACATCCTGTCCCTAGTGGCCCC |
| SEQ ID NO: 62 | |
| AAVS1-gRNA2-TOP | CACCGGGGCCACTAGGGACAGGAT |
| SEQ ID NO: 63 | |
| CD274-gRNA1-BOT | AAACGGGAACTTCAAATTCATTCC |
| SEQ ID NO: 64 | |
| CD274-gRNA1-TOP | CACCGGAATGAATTTGAAGTTCCC |
| SEQ ID NO: 65 | |
| CD274-gRNA2-BOT | AAACTGGGAACTTCAAATTCATTC |
| SEQ ID NO: 66 | |
| CD274-gRNA2-TOP | CACCGAATGAATTTGAAGTTCCCA |
| SEQ ID NO: 67 | |
| CXCR4-gRNA-BOT | AAACCCTCTTTGTCATCACGCTTC |
| SEQ ID NO: 68 | |
| CXCR4-gRNA-TOP | CACCGAAGCGTGATGACAAAGAGG |
| SEQ ID NO: 69 | |
| EMX1-gRNA1-BOT | AAACCCCTAGTCATTGGAGGTGAC |
| SEQ ID NO: 70 | |
| EMX1-gRNA1-TOP | CACCGTCACCTCCAATGACTAGGG |
| SEQ ID NO: 71 | |
| EMX1-gRNA2-BOT | AAACTTCTTCTTCTGCTCGGACTC |
| SEQ ID NO: 72 | |
| EMX1-gRNA2-TOP | CACCGAGTCCGAGCAGAAGAAGAA |
| SEQ ID NO: 73 | |
| GFP-gRNA-BOT | AAACACGGCGTGCAGTGCTTCAGC |
| SEQ ID NO: 74 | |
| GFP-gRNA-TOP | CACCGCTGAAGCACTGCACGCCGT |
| SEQ ID NO: 75 | |
| KRAS-gRNA1-BOT | AAACCGATTATTATCAGCCTCAGC |
| SEQ ID NO: 76 | |
| KRAS-gRNA1-TOP | CACCGCTGAGGCTGATAATAATCG |
| SEQ ID NO: 77 | |
| KRAS-gRNA2-BOT | AAACCCCCGATTATTATCAGCCTC |
| SEQ ID NO: 78 | |
| KRAS-gRNA2-TOP | CACCGAGGCTGATAATAATCGGGG |
| SEQ ID NO: 79 | |
| MYC-gRNA1-BOT | AAACGCGTCGGGAGAGTCGCGTCC |
| SEQ ID NO: 80 | |
| MYC-gRNA1-TOP | CACCGGACGCGACTCTCCCGACGC |
| SEQ ID NO: 81 | |
| MYC-gRNA2-BOT | AAACCGCGTCGGGAGAGTCGCGTC |
| SEQ ID NO: 82 | |
| MYC-gRNA2-TOP | CACCGACGCGACTCTCCCGACGCG |
| SEQ ID NO: 83 | |
| MYC-gRNA3-BOT | CACCGCGACTCTCCCGACGCGGGG |
| SEQ ID NO: 84 | |
| MYC-gRNA3-TOP | AAACCCCCGCGTCGGGAGAGTCGC |
| SEQ ID NO: 85 | |
| STAT1-gRNA1-BOT | AAACCCAGCTGCAAGCATGTCATC |
| SEQ ID NO: 86 | |
| STAT1-gRNA1-TOP | CACCGATGACATGCTTGCAGCTGG |
| SEQ ID NO: 87 | |
| STAT1-gRNA2-BOT | AAACCAGCTGCAAGCATGTCATCC |
| SEQ ID NO: 88 | |
| STAT1-gRNA2-TOP | CACCGGATGACATGCTTGCAGCTG |
| SEQ ID NO: 89 | |
| TGFB1-gRNA-BOT | AAACCTTGGTGGAAGCGCAGGCTC |
| SEQ ID NO: 90 | |
| TGFB1-gRNA-TOP | CACCGAGCCTGCGCTTCCACCAAG |
| SEQ ID NO: 91 | |
| VEGFA-gRNA1-BOT | AAACCACGCACACACTCACTCACC |
| SEQ ID NO: 92 | |
| VEGF A-gRNA1-TOP | CACCGGTGAGTGAGTGTGTGCGTG |
| SEQ ID NO: 93 | |
| VEGFA-gRNA2-BOT | AAACACACGCACACACTCACTCAC |
| SEQ ID NO: 94 | |
| VEGFA-gRNA2-TOP | CACCGTGAGTGAGTGTGTGCGTGT |
| SEQ ID NO: 95 | |
| ASNS-gRNA1-BOT | AAACTGCGCCCCGCGCCAGCATCC |
| SEQ ID NO: 200 | |
| ASNS-gRNA1-TOP | CACCGGATGCTGGCGCGGGGCGCA |
| SEQ ID NO: 201 | |
| HOTAIR-gRNA1-BOT | AAACCACCGCAGTTCTAGGCAAGC |
| SEQ ID NO: 202 | |
| HOTAIR-gRNA1-TOP | CACCGCTTGCCTAGAACTGCGGTG |
| SEQ ID NO: 203 | |
| THORLNC-gRNA1-BOT | AAACCTTTGTTCACATCATCTCAC |
| SEQ ID NO: 204 | |
| THORLNC-gRNA1-TOP | CACCGTGAGATGATGTGAACAAAG |
| SEQ ID NO: 205 | |
| TP53-gRNA1-BOT | AAACTCCAGGTCCCCAGCCCAACC |
| SEQ ID NO: 206 | |
| TP53-gRNA1-TOP | CACCGGTTGGGCTGGGGACCTGGA |
| SEQ ID NO: 207 | |

TABLE 4
Oligodeoxynucleotide sequences used for HDR and MDR templates.

| Name | Sequence (5′-3′) |
|---|---|
| AAVS1- | CAGGGCCGGTTAATGTGGCTCTGGTTCTGGGTACTTTTATCTGT |
| HDRtemplate | CCCCTCCACCCCACAGTGGGGCCGCTAGCAAGCTTGGACAGG |
| SEQ ID NO: 96 | ATTGGTGACAGAAAAGCCCCATCCTTAG |
| CD274- | TATGAAAGATAATGAAAAGCTATGGGAAAGATAACTTAGAAA |
| HDRtemplate | CAAAGAAGGCATGGATCCTCAGCCCTGGGCTAGCAAGCTTCA |
| SEQ ID NO: 97 | AATTCATTCCATCTGCTATATAAGAAACA |
| CXCR4- | ATGGGTTACCAGAAGAAACTGAGAAGCATGACGGACAAGTAC |
| HDRtemplate | AGGCTGCACCTGTCAGTGGCCGACCTGCTAGCAAGCTTCTTTG |
| SEQ ID NO: 98 | TCATCACGCTTCCCTTCTGGGCAGTTGATGC |
| EMX1- | CTTGGGCCCACGCAGGGGCCTGGCCAGCAGCAAGCAGCACTC |
| HDRtemplate | TGCCCTCGTGGGTTTGTGGTTGCCCACCGCTAGCAAGCTTGTC |
| SEQ ID NO: 99 | ATTGGAGGTGACATCGATGTCCTCCCCATTG |
| GFP-BFP- | CCTGAAGTTCATCTGCACCACCGGCAAGCTGCCCGTGCCCTGG |
| HDRtemplate | CCCACCCTCGTGACCACCCTGAGCCACGGGGTGCAGTGCTTCA |
| SEQ ID NO: 100 | GCCGCTACCCCGACCACATGA |
| KRAS- | GGCGACTTCGGGGACTTAGGGAGACCGGGCGGACGATTTCCC |
| HDRtemplate | ACACCGGGGCTGTCTGATCGCCGGCTAGCAAGCTTATTATCAG |
| SEQ ID NO: 101 | CCTCAGCACTTGGGCTGGGAATTTAG |
| MYC- | AGCCTTTCAGAGAAGCGGGTCCTGGCAGCGGCGGGGAAGTGT |
| HDRtemplate | CCCCAAATGGGCAGAATAGCCTCGCTAGCAAGCTTCGGGAGA |
| SEQ ID NO: 102 | GTCGCGTCCTTGCTCGGGTGTTGTAAGT |
| STAT1- | GTATTTCTAATAGACTTGAAAGGACAGCCAGGAGCAAAGATG |
| HDRtemplate | GGCAGAAGGACAACCTGTTTCCCCAAGCTTGCTAGCAAGCAT |
| SEQ ID NO: 103 | GTCATCCTCACATTTGGCCCCTTGGCCC |
| TGFB1- | ACCCTGAGAGGAACTGGGACTTTGGGGTCCAGACTGCCAGCG |
| HDRtemplate | TTTAGCGCAGCGGGGTCCTCCTGCCCCTTGGAAGCTTGCTAGC |
| SEQ ID NO: 104 | GCAGGCTCCTCCCCCCGCGCGTGGCAC |
| VEGFA- | ACACACAGATCTATTGGAATCCTGGAGTGACCCCTGGCCTTCT |
| HDRtemplate | CCCCGCTCCAACGCCCTCAACCCCACGCTAGCAAGCTTACACT |
| SEQ ID NO: 105 | CACTCACCCACACAGACACACACGTCC |
| AAVS1- | AATCCGGTGTCCCTAGTGGCCCCACTG |
| MDRtemplate-BOT | |
| SEQ ID NO: 106 | |
| AAVS1- | GGACACCGGATTGGTGACAGAAAAGCC |
| MDRtemplate-TOP | |
| SEQ ID NO: 107 | |
| CD274- | CTGGGACTTCAAATTCATTCCATCT |
| MDRtemplate-BOT | |
| SEQ ID NO: 108 | |
| CD274- | GAAGTCCCAGGGCTGAGGATCCATG |
| MDRtemplate-TOP | |
| SEQ ID NO: 109 | |
| CXCR4- | ACAAACAGGAGGTCGGCCACTGACAG |
| MDRtemplate-BOT | |
| SEQ ID NO: 110 | |
| CXCR4- | CTCCTGTTTGTCATCACGCTTCCCTT |
| MDRtemplate-TOP | |
| SEQ ID NO: 111 | |
| GFP-BFP-5′0- | CCCGTGGCTCAGGGTGGTCACGAGGGTG |
| 3′20- | |
| MDRtemplate-BOT | |
| SEQ ID NO: 112 | |
| GFP-BFP-5′0- | GCCACGGGGTGCAGTGCTTCAGCCGCTA |
| 3′20- | |
| MDRtemplate-TOP | |
| SEQ ID NO: 113 | |
| GFP-BFP-5′5- | TGCACCCCGTGGCTCAGGGTGGTCACGAGGGTG |
| 3′20- | |
| MDRtemplate-BOT | |
| SEQ ID NO: 114 | |
| GFP-BFP-5′5- | CCTGAGCCACGGGGTGCAGTGCTTCAGCCGCTA |
| 3′20- | |
| MDRtemplate-TOP | |
| SEQ ID NO: 115 | |
| GFP-BFP-5′10- | AGCACTGCACCCCGTGGCTCAGGGTGGTCACGAGGGTG |
| 3′20- | |
| MDRtemplate-BOT | |
| SEQ ID NO: 116 | |
| GFP-BFP-5′10- | ACCACCCTGAGCCACGGGGTGCAGTGCTTCAGCCGCTA |
| 3′20- | |
| MDRtemplate-TOP | |
| SEQ ID NO: 117 | |
| KRAS- | CCGCCTAGATTATTATCAGCCTCAGCA |
| MDRtemplate-BOT | |
| SEQ ID NO: 118 | |
| KRAS- | TAATCTAGGCGGCGATCAGACAGCCCC |
| MDRtemplate-TOP | |
| SEQ ID NO: 119 | |
| MYC- | CCCGCTGTCGGGAGAGTCGCGTCCTT |
| MDRtemplate-BOT | |
| SEQ ID NO: 120 | |
| MYC- | CCGACAGCGGGGAGGCTATTCTGCCC |
| MDRtemplate-TOP | |
| SEQ ID NO: 121 | |
| TGFB1- | CCCCTCTGGTGGAAGCGCAGGCTCCT |
| MDRtemplate-BOT | |
| SEQ ID NO: 122 | |
| TGFB1- | CACCAGAGGGGCAGGAGGACCCCGCT |
| MDRtemplate-TOP | |
| SEQ ID NO: 123 | |
| HOTAIR- | TAGAACTGCGCACACAAAAAACCAACACACAGATCTAATGAA |
| INStemplate-BOT | AATAAAGATCTTTTATTGTGTGGAAGGCGCTGCCCCG |
| SEQ ID NO: 266 | |
| HOTAIR- | CCTTCCACACAATAAAAGATCTTTATTTTCATTAGATCTGTGTG |
| INStemplate-TOP | TTGGTTTTTTGTGTGCGCAGTTCTAGGCAAGCACT |
| SEQ ID NO: 267 | |
| THORLNC- | TTTCCCCCTTCACACAAAAAACCAACACACAGATCTAATGAAA |
| INStemplate-BOT | ATAAAGATCTTTTATTTGTTCACATCATCTCACAAA |
| SEQ ID NO: 268 | |
| THORLNC- | GATGTGAACAAATAAAAGATCTTTATTTTCATTAGATCTGTGT |
| INStemplate-TOP | GTTGGTTTTTTGTGTGAAGGGGGAAAAGTCAATCCA |
| SEQ ID NO: 269 | |

TABLE 5
Oligodeoxynucleotide sequences used for next-generation sequencing.

| Name | Sequence (5′-3′) |
|---|---|
| AAVS1-seq-r1- | ACACTCTTTCCCTACACGACGCTCTTCCGATCTNTCACGGTTAA |
| FWD | TGTGGCTCTGGTTCTGG |
| SEQ ID NO: 124 | |
| AAVS1-seq-r2- | ACACTCTTTCCCTACACGACGCTCTTCCGATCTNAGTCGGTTA |
| FWD | ATGTGGCTCTGGTTCTGG |
| SEQ ID NO: 125 | |
| AAVS1-seq-r3- | ACACTCTTTCCCTACACGACGCTCTTCCGATCTNCAGCGGTTA |
| FWD | ATGTGGCTCTGGTTCTGG |
| SEQ ID NO: 126 | |
| AAVS1-seq-REV | GACTGGAGTTCAGACGTGTGCTCTTCCGATCTGGGGTTAGACC |
| SEQ ID NO: 127 | CAATATCAGGAGACTAG |
| CD274-seq-r1- | ACACTCTTTCCCTACACGACGCTCTTCCGATCTNATCTGTATGT |
| FWD | CTGCTGTGTACTTTGC |
| SEQ ID NO: 128 | |
| CD274-seq-r2- | ACACTCTTTCCCTACACGACGCTCTTCCGATCTNCGATGTATGT |
| FWD | CTGCTGTGTACTTTGC |
| SEQ ID NO: 129 | |
| CD274-seq-r3- | ACACTCTTTCCCTACACGACGCTCTTCCGATCTNTAGTGTATGT |
| FWD | CTGCTGTGTACTTTGC |
| SEQ ID NO: 130 | |
| CD274-seq-REV | GACTGGAGTTCAGACGTGTGCTCTTCCGATCTACTTAACAAAT |
| SEQ ID NO: 131 | GGTGGTTGTCTAAA |
| CXCR4-seq-r1- | ACACTCTTTCCCTACACGACGCTCTTCCGATCTNCGTTGGTCAT |
| FWD | GGGTTACCAGAAGA |
| SEQ ID NO: 132 | |
| CXCR4-seq-r2- | ACACTCTTTCCCTACACGACGCTCTTCCGATCTNACGTGGTCAT |
| FWD | GGGTTACCAGAAGA |
| SEQ ID NO: 133 | |
| CXCR4-seq-r3- | ACACTCTTTCCCTACACGACGCTCTTCCGATCTNGTATGGTCAT |
| FWD | GGGTTACCAGAAGA |
| SEQ ID NO: 134 | |
| CXCR4-seq-REV | GACTGGAGTTCAGACGTGTGCTCTTCCGATCTGACTGATGAAG |
| SEQ ID NO: 135 | GCCAGGATG |
| EMX1-seq-r1- | ACACTCTTTCCCTACACGACGCTCTTCCGATCTNGACCCTGAG |
| FWD | TCCGAGCAGAAGAA |
| SEQ ID NO: 136 | |
| EMX1-seq-r2- | ACACTCTTTCCCTACACGACGCTCTTCCGATCTNTGACCTGAGT |
| FWD | CCGAGCAGAAGAA |
| SEQ ID NO: 137 | |
| EMX1-seq-r3- | ACACTCTTTCCCTACACGACGCTCTTCCGATCTNACTCCTGAGT |
| FWD | CCGAGCAGAAGAA |
| SEQ ID NO: 138 | |
| EMX1-seq-REV | GACTGGAGTTCAGACGTGTGCTCTTCCGATCTAGTGGCCAGAG |
| SEQ ID NO: 139 | TCCAGCTT |
| GFP-seq-r1-FWD | ACACTCTTTCCCTACACGACGCTCTTCCGATCTNATGCCCTGA |
| SEQ ID NO: 140 | AGTTCATCTGCACCAC |
| GFP-seq-r2-FWD | ACACTCTTTCCCTACACGACGCTCTTCCGATCTNGATCCCTGA |
| SEQ ID NO: 141 | AGTTCATCTGCACCAC |
| GFP-seq-r3-FWD | ACACTCTTTCCCTACACGACGCTCTTCCGATCTNTGCCCCTGAA |
| SEQ ID NO: 142 | GTTCATCTGCACCAC |
| GFP-seq-REV | GACTGGAGTTCAGACGTGTGCTCTTCCGATCTTAGTTGCCGTC |
| SEQ ID NO: 143 | GTCCTTGAAGA |
| KRAS-seq-r1- | ACACTCTTTCCCTACACGACGCTCTTCCGATCTNGACTTGAAA |
| FWD | GGGTCTGTCGTGTTTG |
| SEQ ID NO: 144 | |
| KRAS-seq-r2- | ACACTCTTTCCCTACACGACGCTCTTCCGATCTNTGATTGAAA |
| FWD | GGGTCTGTCGTGTTTG |
| SEQ ID NO: 145 | |
| KRAS-seq-r3- | ACACTCTTTCCCTACACGACGCTCTTCCGATCTNACTTTGAAA |
| FWD | GGGTCTGTCGTGTTTG |
| SEQ ID NO: 146 | |
| KRAS-seq-REV | GACTGGAGTTCAGACGTGTGCTCTTCCGATCTAAACAAGCAGT |
| SEQ ID NO: 147 | CACCAAAAGTGG |
| MYC-seq-r1- | ACACTCTTTCCCTACACGACGCTCTTCCGATCTNCGTCACGAA |
| FWD | ACTTTGCCCATAGCA |
| SEQ ID NO: 148 | |
| MYC-seq-r2- | ACACTCTTTCCCTACACGACGCTCTTCCGATCTNACGCACGAA |
| FWD | ACTTTGCCCATAGCA |
| SEQ ID NO: 149 | |
| MYC-seq-r3- | ACACTCTTTCCCTACACGACGCTCTTCCGATCTNGTACACGAA |
| FWD | ACTTTGCCCATAGCA |
| SEQ ID NO: 150 | |
| MYC-seq-REV | GACTGGAGTTCAGACGTGTGCTCTTCCGATCTAAGTGGACTTC |
| SEQ ID NO: 151 | GGTGCTTACC |
| STAT1-seq-r1- | ACACTCTTTCCCTACACGACGCTCTTCCGATCTNATGAAAGTA |
| FWD | GTATGCGTGGGCCTC |
| SEQ ID NO: 152 | |
| STAT1-seq-r2- | ACACTCTTTCCCTACACGACGCTCTTCCGATCTNGATAAAGTA |
| FWD | GTATGCGTGGGCCTC |
| SEQ ID NO: 153 | |
| STAT1-seq-r3- | ACACTCTTTCCCTACACGACGCTCTTCCGATCTNTGCAAAGTA |
| FWD | GTATGCGTGGGCCTC |
| SEQ ID NO: 154 | |
| STAT1-seq-REV | GACTGGAGTTCAGACGTGTGCTCTTCCGATCTGCTCAAAAGCT |
| SEQ ID NO: 155 | GGTAAACCTTCA |
| TGFB1-seq-r1- | ACACTCTTTCCCTACACGACGCTCTTCCGATCTNGACGTGACT |
| FWD | CTACAAGACCGAGGTG |
| SEQ ID NO: 156 | |
| TGFB1-seq-r2- | ACACTCTTTCCCTACACGACGCTCTTCCGATCTNTGAGTGACTC |
| FWD | TACAAGACCGAGGTG |
| SEQ ID NO: 157 | |
| TGFB1-seq-r3- | ACACTCTTTCCCTACACGACGCTCTTCCGATCTNACTGTGACTC |
| FWD | TACAAGACCGAGGTG |
| SEQ ID NO: 158 | |
| TGFB1-seq-REV | GACTGGAGTTCAGACGTGTGCTCTTCCGATCTCCTGAGAGGAA |
| SEQ ID NO: 159 | CTGGGACTTTG |
| VEGF A-seq-r1- | ACACTCTTTCCCTACACGACGCTCTTCCGATCTNATGGCGTCTT |
| FWD | CGAGAGTGAGGAC |
| SEQ ID NO: 160 | |
| VEGFA-seq-r2- | ACACTCTTTCCCTACACGACGCTCTTCCGATCTNGATGCGTCTT |
| FWD | CGAGAGTGAGGAC |
| SEQ ID NO: 161 | |
| VEGF A-seq-r3- | ACACTCTTTCCCTACACGACGCTCTTCCGATCTNTGCGCGTCTT |
| FWD | CGAGAGTGAGGAC |
| SEQ ID NO: 162 | |
| VEGF A-seq-REV | GACTGGAGTTCAGACGTGTGCTCTTCCGATCTGGGGAGAGGG |
| SEQ ID NO: 163 | ACACACAGAT |
| GFP-seq-r1-FWD | ACACTCTTTCCCTACACGACGCTCTTCCGATCTNATGCGTAAA |
| SEQ ID NO: 270 | CGGCCACAAGTTCAGC |
| GFP-seq-r2-FWD | ACACTCTTTCCCTACACGACGCTCTTCCGATCTNGATCGTAAA |
| SEQ ID NO: 271 | CGGCCACAAGTTCAGC |
| GFP-seq-r3-FWD | ACACTCTTTCCCTACACGACGCTCTTCCGATCTNTGCCGTAAA |
| SEQ ID NO: 272 | CGGCCACAAGTTCAGC |
| HOTAIR-seq-r1- | ACACTCTTTCCCTACACGACGCTCTTCCGATCTNGACCAGTGA |
| FWD | AATCTGGCGAGAGCAG |
| SEQ ID NO: 273 | |
| HOTAIR-seq-r2- | ACACTCTTTCCCTACACGACGCTCTTCCGATCTNTGACAGTGA |
| FWD | AATCTGGCGAGAGCAG |
| SEQ ID NO: 274 | |
| HOTAIR-seq-r3- | ACACTCTTTCCCTACACGACGCTCTTCCGATCTNACTCAGTGA |
| FWD | AATCTGGCGAGAGCAG |
| SEQ ID NO: 275 | |
| HOTAIR-seq- | GACTGGAGTTCAGACGTGTGCTCTTCCGATCTTCAAACTATGT |
| REV | GTTCGCGGGTC |
| SEQ ID NO: 276 | |
| THORLNC-seq- | ACACTCTTTCCCTACACGACGCTCTTCCGATCTNCGTTCTCCGG |
| r1-FWD | AGCAGAAATAGAACAG |
| SEQ ID NO: 277 | |
| THORLNC-seq- | ACACTCTTTCCCTACACGACGCTCTTCCGATCTNACGTCTCCGG |
| 12-FWD | AGCAGAAATAGAACAG |
| SEQ ID NO: 278 | |
| THORLNC-seq- | ACACTCTTTCCCTACACGACGCTCTTCCGATCTNGTATCTCCGG |
| r3-FWD | AGCAGAAATAGAACAG |
| SEQ ID NO: 279 | |
| THORLNC-seq- | GACTGGAGTTCAGACGTGTGCTCTTCCGATCTTTCATGCCGTC |
| REV | AAGTCTCATTT |
| SEQ ID NO: 280 | |
| TP53-seq-r1- | ACACTCTTTCCCTACACGACGCTCTTCCGATCTNATGATTCCAT |
| FWD | GGGACTGACTTTCTGC |
| SEQ ID NO: 281 | |
| TP53-seq-r2- | ACACTCTTTCCCTACACGACGCTCTTCCGATCTNGATATTCCAT |
| FWD | GGGACTGACTTTCTGC |
| SEQ ID NO: 282 | |
| TP53-seq-r3- | ACACTCTTTCCCTACACGACGCTCTTCCGATCTNTGCATTCCAT |
| FWD | GGGACTGACTTTCTGC |
| SEQ ID NO: 283 | |
| TP53-seq-REV | GACTGGAGTTCAGACGTGTGCTCTTCCGATCTCATCTGGACCT |
| SEQ ID NO: 284 | GGGTCTTCAGT |
| ASNS-seq-r1- | ACACTCTTTCCCTACACGACGCTCTTCCGATCTNGACTTACAG |
| FWD | GAGCCAGGTCGGTAT |
| SEQ ID NO: 289 | |
| ASNS-seq-r2- | ACACTCTTTCCCTACACGACGCTCTTCCGATCTNTGATTACAG |
| FWD | GAGCCAGGTCGGTAT |
| SEQ ID NO: 290 | |
| ASNS-seq-r3- | ACACTCTTTCCCTACACGACGCTCTTCCGATCTNACTTTACAGG |
| FWD | AGCCAGGTCGGTAT |
| SEQ ID NO: 291 | |
| ASNS-seq-REV | GACTGGAGTTCAGACGTGTGCTCTTCCGATCTGTCAGGTGCGT |
| SEQ ID NO: 292 | AACAATCGC |

TABLE 6
Oligodeoxynucleotide sequences used for Sanger sequencing.

| Name | Sequence (5′-3′) |
|---|---|
| CXCR4-FWD | AGCTGGAGTGAAAACTTGAAGACTCAG |
| SEQ ID NO: 164 | |
| CXCR4-REV | GTTTGTATTTAGGCAGGCGTGGGAAA |
| SEQ ID NO: 165 | |
| CXCR4-seq | CTACACCGAGGAAATGGGCTCAG |
| SEQ ID NO: 166 | |
| EMX1-FWD | GGCTCCCTGGGTTCAAAGTA |
| SEQ ID NO: 167 | |
| EMX1-REV | AGAGGGGTCTGGATGTCGTAA |
| SEQ ID NO: 168 | |
| EMX1-seq | GGCCTCCTGAGTTTCTCATCTGTG |
| SEQ ID NO: 169 | |
| EMX1-T-FWD | GGCTCCCTGGGTTCAAAGTA |
| SEQ ID NO: 170 | |
| EMX1-T-REV | AGAGGGGTCTGGATGTCGTAA |
| SEQ ID NO: 171 | |
| EMX1-T-seq | AACCCTATGTAGCCTCAGTCTTCCC |
| SEQ ID NO: 172 | |
| EMX1-OT1-FWD | TTATCCCCTACTCCTTCATCCCA |
| SEQ ID NO: 173 | |
| EMX1-OT1-REV | AAGGACAGCTTCTTATCCCTGTC |
| SEQ ID NO: 174 | |
| EMX1-OT1-seq | GGAGATTTGCATCTGTGGAGGC |
| SEQ ID NO: 175 | |
| EMX1-OT2-FWD | ACTCCTGGGACAATTATGAACGGTG |
| SEQ ID NO: 176 | |
| EMX1-OT2-REV | ACTATCCTTCTAGTCTTGGGCTAAATTC |
| SEQ ID NO: 177 | |
| EMX1-OT2-seq | GCTTCTTGTTCTTTGGCTTTCTTAATGAACTG |
| SEQ ID NO: 178 | |
| EMX1-OT3-FWD | GCACTGATTCATTAGGAGCTGG |
| SEQ ID NO: 179 | |
| EMX1-OT3-REV | AGTCCTATAGATTCACCCACCCA |
| SEQ ID NO: 180 | |
| EMX1-OT3-seq | TCCTGGTTCTGCCACTTGCTG |
| SEQ ID NO: 181 | |
| VEGF A-FWD | GCTCCAGATGGCACATTGTCAG |
| SEQ ID NO: 182 | |
| VEGFA-REV | AGGGAGCAGGAAAGTGAGGT |
| SEQ ID NO: 183 | |
| VEGFA-seq | CAAATATGTAGCTGTTTGGGAGGTCAG |
| SEQ ID NO: 184 | |
| VEGF1-OT1-FWD | TTCCCACCAAGGAGGGTTTCTT |
| SEQ ID NO: 185 | |
| VEGFA-OT1-REV | CCTCCCTCAAGGGAAGGTTGT |
| SEQ ID NO: 186 | |
| VEGFA-OT1-seq | CAAGTAGCTGAGATTACAGGCATGTGC |
| SEQ ID NO: 187 | |
| VEGFA-OT2-FWD | ATTCCTCAGGTGGGTTGATGGG |
| SEQ ID NO: 188 | |
| VEGFA-OT2-REV | AGAAAGGAGCCTCGACCAAGTC |
| SEQ ID NO: 189 | |
| VEGFA-OT2-seq | GCCTCCCTGCTGGTTCTCAGAG |
| SEQ ID NO: 190 | |
| VEGFA-OT3-FWD | TCCCATCCCACTTTAGTGTTCC |
| SEQ ID NO: 191 | |
| VEGFA-OT3-REV | TAACCCAGAACATCCAGGCAAC |
| SEQ ID NO: 192 | |
| VEGFA-OT3-seq | TACCATGAACGCAGCCATGC |
| SEQ ID NO: 193 | |

TABLE 7
Oligodeoxynucleotide sequences used for pegRNA clonging.

| Name | Sequence (5′-3′) |
|---|---|
| pegRNA-scaffold- | GCACCGACTCGGTGCCACTTTTTCAAGTTGATAACGGACTAGC |
| BOT | CTTATTTTAACTTGCTATTTCTAG |
| SEQ ID NO: 208 | |
| pegRNA-scaffold- | AGAGCTAGAAATAGCAAGTTAAAATAAGGCTAGTCCGTTATC |
| TOP | AACTTGAAAAAGTGGCACCGAGTCG |
| SEQ ID NO: 209 | |
| AAVS1-pegS1-BOT | CTCTAAAACCACTGTGGGGTGGAGGGGAC |
| SEQ ID NO: 210 | |
| AAVS1-pegS1-TOP | CACCGTCCCCTCCACCCCACAGTGGTTTT |
| SEQ ID NO: 211 | |
| AAVS1--14sub5- | AAAACCCTCCACCCCACAGTGGGGCCGCTAGCAAGCTTGGAC |
| pegT-BOT | AGGATT |
| SEQ ID NO: 212 | |
| AAVS1--14sub5- | GTGCAATCCTGTCCAAGCTTGCTAGCGGCCCCACTGTGGGGTG |
| pegT-TOP | GAGGG |
| SEQ ID NO: 213 | |
| AAVS1- | AAACATCCTTAGGCCTCCTCCTTC |
| nickgRNA1-BOT | |
| SEQ ID NO: 214 | |
| AAVS1- | CACCGAAGGAGGAGGCCTAAGGAT |
| nickgRNA1-TOP | |
| SEQ ID NO: 215 | |
| CXCR4-pegS1-BOT | CTCTAAAACCCTCTTTGTCATCACGCTTC |
| SEQ ID NO: 216 | |
| CXCR4-pegS1-TOP | CACCGAAGCGTGATGACAAAGAGGGTTTT |
| SEQ ID NO: 217 | |
| CXCR4-+3G > A- | AAAAAGCGTGATGACAAAGAGGAGATCGGCCACTGACA |
| pegT-BOT | |
| SEQ ID NO: 218 | |
| CXCR4-+3G > A- | GTGCTGTCAGTGGCCGATCTCCTCTTTGTCATCACGCT |
| pegT-TOP | |
| SEQ ID NO: 219 | |
| CXCR4- | AAACGTACTTGTCCGTCATGCTTC |
| nickgRNA1-BOT | |
| SEQ ID NO: 220 | |
| CXCR4- | CACCGAAGCATGACGGACAAGTAC |
| nickgRNA1-TOP | |
| SEQ ID NO: 221 | |
| CXCR4-pegS2-BOT | CTCTAAAACCTGACAGGTGCAGCCTGTAC |
| SEQ ID NO: 222 | |
| CXCR4-pegS2-TOP | CACCGTACAGGCTGCACCTGTCAGGTTTT |
| SEQ ID NO 223 | |
| CXCR4--3sub3- | AAAACAGGCTGCACCTGTCAGTGGCCGACCTGCTAGCAAGCTT |
| pegT-BOT SEQ | CTTT |
| ID NO: 224 | |
| CXCR4--3sub3- | GTGCAAAGAAGCTTGCTAGCAGGTCGGCCACTGACAGGTGCA |
| pegT-TOP | GCCTG |
| SEQ ID NO: 225 | |
| CXCR4- | AAACTGTCATCTACACAGTCAACC |
| nickgRNA2-BOT | |
| SEQ ID NO: 226 | |
| CXCR4- | CACCGGTTGACTGTGTAGATGACA |
| nickgRNA2-TOP | |
| SEQ ID NO: 227 | |
| EMX1-pegS1-BOT | CTCTAAAACCCCTAGTCATTGGAGGTGAC |
| SEQ ID NO: 228 | |
| EMX1-pegS1-TOP | CACCGTCACCTCCAATGACTAGGGGTTTT |
| SEQ ID NO: 229 | |
| EMX1--1de1G- | AAAACACCTCCAATGACTAGGTGGGCAACCACAAACCC |
| pegT-BOT | |
| SEQ ID NO: 230 | |
| EMX1--1de1G- | GTGCGGGTTTGTGGTTGCCCACCTAGTCATTGGAGGTG |
| pegT-TOP | |
| SEQ ID NO: 231 | |
| EMX1- | AAACCGAGGGCAGAGTGCTGCTTGC |
| nickgRNA1-BOT | |
| SEQ ID NO: 232 | |
| EMX1- | CACCGCAAGCAGCACTCTGCCCTCG |
| nickgRNA1-TOP | |
| SEQ ID NO: 233 | |
| GFP-pegS1-BOT | CTCTAAAACACGGCGTGCAGTGCTTCAGC |
| SEQ ID NO: 234 | |
| GFP-pegS1-TOP | CACCGCTGAAGCACTGCACGCCGTGTTTT |
| SEQ ID NO: 235 | |
| GFP- | AAAAGCACTGCACGCCGTGGCTCAGGGTGGTCACGA |
| +1AGG > GGC- | |
| pegT-BOT | |
| SEQ ID NO: 236 | |
| GFP- | GTGCTCGTGACCACCCTGAGCCACGGCGTGCAGTGC |
| +1AGG > GGC- | |
| pegT-TOP | |
| SEQ ID NO: 237 | |
| GFP-nickgRNA1-BOT | AAACTAGGTGGCATCGCCCTCGCC |
| SEQ ID NO: 238 | |
| GFP-nickgRNA1-TOP | CACCGGCGAGGGCGATGCCACCTA |
| SEQ ID NO: 239 | |
| KRAS-pegS1-BOT | CTCTAAAACCGATTATTATCAGCCTCAGC |
| SEQ ID NO: 240 | |
| KRAS-pegS1-TOP | CACCGCTGAGGCTGATAATAATCGGTTTT |
| SEQ ID NO: 241 | |
| KRAS--6sub6- | AAAATGAGGCTGATAATAAGCTTGCTAGCCGGCGATCAGACA |
| pegT-BOT | GCCCCGGT |
| SEQ ID NO: 242 | |
| KRAS--6sub6- | GTGCACCGGGGCTGTCTGATCGCCGGCTAGCAAGCTTATTATC |
| pegT-TOP | AGCCTCA |
| SEQ ID NO: 243 | |
| KRAS-- | AAAAAGGCTGATAATAATCTAGGCGGCGATCAGACA |
| 1GG > TA-pegT-BOT | |
| SEQ ID NO: 244 | |
| KRAS-- | GTGCTGTCTGATCGCCGCCTAGATTATTATCAGCCT |
| 1GG > TA-pegT-TOP | |
| SEQ ID NO: 245 | |
| KRAS- | AAACCGGTGTGGGAAATCGTCCGC |
| nickgRNA1-BOT | |
| SEQ ID NO: 246 | |
| KRAS- | CACCGCGGACGATTTCCCACACCG |
| nickgRNA1-TOP | |
| SEQ ID NO: 247 | |
| MYC-pegS1-BOT | CTCTAAAACGCGTCGGGAGAGTCGCGTCC |
| SEQ ID NO: 248 | |
| MYC-pegS1-TOP | CACCGGACGCGACTCTCCCGACGCGTTTT |
| SEQ ID NO: 249 | |
| MYC--2insA- | AAAAACGCGACTCTCCCGACAGCGGGGAGGCTATTCTGC |
| pegT-BOT | |
| SEQ ID NO: 250 | |
| MYC--2insA- | GTGCGCAGAATAGCCTCCCCGCTGTCGGGAGAGTCGCGT |
| pegT-TOP | |
| SEQ ID NO: 251 | |
| MYC- | AAACGCTTCTCTGAAAGGCTCTCC |
| nickgRNA1-BOT | |
| SEQ ID NO: 252 | |
| MYC- | CACCGGAGAGCCTTTCAGAGAAGC |
| nickgRNA1-TOP | |
| SEQ ID NO: 253 | |
| STAT1-pegS1-BOT | CTCTAAAACCCAGCTGCAAGCATGTCATC |
| SEQ ID NO: 254 | |
| STAT1-pegS1-TOP | CACCGATGACATGCTTGCAGCTGGGTTTT |
| SEQ ID NO: 255 | |
| STAT1-- | AAAATGACATGCTTGCAGCTGTACGAAACAGGTTGTCCT |
| 1GGG > TAC- | |
| pegT-BOT | |
| SEQ ID NO: 256 | |
| STAT1-- | GTGCAGGACAACCTGTTTCGTACAGCTGCAAGCATGTCA |
| 1GGG > TAC- | |
| pegT-TOP | |
| SEQ ID NO: 257 | |
| STAT1- | AAACATCTTTGCTCCTGGCTGTCC |
| nickgRNA1-BOT | |
| SEQ ID NO: 258 | |
| STAT1- | CACCGGACAGCCAGGAGCAAAGAT |
| nickgRNA1-TOP | |
| SEQ ID NO: 259 | |
| TGFB1-pegS1-BOT | CTCTAAAACCTTGGTGGAAGCGCAGGCTC |
| SEQ ID NO: 260 | |
| TGFB1-pegS1-TOP | CACCGAGCCTGCGCTTCCACCAAGGTTTT |
| SEQ ID NO: 261 | |
| TGFB1-+3delGC- | AAAAGCCTGCGCTTCCACCAAGGGAGGAGGACCCCGCT |
| pegT-BOT | |
| SEQ ID NO: 262 | |
| TGFB1-+3delGC- | GTGCAGCGGGGTCCTCCTCCCTTGGTGGAAGCGCAGGC |
| pegT-TOP | |
| SEQ ID NO: 263 | |
| TGFB1- | AAACCTGCGCTAAACGCTGGCAGTC |
| nickgRNA1-BOT | |
| SEQ ID NO: 264 | |
| TGFB1- | CACCGACTGCCAGCGTTTAGCGCAG |
| nickgRNA1-TOP | |
| SEQ ID NO: 265 | |

### EXAMPLES

While several experimental Examples are contemplated, these Examples are intended to be non-limiting.

### Example 1. Examination of Competition Between Precise and Semi-Random Editing Pathways

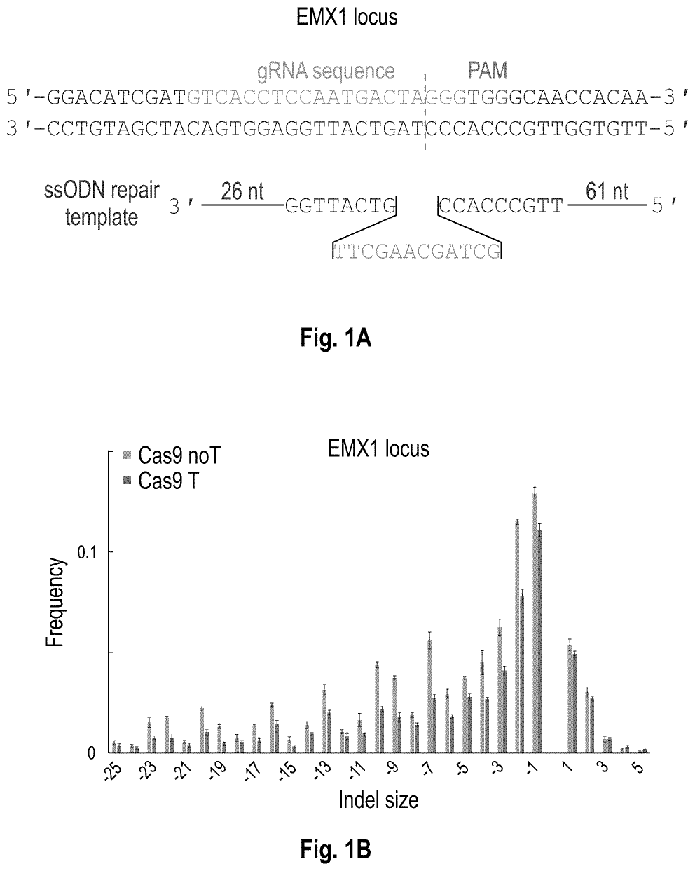

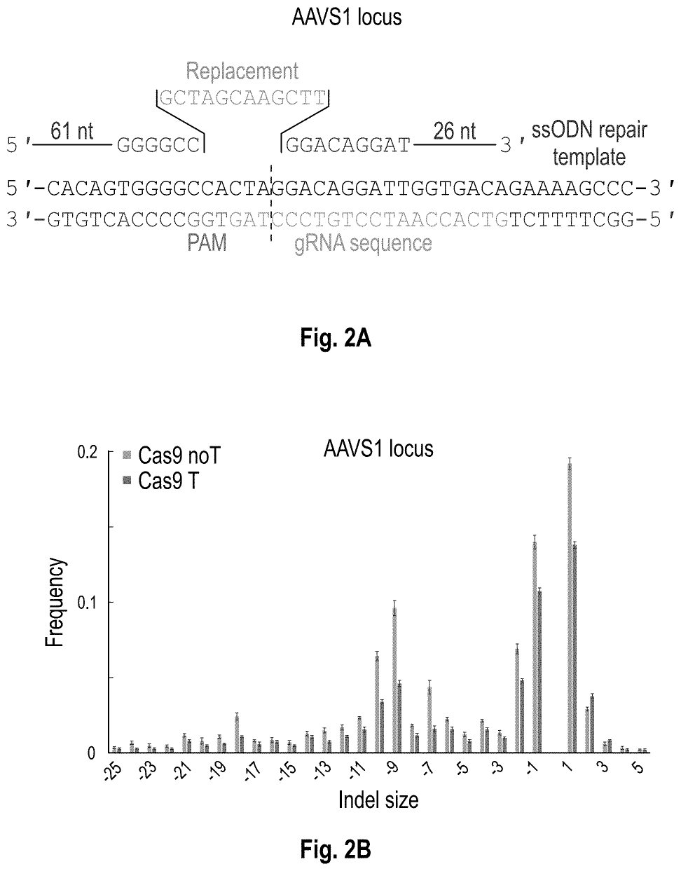

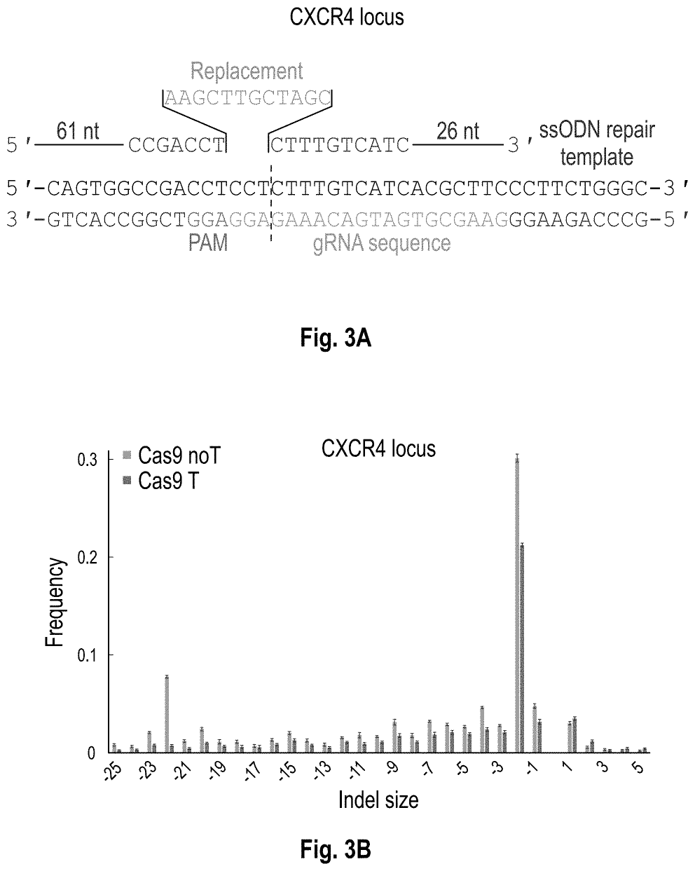

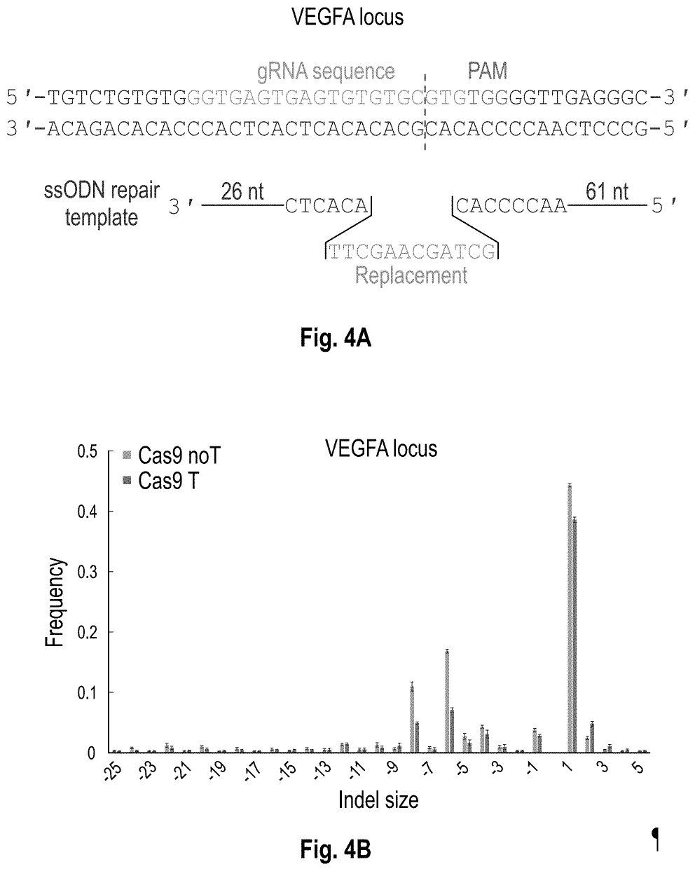

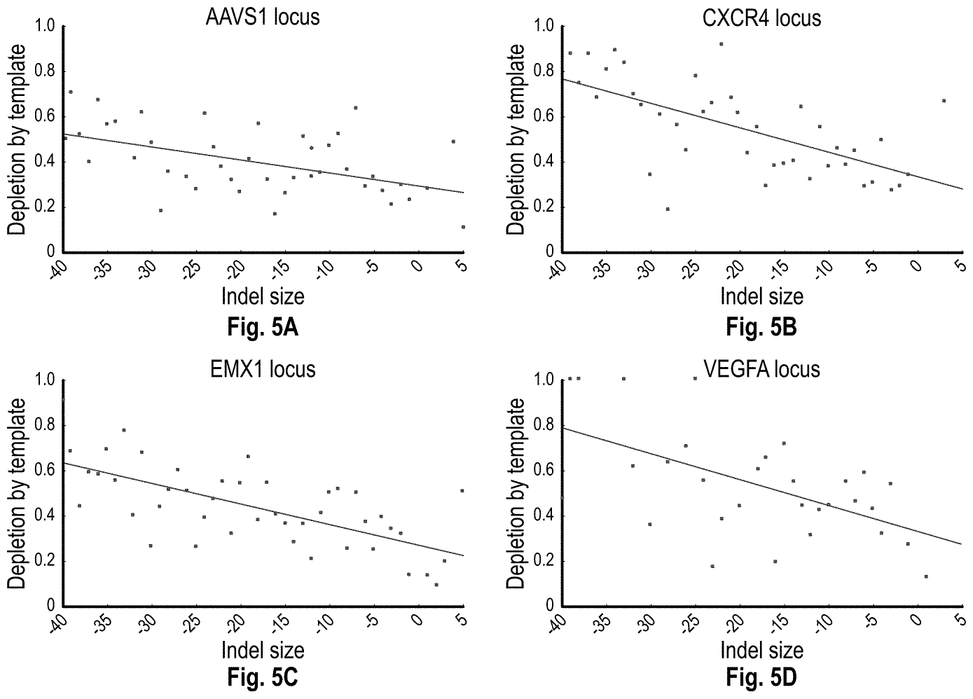

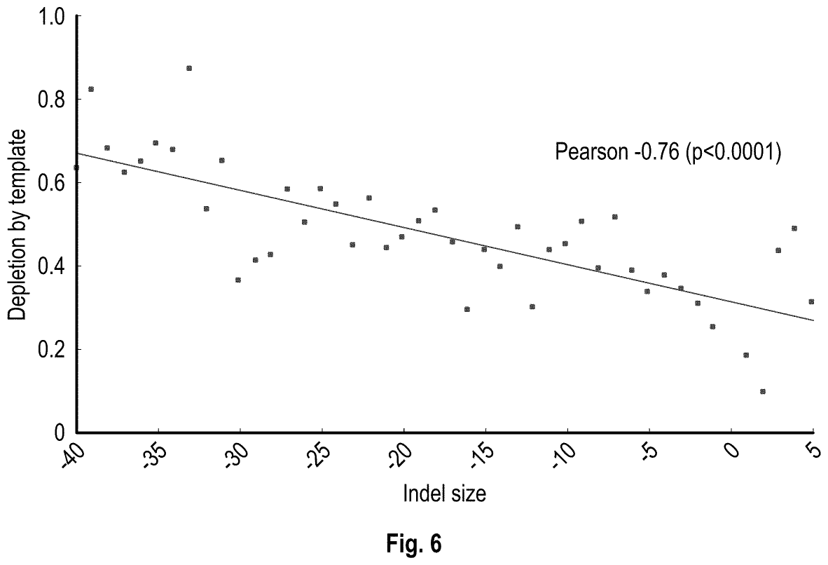

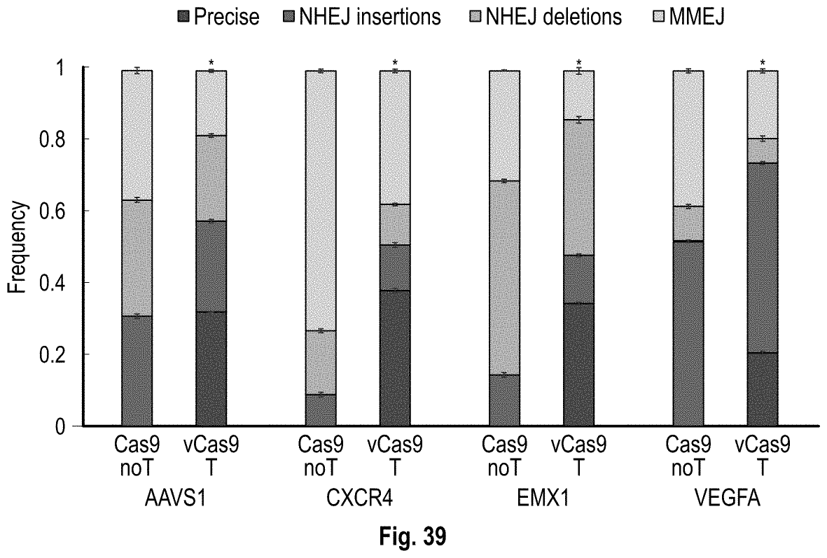

To examine the balance between precise homology related repair and semi-random indel repair, indel size and frequency were examined in HEK293T cells that were exposed to a Cas9 from Streptococcus pyogenes (SEQ ID NO: 1) in the presence or absence of a single-stranded oligodeoxynucleotide (ssODN) for use in HDR. gRNAs in the presence or absence of ssODN HDR templates were targeted to the EMX1 locus (FIG. 1A; FIG. 39), the AAVS1 locus (FIG. 2A), the CXCR4 locus (FIG. 3A), and the VEGFA locus (FIG. 4A). Smaller indels were observed more frequently (FIG. 1B, FIG. 2B, FIG. 3B, FIG. 4B). As indel size increased, the depletion of indels in favor of HDR from the template also increased (FIG. 5). When all 4 loci were averaged together, the depletion of indels by HDR from the templates was shown to increase as indel size increased (FIG. 6).

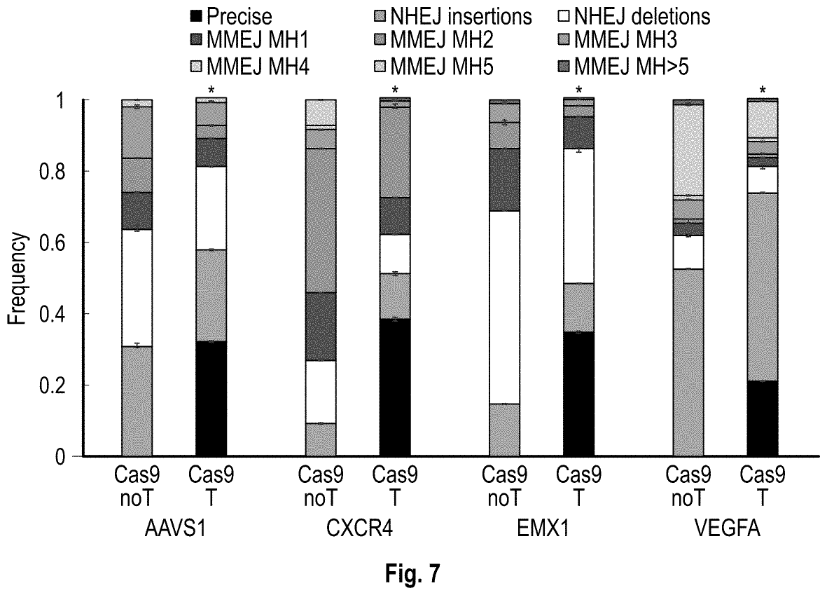

The specific repair pathway engaged in the presence or absence of repair template was also examined. In HEK293T cells in which no repair template was supplied, there was no precise editing by HDR. In three of four loci examined without repair template, the majority of DNA repair was the result of NHEJ. The remainder is the result of MMEJ of varying lengths of microhomology (FIG. 7). When supplied with a repair template, a significant portion of the DNA repair was attributed to precise editing as a result of HDR. When HDR was engaged, cells were less likely to engage deletions resulting from the MMEJ pathway or small deletions as a result of NHEJ. Insertions as a result of NHEJ were not affected when HDR was engaged.

### Example 2. Engaging MMEJ Repair by Increasing Indel Size

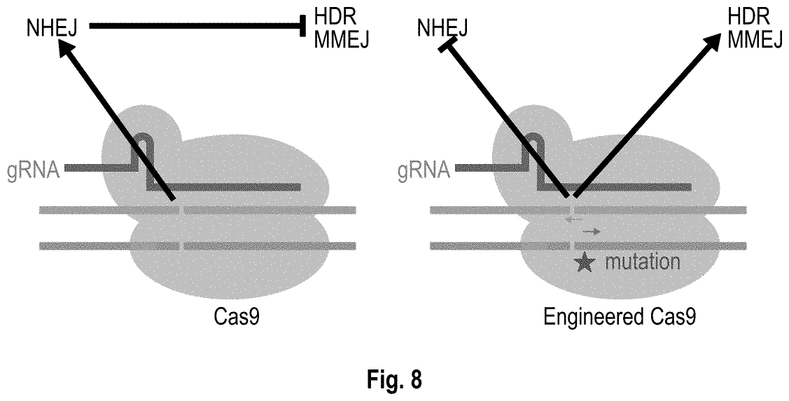

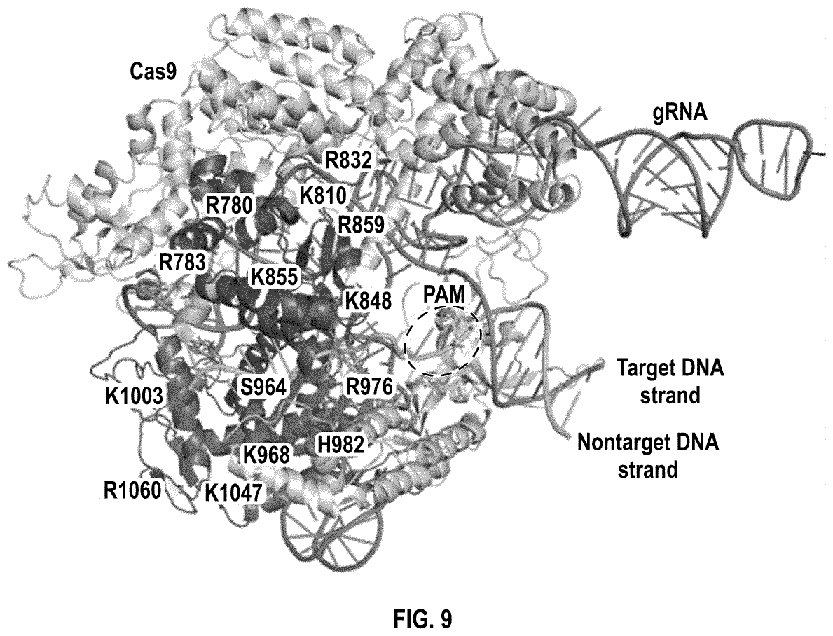

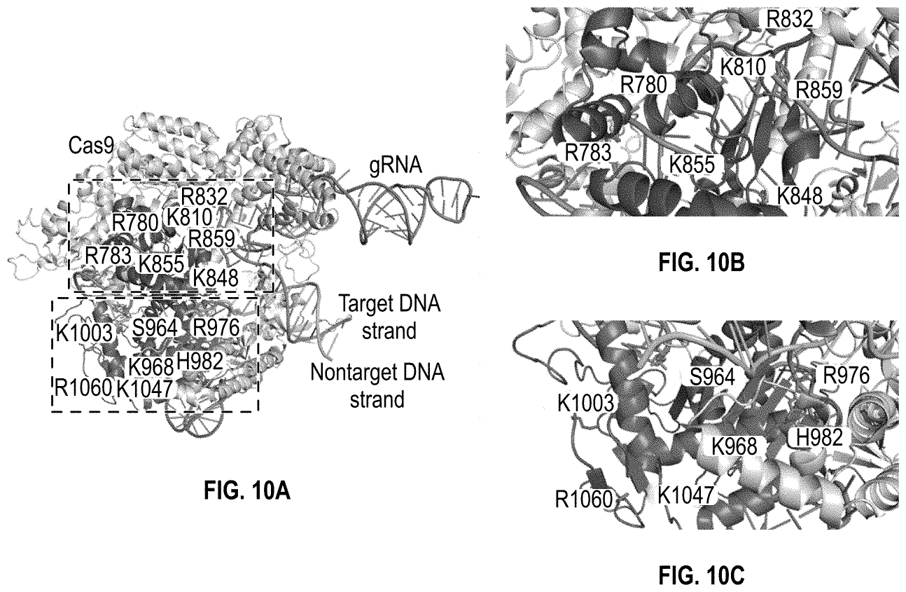

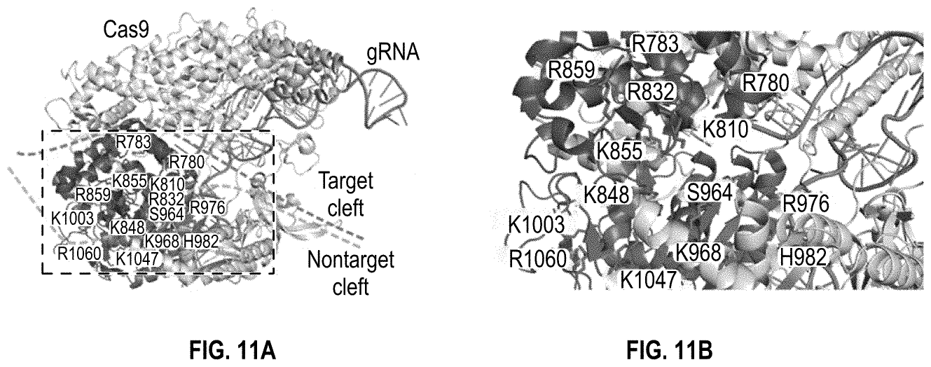

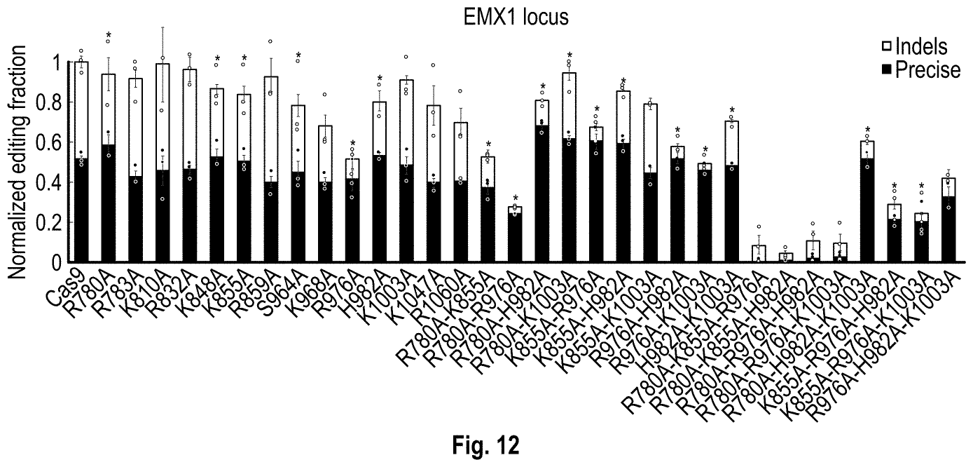

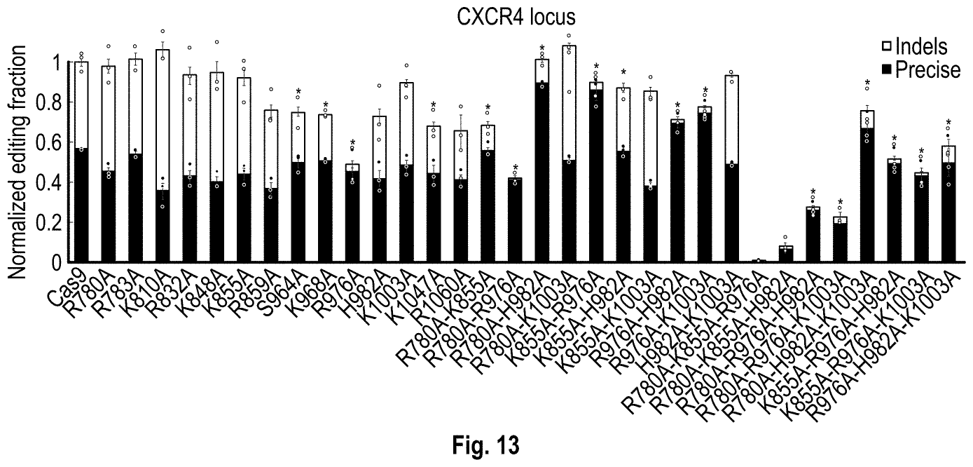

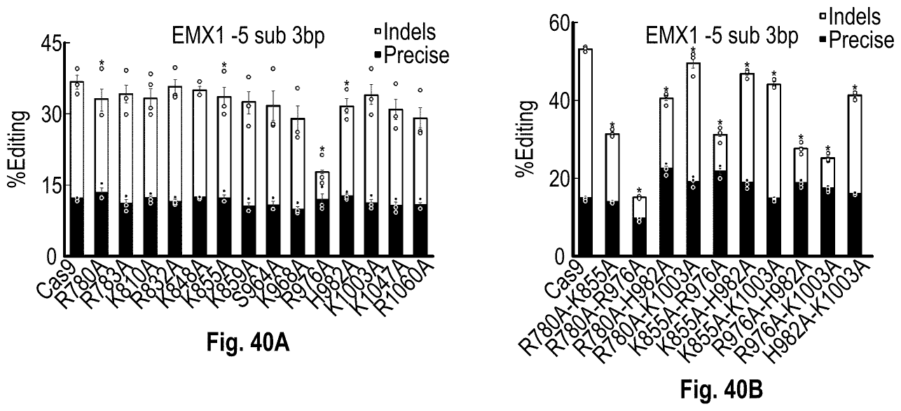

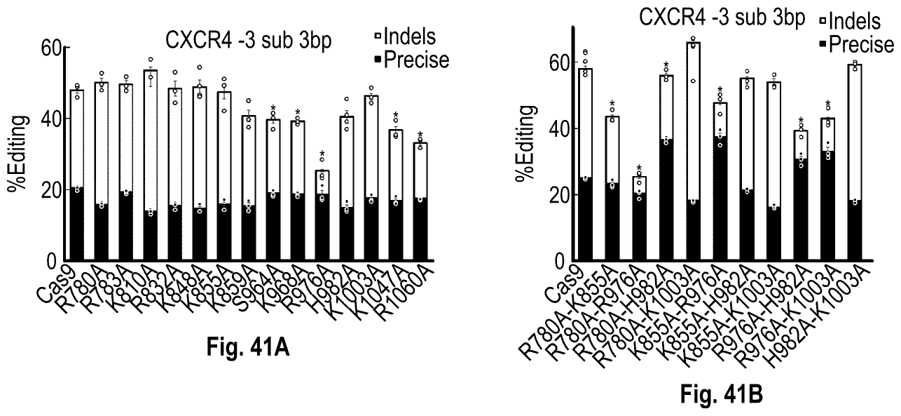

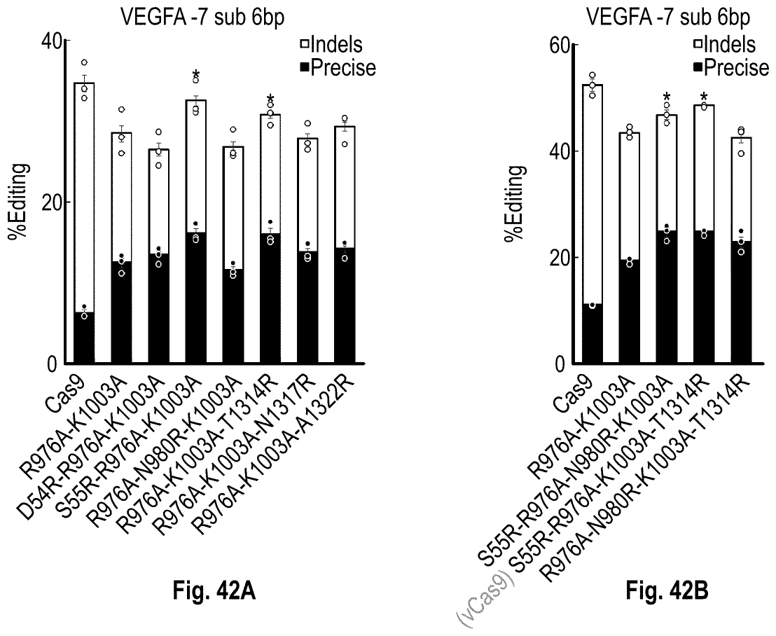

MMEJ repair was competitively inhibited to a greater degree than NHEJ when HDR was engaged using a template. It was hypothesized that if MMEJ was promoted over NHEJ, a greater proportion of HDR template would be utilized in DNA repair. Although no known Cas9 variants alter the frequency of DNA repair pathways, different Cas9 structures are known to produce different double strand break structures upon cutting of the DNA (FIG. 8). Additionally, double strand break structure is known to be able to influence DNA repair mechanisms. Alterations were therefore made to the DNA binding cleft of S. pyogenes Cas9 to change the structure of the double stranded breaks. Fourteen basic or polar residues were mutated to alanine to reduce putative DNA interactions (FIGS. 9-11; R780, R783, K810, R832, R859, K848, K855, 5964, K968, R976, H982, K1003, K1047, and R1060). Following these alterations, HEK293T cells were supplied with gRNAs and short HDR templates with regions homologous to either the EMX1, CXCR4, or VEGFA locus (FIG. 1A, FIG. 3A, FIG. 4A). Engagement of varying DNA repair pathways was examined by Sanger sequencing using TIDE analysis. Single mutants, double mutants, and triple mutants of the DNA binding cleft of Cas9 were examined for either precise HDR editing (determined by the presence of the HDR template) or semi-random indels. Several of these mutants exhibited a significant increase in the frequency of precise editing as compared to the semi-random indels indicative of NHEJ at both the EMX1 locus (FIG. 12; FIG. 40A, B), the CXCR4 locus (FIG. 13), or the VEGFA locus (FIG. 41A, B).

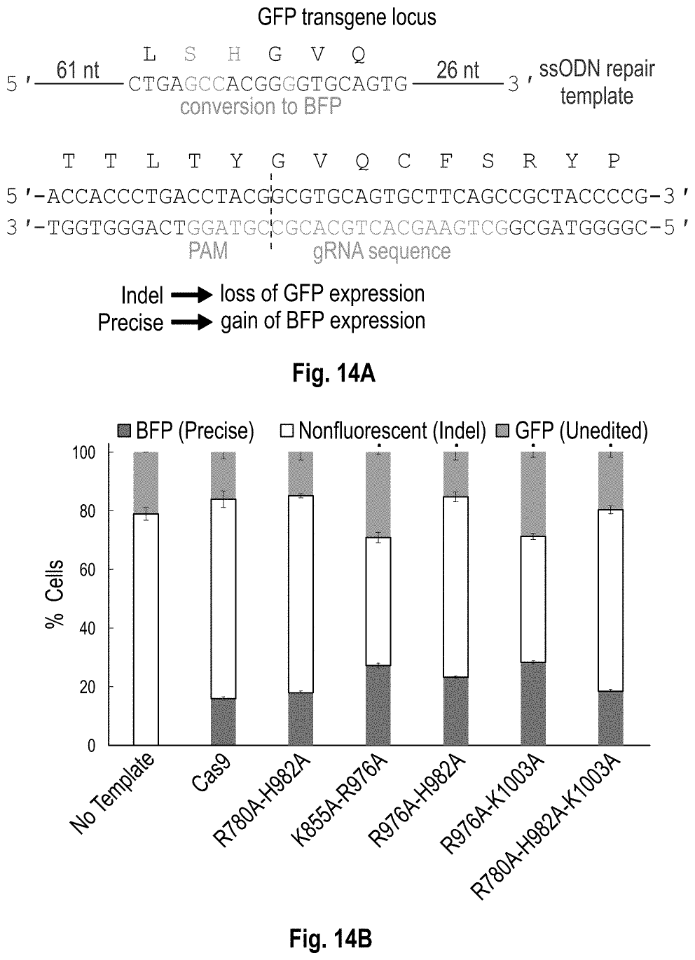

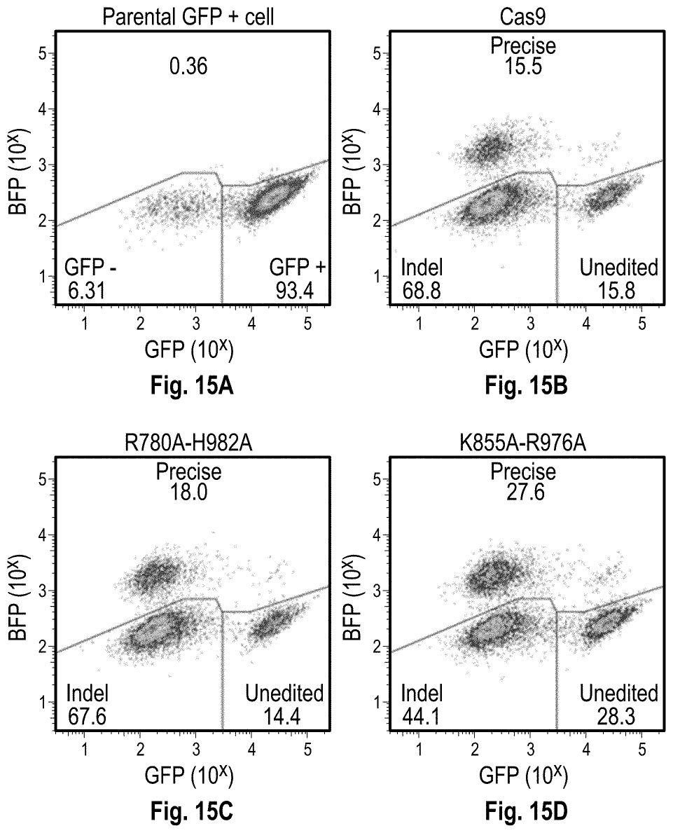

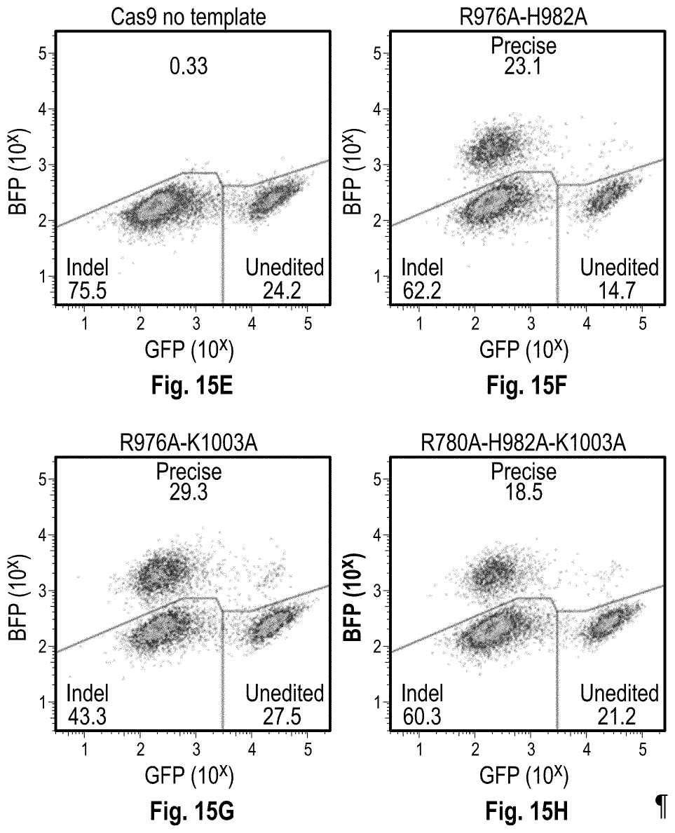

To further examine these Cas9 variants in their editing capability, a GFP transgene was inserted into the genomes of HEK293T cells. An HDR ssODN template was supplied to the cells that, if inserted, would convert the GFP transgene to a blue fluorescent protein (BFP) (FIG. 14A). In contrast, if NHEJ was engaged the resulting indel would lead to a loss of GFP expression. HEK293T cells not supplied with HDR template acted as a control. Fluorescence was measured by flow cytometry to determine the expression GFP or BFP. Introduction of Cas9 from Streptococcus pyogenes (SEQ ID NO: 1) led to precise editing of the GFP transgene to BFP, indicated by blue fluorescence (FIG. 14B). When compared to Cas9 from Streptococcus pyogenes (SEQ ID NO: 1), each of the tested double and triple mutants exhibited an increase in blue fluorescence (FIG. 14B, FIG. 15), indicating an increased engagement of the HDR pathway and precise editing. However, although these mutants were capable of shifting the frequency of DNA repair pathways the cell engages, they exhibited an overall decrease in editing activity (See FIGS. 12-15).

### Example 3. Increasing Editing Activity in HDR-Preferring Cas9 Variants

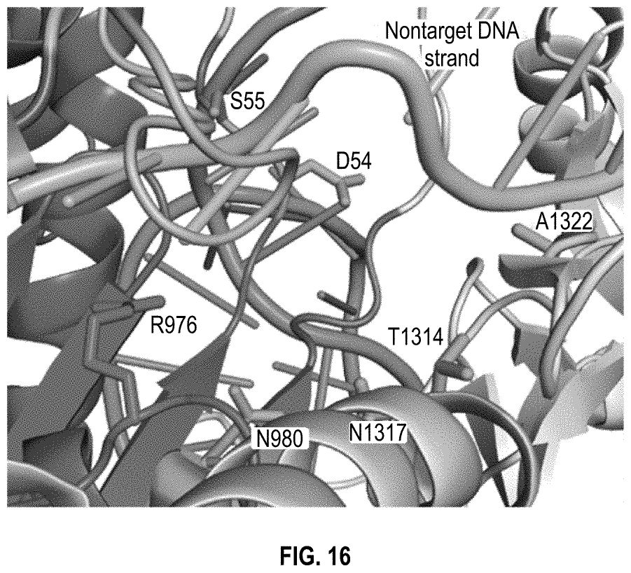

The R976A mutation produced the greatest change in repair pathway frequency along with the largest drop in total editing activity and sits at one end of the nontarget cleft near where the substrate DNA strands separate (FIG. 16). It was hypothesized therefore that R976 might regulate repair pathway frequency by affecting how the nontarget strand sits in its cleft and where it is cut, while also controlling total activity by aiding in substrate strand separation prior to cutting. To test whether mutating residues nearby A976 to Arginine could rescue activity without reverting changes to repair pathway frequency, six residues in the R976A-K1003A variant were mutated to produce a set of triple-, quadruple-, and quintuple-mutants (see Table 6; FIG. 16).

TABLE 7
Arginine Mutations to Test For Increased Activity

| SEQ ID NO: | Mutations |
|---|---|
| SEQ ID NO: 4 | R976A |
| | K1003A |
| SEQ ID NO: 5 | D54R |
| | R976A |
| | K1003A |
| SEQ ID NO: 6 | S55R |
| | R976A |
| | K1003A |
| SEQ ID NO: 7 | R976A |
| | N980R |
| | K1003A |
| SEQ ID NO: 8 | R976A |
| | K1003A |
| | T1314R |
| SEQ ID NO: 9 | R976A |
| | K1003A |
| | N1317R |
| SEQ ID NO: 10 | R976A |
| | K1003A |
| | A1322R |
| SEQ ID NO: 11 | S55R |
| | R976A |
| | N980R |
| | K1003A |
| SEQ ID NO: 3 | S55R |
| | R976A |
| | K1003A |
| | T1314R |
| SEQ ID NO: 12 | R976A |
| | N980R |
| | K1003A |
| | T1314R |
| SEQ ID NO: 13 | S55R |
| | N976A |
| | N980R |
| | K1003A |
| | T1314R |

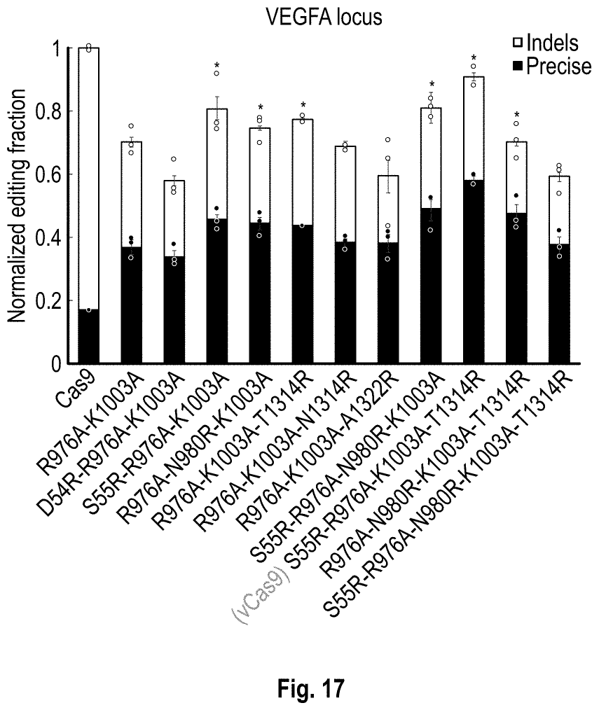

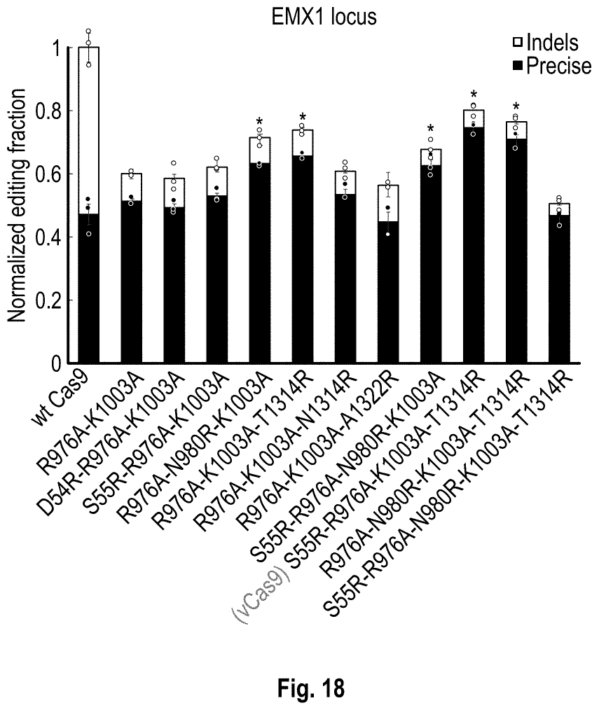

These triple, quadruple, and quintuple mutants were analyzed in HEK293T cells with gRNAs and HDR templates (FIG. 1A, FIG. 4A) for engagement of either precise HDR editing or engagement of DNA repair resulting in indels as measured by Sanger sequencing. When compared to Cas9 from Streptococcus pyogenes (SEQ ID NO: 1), each of these multiple mutants displayed an increase in the fraction of editing that was indicative of precise editing. When compared to the base double mutant (R976A-K1003A), 5/10 mutants (50%) displayed increased activity at the VEGFA locus (FIG. 17), and 7/10 (70%) displayed increased activity at the EMX1A locus (FIG. 18). The mechanisms regulating the locus specific interaction with Cas9 mutants that control increased activity at a certain locus remain unclear. However, 5 of these mutants exhibited an increase in activity in both loci (S55R-R976A-K1003A; R976A-N980R-K1003A; R976A-K1003A-T1314R; S55R-R976A-N980R-K1003A; S55R-R976A-K1003A-T1314R). The S55R-R976A-K1003A-T1314R quadruple mutant exhibited the most activity and was subsequently named vCas9.

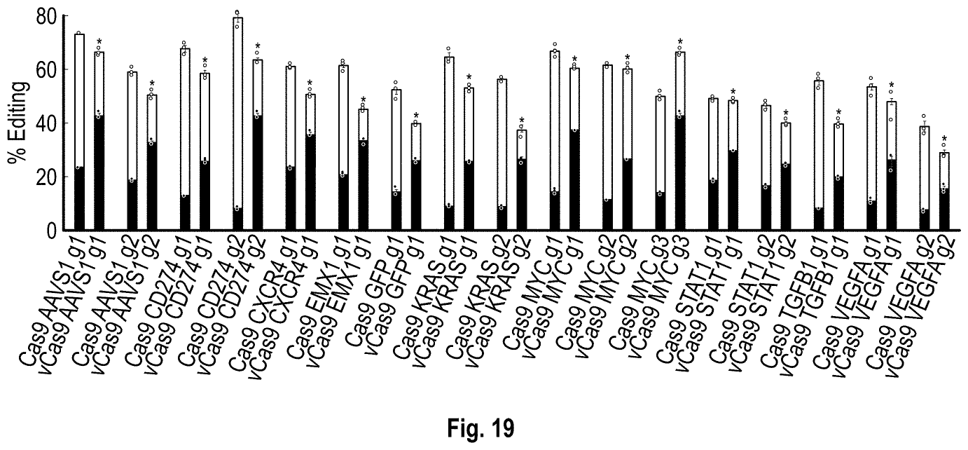

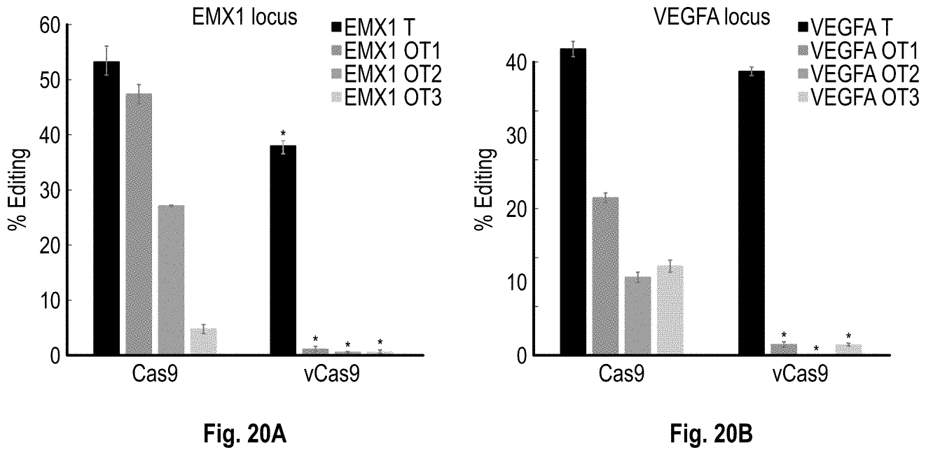

vCas9 activity across numerous loci was compared to Cas9 from Streptococcus pyogenes (SEQ ID NO: 1). Whereas Cas9 demonstrated precise editing frequencies of 9.9-37.5% (mean 24.1%), vCas9 increased these to 43.3-73.7% (mean 58.3%), corresponding to a 1.4- to 2.8-fold (mean 1.9-fold) suppression of indel frequency. Further highlighting the unique nature of locus-specific activity issues, the editing percentage varied greatly across the tested loci. Compared to Cas9, vCas9 exhibited and increased fraction of editing attributed to precise HDR editing, and a decreased percentage of indels (FIG. 19). vCas9 did not increase non-specific genome activity but surprisingly improved on-target versus off-target editing specificity compared to Cas9 from Streptococcus pyogenes (SEQ ID NO: 1) (FIG. 20).

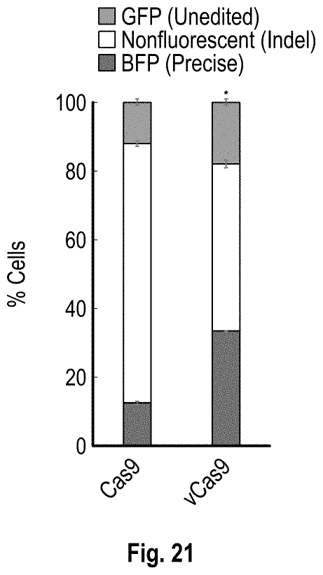

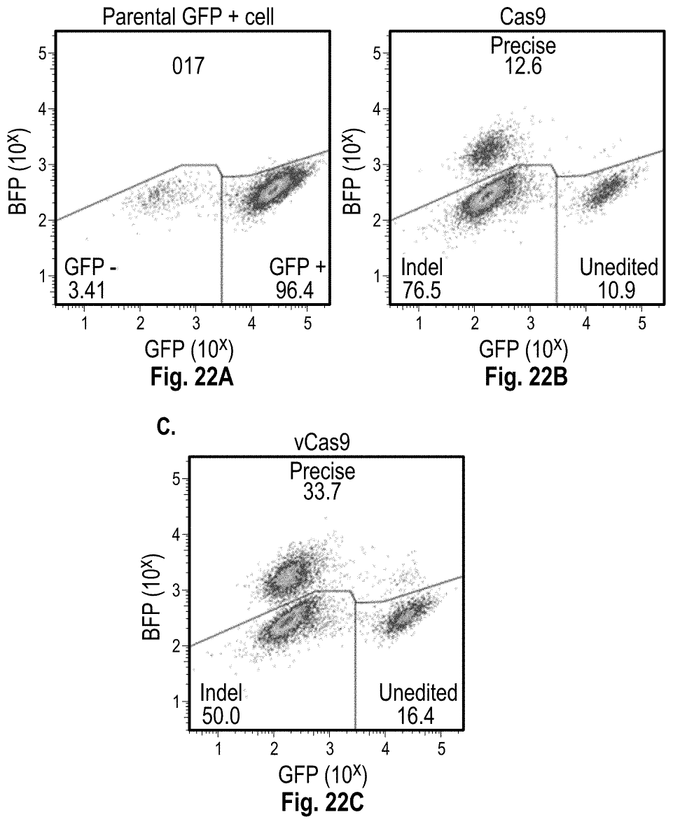

When vCas9 and Cas9 from Streptococcus pyogenes (SEQ ID NO: 1) were tested for their ability to precisely edit a GFP transgene to BFP (as described above), vCas9 exhibited a significant increase in the level of blue fluorescence observed by flow cytometry, indicating a significant increase in precise HDR editing (FIGS. 21, 22A-C). Indeed, vCas9 greatly increased precise gene conversion while suppressing indel frequency.

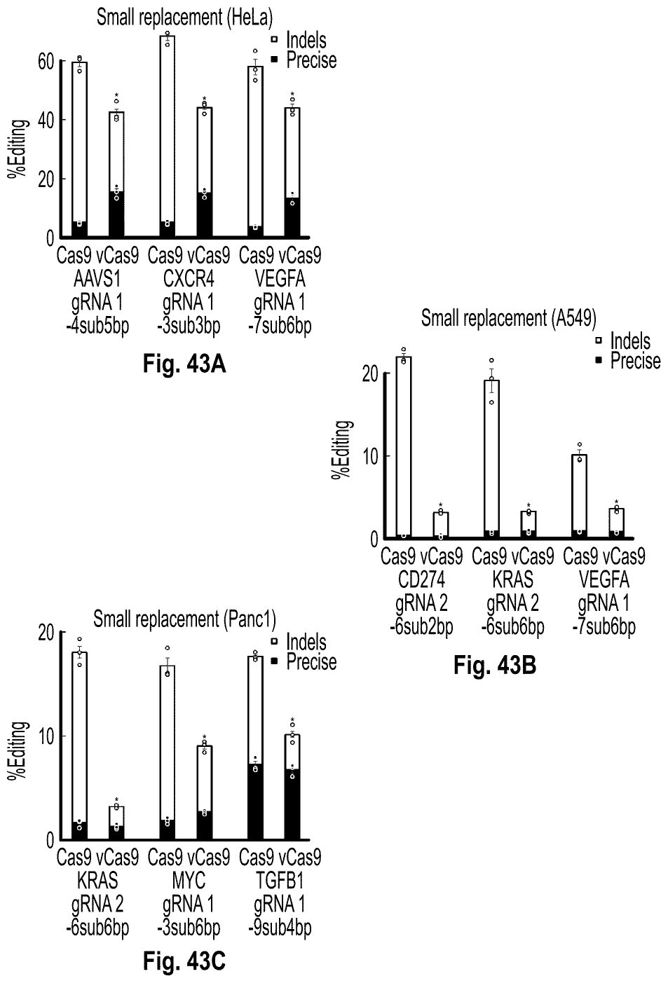

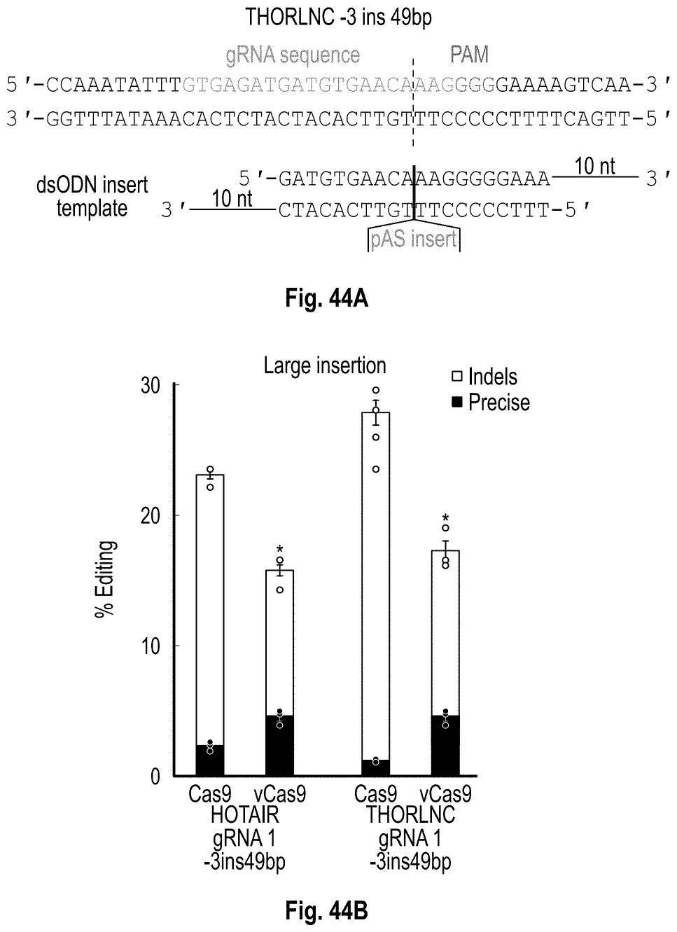

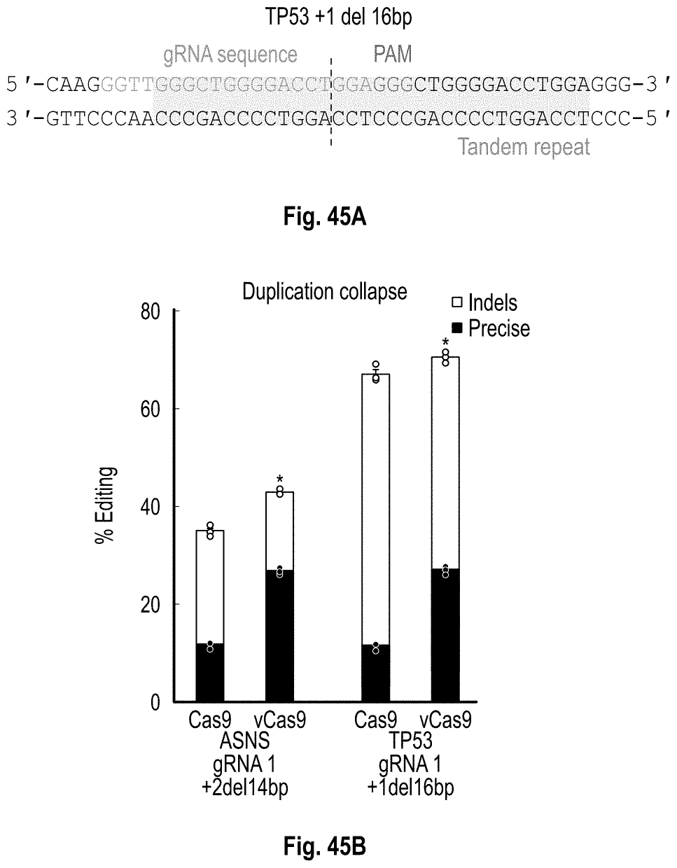

We also examined the cell-type dependence of precise editing of vCas9 compared to unmutated Cas9. We applied similar HDR templates to produce small edits at several loci in HeLa, A549, and Panc1 cells. In each cell model and locus, vCas9 consistently altered precise editing and indel frequencies (FIG. 43A-C). We further tested the efficiency of vCas9 to perform two other precise edit types of interest: large insertions from double-stranded templates (FIG. 44A) and untemplated, precise collapse of duplications (FIG. 45A). For both edit types, vCas9 similarly favored precise editing and suppressed indels (FIG. 44B, FIG. 45B).

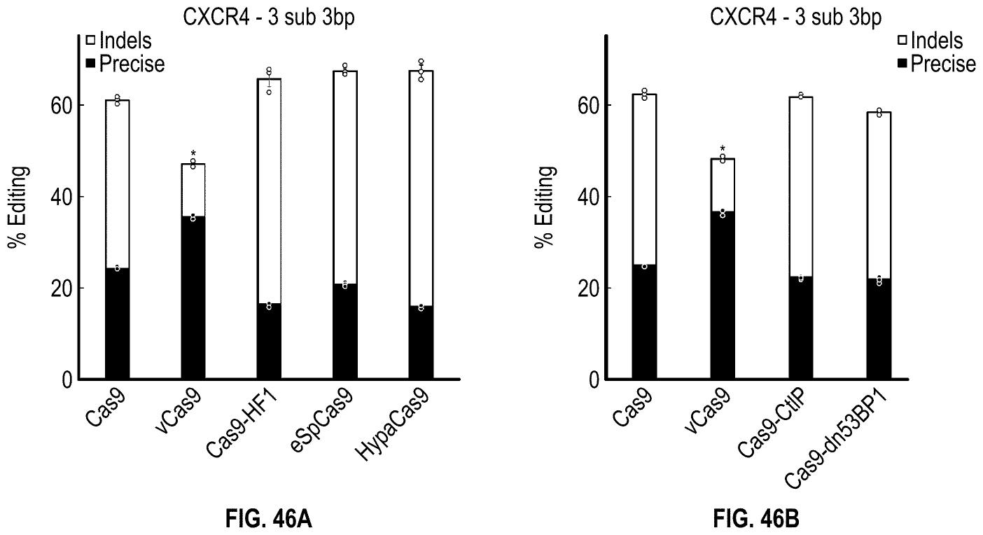

Finally, we also compared other engineered Cas9 variants and fusions and found that repair outcomes for vCas9 are far more biased toward precise editing (FIG. 46). These data indicate that vCas9 is capable of robustly suppressing indels and promoting precise editing across varied loci, cells, and edit types.

### Example 4. Effect of vCas9 on Frequency of DNA Repair Pathway Engagement

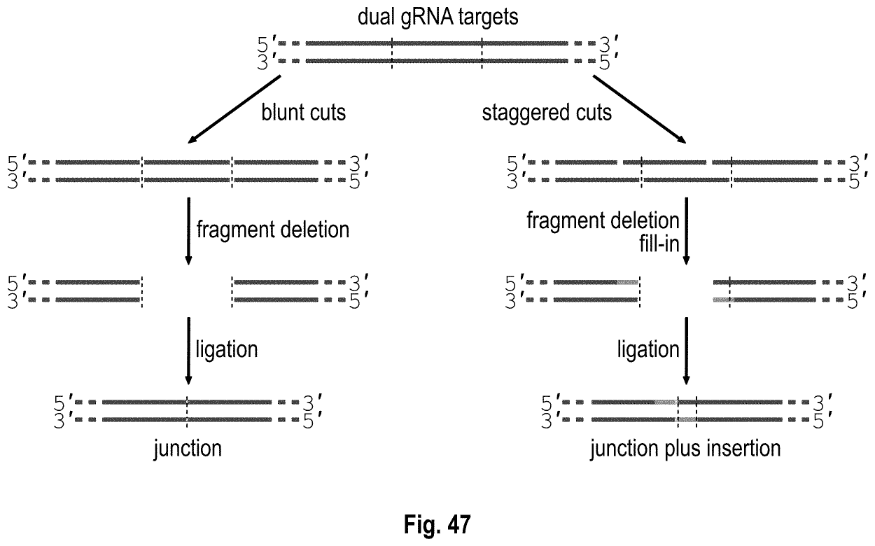

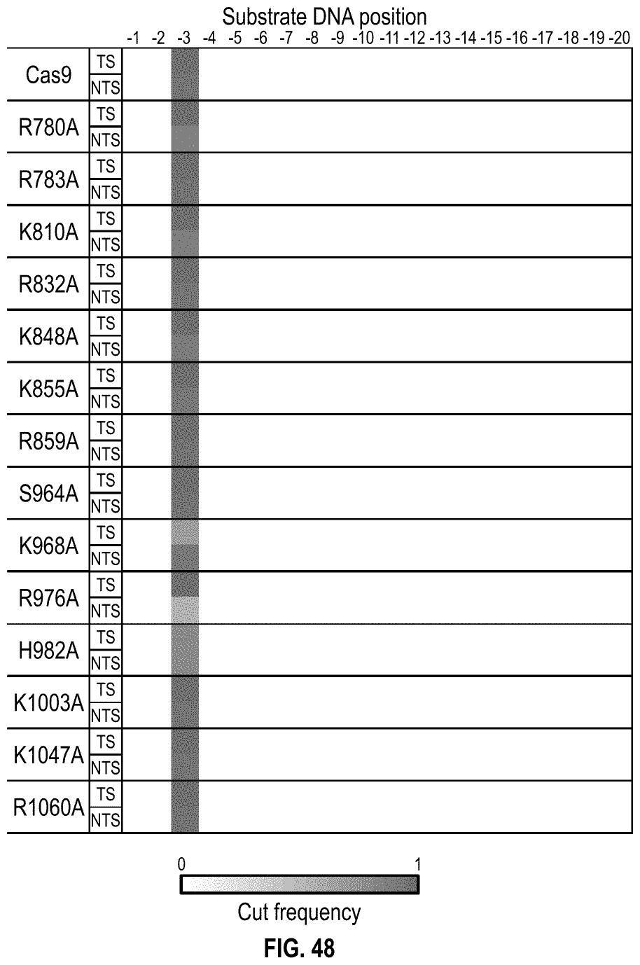

We first studied the effect of Cas variants on DNA break structure and the downstream effect of regulation of repair pathway outcomes. Break structures in cells were assessed by creating two concurrent DSBs and analyzing junctions of the DNA ends for sequences resulting from blunt versus staggered cutting (FIG. 47). While wild-type Cas9 almost uniformly produced blunt cuts, several Cas9 variants induced staggered cuts from 2-6 bp (FIG. 48).

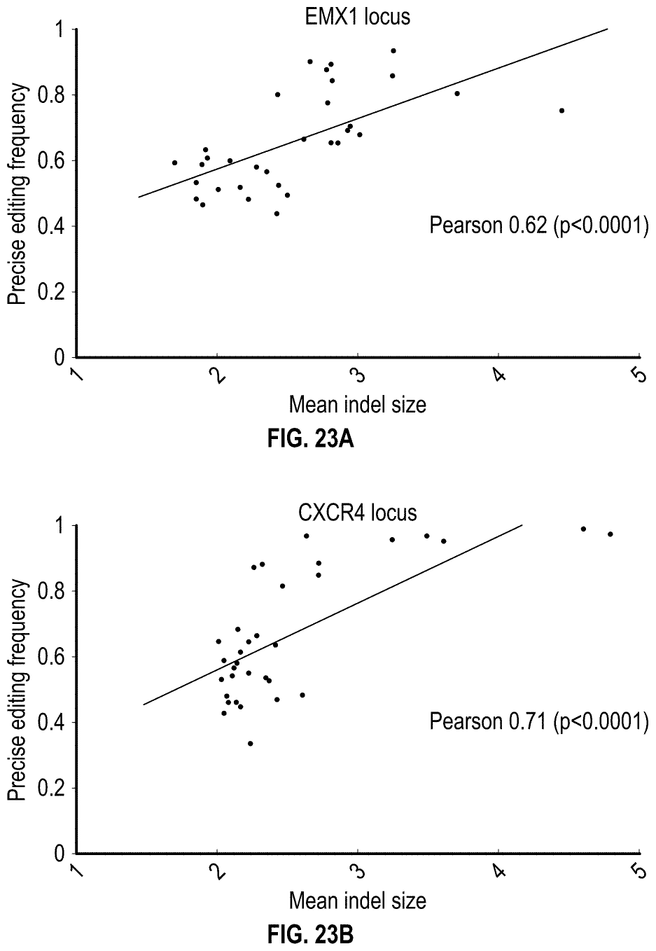

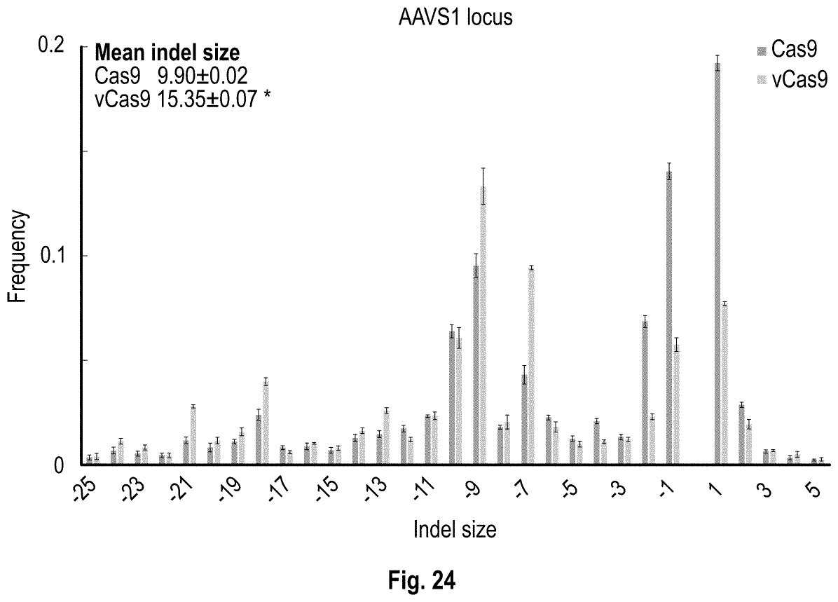

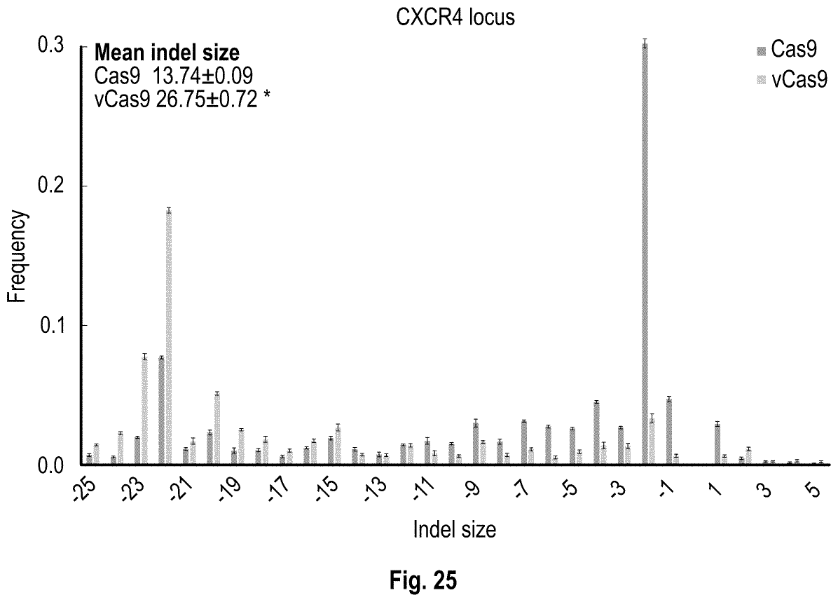

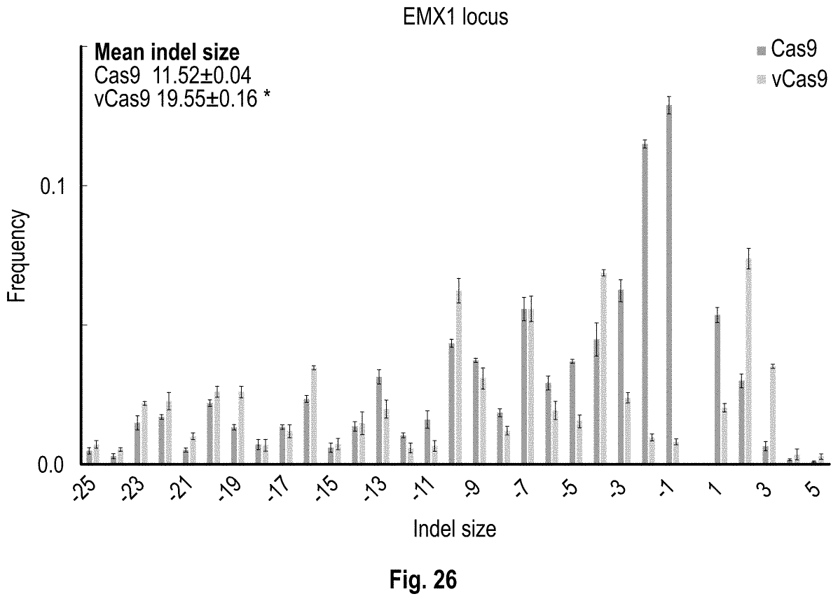

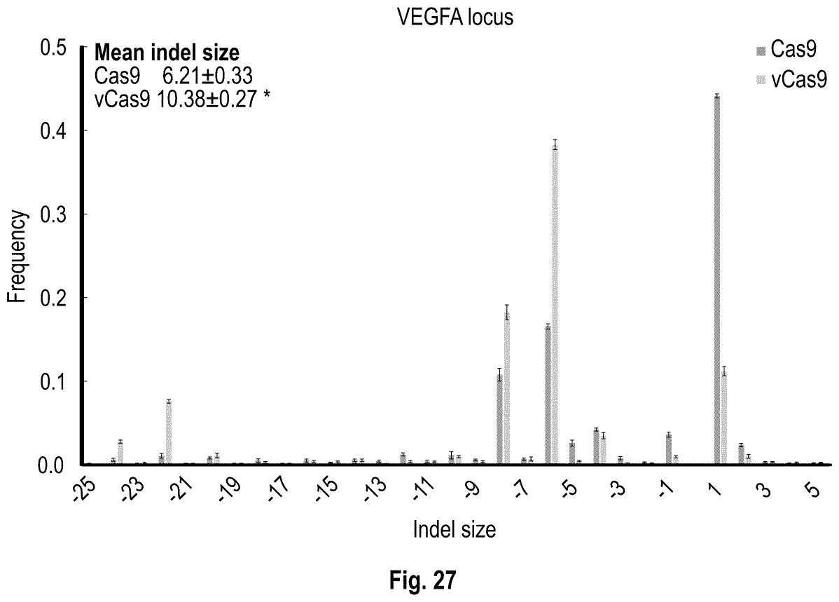

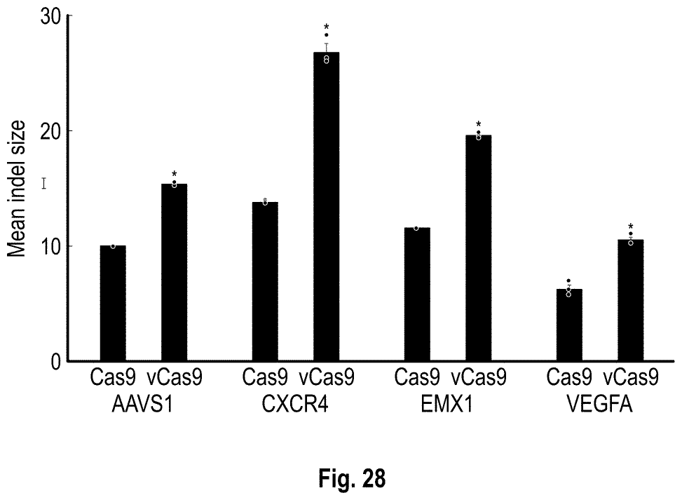

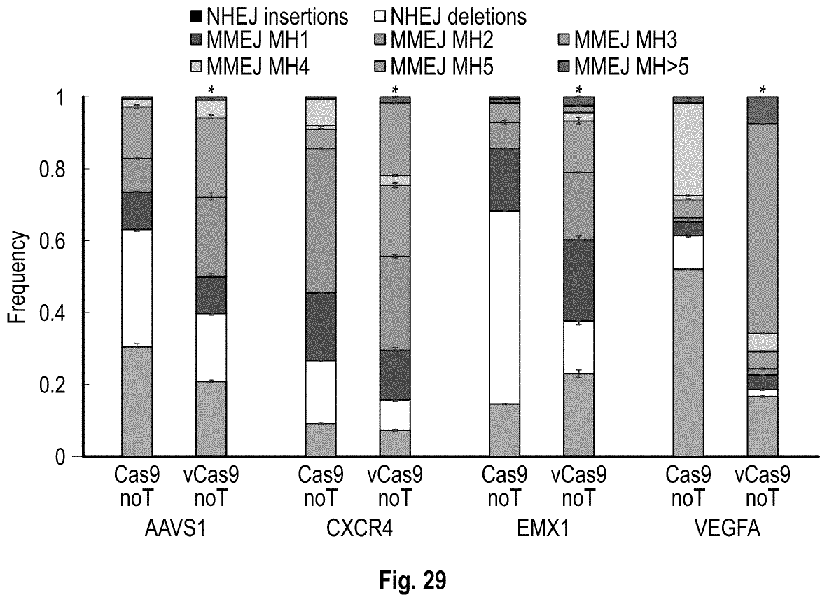

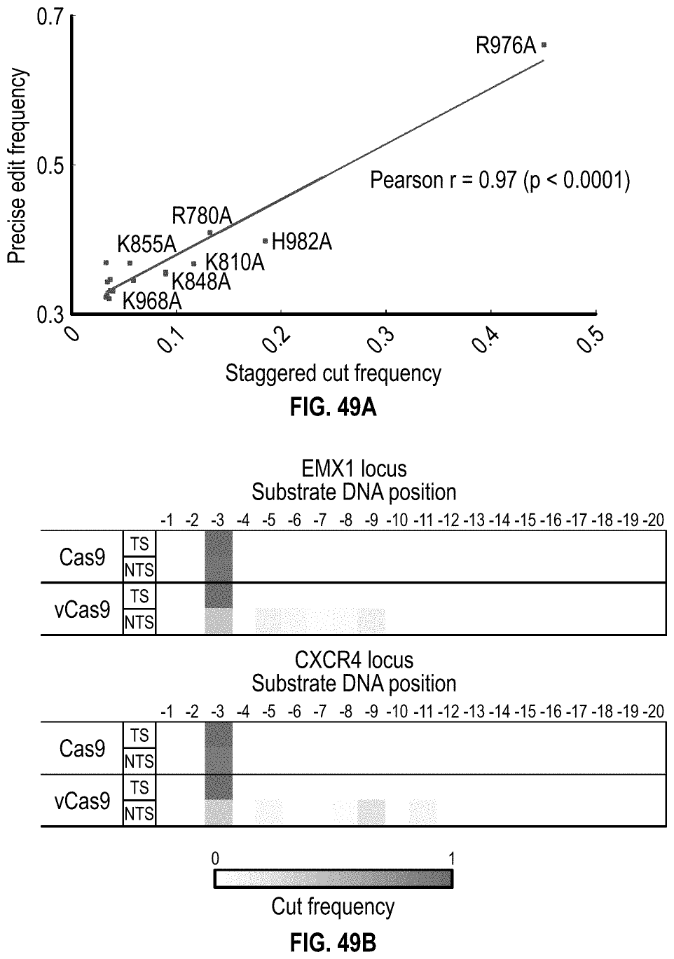

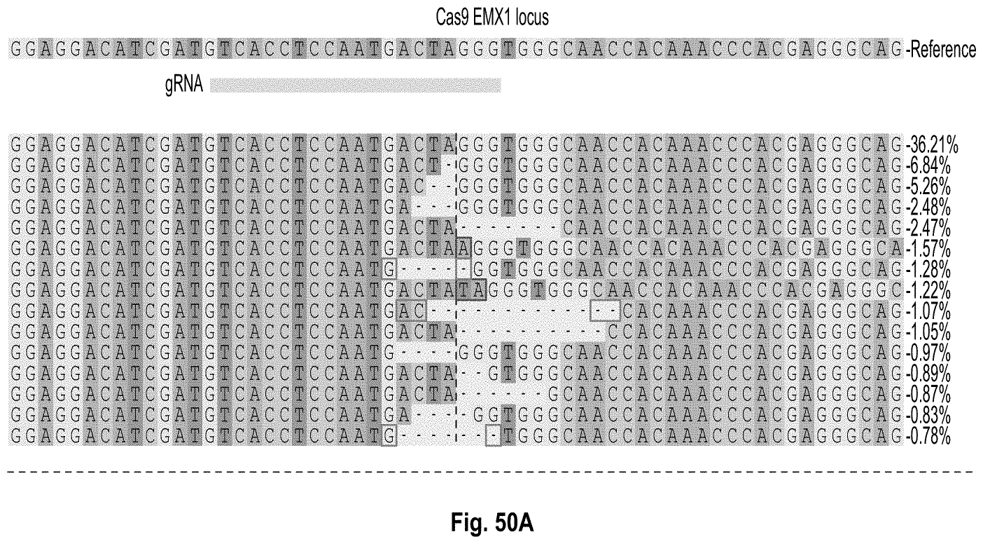

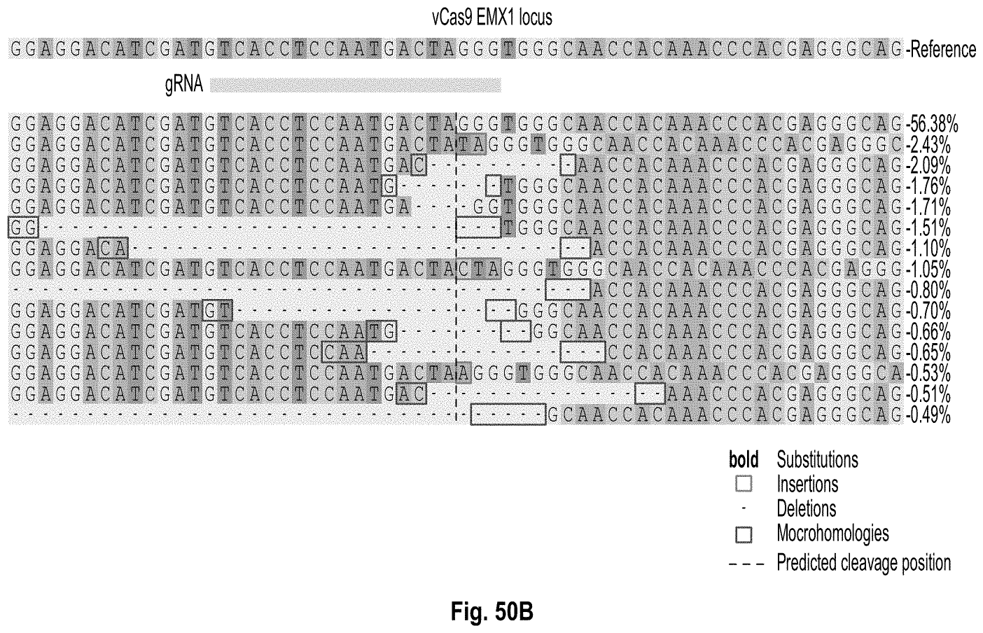

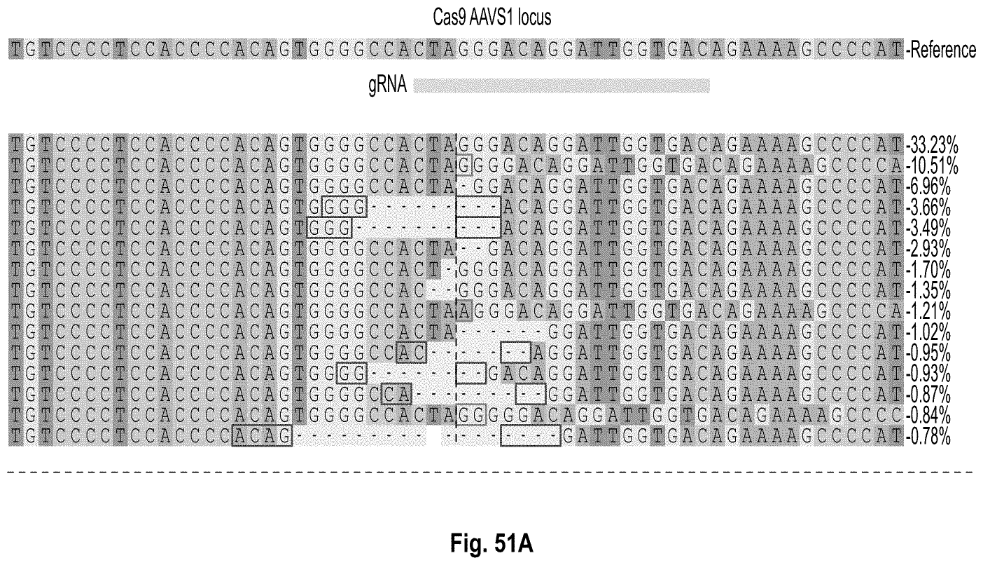

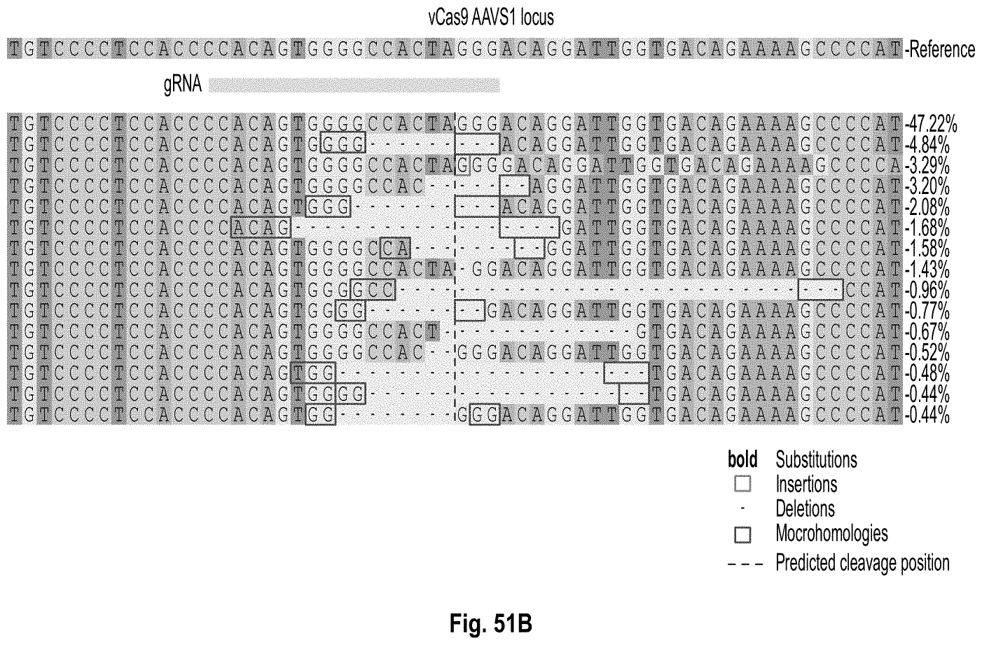

Editing outcomes for each engineered Cas9 variant from the Alanine substitution screen were studied with a gRNA and a precise HDR editing template. Methods were the same as described in Example 3. There was a strong correlation between precise editing frequency and mean indel size (FIG. 23A-B) as well as staggered cutting (FIG. 49A) for Cas9 variants. vCas9 showed the largest shift in break structure by predominantly making staggered cuts of 6 bp or larger (FIG. 49B). Indel size, distributions, and mechanisms for Cas9 from Streptococcus pyogenes (SEQ ID NO: 1) and vCas9 with gRNAs in the absence of HDR templates were analyzed at the AAVS1 locus (FIG. 24), CXCR4 locus (FIG. 25), EMX1 locus (FIG. 26), and VEGFA locus (FIG. 27) in HEK293T cells using deep next-generation sequencing. vCas9 induced indels of larger size than those by Cas9 from Streptococcus pyogenes (SEQ ID NO: 1) at all four loci (FIGS. 24-28) and greatly suppressed NHEJ deletions and insertions while promoting MMEJ deletions (FIG. 29; FIGS. 50-51). Specifically, vCas9 shifted indels from NHEJ repair to MMEJ repair utilizing larger microhomologies (FIG. 29) in the absence of an ssODN template.

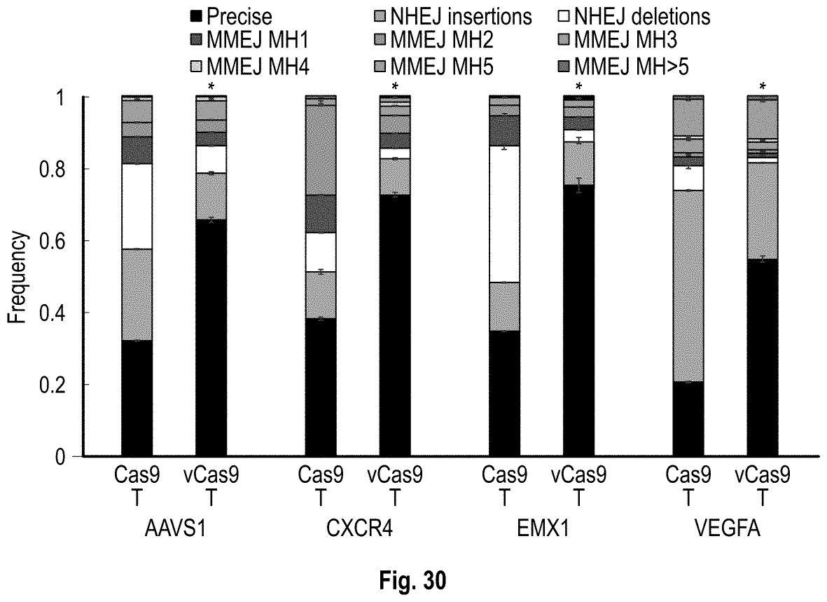

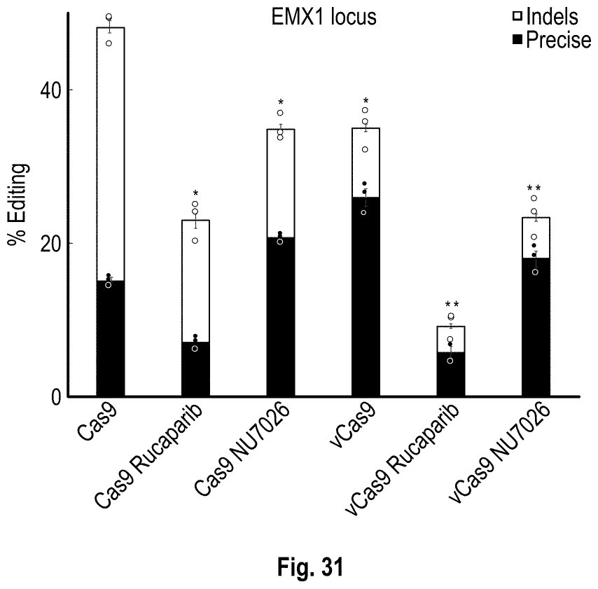

When both Cas9 from Streptococcus pyogenes (SEQ ID NO: 1) and vCas9 were introduced into HEK293T cells in the presence of a gRNA and an ssODN HDR template, vCas9 consistently made HDR dominant, with the remaining minor repair outcomes comprised mostly of NHEJ insertions and MMEJ deletions (FIG. 30).

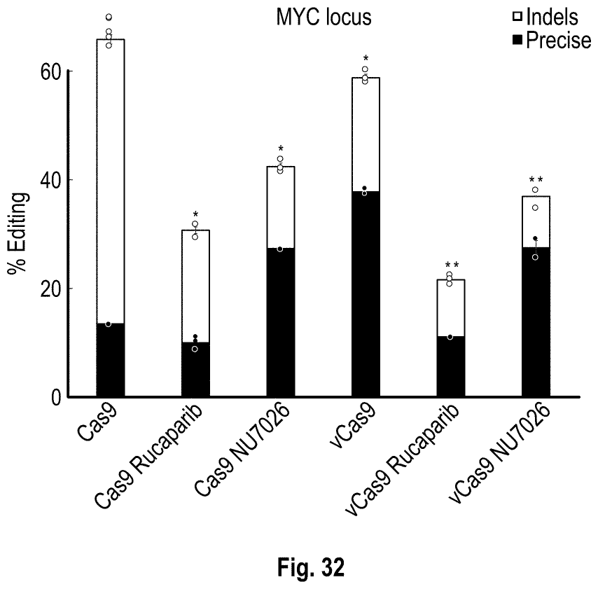

Engagement of repair pathways was also tested in the presence of NHEJ and MMEJ inhibitors. Cas9 from Streptococcus pyogenes (SEQ ID NO: 1) and vCas9 along with a gRNA and ssODN HDR template were introduced into HEK293T cells that were also treated with either an inhibitor of MMEJ (Rucaparib) or an inhibitor of NHEJ (NU7026). Cas9 from Streptococcus pyogenes (SEQ ID NO: 1) combined with the NHEJ inhibitor NU7026 led to editing patterns that resembled vCas9 at both the EMX1 locus (FIG. 31, 3^{rd} and 4^{th} column) and the MYC locus (FIG. 32, 3^{rd} and 4^{th} column), while inhibiting MMEJ with Rucaparib largely ablated editing by vCas9 but not Cas9 from Streptococcus pyogenes (SEQ ID NO: 1) (FIG. 31 and FIG. 32, 2^{nd} and 5^{th} columns).

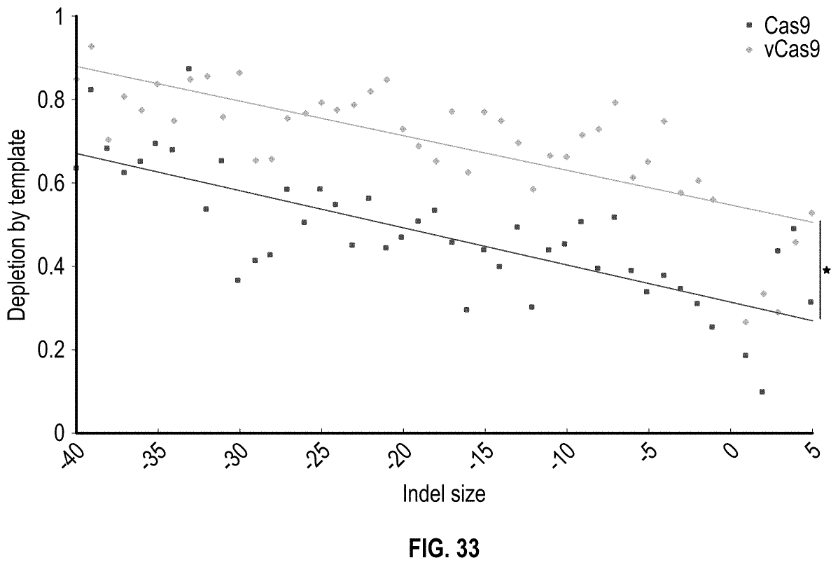

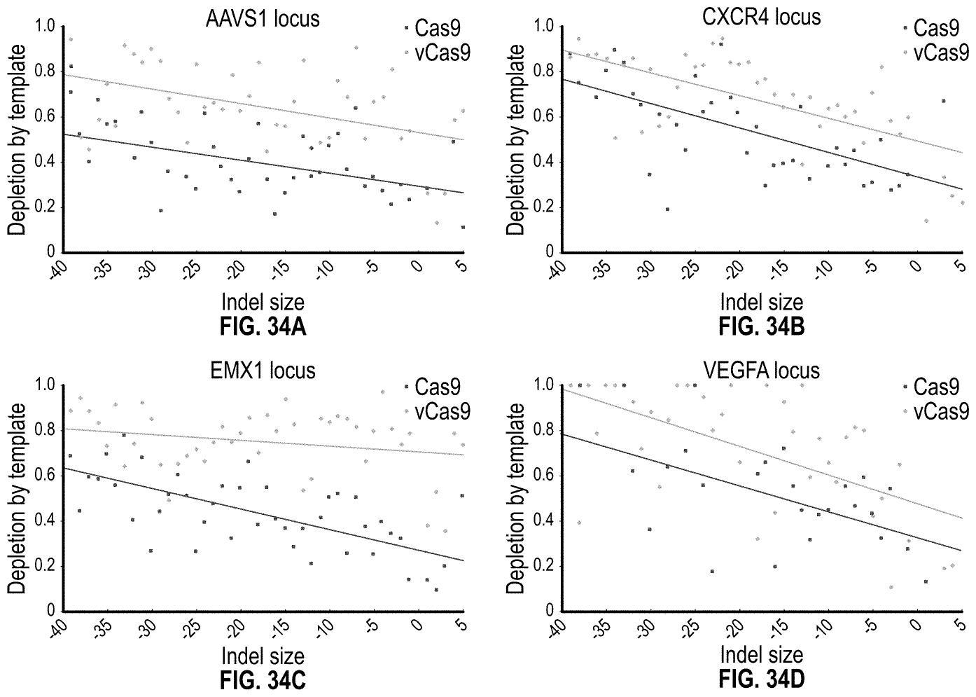

Comparing repair frequencies in HEK293T cells exposed to a gRNA with or without an HDR template, vCas9 exhibited an increased degree to which precise editing outcompeted any size indel (FIG. 33 (averaged over 4 loci) and FIG. 34A-D (individual loci)). Together, these data demonstrate that vCas9 suppresses NHEJ in favor of repair pathways that use homologous sequences, hence promoting HDR or MMEJ.

### Example 5. Cas9 Variant Engagement of Precision Editing in Non-Dividing Cells

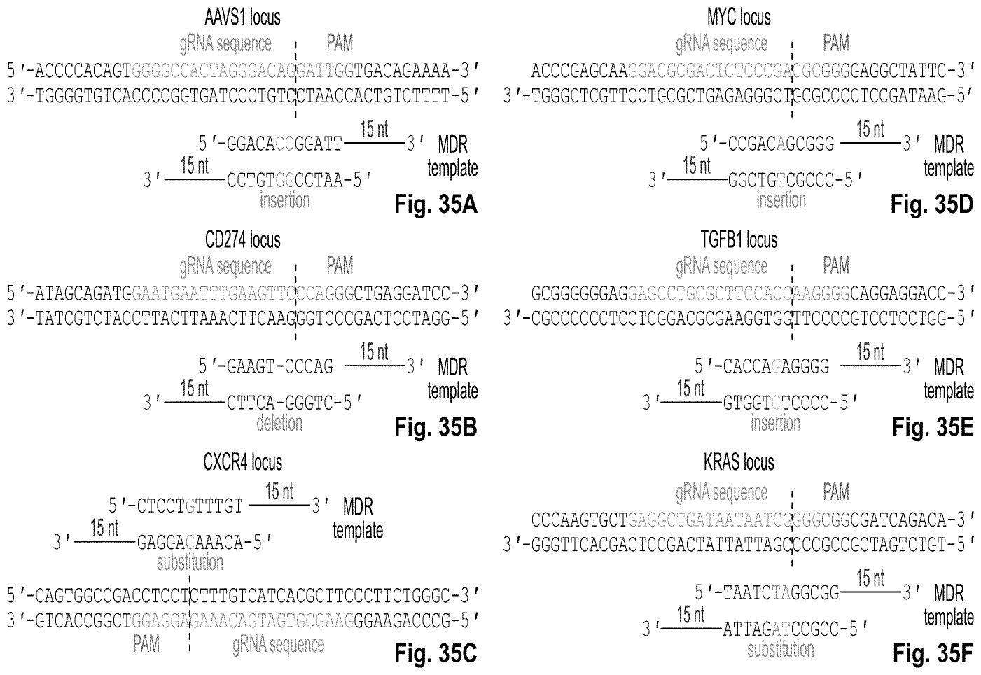

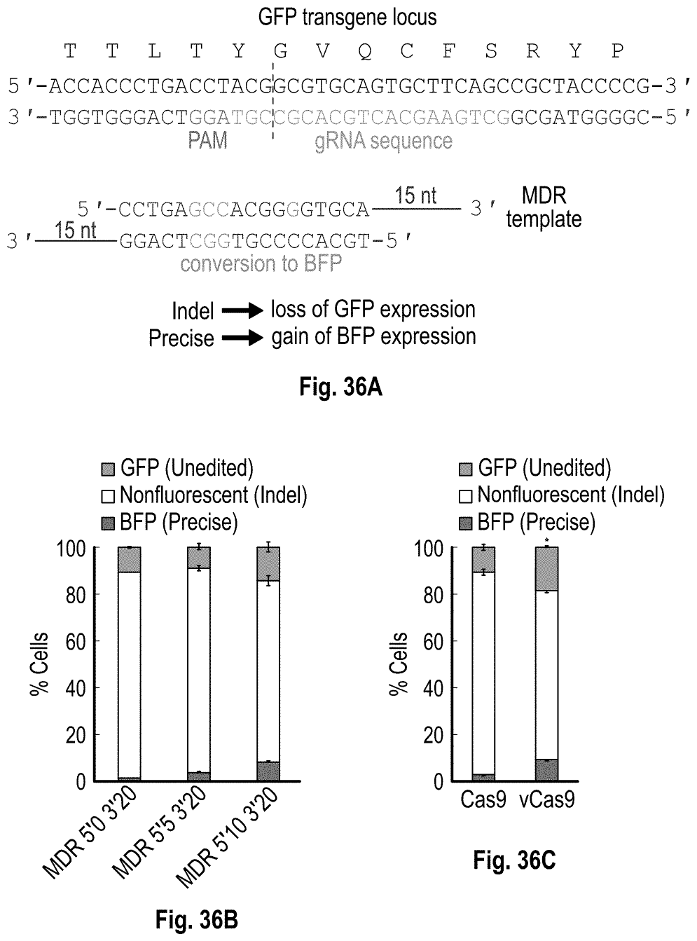

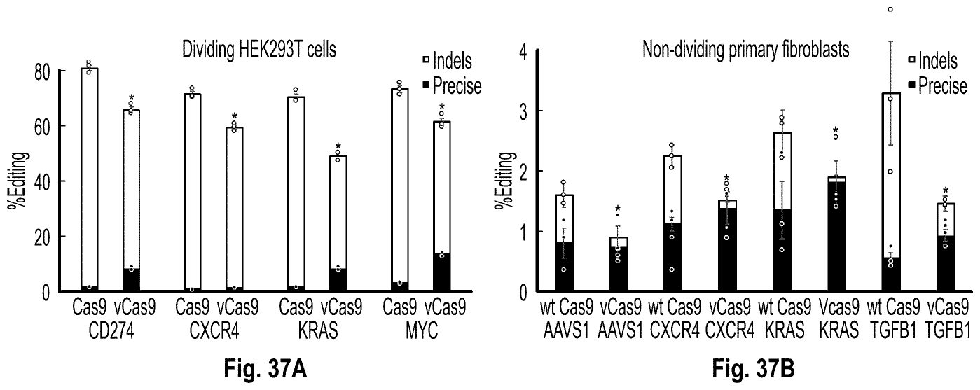

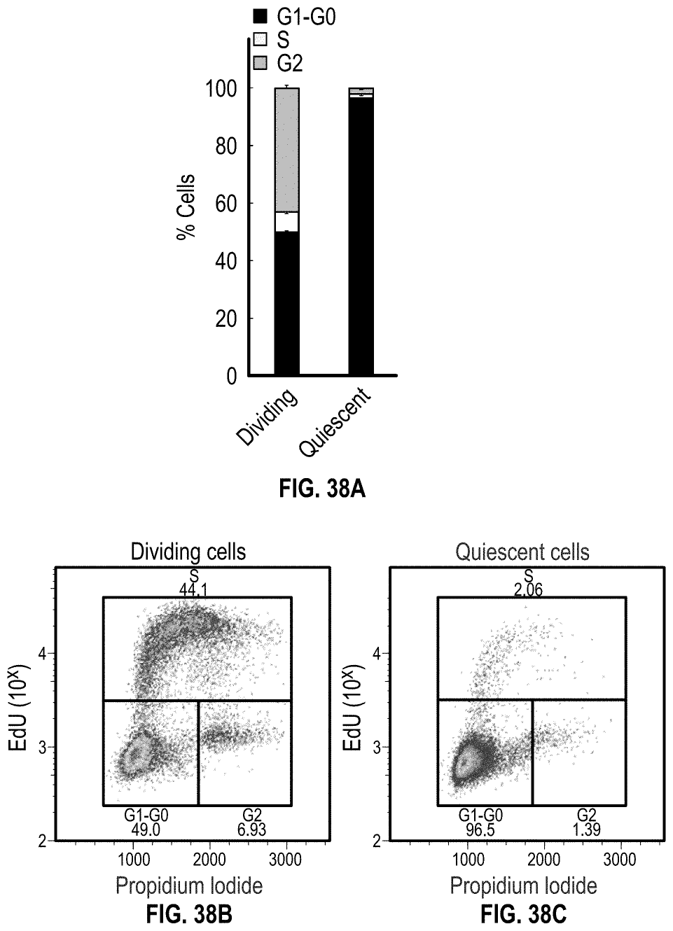

A major limitation of CRISPR technology for precise editing to generate genetic models or treat certain diseases is the lack of HDR in non-dividing cells. The major pathways engaged in DNA repair in non-dividing cells are MMEJ and NHEJ. As such, the ability to precisely edit the genome of non-dividing cells is a critical and undermet need in the gene editing field. To determine if vCas9 might be reliably engage precise HDR repair in non-dividing cells, an MMEJ-driven template strategy was developed. This hybrid strategy was termed “microhomology-directed recombination” (MDR). It utilizes a partially double-stranded DNA templates with single-stranded microhomology arms complementary to sequences distal to the DSB ends (FIG. 35). These MDR templates putatively enable replacements of small segments of genomic DNA situated between the two microhomology arms. Using the GFP->BFP gene conversion assay described above as a measurement of MDR in HEK293T, varied MDR template designs were analyzed (FIG. 36A). An MDR template with no 5′ microhomology arm and a 20 nt 3′ microhomology arm (FIG. 36B, column 1) exhibited editing almost exclusively attributed to indels leading to a lack of fluorescence. By contrast, an MDR template with a 5 nt 5′ microhomology arm and a 20 nt 3′ microhomology arm (FIG. 36B, column 2) exhibited an increase in Blue fluorescence, indicative of an increase in precise MDR engagement. Further, an MDR template with a 10 nt 5′ homology arm and a 20 nt 3′ homology arm exhibited both a further increase in blue fluorescence, indicating an increase in precise MDR engagement, as well as a decrease in nonfluorescent cells. This decrease is indicative of a greater shutdown of non-HDR pathways by using the MDR system.

When Cas9 from Streptococcus pyogenes (SEQ ID NO: 1) and vCas9 was paired with an MDR template with a 5 nt 5′ homology arm and a 20 nt 3′ homology arm, cells transfected with vCas9 exhibited significantly increased precise editing as measured by blue fluorescence, as well as a significant decrease in non-precise editing as measured by the presence of indels (FIG. 36C).

To examine engagement of precise HDR/MDR editing pathways in both dividing and non-dividing cells, MDR templates from several loci (FIG. 35) were supplied to either dividing HEK293T cells or quiescent primary human dermal fibroblasts (a model of G0 non-dividing cells) in the presence of Cas9 from Streptococcus pyogenes (SEQ ID NO: 1) or vCas9. In dividing cells, the majority of edits, regardless of Cas protein within the cell, were non-precise edits. vCas9 still exhibited a significant increase in precise editing in these cells. Cas9 from Streptococcus pyogenes (SEQ ID NO: 1) in the non-dividing primary fibroblasts exhibited a range of edits. However, editing by Cas9 from Streptococcus pyogenes (SEQ ID NO: 1) in these cells mostly displayed a split between precise editing and indels, with at least one locus showing a clear engagement preference for non-precise editing. In contrast, the overwhelming majority of editing in cells exposed to vCas9 was precise, across all loci. The ability to engineer Cas9 nucleases, such as those disclosed herein, which are capable of precise gene editing in non-dividing cells provides a critical advancement in the ability to harness genetic editing systems.

### Example 6. Effect of Cas9 Mutations on Prime Editors

We next set out to determine if altered Cas9 variants could be employed in other genome editing systems to similarly suppress indels and favor precise gene editing. We first examined the use of these Cas9 variants in a prime editing system. Prime editing systems employ Cas9 nickases fused to a reverse transcriptase, which is then paired with an engineered guide RNA which contains a sequence complementary to target DNA, as well as an extension region which encodes the desired change (pegRNA). Although prime editors favor precise editing and decrease indels relative to Cas9 nuclease editing by HDR, they do not completely eliminate indel production.

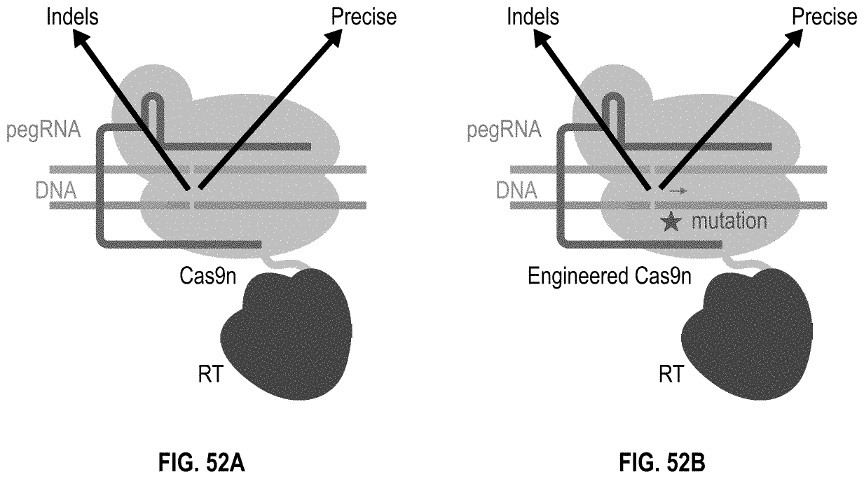

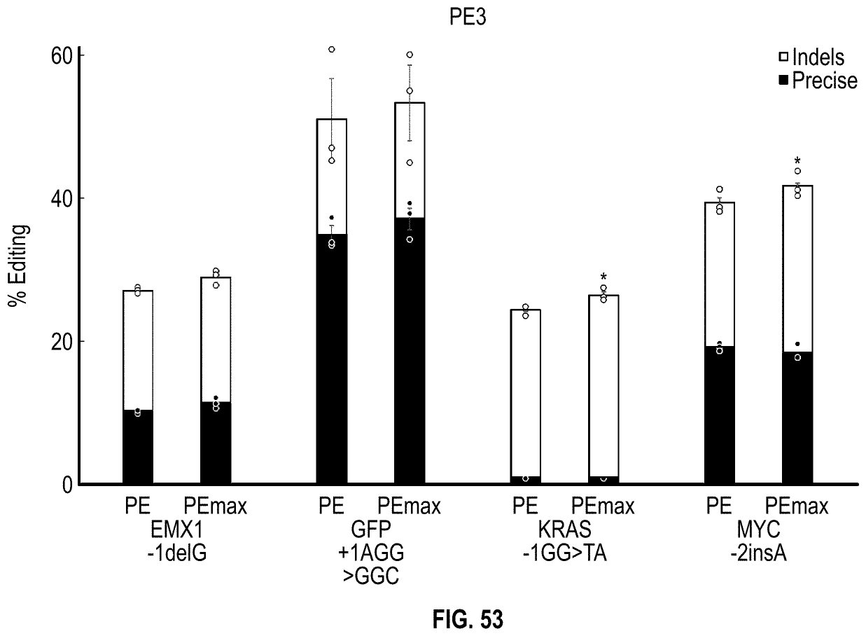

We determined whether mutations incorporated into a Cas9 nickase established within a prime editing system could alter the frequency of repair outcomes (FIG. 52). We compared the optimized prime editing system PEmax (Chen et al. Cell 184, 5635-5652.e29 (2021)), which has an R221K and an N394K mutation in the Cas9 nickase, to PE, a variant of PEmax in which we reverted the R221K and N394K mutations. We compared PEmax and PE for creating small edits at the EMX1, GFP, KRAS, and MYC loci in HEK293T cells. We found that PEmax displayed a slightly elevated indel frequency relative to PE, indicating that the R221K and N394K mutations lead to increased indel production (FIG. 53) This demonstrates that mutations such as R221K and N394K, which can increase the activity of Cas9 nickases in prime editor systems, nevertheless increase indel frequency.

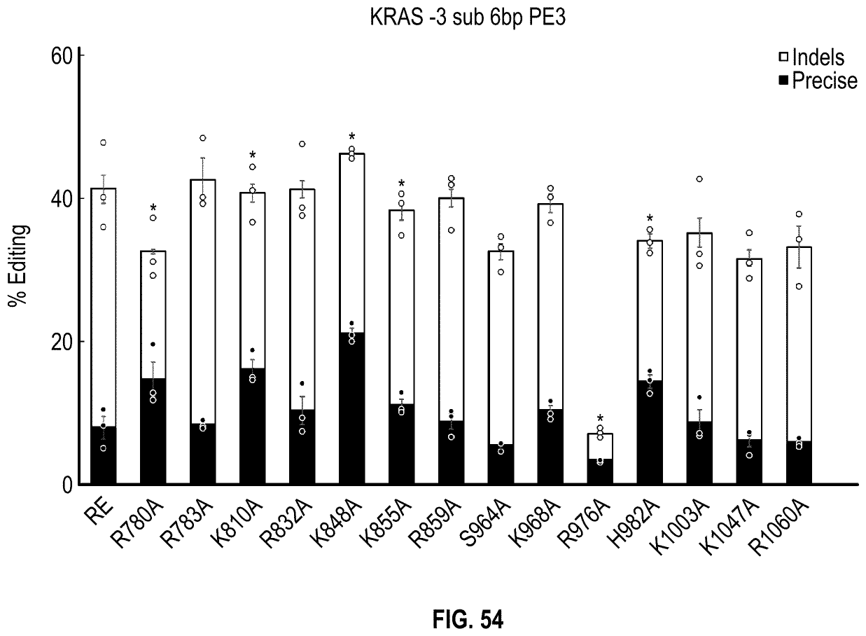

Since the PE prime editor system has slightly reduced indel frequency relative to PEmax, we used PE as a basis for further engineering of Cas9 within prime editor systems. We next introduced the 14 mutations that we previously introduced into Cas9 (R780A, R783A, K810A, R832A, K848A, K855A, R859A, S964A, K968A, R976A, H982A, K1003A, K1047A, and R1060A) into the Cas9 nickase within the PE prime editor system. To measure precise editing and indel frequencies for these mutants, we combined them with a pegRNA and nicking gRNA (double-nicking prime editing, or PE3) that produces a small sequence replacement at the KRAS locus in HEK293T cells by prime editing and analyzed alterations using deep sequencing of the amplified locus. Several PE single-mutant variants demonstrated reduced indel frequency and increased precise editing frequency (FIG. 54A).

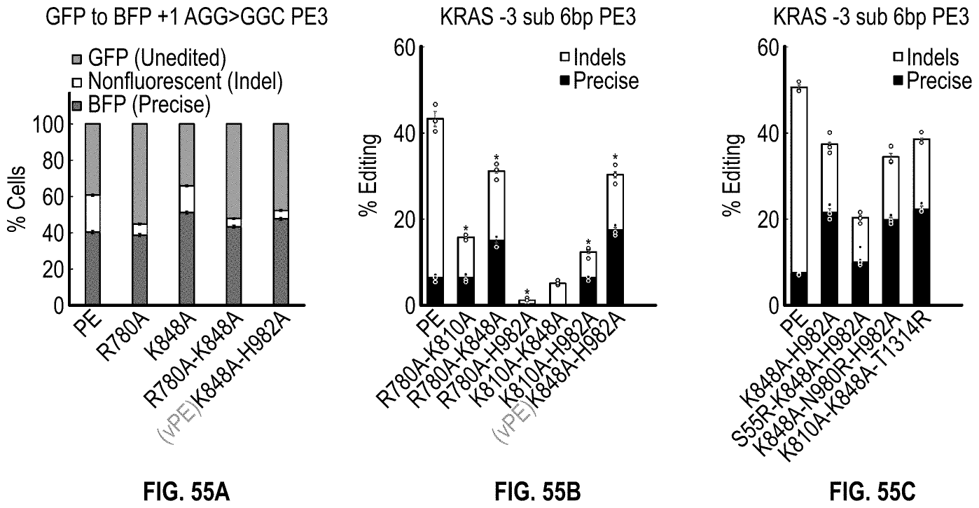

We further created and analyzed several dual-mutation variants of the Cas9 nickase within the PE prime editor system (R780A-K810A; R780A-K848A; R780A-H982A; K810A-K848A; K810A-H982A; K848A-H982A). We tested these engineered variants by applying prime editing with a pegRNA and nicking gRNA to convert a GFP transgene to BFP, similar to the HDR assay. One variant, K848A-H982A, nearly eliminated indels while promoting precise editing (FIG. 55A). When editing of the KRAS locus was examined by these prime editor variants, several combinations displayed an increased precise editing frequency and reduced indel frequency relative to PE, including the K848A-H982A variant (FIG. 55B). Further engineering by Arginine substitution did not significantly affect prime editor activity (FIG. 55C). Since PE K848A-H982A was the most precise variant of PE, we named it vPE.

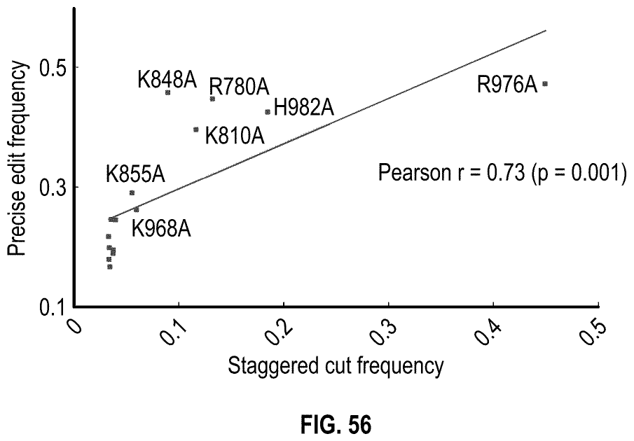

We next explored mechanisms and robustness for these effects on prime editor repair outcomes. We compared the precise editing frequencies of each PE single-mutant with the break structure alterations observed for corresponding Cas9 single-mutants. We again found a strong correlation between precise editing frequency and altered break structure for these PE variants (FIG. 56). Remarkably, the mutations that affected break structure (R780A, K810A, K848A, K855A, K968A, R976A, and H982A) were largely the same mutations that altered repair outcomes for both Cas9 and PE.

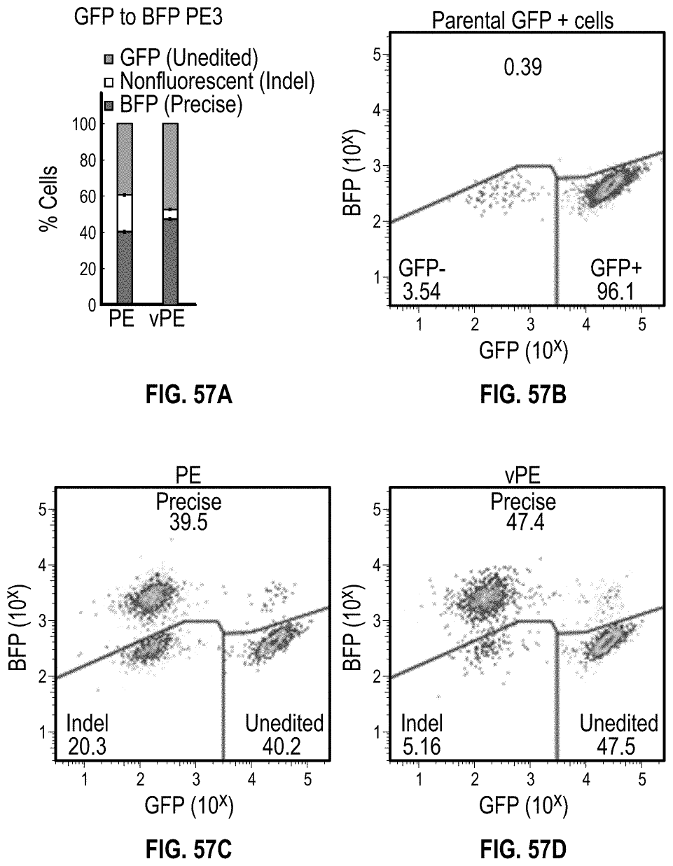

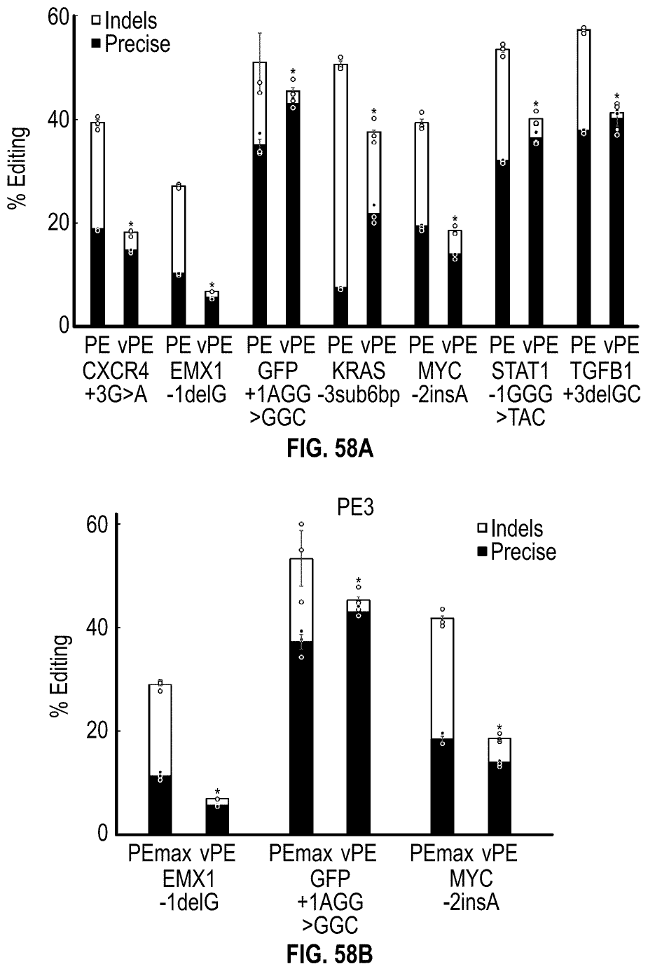

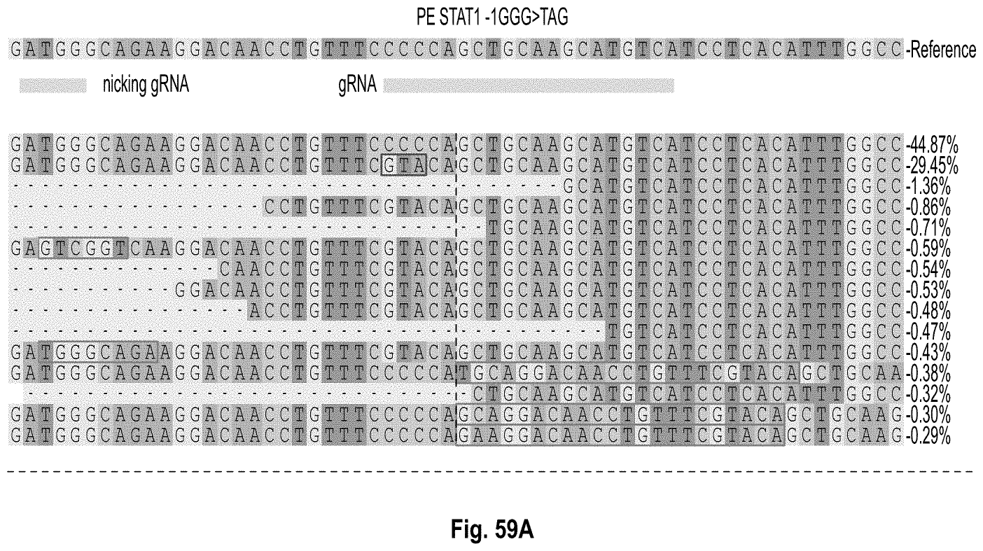

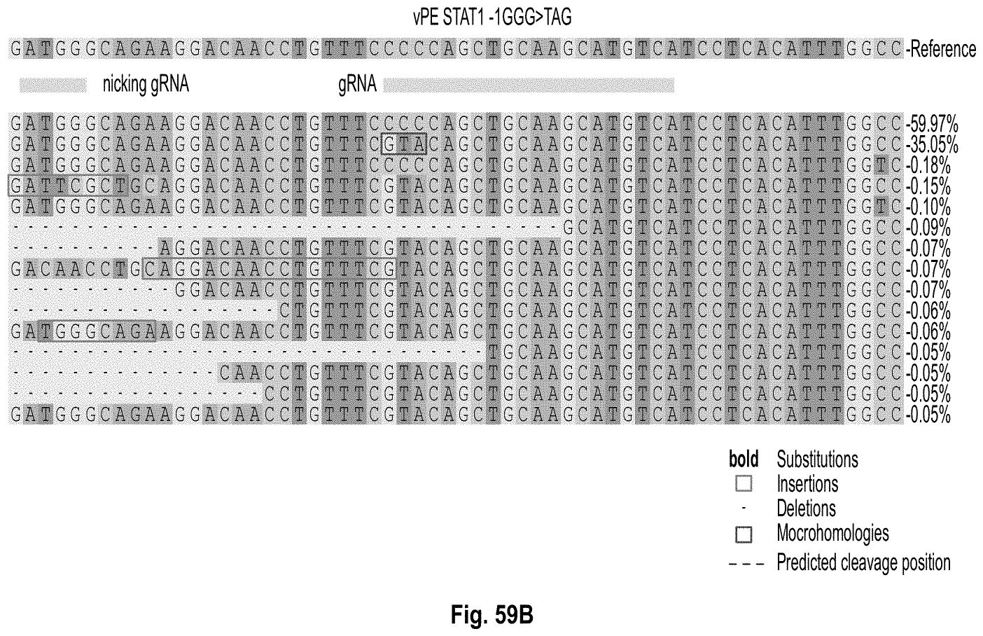

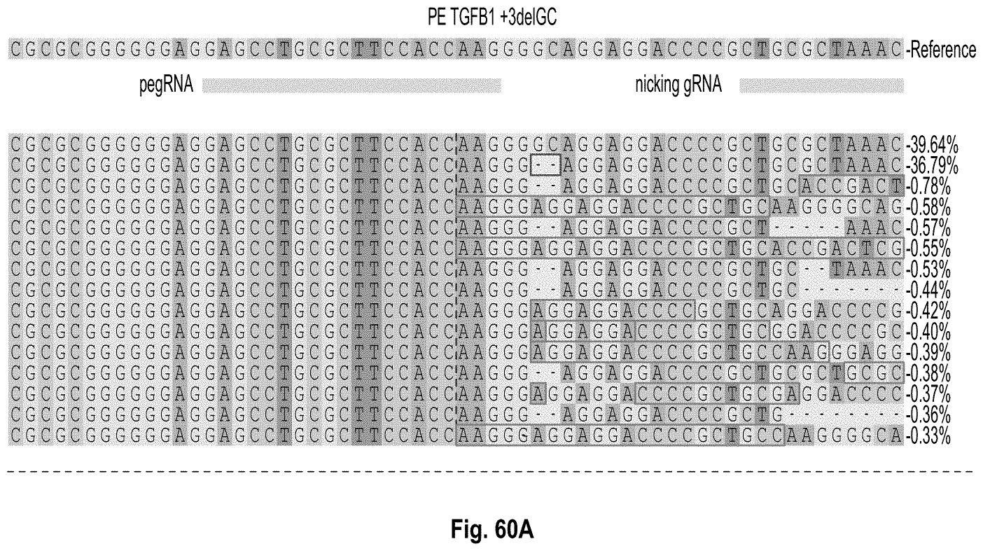

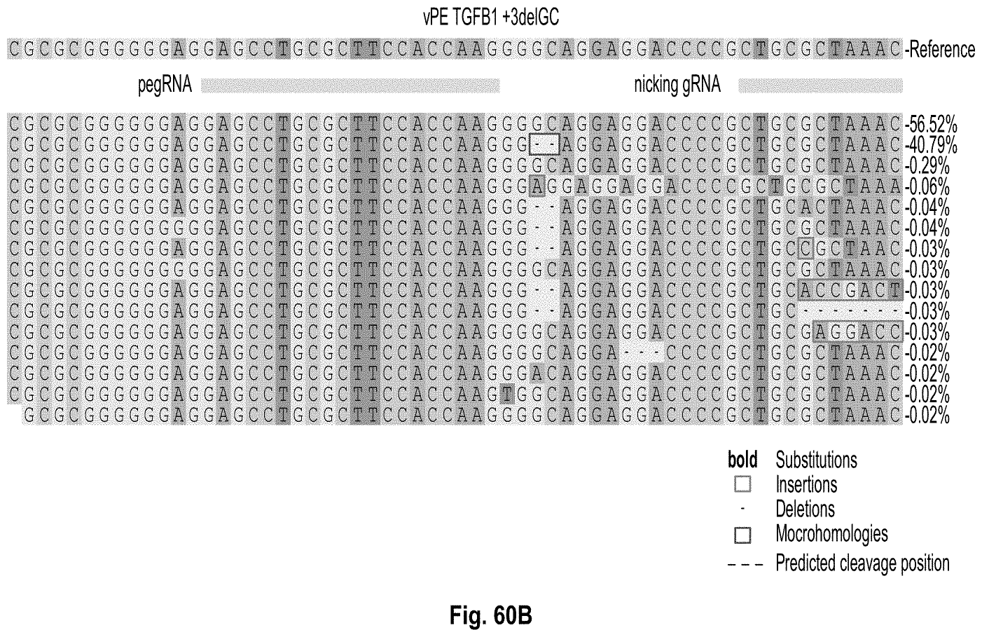

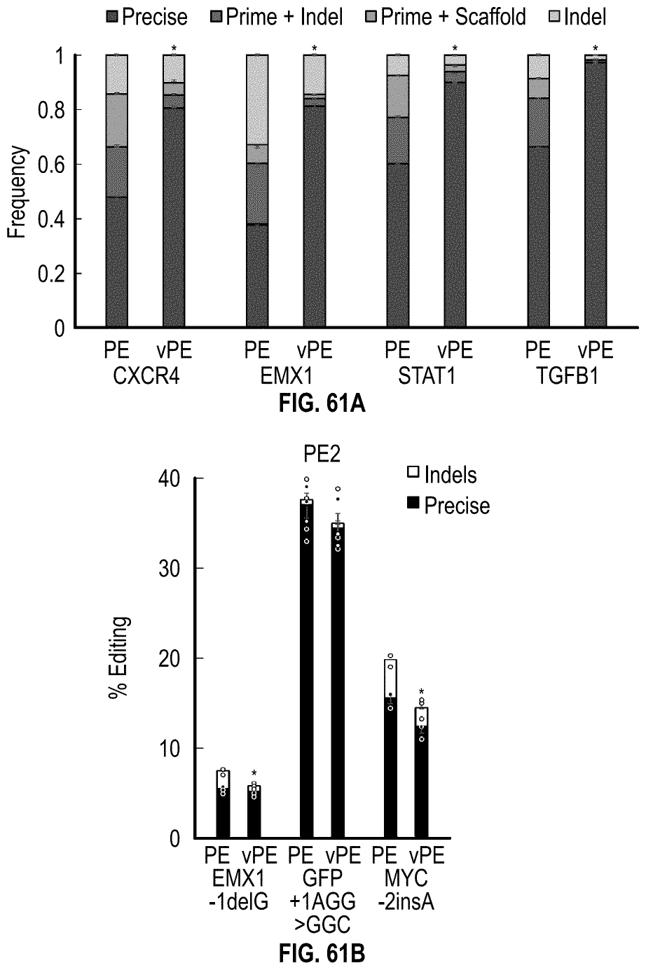

We next examined whether vPE produces precise editing by again applying prime editing to convert a GFP transgene to BFP. Here, vPE resulted in efficient precise gene conversion with limited indels (FIG. 57A-D). When we tested vPE editing at several loci in HEK293T cells using pegRNAs and nicking gRNAs, vPE nearly eliminated indels resulting from prime editing at all loci (FIG. 58A-B; FIG. 59; FIG. 60). While PE displayed precise editing frequencies of 14.7-69.8% (mean 49.1%), vPE increased these to 57.7-97.4% (mean 82.5%), corresponding to a 2.0- to 13.0-fold (mean 4.7-fold) suppression of indel frequency. As prime editors can produce indels without the prime edit, with the prime edit, or with the prime edit and pegRNA scaffold incorporation (Chen et al. Cell 184, 5635-5652.e29 (2021)), we further determined how these different indel types were affected. At each locus, vPE increased precise editing while decreasing the frequencies of each indel type (FIG. 61A).

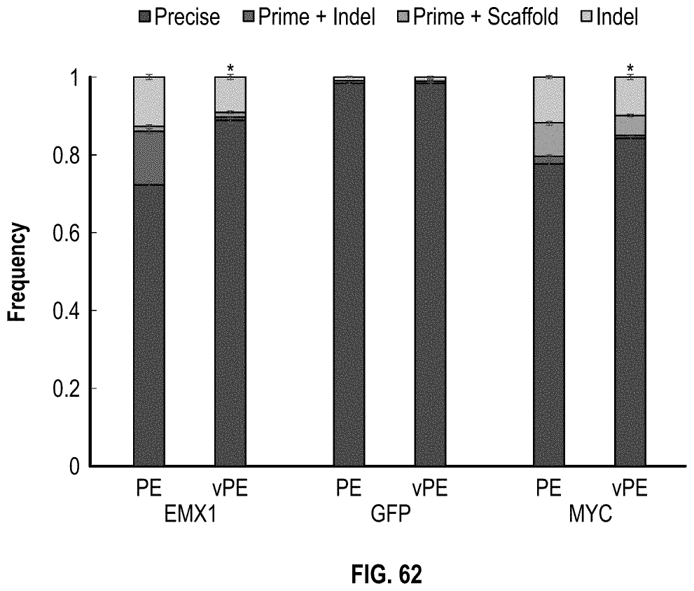

Considering this broad suppression of all indel types, we also studied whether vPE reduced indel frequency at several loci in HEK293T cells using pegRNAs without nicking gRNAs (single-nicking prime editing, or PE2). Intriguingly, though PE resulted in fairly low indel frequencies at all loci, vPE produced lower indel frequencies (FIG. 61B). Here PE produced precise editing frequencies of 72.3-98.2% (mean 82.7%) and vPE increased these to 84.2-98.4% (mean 90.5%), corresponding to a 1.1- to 2.5-fold (mean 1.7-fold) suppression of indel frequency. This corresponded to reductions in all indel types (FIG. 62). These findings establish that engineering Cas9 to alter repair pathway frequency can significantly enhance the precision of prime editors and may be broadly applicable to many classes of genome editors.

Except as otherwise stated in the above Examples, the following general methods were employed throughout.

### Mammalian Cell Culture

All mammalian cell cultures were maintained in a 37° C. incubator at 5% CO2. HEK293T human embryonic kidney, HeLa human cervical cancer, A549 human lung cancer, and Panc1 human pancreatic cancer cells were maintained in Dulbecco's Modified Eagle's Medium with high glucose, sodium pyruvate, and GlutaMAX (DMEM; ThermoFisher, 10569) supplemented with 10% Fetal Bovine Serum (FBS; ThermoFisher, 10438), and 100 U/mL Penicillin-Streptomycin (ThermoFisher, 15140). For inhibitor studies, cell media was supplemented with 20 μM Rucaparib (MilliporeSigma, PZ0036) or NU7026 (MilliporeSigma, N1537) dissolved in DMSO (MilliporeSigma, D8418).

### Mutagenesis and Cloning

Wild-type Cas9 was obtained from pSpCas9 (pX165) and a cloning backbone for gRNA expression was obtained from pX330-U6-Chimeric BB-CBh-hSpCas9 (pX330). Cas9 mutagenesis was performed using PCR-driven splicing by overlap extension using primers listed in Supplementary Table 1. Briefly, one fragment was amplified by PCR from pX165 using the cas9-mut-FWD or cas9-mid-FWD and mutant-BOT primers and a second fragment was amplified using the mutant-TOP and cas9-mid-REV or cas9-mut-REV primers for each mutant. Each pair of fragments was then spliced by overlap extension PCR using the cas9-mut-FWD and cas9-mid-REV or cas9-mid-FWD and cas9-mut-REV primers to create a Cas9 gene fragment with a single residue mutation. These Cas9 gene fragments were then each cloned back into pX165 using unique BshTI, ApaI, and EcoRI restriction sites to replace the wild-type sequence with the mutant sequence. Additional mutants (double-, triple-, and quadruple-mutants) were made iteratively starting from these single-mutant plasmids. A custom gRNA cloning backbone vector was created by PCR amplification from pX330 using the gRNA-scaffold-NheI-FWD and gRNA-scaffold-EcoRI-REV primers and restriction cloning into pUC19 (ThermoFisher) using NheI and EcoRI digestion. The gRNA spacer sequence oligos, listed in Supplementary Table 2, were phosphorylated with T4 polynucleotide kinase (NEB) and cloned into gRNA cloning backbone by Golden Gate cloning with BpiI digestion.

PE2 and PEmax prime editors were obtained from pCMV-PE2 and pCMV-PEmax, and a cloning backbone for pegRNA expression was obtained from pU6-pegRNA-GG-acceptor. PE was created by restriction cloning of Cas9n (H840A) from pCMV-PE2 into pCMV-PEmax using NotI and SacI digestion. PE mutagenesis was performed using PCR-driven splicing by overlap extension using primers listed in Supplementary Table 1. Briefly, one fragment was amplified by PCR from PE using the pe-FWD or pe-mid-FWD and mutant-BOT primers and a second fragment was amplified using the mutant-TOP and pe-mid-REV or pe-rt-REV primers for each mutant. Each pair of fragments was then spliced by overlap extension PCR using the pe-FWD and pe-mid-REV or pe-mid-FWD and pe-rt-REV primers to create a PE gene fragment with a single residue mutation. These PE gene fragments were then each cloned back into PE using unique NotI, SacI, and BamHI restriction sites to replace the PE sequence with the mutant sequence. Additional mutants (double- and triple-mutants) were made iteratively starting from these single-mutant plasmids. The pegRNA oligos, listed in Supplementary Table 3, were phosphorylated with T4 polynucleotide kinase (NEB) and cloned into pU6-pegRNA-GG-acceptor by Golden Gate cloning with Eco31I digestion. Primers were synthesized by IDT. Restriction enzymes were obtained from ThermoFisher. T7 DNA ligase was obtained from NEB. Plasmids were transformed into competent Stbl3 chemically competent E. coli (ThermoFisher). Sequences for the wild-type Cas9, vCas9, gRNA cloning backbone, PE, and vPE vectors are presented in the Sequences section.

High-fidelity Cas9 variants were obtained from pX165-Cas9-HF1, pX165-eSpCas9, and pX165-HypaCas9. Cas9 fusions Cas9-CtIP and Cas9-dn53bp1 were created by restriction cloning of custom geneblocks synthesized by IDT into pX165 at unique KflI and EcoRI restriction sites.

### Structure Analysis

Crystal structures of Cas9 with substrate DNA bound (5F9R) or without substrate DNA bound (4ZTO) were analyzed using PyMol (Schrödinger).

### Cell Transfection

Cells were seeded in the maintenance medium without Pen-Strep into 24-well plates at 100,000 cells/well or 48-well plates at 50,000 cells/well. Transfections of HEK293T without repair templates were carried out 24 hrs after seeding using 400 ng Cas9 expression vector and 144 ng gRNA expression vector formulated with 1.36 μL Lipofectamine 2000 (ThermoFisher) at a total volume of 54.4 μL in OptiMEM I (ThermoFisher) per well for 24-well plates, or half these volumes for 48-well plates. Transfections of HEK293T, HeLa, A549, and Panc1 with HDR templates were carried out 24 hrs after seeding using 400 ng Cas9 expression vector, 144 ng gRNA expression vector, and 400 ng ssODN HDR template formulated with 2.11 μL Lipofectamine 2000 at a total volume of 84.4 μL in OptiMEM I per well for 24-well plates, or half these volumes for 48-well plates. Transfections of HEK293T with dual gRNAs were carried out 24 hrs after seeding using 400 ng Cas9 expression vector and 144 ng of each gRNA expression vector formulated with 1.72 μL Lipofectamine 2000 (ThermoFisher) at a total volume of 68.8 μL in OptiMEM I (ThermoFisher) per well for 24-well plates, or half these volumes for 48-well plates. Transfections of HEK293T with prime editing vectors were carried out 24 hrs after seeding using 475 ng PE expression vector, 114 ng pegRNA expression vector, and 144 ng nicking gRNA expression vector (for PE3) formulated with 1.47-1.83 μL (equal volume/DNA) Lipofectamine 2000 at a total volume of 58.9-73.3 μL (equal DNA concentration) in OptiMEM I (ThermoFisher) per well for 24-well plates, or half these volumes for 48-well plates. For sequencing assays, genomic DNA was extracted 72 hrs after transfection using QuickExtract (Epicentre). For flow cytometry assays, cells were transferred to 6-well plates 72 hrs after transfection, split 7 days after transfection, and harvested 10 days after transfection in PBS with 5% FBS (ThermoFisher). Repair templates, listed in Supplementary Table 4, were synthesized by IDT.

### High-Throughput Sequencing

The targeted loci were amplified from extracted genomic DNA by PCR using Herculase II polymerase (Agilent). The PCR primers included Illumina sequencing handles as well as replicate-specific barcodes. These PCR products were then tagged with sample-specific barcodes and sequenced on an Illumina MiSeq. Primers, listed in Supplementary Table 5, were synthesized by IDT.

### Sanger Sequencing

The targeted loci were amplified from extracted genomic DNA by PCR using Herculase II polymerase (Agilent). PCR amplicons were sequenced using primers ˜200 bp from the expected cut site. To measure editing frequencies, the sequencing traces were analyzed using TIDE 24. Primers, listed in Supplementary Table 6, were synthesized by IDT.

### Flow Cytometry

Flow cytometry analysis was performed on an LSR Fortessa analyzer and data was collected using FACSDiva (BD Biosciences). Cells were first gated comparing SSC-A and FSC-A, then SSC-H and SSC-W, then FSC-H and FSC-W parameters to select for single cells. To assess editing frequencies, cells were gated for GFP (488 nm laser excitation, 530/30 nm filter detection) and BFP (405 nm laser excitation, 450/50 nm filter detection). To profile cell cycle stage, cells were gated for propidium iodide (561 nm laser excitation, 610/20 nm filter detection) and Alexa Fluor 647 (640 nm laser excitation, 670/30 nm filter detection). Flow cytometry data were analyzed using FlowJo (FlowJo).

### Genome Editing Analysis

To measure editing outcomes, the high-throughput sequencing data were analyzed using CRISPResso2 25. Total editing rates were quantified as the fraction of edited reads out of total sequencing reads. Indel rates were quantified as the fraction of reads containing indels out of total sequencing reads. Precise editing rates were quantified as the fraction of reads containing a perfect match to the expected edit out of total sequencing reads. Frequencies of specific indel sizes were quantified as the fraction of reads containing these sizes out of all edited reads. Depletion of specific indel sizes by templated repair was quantified as the fractional reduction in the frequency of that indel size, comparing frequencies for when a template was present versus absent. Mean indel sizes were calculated as the mean of the absolute values of indel sizes weighted by their indel fractions.

### DNA Break Structure Analysis

To measure DNA break structures for Cas9 variants, editing outcomes for dual-gRNA cutting of genomic DNA were analyzed as previously described 13. HEK293T cells were edited with pairs of gRNAs targeting the EMX1 (EMX1 gRNA 1 and gRNA 2) or CXCR4 (CXCR4 gRNA 1 and gRNA 2) loci. The gRNA pairs were complementary to the same strand at each locus and were expected to make cuts 84 bp apart, resulting in large precise deletions. The loci were amplified and sequenced by high-throughput sequencing. The high-throughput sequencing data were analyzed using CRISPResso2 25, using the expected 84 bp deletion junction as a reference sequence. To assess DNA break structure, sequencing reads aligned to the deletion junction reference were analyzed for insertion sequences perfectly matching the sequences flanking the expected gRNA cut sites. The positions of these matching sequences at the two gRNA sites were used to determine cut positions leading to each read. Frequencies of these cut positions were quantified as the fraction of reads resulting from these specific cut positions out of all reads containing the deletion junctions with or without insertions.

### Repair Pathway Outcome Analysis

For repair pathway analysis, next-generation sequencing reads were classified using annotated repair mechanisms determined by Indelphi 4. The high-throughput sequencing data for each editing experiment were analyzed using CRISPResso2 25. The same gRNA and locus sequences were also analyzed using Indelphi to identify whether each predicted indel was associated with a microhomology (MMEJ) or not (NHEJ), along with microhomology sizes. These repair pathway labels for each edited sequence from Indelphi analysis were then applied to the matching sequencing reads for the editing experiment. Frequencies of NHEJ, MMEJ, and precise editing were quantified as the fraction of reads containing these types of edits out of all edited reads.

### Off-Target Activity Analysis

To assess off-target cutting activity, indel rates were analyzed at known off-target sites previously reported for two gRNAs (EMX1 gRNA 2 and VEGFA gRNA 1) 19. Indel rates were determined by analysis of Sanger sequencing traces at these on-target and off-target loci using TIDE 24.

### Statistical Analysis

Specific statistical comparisons are indicated in the figure legends. Error bars indicate the standard error for three independent replicates. In most comparisons, significance was assessed using unpaired, two-tailed Student's t-tests. For correlations, significance was assessed using Pearson's tests. For linear regressions, significance was assessed using ANCOVA tests.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety. The publications discussed herein are provided solely for their disclosure prior to the filing date of the present application. Nothing herein is to be construed as an admission that the present disclosure is not entitled to antedate such publication. Further, the dates of publication provided may be different from the actual publication dates which may need to be independently confirmed.

### REFERENCES

1. L. Cong et al., Multiplex genome engineering using CRISPR/Cas systems. Science. 339, 819-823 (2013).
2. P. Mali et al., RNA-guided human genome engineering via Cas9. Science. 339, 823-826 (2013).
3. S. F. Bunting et al., 53BP1 inhibits homologous recombination in Brca1-deficient cells by blocking resection of DNA breaks. Cell. 141, 243-254 (2010).
4. E. Cannavo, P. Cejka, Sae2 promotes dsDNA endonuclease activity within Mre11-Rad50-Xrs2 to resect DNA breaks. Nature. 514, 122 (2014).
5. T. Costelloe et al., The yeast Fun30 and human SMARCAD1 chromatin remodelers promote DNA end resection. Nature. 489, 581 (2012).
6. A. A. Sartori et al., Human CtIP promotes DNA end resection. Nature. 450, 509 (2007).
7. M. Zimmermann, F. Lottersberger, S. B. Buonomo, A. Sfeir, T. de Lange, 53BP1 regulates DSB repair using Rif1 to control 5′ end resection. Science. 339, 700-704 (2013).
8. J. R. Chapman, M. R. G. Taylor, S. J. Boulton, Playing the end game: DNA double-strand break repair pathway choice. Mol. Cell. 47, 497-510 (2012).
9. L. S. Symington, J. Gautier, Double-strand break end resection and repair pathway choice. Annu. Rev. Genet. 45, 247-271 (2011).
10. F. Allen et al., Predicting the mutations generated by repair of Cas9-induced double-strand breaks. Nat. Biotechnol. (2019).
11. M. W. Shen et al., Predictable and precise template-free CRISPR editing of pathogenic variants. Nature (2018).
12. J. Shou, J. Li, Y. Liu, Q. Wu, Precise and Predictable CRISPR Chromosomal Rearrangements Reveal Principles of Cas9-Mediated Nucleotide Insertion. Mol. Cell (2018).
13. G. Gasiunas et al., A catalogue of biochemically diverse CRISPR-Cas9 orthologs. Nat. Commun. 11, 5512 (2020).
14. Z. Liang, S. Sunder, S. Nallasivam, T. E. Wilson, Overhang polarity of chromosomal double-strand breaks impacts kinetics and fidelity of yeast non-homologous end joining. Nucleic Acids Res. 44, 2769-2781 (2016).
15. C. C. So, A. Martin, DSB structure impacts DNA recombination leading to class switching and chromosomal translocations in human B cells. PLOS Genet. 15, e1008101 (2019).
16. B. P. Kleinstiver et al., High-fidelity CRISPR-Cas9 nucleases with no detectable genome-wide off-target effects. Nature. 529, 490-495 (2016).
17. I. M. Slaymaker et al., Rationally engineered Cas9 nucleases with improved specificity. Science. 351, 84-88 (2016).
18. F. Jiang et al., Structures of a CRISPR-Cas9 R-loop complex primed for DNA cleavage. Science. 351, 867-871 (2016).
19. E. K. Brinkman et al., Easy quantification of template-directed CRISPR/Cas9 editing. Nucleic Acids Res. (2018).
20. V. T. Chu et al., Increasing the efficiency of homology-directed repair for CRISPR-Cas9-induced precise gene editing in mammalian cells. Nat. Biotechnol. 33, 543-548 (2015).
21. A. Orthwein et al., A mechanism for the suppression of homologous recombination in G1 cells. Nature. 528, 422 (2015).
22. L. N. Truong et al., Microhomology-mediated End Joining and Homologous Recombination share the initial end resection step to repair DNA double-strand breaks in mammalian cells. Proc. Natl. Acad. Sci. (2013).
23. M. Mitra, L. D. Ho, H. A. Coller, An In Vitro Model of Cellular Quiescence in Primary Human Dermal Fibroblasts. Methods Mol. Biol. 1686, 27-47 (2018).
24. K. Clement et al., CRISPResso2 provides accurate and rapid genome editing sequence analysis. Nat. Biotechnol. 37, 224-226 (2019).

We claim:

1. A method of editing a genome in a cell, the method comprising:
exposing the cell to an engineered Cas nuclease comprising one or more mutations within the DNA binding cleft of the Cas nuclease,
wherein exposure to the engineered Cas nuclease decreases, inhibits, or prevents an indel-producing DNA repair pathway in the cell, and
wherein exposure to the engineered Cas nuclease increases one or more precise editing repair pathways within the cell.

2. The method of claim 1, wherein the engineered Cas nuclease is an engineered Cas9 nuclease.

3. The method of claim 1, wherein the precise editing repair pathway is homology directed repair (HDR), non-homologous end joining (NHEJ), or microhomology mediated end-joining (MMEJ).

4. The method of claim 1, wherein the precise editing repair pathway is a combination of micro-homology end joining (MMEJ) and homology directed repair (HDR).

5. The method of claim 4, wherein the ratio of NHEJ to MMEJ is decreased compared to that of a cell exposed to a reference Cas nuclease lacking the same mutations in the DNA binding cleft.

6. The method of claim 1, wherein the genome is in a non-dividing cell.

7. The method of claim 6, wherein the non-dividing cell is a quiescent cell, a senescent cell, or a fully differentiated cell.

8. The method of claim 1, wherein the one or more mutations comprise mutations of an amino acid residue at a position corresponding to D54, S55, K848, R976, N980, H982, K1003, T1314, N1317, or A1322 of SEQ ID NO: 2.

9. The method of claim 1, wherein the one or more mutations comprise mutations of one or more amino acid residues that occupy the same position in the three-dimensional structure of the DNA binding cleft as amino acids S55, R976, K1003, or T1314 from a Streptococcus pyogenes Cas9 protein.

10. The method of claim 2, wherein the engineered Cas9 nuclease comprises one or more mutations in the DNA binding cleft.

11. The method of claim 2, wherein the engineered Cas9 nuclease comprises a replacement of a sequence in the DNA binding cleft, wherein two or more non-sequential or sequential amino acids in the DNA binding cleft are replaced.

12. The method of claim 2, wherein the engineered Cas9 nuclease comprises one or more of S55, R976, K1003, or T1314 mutations.

13. The method of claim 1, wherein the engineered Cas nuclease decreases, inhibits, or prevents non-homologous end joining when compared to that of a reference Cas nuclease lacking said mutations.

14. The method of claim 1, wherein the reference Cas9 comprises mutations, insertions, or deletions of amino acids outside of the DNA binding cleft.

15. The method of claim 1, wherein the Cas nuclease is a fusion protein.

16. A method of precisely editing the genome of a non-dividing cell, the method comprising administering to the cell an agent capable of inhibiting or preventing non-homologous end joining (NHEJ) and increasing homology-driven repair (HDR).

17. The method of claim 16, wherein the agent is a modified Cas9 nuclease.

18. The method of claim 17, wherein the modified Cas9 nuclease comprises mutations at one or more amino acid residues in the DNA binding cleft.

19. An engineered Cas nuclease variant comprising two or more amino acid substitutions, mutations, or deletions in the DNA binding cleft such that the engineered Cas nuclease variant predominantly engages a homology-driven DNA repair pathway.

20. The engineered Cas nuclease of claim 19, wherein the Cas nuclease is a Cas9 nuclease.

21. A method of editing a genome in a cell, the method comprising:
exposing the cell to an engineered Cas nuclease comprising one or more mutations within the DNA binding cleft of the Cas nuclease,
wherein the engineered Cas nuclease is fused or otherwise associated with a polymerase to form a prime editor,
wherein exposure to the engineered Cas nuclease increases precise genome editing initiated by action of the associated polymerase, and
wherein exposure to the engineered Cas nuclease decreases, inhibits, or prevents byproduct indel formation in the cell.

22. The method of claim 21, wherein the wherein the engineered Cas nuclease is an engineered Cas9 nuclease within a prime editing system.

23. The method of claim 21, wherein the ratio of byproduct indels to precise genome edits is decreased compared to that of a cell exposed to a reference Cas nuclease within a prime editing system lacking the same mutations in the DNA binding cleft.

24. The method of claim 21, wherein the engineered Cas9 nuclease comprises mutations at one or more amino acid residues in the DNA binding cleft.

25. The method of claim 21, wherein the one or more mutations comprise mutations of an amino acid residue at a position corresponding to R780, K810, K848, K855, R976, H982, or T1314 of SEQ ID NO: 2.

26. The method of claim 21, wherein the one or more mutations comprise mutations of one or more amino acid residues that occupy the same position in the three-dimensional structure of the DNA binding cleft as amino acids R780, K810, K848, K855, R976, H982, or T1314 from a Streptococcus pyogenes Cas9 protein.

27. The method of claim 21, wherein the engineered Cas nuclease comprises the amino acids R221, N394, or both R221 and N394.

28. The method claim 21, wherein the engineered Cas nuclease is vPE.

29. An engineered Cas nuclease variant comprising one or more amino acid substitutions, mutations, or deletions in the DNA binding cleft, wherein the engineered Cas nuclease is fused or otherwise associated with a polymerase to form a prime editor.


SEQUENCE LISTING (294 sequences)

SEQ ID NO: 1 - DNA, 4107 bp, Streptococcus pyogenes
  source 1..4107: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactaa

SEQ ID NO: 2 - protein, 1368 aa, Streptococcus pyogenes [sequence omitted]

SEQ ID NO: 3 - protein, 1368 aa, Streptococcus pyogenes
MDKKYSIGLDIGTNSVGWAVITDEYKVPSKKFKVLGNTDRHSIKKNLIGALLFDRGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVAEINNYHHAHDAYLNAVVGTALIKKYPALESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFRLTNLGAPAAFKYFDTTIDRKRYTSTKEVLDATLIHQSITGLYETRIDLSQLGGD

SEQ ID NO: 4 - protein, 1368 aa, synthetic construct
  REGION 1..1368: note = R976A K1003A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVAEINNYHHAHDAYLNAVVGTALIKKYPALESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1368 aa, synthetic construct
  REGION 1..1368: note = D54R R976A K1003A
MDKKYSIGLDIGTNSVGWAVITDEYKVPSKKFKVLGNTDRHSIKKNLIGALLFRSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVAEINNYHHAHDAYLNAVVGTALIKKYPALESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 1368 aa, synthetic construct
  REGION 1..1368: note = S55R R976A K1003A
MDKKYSIGLDIGTNSVGWAVITDEYKVPSKKFKVLGNTDRHSIKKNLIGALLFDRGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVAEINNYHHAHDAYLNAVVGTALIKKYPALESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 1368 aa, synthetic construct
  REGION 1..1368: note = R976A N980R K1003A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVAEINRYHHAHDAYLNAVVGTALIKKYPALESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 8 - protein, 1368 aa, synthetic construct
  REGION 1..1368: note = R976A K1003A T1314R
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVAEINNYHHAHDAYLNAVVGTALIKKYPALESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFRLTNLGAPAAFKYFDTTIDRKRYTSTKEVLDATLIHQSITGLYETRIDLSQLGGD

SEQ ID NO: 9 - protein, 1368 aa, synthetic construct
  REGION 1..1368: note = R976A K1003A N1317R
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVAEINNYHHAHDAYLNAVVGTALIKKYPALESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPAAFKYFDTTIDRKRYTSTKEVLDATLIHQSITGLYETRIDLSQLGGD

SEQ ID NO: 10 - protein, 1368 aa, synthetic construct
  REGION 1..1368: note = R976A K1003A A1322R
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVAEINNYHHAHDAYLNAVVGTALIKKYPALESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKRYTSTKEVLDATLIHQSITGLYETRIDLSQLGGD

SEQ ID NO: 11 - protein, 1368 aa, synthetic construct
  REGION 1..1368: note = S55R
MDKKYSIGLDIGTNSVGWAVITDEYKVPSKKFKVLGNTDRHSIKKNLIGALLFDRGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVAEINRYHHAHDAYLNAVVGTALIKKYPALESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 12 - protein, 1368 aa, synthetic construct
  REGION 1..1368: note = R976A N980R K1003A T1314R
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVAEINRYHHAHDAYLNAVVGTALIKKYPALESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFRLTNLGAPAAFKYFDTTIDRKRYTSTKEVLDATLIHQSITGLYETRIDLSQLGGD

SEQ ID NO: 13 - protein, 1368 aa, synthetic construct
  REGION 1..1368: note = S55R R976A N980R K1003A T1314R
MDKKYSIGLDIGTNSVGWAVITDEYKVPSKKFKVLGNTDRHSIKKNLIGALLFDRGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVAEINRYHHAHDAYLNAVVGTALIKKYPALESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFRLTNLGAPAAFKYFDTTIDRKRYTSTKEVLDATLIHQSITGLYETRIDLSQLGGD

SEQ ID NO: 14 - DNA, 18 bp, synthetic construct
  misc_feature 1..18: note = cas9-mut-FWD
  source 1..18: mol_type = other DNA
ggttggaccggtgccacc

SEQ ID NO: 15 - DNA, 23 bp, synthetic construct
  misc_feature 1..23: note = cas9-mid-REV
  source 1..23: mol_type = other DNA
ggccagagggcccacgtagtagg

SEQ ID NO: 16 - DNA, 23 bp, synthetic construct
  misc_feature 1..23: note = cas9-mid-FWD
  source 1..23: mol_type = other DNA
cctactacgtgggccctctggcc

SEQ ID NO: 17 - DNA, 36 bp, synthetic construct
  misc_feature 1..36: note = cas9-mut-REV
  source 1..36: mol_type = other DNA
ctctaggaattcttactttttcttttttgcctggcc

SEQ ID NO: 18 - DNA, 44 bp, synthetic construct
  misc_feature 1..44: note = R780A-BOT
  source 1..44: mol_type = other DNA
ccctcttcgatccgcttcatggcctcgcggctgttcttctgtcc

SEQ ID NO: 19 - DNA, 44 bp, synthetic construct
  misc_feature 1..44: note = R780A-TOP
  source 1..44: mol_type = other DNA
ggacagaagaacagccgcgaggccatgaagcggatcgaagaggg

SEQ ID NO: 20 - DNA, 47 bp, synthetic construct
  misc_feature 1..47: note = R783A-BOT
  source 1..47: mol_type = other DNA
gctctttgatgccctcttcgatggccttcattctctcgcggctgttc

SEQ ID NO: 21 - DNA, 47 bp, synthetic construct
  misc_feature 1..47: note = R783A-TOP
  source 1..47: mol_type = other DNA
gaacagccgcgagagaatgaaggccatcgaagagggcatcaaagagc

SEQ ID NO: 22 - DNA, 47 bp, synthetic construct
  misc_feature 1..47: note = K810A-BOT
  source 1..47: mol_type = other DNA
tctgcaggtagtacaggtacagggcctcgttctgcagctgggtgttt

SEQ ID NO: 23 - DNA, 47 bp, synthetic construct
  misc_feature 1..47: note = K810A-TOP
  source 1..47: mol_type = other DNA
aaacacccagctgcagaacgaggccctgtacctgtactacctgcaga

SEQ ID NO: 24 - DNA, 47 bp, synthetic construct
  misc_feature 1..47: note = R832A-BOT
  source 1..47: mol_type = other DNA
ggtccacatcgtagtcggacagggcgttgatgtccagttcctggtcc

SEQ ID NO: 25 - DNA, 47 bp, synthetic construct
  misc_feature 1..47: note = R832A-TOP
  source 1..47: mol_type = other DNA
ggaccaggaactggacatcaacgccctgtccgactacgatgtggacc

SEQ ID NO: 26 - DNA, 47 bp, synthetic construct
  misc_feature 1..47: note = K848A-BOT
  source 1..47: mol_type = other DNA
ccttgttgtcgatggagtcgtcggccagaaagctctgaggcacgata

SEQ ID NO: 27 - DNA, 47 bp, synthetic construct
  misc_feature 1..47: note = K848A-TOP
  source 1..47: mol_type = other DNA
tatcgtgcctcagagctttctggccgacgactccatcgacaacaagg

SEQ ID NO: 28 - DNA, 43 bp, synthetic construct
  misc_feature 1..43: note = K855A-BOT
  source 1..43: mol_type = other DNA
ttgtcgcttctggtcagcacggcgttgtcgatggagtcgtcct

SEQ ID NO: 29 - DNA, 43 bp, synthetic construct
  misc_feature 1..43: note = K855A-TOP
  source 1..43: mol_type = other DNA
aggacgactccatcgacaacgccgtgctgaccagaagcgacaa

SEQ ID NO: 30 - DNA, 38 bp, synthetic construct
  misc_feature 1..38: note = S964A-BOT
  source 1..38: mol_type = other DNA
aaatccttccggaaatcggccaccagcttggacttcag

SEQ ID NO: 31 - DNA, 38 bp, synthetic construct
  misc_feature 1..38: note = S964A-TOP
  source 1..38: mol_type = other DNA
ctgaagtccaagctggtggccgatttccggaaggattt

SEQ ID NO: 32 - DNA, 49 bp, synthetic construct
  misc_feature 1..49: note = K968A-BOT
  source 1..49: mol_type = other DNA
cgcactttgtaaaactggaaatcggcccggaaatcggacaccagcttgg

SEQ ID NO: 33 - DNA, 49 bp, synthetic construct
  misc_feature 1..49: note = K968A-TOP
  source 1..49: mol_type = other DNA
ccaagctggtgtccgatttccgggccgatttccagttttacaaagtgcg

SEQ ID NO: 34 - DNA, 49 bp, synthetic construct
  misc_feature 1..49: note = R976A-BOT
  source 1..49: mol_type = other DNA
gcgtggtggtagttgttgatctcggccactttgtaaaactggaaatcct

SEQ ID NO: 35 - DNA, 49 bp, synthetic construct
  misc_feature 1..49: note = R976A-TOP
  source 1..49: mol_type = other DNA
aggatttccagttttacaaagtggccgagatcaacaactaccaccacgc

SEQ ID NO: 36 - DNA, 39 bp, synthetic construct
  misc_feature 1..39: note = H982A-BOT
  source 1..39: mol_type = other DNA
gtaggcgtcgtgggcgtgggcgtagttgttgatctcgcg

SEQ ID NO: 37 - DNA, 39 bp, synthetic construct
  misc_feature 1..39: note = H982A-TOP
  source 1..39: mol_type = other DNA
cgcgagatcaacaactacgcccacgcccacgacgcctac

SEQ ID NO: 38 - DNA, 47 bp, synthetic construct
  misc_feature 1..47: note = K1003A-BOT
  source 1..47: mol_type = other DNA
cgtacacgaactcgctttccagggcagggtactttttgatcagggcg

SEQ ID NO: 39 - DNA, 47 bp, synthetic construct
  misc_feature 1..47: note = K1003A-TOP
  source 1..47: mol_type = other DNA
cgccctgatcaaaaagtaccctgccctggaaagcgagttcgtgtacg

SEQ ID NO: 40 - DNA, 49 bp, synthetic construct
  misc_feature 1..49: note = K1047A-BOT
  source 1..49: mol_type = other DNA
gccgttggccagggtaatctcggtggcgaaaaagttcatgatgttgctg

SEQ ID NO: 41 - DNA, 49 bp, synthetic construct
  misc_feature 1..49: note = K1047A-TOP
  source 1..49: mol_type = other DNA
cagcaacatcatgaactttttcgccaccgagattaccctggccaacggc

SEQ ID NO: 42 - DNA, 43 bp, synthetic construct
  misc_feature 1..43: note = R1060A-BOT
  source 1..43: mol_type = other DNA
ccgtttgtctcgatcagaggggccttccggatctcgccgttgg

SEQ ID NO: 43 - DNA, 43 bp, synthetic construct
  misc_feature 1..43: note = R1060A-TOP
  source 1..43: mol_type = other DNA
ccaacggcgagatccggaaggcccctctgatcgagacaaacgg

SEQ ID NO: 44 - DNA, 39 bp, synthetic construct
  misc_feature 1..39: note = R976A-H982A-BOT
  source 1..39: mol_type = other DNA
gtaggcgtcgtgggcgtgggcgtagttgttgatctcggc

SEQ ID NO: 45 - DNA, 39 bp, synthetic construct
  misc_feature 1..39: note = R976A-H982A-TOP
  source 1..39: mol_type = other DNA
gccgagatcaacaactacgcccacgcccacgacgcctac

SEQ ID NO: 46 - DNA, 35 bp, synthetic construct
  misc_feature 1..35: note = D54R-TOP
  source 1..35: mol_type = other DNA
cggagccctgctgttccggagcggcgaaacagccg

SEQ ID NO: 47 - DNA, 35 bp, synthetic construct
  misc_feature 1..35: note = D54R-BOT
  source 1..35: mol_type = other DNA
cggctgtttcgccgctccggaacagcagggctccg

SEQ ID NO: 48 - DNA, 33 bp, synthetic construct
  misc_feature 1..33: note = S55R-TOP
  source 1..33: mol_type = other DNA
gccctgctgttcgaccggggcgaaacagccgag

SEQ ID NO: 49 - DNA, 33 bp, synthetic construct
  misc_feature 1..33: note = S55R-BOT
  source 1..33: mol_type = other DNA
ctcggctgtttcgccccggtcgaacagcagggc

SEQ ID NO: 50 - DNA, 41 bp, synthetic construct
  misc_feature 1..41: note = N980R-TOP
  source 1..41: mol_type = other DNA
caaagtggccgagatcaaccggtaccaccacgcccacgacg

SEQ ID NO: 51 - DNA, 41 bp, synthetic construct
  misc_feature 1..41: note = N980R-BOT
  source 1..41: mol_type = other DNA
cgtcgtgggcgtggtggtaccggttgatctcggccactttg

SEQ ID NO: 52 - DNA, 47 bp, synthetic construct
  misc_feature 1..47: note = T1314R-TOP
  source 1..47: mol_type = other DNA
cgagaatatcatccacctgtttcggctgaccaatctgggagcccctg

SEQ ID NO: 53 - DNA, 47 bp, synthetic construct
  misc_feature 1..47: note = T1314R-BOT
  source 1..47: mol_type = other DNA
caggggctcccagattggtcagccgaaacaggtggatgatattctcg

SEQ ID NO: 54 - DNA, 41 bp, synthetic construct
  misc_feature 1..41: note = N1317R-TOP
  source 1..41: mol_type = other DNA
ccacctgtttaccctgacccggctgggagcccctgccgcct

SEQ ID NO: 55 - DNA, 41 bp, synthetic construct
  misc_feature 1..41: note = N1317R-BOT
  source 1..41: mol_type = other DNA
aggcggcaggggctcccagccgggtcagggtaaacaggtgg

SEQ ID NO: 56 - DNA, 49 bp, synthetic construct
  misc_feature 1..49: note = A1322R-TOP
  source 1..49: mol_type = other DNA
ccctgaccaatctgggagcccctcgggccttcaagtactttgacaccac

SEQ ID NO: 57 - DNA, 49 bp, synthetic construct
  misc_feature 1..49: note = A1322R-BOT
  source 1..49: mol_type = other DNA
gtggtgtcaaagtacttgaaggcccgaggggctcccagattggtcaggg

SEQ ID NO: 58 - DNA, 44 bp, synthetic construct
  misc_feature 1..44: note = gRNA-scaffold-NheI-FWD
  source 1..44: mol_type = other DNA
ctcagctagcgagggcctatttcccatgattccttcatatttgc

SEQ ID NO: 59 - DNA, 37 bp, synthetic construct
  misc_feature 1..37: note = gRNA-scaffold-EcoRI-REV
  source 1..37: mol_type = other DNA
atcagaattcaaaaaaagcaccgactcggtgccactt

SEQ ID NO: 60 - DNA, 24 bp, synthetic construct
  misc_feature 1..24: note = AAVS1-gRNA1-BOT
  source 1..24: mol_type = other DNA
aaacctagggacaggattggtgac

SEQ ID NO: 61 - DNA, 24 bp, synthetic construct
  misc_feature 1..24: note = AAVS1-gRNA1-TOP
  source 1..24: mol_type = other DNA
caccgtcaccaatcctgtccctag

SEQ ID NO: 62 - DNA, 24 bp, synthetic construct
  misc_feature 1..24: note = AAVS1-gRNA2-BOT
  source 1..24: mol_type = other DNA
aaacatcctgtccctagtggcccc

SEQ ID NO: 63 - DNA, 24 bp, synthetic construct
  misc_feature 1..24: note = AAVS1-gRNA2-TOP
  source 1..24: mol_type = other DNA
caccggggccactagggacaggat

SEQ ID NO: 64 - DNA, 24 bp, synthetic construct
  misc_feature 1..24: note = CD274-gRNA1-BOT
  source 1..24: mol_type = other DNA
aaacgggaacttcaaattcattcc

SEQ ID NO: 65 - DNA, 24 bp, synthetic construct
  misc_feature 1..24: note = CD274-gRNA1-TOP
  source 1..24: mol_type = other DNA
caccggaatgaatttgaagttccc

SEQ ID NO: 66 - DNA, 24 bp, synthetic construct
  misc_feature 1..24: note = CD274-gRNA2-BOT
  source 1..24: mol_type = other DNA
aaactgggaacttcaaattcattc

SEQ ID NO: 67 - DNA, 24 bp, synthetic construct
  misc_feature 1..24: note = CD274-gRNA2-TOP
  source 1..24: mol_type = other DNA
caccgaatgaatttgaagttccca

SEQ ID NO: 68 - DNA, 24 bp, synthetic construct
  misc_feature 1..24: note = CXCR4-gRNA-BOT
  source 1..24: mol_type = other DNA
aaaccctctttgtcatcacgcttc

SEQ ID NO: 69 - DNA, 24 bp, synthetic construct
  misc_feature 1..24: note = CXCR4-gRNA-TOP
  source 1..24: mol_type = other DNA
caccgaagcgtgatgacaaagagg

SEQ ID NO: 70 - DNA, 24 bp, synthetic construct
  misc_feature 1..24: note = EMX1-gRNA1-BOT
  source 1..24: mol_type = other DNA
aaacccctagtcattggaggtgac

SEQ ID NO: 71 - DNA, 24 bp, synthetic construct
  misc_feature 1..24: note = EMX1-gRNA1-TOP
  source 1..24: mol_type = other DNA
caccgtcacctccaatgactaggg

SEQ ID NO: 72 - DNA, 24 bp, synthetic construct
  misc_feature 1..24: note = EMX1-gRNA2-BOT
  source 1..24: mol_type = other DNA
aaacttcttcttctgctcggactc

SEQ ID NO: 73 - DNA, 24 bp, synthetic construct
  misc_feature 1..24: note = EMX1-gRNA2-TOP
  source 1..24: mol_type = other DNA
caccgagtccgagcagaagaagaa

SEQ ID NO: 74 - DNA, 24 bp, synthetic construct
  misc_feature 1..24: note = GFP-gRNA-BOT
  source 1..24: mol_type = other DNA
aaacacggcgtgcagtgcttcagc

SEQ ID NO: 75 - DNA, 24 bp, synthetic construct
  misc_feature 1..24: note = GFP-gRNA-TOP
  source 1..24: mol_type = other DNA
caccgctgaagcactgcacgccgt

SEQ ID NO: 76 - DNA, 24 bp, synthetic construct
  misc_feature 1..24: note = KRAS-gRNA1-BOT
  source 1..24: mol_type = other DNA
aaaccgattattatcagcctcagc

SEQ ID NO: 77 - DNA, 24 bp, synthetic construct
  misc_feature 1..24: note = KRAS-gRNA1-TOP
  source 1..24: mol_type = other DNA
caccgctgaggctgataataatcg

SEQ ID NO: 78 - DNA, 24 bp, synthetic construct
  misc_feature 1..24: note = KRAS-gRNA2-BOT
  source 1..24: mol_type = other DNA
aaacccccgattattatcagcctc

SEQ ID NO: 79 - DNA, 24 bp, synthetic construct
  misc_feature 1..24: note = KRAS-gRNA2-TOP
  source 1..24: mol_type = other DNA
caccgaggctgataataatcgggg

SEQ ID NO: 80 - DNA, 24 bp, synthetic construct
  misc_feature 1..24: note = MYC-gRNA1-BOT
  source 1..24: mol_type = other DNA
aaacgcgtcgggagagtcgcgtcc

SEQ ID NO: 81 - DNA, 24 bp, synthetic construct
  misc_feature 1..24: note = MYC-gRNA1-TOP
  source 1..24: mol_type = other DNA
caccggacgcgactctcccgacgc

SEQ ID NO: 82 - DNA, 24 bp, synthetic construct
  misc_feature 1..24: note = MYC-gRNA2-BOT
  source 1..24: mol_type = other DNA
aaaccgcgtcgggagagtcgcgtc

SEQ ID NO: 83 - DNA, 24 bp, synthetic construct
  misc_feature 1..24: note = MYC-gRNA2-TOP
  source 1..24: mol_type = other DNA
caccgacgcgactctcccgacgcg

SEQ ID NO: 84 - DNA, 24 bp, synthetic construct
  misc_feature 1..24: note = MYC-gRNA3-BOT
  source 1..24: mol_type = other DNA
caccgcgactctcccgacgcgggg

SEQ ID NO: 85 - DNA, 24 bp, synthetic construct
  misc_feature 1..24: note = MYC-gRNA3-TOP
  source 1..24: mol_type = other DNA
aaacccccgcgtcgggagagtcgc

SEQ ID NO: 86 - DNA, 24 bp, synthetic construct
  misc_feature 1..24: note = STAT1-gRNA1-BOT
  source 1..24: mol_type = other DNA
aaacccagctgcaagcatgtcatc

SEQ ID NO: 87 - DNA, 24 bp, synthetic construct
  misc_feature 1..24: note = STAT1-gRNA1-TOP
  source 1..24: mol_type = other DNA
caccgatgacatgcttgcagctgg

SEQ ID NO: 88 - DNA, 24 bp, synthetic construct
  misc_feature 1..24: note = STAT1-gRNA2-BOT
  source 1..24: mol_type = other DNA
aaaccagctgcaagcatgtcatcc

SEQ ID NO: 89 - DNA, 24 bp, synthetic construct
  misc_feature 1..24: note = STAT1-gRNA2-TOP
  source 1..24: mol_type = other DNA
caccggatgacatgcttgcagctg

SEQ ID NO: 90 - DNA, 24 bp, synthetic construct
  misc_feature 1..24: note = TGFB1-gRNA-BOT
  source 1..24: mol_type = other DNA
aaaccttggtggaagcgcaggctc

SEQ ID NO: 91 - DNA, 24 bp, synthetic construct
  misc_feature 1..24: note = TGFB1-gRNA-TOP
  source 1..24: mol_type = other DNA
caccgagcctgcgcttccaccaag

SEQ ID NO: 92 - DNA, 24 bp, synthetic construct
  misc_feature 1..24: note = VEGFA-gRNA1-BOT
  source 1..24: mol_type = other DNA
aaaccacgcacacactcactcacc

SEQ ID NO: 93 - DNA, 24 bp, synthetic construct
  misc_feature 1..24: note = VEGFA-gRNA1-TOP
  source 1..24: mol_type = other DNA
caccggtgagtgagtgtgtgcgtg

SEQ ID NO: 94 - DNA, 24 bp, synthetic construct
  misc_feature 1..24: note = VEGFA-gRNA2-BOT
  source 1..24: mol_type = other DNA
aaacacacgcacacactcactcac

SEQ ID NO: 95 - DNA, 24 bp, synthetic construct
  misc_feature 1..24: note = VEGFA-gRNA2-TOP
  source 1..24: mol_type = other DNA
caccgtgagtgagtgtgtgcgtgt

SEQ ID NO: 96 - DNA, 114 bp, synthetic construct
  misc_feature 1..114: note = AAVS1-HDRtemplate
  source 1..114: mol_type = other DNA
cagggccggttaatgtggctctggttctgggtacttttatctgtcccctccaccccacagtggggccgctagcaagcttggacaggattggtgacagaaaagccccatccttag

SEQ ID NO: 97 - DNA, 113 bp, synthetic construct
  misc_feature 1..113: note = CD274-HDRtemplate
  source 1..113: mol_type = other DNA
tatgaaagataatgaaaagctatgggaaagataacttagaaacaaagaaggcatggatcctcagccctgggctagcaagcttcaaattcattccatctgctatataagaaaca

SEQ ID NO: 98 - DNA, 116 bp, synthetic construct
  misc_feature 1..116: note = CXCR4-HDRtemplate
  source 1..116: mol_type = other DNA
atgggttaccagaagaaactgagaagcatgacggacaagtacaggctgcacctgtcagtggccgacctgctagcaagcttctttgtcatcacgcttcccttctgggcagttgatgc

SEQ ID NO: 99 - DNA, 116 bp, synthetic construct
  misc_feature 1..116: note = EMX1-HDRtemplate
  source 1..116: mol_type = other DNA
cttgggcccacgcaggggcctggccagcagcaagcagcactctgccctcgtgggtttgtggttgcccaccgctagcaagcttgtcattggaggtgacatcgatgtcctccccattg

SEQ ID NO: 100 - DNA, 107 bp, synthetic construct
  misc_feature 1..107: note = GFP-BFP-HDRtemplate
  source 1..107: mol_type = other DNA
cctgaagttcatctgcaccaccggcaagctgcccgtgccctggcccaccctcgtgaccaccctgagccacggggtgcagtgcttcagccgctaccccgaccacatga

SEQ ID NO: 101 - DNA, 111 bp, synthetic construct
  misc_feature 1..111: note = KRAS-HDRtemplate
  source 1..111: mol_type = other DNA
ggcgacttcggggacttagggagaccgggcggacgatttcccacaccggggctgtctgatcgccggctagcaagcttattatcagcctcagcacttgggctgggaatttag

SEQ ID NO: 102 - DNA, 112 bp, synthetic construct
  misc_feature 1..112: note = MYC-HDRtemplate
  source 1..112: mol_type = other DNA
agcctttcagagaagcgggtcctggcagcggcggggaagtgtccccaaatgggcagaatagcctcgctagcaagcttcgggagagtcgcgtccttgctcgggtgttgtaagt

SEQ ID NO: 103 - DNA, 112 bp, synthetic construct
  misc_feature 1..112: note = STAT1-HDRtemplate
  source 1..112: mol_type = other DNA
gtatttctaatagacttgaaaggacagccaggagcaaagatgggcagaaggacaacctgtttccccaagcttgctagcaagcatgtcatcctcacatttggccccttggccc

SEQ ID NO: 104 - DNA, 112 bp, synthetic construct
  misc_feature 1..112: note = TGFB1-HDRtemplate
  source 1..112: mol_type = other DNA
accctgagaggaactgggactttggggtccagactgccagcgtttagcgcagcggggtcctcctgccccttggaagcttgctagcgcaggctcctccccccgcgcgtggcac

SEQ ID NO: 105 - DNA, 113 bp, synthetic construct
  misc_feature 1..113: note = VEGFA-HDRtemplate
  source 1..113: mol_type = other DNA
acacacagatctattggaatcctggagtgacccctggccttctccccgctccaacgccctcaaccccacgctagcaagcttacactcactcacccacacagacacacacgtcc

SEQ ID NO: 106 - DNA, 27 bp, synthetic construct
  misc_feature 1..27: note = AAVS1-MDRtemplate-BOT
  source 1..27: mol_type = other DNA
aatccggtgtccctagtggccccactg

SEQ ID NO: 107 - DNA, 27 bp, synthetic construct
  misc_feature 1..27: note = AAVS1-MDRtemplate-TOP
  source 1..27: mol_type = other DNA
ggacaccggattggtgacagaaaagcc

SEQ ID NO: 108 - DNA, 25 bp, synthetic construct
  misc_feature 1..25: note = CD274-MDRtemplate-BOT
  source 1..25: mol_type = other DNA
ctgggacttcaaattcattccatct

SEQ ID NO: 109 - DNA, 25 bp, synthetic construct
  misc_feature 1..25: note = CD274-MDRtemplate-TOP
  source 1..25: mol_type = other DNA
gaagtcccagggctgaggatccatg

SEQ ID NO: 110 - DNA, 26 bp, synthetic construct
  misc_feature 1..26: note = CXCR4-MDRtemplate-BOT
  source 1..26: mol_type = other DNA
acaaacaggaggtcggccactgacag

SEQ ID NO: 111 - DNA, 26 bp, synthetic construct
  misc_feature 1..26: note = CXCR4-MDRtemplate-TOP
  source 1..26: mol_type = other DNA
ctcctgtttgtcatcacgcttccctt

SEQ ID NO: 112 - DNA, 28 bp, synthetic construct
  misc_feature 1..28: note = GFP-BFP-5'0-3'20-MDRtemplate-BOT
  source 1..28: mol_type = other DNA
cccgtggctcagggtggtcacgagggtg

SEQ ID NO: 113 - DNA, 28 bp, synthetic construct
  misc_feature 1..28: note = GFP-BFP-5'0-3'20-MDRtemplate-TOP
  source 1..28: mol_type = other DNA
gccacggggtgcagtgcttcagccgcta

SEQ ID NO: 114 - DNA, 33 bp, synthetic construct
  misc_feature 1..33: note = GFP-BFP-5'5-3'20-MDRtemplate-BOT
  source 1..33: mol_type = other DNA
tgcaccccgtggctcagggtggtcacgagggtg

SEQ ID NO: 115 - DNA, 33 bp, synthetic construct
  misc_feature 1..33: note = GFP-BFP-5'5-3'20-MDRtemplate-TOP
  source 1..33: mol_type = other DNA
cctgagccacggggtgcagtgcttcagccgcta

SEQ ID NO: 116 - DNA, 38 bp, synthetic construct
  misc_feature 1..38: note = GFP-BFP-5'10-3'20-MDRtemplate-BOT
  source 1..38: mol_type = other DNA
agcactgcaccccgtggctcagggtggtcacgagggtg

SEQ ID NO: 117 - DNA, 38 bp, synthetic construct
  misc_feature 1..38: note = GFP-BFP-5'10-3'20-MDRtemplate-TOP
  source 1..38: mol_type = other DNA
accaccctgagccacggggtgcagtgcttcagccgcta

SEQ ID NO: 118 - DNA, 27 bp, synthetic construct
  misc_feature 1..27: note = KRAS-MDRtemplate-BOT
  source 1..27: mol_type = other DNA
ccgcctagattattatcagcctcagca

SEQ ID NO: 119 - DNA, 27 bp, synthetic construct
  misc_feature 1..27: note = KRAS-MDRtemplate-TOP
  source 1..27: mol_type = other DNA
taatctaggcggcgatcagacagcccc

SEQ ID NO: 120 - DNA, 26 bp, synthetic construct
  misc_feature 1..26: note = MYC-MDRtemplate-BOT
  source 1..26: mol_type = other DNA
cccgctgtcgggagagtcgcgtcctt

SEQ ID NO: 121 - DNA, 26 bp, synthetic construct
  misc_feature 1..26: note = MYC-MDRtemplate-TOP
  source 1..26: mol_type = other DNA
ccgacagcggggaggctattctgccc

SEQ ID NO: 122 - DNA, 26 bp, synthetic construct
  misc_feature 1..26: note = TGFB1-MDRtemplate-BOT
  source 1..26: mol_type = other DNA
cccctctggtggaagcgcaggctcct

SEQ ID NO: 123 - DNA, 26 bp, synthetic construct
  misc_feature 1..26: note = TGFB1-MDRtemplate-TOP
  source 1..26: mol_type = other DNA
caccagaggggcaggaggaccccgct

SEQ ID NO: 124 - DNA, 61 bp, synthetic construct
  misc_feature 1..61: note = AAVS1-seq-r1-FWD
  misc_feature 34: note = n is a, c, g, or t
  source 1..61: mol_type = other DNA
acactctttccctacacgacgctcttccgatctntcacggttaatgtggctctggttctgg

SEQ ID NO: 125 - DNA, 61 bp, synthetic construct
  misc_feature 1..61: note = AAVS1-seq-r2-FWD
  misc_feature 34: note = n is a, c, g, or t
  source 1..61: mol_type = other DNA
acactctttccctacacgacgctcttccgatctnagtcggttaatgtggctctggttctgg

SEQ ID NO: 126 - DNA, 61 bp, synthetic construct
  misc_feature 1..61: note = AAVS1-seq-r3-FWD
  misc_feature 34: note = n is a, c, g, or t
  source 1..61: mol_type = other DNA
acactctttccctacacgacgctcttccgatctncagcggttaatgtggctctggttctgg

SEQ ID NO: 127 - DNA, 60 bp, synthetic construct
  misc_feature 1..60: note = AAVS1-seq-REV
  source 1..60: mol_type = other DNA
gactggagttcagacgtgtgctcttccgatctggggttagacccaatatcaggagactag

SEQ ID NO: 128 - DNA, 60 bp, synthetic construct
  misc_feature 1..60: note = CD274-seq-r1-FWD
  misc_feature 34: note = n is a, c, g, or t
  source 1..60: mol_type = other DNA
acactctttccctacacgacgctcttccgatctnatctgtatgtctgctgtgtactttgc

SEQ ID NO: 129 - DNA, 60 bp, synthetic construct
  misc_feature 1..60: note = CD274-seq-r2-FWD
  misc_feature 34: note = n is a, c, g, or t
  source 1..60: mol_type = other DNA
acactctttccctacacgacgctcttccgatctncgatgtatgtctgctgtgtactttgc

SEQ ID NO: 130 - DNA, 60 bp, synthetic construct
  misc_feature 1..60: note = CD274-seq-r3-FWD
  misc_feature 34: note = n is a, c, g, or t
  source 1..60: mol_type = other DNA
acactctttccctacacgacgctcttccgatctntagtgtatgtctgctgtgtactttgc

SEQ ID NO: 131 - DNA, 57 bp, synthetic construct
  misc_feature 1..57: note = CD274-seq-REV
  source 1..57: mol_type = other DNA
gactggagttcagacgtgtgctcttccgatctacttaacaaatggtggttgtctaaa

SEQ ID NO: 132 - DNA, 58 bp, synthetic construct
  misc_feature 1..58: note = CXCR4-seq-r1-FWD
  misc_feature 34: note = n is a, c, g, or t
  source 1..58: mol_type = other DNA
acactctttccctacacgacgctcttccgatctncgttggtcatgggttaccagaaga

SEQ ID NO: 133 - DNA, 58 bp, synthetic construct
  misc_feature 1..58: note = CXCR4-seq-r2-FWD
  misc_feature 34: note = n is a, c, g, or t
  source 1..58: mol_type = other DNA
acactctttccctacacgacgctcttccgatctnacgtggtcatgggttaccagaaga

SEQ ID NO: 134 - DNA, 58 bp, synthetic construct
  misc_feature 1..58: note = CXCR4-seq-r3-FWD
  misc_feature 34: note = n is a, c, g, or t
  source 1..58: mol_type = other DNA
acactctttccctacacgacgctcttccgatctngtatggtcatgggttaccagaaga

SEQ ID NO: 135 - DNA, 52 bp, synthetic construct
  misc_feature 1..52: note = CXCR4-seq-REV
  source 1..52: mol_type = other DNA
gactggagttcagacgtgtgctcttccgatctgactgatgaaggccaggatg

SEQ ID NO: 136 - DNA, 57 bp, synthetic construct
  misc_feature 1..57: note = EMX1-seq-r1-FWD
  misc_feature 34: note = n is a, c, g, or t
  source 1..57: mol_type = other DNA
acactctttccctacacgacgctcttccgatctngaccctgagtccgagcagaagaa

SEQ ID NO: 137 - DNA, 57 bp, synthetic construct
  misc_feature 1..57: note = EMX1-seq-r2-FWD
  misc_feature 34: note = n is a, c, g, or t
  source 1..57: mol_type = other DNA
acactctttccctacacgacgctcttccgatctntgacctgagtccgagcagaagaa

SEQ ID NO: 138 - DNA, 57 bp, synthetic construct
  misc_feature 1..57: note = EMX1-seq-r3-FWD
  misc_feature 34: note = n is a, c, g, or t
  source 1..57: mol_type = other DNA
acactctttccctacacgacgctcttccgatctnactcctgagtccgagcagaagaa

SEQ ID NO: 139 - DNA, 51 bp, synthetic construct
  misc_feature 1..51: note = EMX1-seq-REV
  source 1..51: mol_type = other DNA
gactggagttcagacgtgtgctcttccgatctagtggccagagtccagctt

SEQ ID NO: 140 - DNA, 59 bp, synthetic construct
  misc_feature 1..59: note = GFP-seq-r1-FWD
  misc_feature 34: note = n is a, c, g, or t
  source 1..59: mol_type = other DNA
acactctttccctacacgacgctcttccgatctnatgccctgaagttcatctgcaccac

SEQ ID NO: 141 - DNA, 59 bp, synthetic construct
  misc_feature 1..59: note = GFP-seq-r2-FWD
  misc_feature 34: note = n is a, c, g, or t
  source 1..59: mol_type = other DNA
acactctttccctacacgacgctcttccgatctngatccctgaagttcatctgcaccac

SEQ ID NO: 142 - DNA, 59 bp, synthetic construct
  misc_feature 1..59: note = GFP-seq-r3-FWD
  misc_feature 34: note = n is a, c, g, or t
  source 1..59: mol_type = other DNA
acactctttccctacacgacgctcttccgatctntgcccctgaagttcatctgcaccac

SEQ ID NO: 143 - DNA, 54 bp, synthetic construct
  misc_feature 1..54: note = GFP-seq-REV
  source 1..54: mol_type = other DNA
gactggagttcagacgtgtgctcttccgatcttagttgccgtcgtccttgaaga

SEQ ID NO: 144 - DNA, 59 bp, synthetic construct
  misc_feature 1..59: note = KRAS-seq-r1-FWD
  misc_feature 34: note = n is a, c, g, or t
  source 1..59: mol_type = other DNA
acactctttccctacacgacgctcttccgatctngacttgaaagggtctgtcgtgtttg

SEQ ID NO: 145 - DNA, 59 bp, synthetic construct
  misc_feature 1..59: note = KRAS-seq-r2-FWD
  misc_feature 34: note = n is a, c, g, or t
  source 1..59: mol_type = other DNA
acactctttccctacacgacgctcttccgatctntgattgaaagggtctgtcgtgtttg

SEQ ID NO: 146 - DNA, 59 bp, synthetic construct
  misc_feature 1..59: note = KRAS-seq-r3-FWD
  misc_feature 34: note = n is a, c, g, or t
  source 1..59: mol_type = other DNA
acactctttccctacacgacgctcttccgatctnactttgaaagggtctgtcgtgtttg

SEQ ID NO: 147 - DNA, 55 bp, synthetic construct
  misc_feature 1..55: note = KRAS-seq-REV
  source 1..55: mol_type = other DNA
gactggagttcagacgtgtgctcttccgatctaaacaagcagtcaccaaaagtgg

SEQ ID NO: 148 - DNA, 58 bp, synthetic construct
  misc_feature 1..58: note = MYC-seq-r1-FWD
  misc_feature 34: note = n is a, c, g, or t
  source 1..58: mol_type = other DNA
acactctttccctacacgacgctcttccgatctncgtcacgaaactttgcccatagca

SEQ ID NO: 149 - DNA, 58 bp, synthetic construct
  misc_feature 1..58: note = MYC-seq-r2-FWD
  misc_feature 34: note = n is a, c, g, or t
  source 1..58: mol_type = other DNA
acactctttccctacacgacgctcttccgatctnacgcacgaaactttgcccatagca

SEQ ID NO: 150 - DNA, 58 bp, synthetic construct
  misc_feature 1..58: note = MYC-seq-r3-FWD
  misc_feature 34: note = n is a, c, g, or t
  source 1..58: mol_type = other DNA
acactctttccctacacgacgctcttccgatctngtacacgaaactttgcccatagca

SEQ ID NO: 151 - DNA, 53 bp, synthetic construct
  misc_feature 1..53: note = MYC-seq-REV
  source 1..53: mol_type = other DNA
gactggagttcagacgtgtgctcttccgatctaagtggacttcggtgcttacc

SEQ ID NO: 152 - DNA, 58 bp, synthetic construct
  misc_feature 1..58: note = STAT1-seq-r1-FWD
  misc_feature 34: note = n is a, c, g, or t
  source 1..58: mol_type = other DNA
acactctttccctacacgacgctcttccgatctnatgaaagtagtatgcgtgggcctc

SEQ ID NO: 153 - DNA, 58 bp, synthetic construct
  misc_feature 1..58: note = STAT1-seq-r2-FWD
  misc_feature 34: note = n is a, c, g, or t
  source 1..58: mol_type = other DNA
acactctttccctacacgacgctcttccgatctngataaagtagtatgcgtgggcctc

SEQ ID NO: 154 - DNA, 58 bp, synthetic construct
  misc_feature 1..58: note = STAT1-seq-r3-FWD
  misc_feature 34: note = n is a, c, g, or t
  source 1..58: mol_type = other DNA
acactctttccctacacgacgctcttccgatctntgcaaagtagtatgcgtgggcctc

SEQ ID NO: 155 - DNA, 55 bp, synthetic construct
  misc_feature 1..55: note = STAT1-seq-REV
  source 1..55: mol_type = other DNA
gactggagttcagacgtgtgctcttccgatctgctcaaaagctggtaaaccttca

SEQ ID NO: 156 - DNA, 59 bp, synthetic construct
  misc_feature 1..59: note = TGFB1-seq-r1-FWD
  misc_feature 34: note = n is a, c, g, or t
  source 1..59: mol_type = other DNA
acactctttccctacacgacgctcttccgatctngacgtgactctacaagaccgaggtg

SEQ ID NO: 157 - DNA, 59 bp, synthetic construct
  misc_feature 1..59: note = TGFB1-seq-r2-FWD
  misc_feature 34: note = n is a, c, g, or t
  source 1..59: mol_type = other DNA
acactctttccctacacgacgctcttccgatctntgagtgactctacaagaccgaggtg

SEQ ID NO: 158 - DNA, 59 bp, synthetic construct
  misc_feature 1..59: note = TGFB1-seq-r3-FWD
  misc_feature 34: note = n is a, c, g, or t
  source 1..59: mol_type = other DNA
acactctttccctacacgacgctcttccgatctnactgtgactctacaagaccgaggtg

SEQ ID NO: 159 - DNA, 54 bp, synthetic construct
  misc_feature 1..54: note = TGFB1-seq-REV
  source 1..54: mol_type = other DNA
gactggagttcagacgtgtgctcttccgatctcctgagaggaactgggactttg

SEQ ID NO: 160 - DNA, 57 bp, synthetic construct
  misc_feature 1..57: note = VEGFA-seq-r1-FWD
  misc_feature 34: note = n is a, c, g, or t
  source 1..57: mol_type = other DNA
acactctttccctacacgacgctcttccgatctnatggcgtcttcgagagtgaggac

SEQ ID NO: 161 - DNA, 57 bp, synthetic construct
  misc_feature 1..57: note = VEGFA-seq-r2-FWD
  misc_feature 34: note = n is a, c, g, or t
  source 1..57: mol_type = other DNA
acactctttccctacacgacgctcttccgatctngatgcgtcttcgagagtgaggac

SEQ ID NO: 162 - DNA, 57 bp, synthetic construct
  misc_feature 1..57: note = VEGFA-seq-r3-FWD
  misc_feature 34: note = n is a, c, g, or t
  source 1..57: mol_type = other DNA
acactctttccctacacgacgctcttccgatctntgcgcgtcttcgagagtgaggac

SEQ ID NO: 163 - DNA, 52 bp, synthetic construct
  misc_feature 1..52: note = VEGFA-seq-REV
  source 1..52: mol_type = other DNA
gactggagttcagacgtgtgctcttccgatctggggagagggacacacagat

SEQ ID NO: 164 - DNA, 27 bp, synthetic construct
  misc_feature 1..27: note = CXCR4-FWD
  source 1..27: mol_type = other DNA
agctggagtgaaaacttgaagactcag

SEQ ID NO: 165 - DNA, 26 bp, synthetic construct
  misc_feature 1..26: note = CXCR4-REV
  source 1..26: mol_type = other DNA
gtttgtatttaggcaggcgtgggaaa

SEQ ID NO: 166 - DNA, 23 bp, synthetic construct
  misc_feature 1..23: note = CXCR4-seq
  source 1..23: mol_type = other DNA
ctacaccgaggaaatgggctcag

SEQ ID NO: 167 - DNA, 20 bp, synthetic construct
  misc_feature 1..20: note = EMX1-FWD
  source 1..20: mol_type = other DNA
ggctccctgggttcaaagta

SEQ ID NO: 168 - DNA, 21 bp, synthetic construct
  misc_feature 1..21: note = EMX1-REV
  source 1..21: mol_type = other DNA
agaggggtctggatgtcgtaa

SEQ ID NO: 169 - DNA, 24 bp, synthetic construct
  misc_feature 1..24: note = EMX1-seq
  source 1..24: mol_type = other DNA
ggcctcctgagtttctcatctgtg

SEQ ID NO: 170 - DNA, 20 bp, synthetic construct
  misc_feature 1..20: note = EMX1-T-FWD
  source 1..20: mol_type = other DNA
ggctccctgggttcaaagta

SEQ ID NO: 171 - DNA, 21 bp, synthetic construct
  misc_feature 1..21: note = EMX1-T-REV
  source 1..21: mol_type = other DNA
agaggggtctggatgtcgtaa

SEQ ID NO: 172 - DNA, 25 bp, synthetic construct
  misc_feature 1..25: note = EMX1-T-seq
  source 1..25: mol_type = other DNA
aaccctatgtagcctcagtcttccc

SEQ ID NO: 173 - DNA, 23 bp, synthetic construct
  misc_feature 1..23: note = EMX1-OT1-FWD
  source 1..23: mol_type = other DNA
ttatcccctactccttcatccca

SEQ ID NO: 174 - DNA, 23 bp, synthetic construct
  misc_feature 1..23: note = EMX1-OT1-REV
  source 1..23: mol_type = other DNA
aaggacagcttcttatccctgtc

SEQ ID NO: 175 - DNA, 22 bp, synthetic construct
  misc_feature 1..22: note = EMX1-OT1-seq
  source 1..22: mol_type = other DNA
ggagatttgcatctgtggaggc

SEQ ID NO: 176 - DNA, 25 bp, synthetic construct
  misc_feature 1..25: note = EMX1-OT2-FWD
  source 1..25: mol_type = other DNA
actcctgggacaattatgaacggtg

SEQ ID NO: 177 - DNA, 28 bp, synthetic construct
  misc_feature 1..28: note = EMX1-OT2-REV
  source 1..28: mol_type = other DNA
actatccttctagtcttgggctaaattc

SEQ ID NO: 178 - DNA, 32 bp, synthetic construct
  misc_feature 1..32: note = EMX1-OT2-seq
  source 1..32: mol_type = other DNA
gcttcttgttctttggctttcttaatgaactg

SEQ ID NO: 179 - DNA, 22 bp, synthetic construct
  misc_feature 1..22: note = EMX1-OT3-FWD
  source 1..22: mol_type = other DNA
gcactgattcattaggagctgg

SEQ ID NO: 180 - DNA, 23 bp, synthetic construct
  misc_feature 1..23: note = EMX1-OT3-REV
  source 1..23: mol_type = other DNA
agtcctatagattcacccaccca

SEQ ID NO: 181 - DNA, 21 bp, synthetic construct
  misc_feature 1..21: note = EMX1-OT3-seq
  source 1..21: mol_type = other DNA
tcctggttctgccacttgctg

SEQ ID NO: 182 - DNA, 22 bp, synthetic construct
  misc_feature 1..22: note = VEGFA-FWD
  source 1..22: mol_type = other DNA
gctccagatggcacattgtcag

SEQ ID NO: 183 - DNA, 20 bp, synthetic construct
  misc_feature 1..20: note = VEGFA-REV
  source 1..20: mol_type = other DNA
agggagcaggaaagtgaggt

SEQ ID NO: 184 - DNA, 27 bp, synthetic construct
  misc_feature 1..27: note = VEGFA-seq
  source 1..27: mol_type = other DNA
caaatatgtagctgtttgggaggtcag

SEQ ID NO: 185 - DNA, 22 bp, synthetic construct
  misc_feature 1..22: note = VEGF1-OT1-FWD
  source 1..22: mol_type = other DNA
ttcccaccaaggagggtttctt

SEQ ID NO: 186 - DNA, 21 bp, synthetic construct
  misc_feature 1..21: note = VEGFA-OT1-REV
  source 1..21: mol_type = other DNA
cctccctcaagggaaggttgt

SEQ ID NO: 187 - DNA, 27 bp, synthetic construct
  misc_feature 1..27: note = VEGFA-OT1-seq
  source 1..27: mol_type = other DNA
caagtagctgagattacaggcatgtgc

SEQ ID NO: 188 - DNA, 22 bp, synthetic construct
  misc_feature 1..22: note = VEGFA-OT2-FWD
  source 1..22: mol_type = other DNA
attcctcaggtgggttgatggg

SEQ ID NO: 189 - DNA, 22 bp, synthetic construct
  misc_feature 1..22: note = VEGFA-OT2-REV
  source 1..22: mol_type = other DNA
agaaaggagcctcgaccaagtc

SEQ ID NO: 190 - DNA, 22 bp, synthetic construct
  misc_feature 1..22: note = VEGFA-OT2-seq
  source 1..22: mol_type = other DNA
gcctccctgctggttctcagag

SEQ ID NO: 191 - DNA, 22 bp, synthetic construct
  misc_feature 1..22: note = VEGFA-OT3-FWD
  source 1..22: mol_type = other DNA
tcccatcccactttagtgttcc

SEQ ID NO: 192 - DNA, 22 bp, synthetic construct
  misc_feature 1..22: note = VEGFA-OT3-REV
  source 1..22: mol_type = other DNA
taacccagaacatccaggcaac

SEQ ID NO: 193 - DNA, 20 bp, synthetic construct
  misc_feature 1..20: note = VEGFA-OT3-seq
  source 1..20: mol_type = other DNA
taccatgaacgcagccatgc

SEQ ID NO: 194 - DNA, 25 bp, synthetic construct
  source 1..25: mol_type = other DNA
gctagagatccgcggccgctaatac

SEQ ID NO: 195 - DNA, 24 bp, synthetic construct
  source 1..24: mol_type = other DNA
cactttcacgagctcgtccaccac

SEQ ID NO: 196 - DNA, 24 bp, Synthetic construct
  source 1..24: mol_type = other DNA
gtggtggacgagctcgtgaaagtg

SEQ ID NO: 197 - DNA, 24 bp, Synthetic construct
  source 1..24: mol_type = other DNA
ctgggtgctggatccggaaatctg

SEQ ID NO: 198 - DNA, 41 bp, Synthetic construct
  source 1..41: mol_type = other DNA
cgtcgtgggcgtgggcgtaccggttgatctcgcgcactttg

SEQ ID NO: 199 - DNA, 41 bp, Synthetic construct
  source 1..41: mol_type = other DNA
caaagtgcgcgagatcaaccggtacgcccacgcccacgacg

SEQ ID NO: 200 - DNA, 24 bp, Synthetic construct
  source 1..24: mol_type = other DNA
aaactgcgccccgcgccagcatcc

SEQ ID NO: 201 - DNA, 24 bp, Synthetic construct
  source 1..24: mol_type = other DNA
caccggatgctggcgcggggcgca

SEQ ID NO: 202 - DNA, 24 bp, Synthetic construct
  source 1..24: mol_type = other DNA
aaaccaccgcagttctaggcaagc

SEQ ID NO: 203 - DNA, 24 bp, Synthetic construct
  source 1..24: mol_type = other DNA
caccgcttgcctagaactgcggtg

SEQ ID NO: 204 - DNA, 24 bp, Synthetic construct
  source 1..24: mol_type = other DNA
aaacctttgttcacatcatctcac

SEQ ID NO: 205 - DNA, 24 bp, Synthetic construct
  source 1..24: mol_type = other DNA
caccgtgagatgatgtgaacaaag

SEQ ID NO: 206 - DNA, 24 bp, Synthetic construct
  source 1..24: mol_type = other DNA
aaactccaggtccccagcccaacc

SEQ ID NO: 207 - DNA, 24 bp, Synthetic construct
  source 1..24: mol_type = other DNA
caccggttgggctggggacctgga

SEQ ID NO: 208 - DNA, 67 bp, Synthetic construct
  source 1..67: mol_type = other DNA
gcaccgactcggtgccactttttcaagttgataacggactagccttattttaacttgctatttctag

SEQ ID NO: 209 - DNA, 67 bp, Synthetic construct
  source 1..67: mol_type = other DNA
agagctagaaatagcaagttaaaataaggctagtccgttatcaacttgaaaaagtggcaccgagtcg

SEQ ID NO: 210 - DNA, 29 bp, Synthetic construct
  source 1..29: mol_type = other DNA
ctctaaaaccactgtggggtggaggggac

SEQ ID NO: 211 - DNA, 29 bp, Synthetic construct
  source 1..29: mol_type = other DNA
caccgtcccctccaccccacagtggtttt

SEQ ID NO: 212 - DNA, 48 bp, Synthetic construct
  source 1..48: mol_type = other DNA
aaaaccctccaccccacagtggggccgctagcaagcttggacaggatt

SEQ ID NO: 213 - DNA, 48 bp, Synthetic construct
  source 1..48: mol_type = other DNA
gtgcaatcctgtccaagcttgctagcggccccactgtggggtggaggg

SEQ ID NO: 214 - DNA, 24 bp, Synthetic construct
  source 1..24: mol_type = other DNA
aaacatccttaggcctcctccttc

SEQ ID NO: 215 - DNA, 24 bp, Synthetic construct
  source 1..24: mol_type = other DNA
caccgaaggaggaggcctaaggat

SEQ ID NO: 216 - DNA, 29 bp, Synthetic construct
  source 1..29: mol_type = other DNA
ctctaaaaccctctttgtcatcacgcttc

SEQ ID NO: 217 - DNA, 29 bp, Synthetic construct
  source 1..29: mol_type = other DNA
caccgaagcgtgatgacaaagagggtttt

SEQ ID NO: 218 - DNA, 38 bp, Synthetic construct
  source 1..38: mol_type = other DNA
aaaaagcgtgatgacaaagaggagatcggccactgaca

SEQ ID NO: 219 - DNA, 38 bp, Synthetic construct
  source 1..38: mol_type = other DNA
gtgctgtcagtggccgatctcctctttgtcatcacgct

SEQ ID NO: 220 - DNA, 24 bp, Synthetic construct
  source 1..24: mol_type = other DNA
aaacgtacttgtccgtcatgcttc

SEQ ID NO: 221 - DNA, 24 bp, Synthetic construct
  source 1..24: mol_type = other DNA
caccgaagcatgacggacaagtac

SEQ ID NO: 222 - DNA, 29 bp, Synthetic construct
  source 1..29: mol_type = other DNA
ctctaaaacctgacaggtgcagcctgtac

SEQ ID NO: 223 - DNA, 29 bp, Synthetic construct
  source 1..29: mol_type = other DNA
caccgtacaggctgcacctgtcaggtttt

SEQ ID NO: 224 - DNA, 47 bp, Synthetic construct
  source 1..47: mol_type = other DNA
aaaacaggctgcacctgtcagtggccgacctgctagcaagcttcttt

SEQ ID NO: 225 - DNA, 47 bp, Synthetic construct
  source 1..47: mol_type = other DNA
gtgcaaagaagcttgctagcaggtcggccactgacaggtgcagcctg

SEQ ID NO: 226 - DNA, 24 bp, Synthetic construct
  source 1..24: mol_type = other DNA
aaactgtcatctacacagtcaacc

SEQ ID NO: 227 - DNA, 24 bp, Synthetic construct
  source 1..24: mol_type = other DNA
caccggttgactgtgtagatgaca

SEQ ID NO: 228 - DNA, 29 bp, Synthetic construct
  source 1..29: mol_type = other DNA
ctctaaaacccctagtcattggaggtgac

SEQ ID NO: 229 - DNA, 29 bp, Synthetic construct
  source 1..29: mol_type = other DNA
caccgtcacctccaatgactaggggtttt

SEQ ID NO: 230 - DNA, 38 bp, Synthetic construct
  source 1..38: mol_type = other DNA
aaaacacctccaatgactaggtgggcaaccacaaaccc

SEQ ID NO: 231 - DNA, 38 bp, Synthetic construct
  source 1..38: mol_type = other DNA
gtgcgggtttgtggttgcccacctagtcattggaggtg

SEQ ID NO: 232 - DNA, 25 bp, Synthetic construct
  source 1..25: mol_type = other DNA
aaaccgagggcagagtgctgcttgc

SEQ ID NO: 233 - DNA, 25 bp, Synthetic construct
  source 1..25: mol_type = other DNA
caccgcaagcagcactctgccctcg

SEQ ID NO: 234 - DNA, 29 bp, Synthetic construct
  source 1..29: mol_type = other DNA
ctctaaaacacggcgtgcagtgcttcagc

SEQ ID NO: 235 - DNA, 29 bp, Synthetic construct
  source 1..29: mol_type = other DNA
caccgctgaagcactgcacgccgtgtttt

SEQ ID NO: 236 - DNA, 36 bp, Synthetic construct
  source 1..36: mol_type = other DNA
aaaagcactgcacgccgtggctcagggtggtcacga

SEQ ID NO: 237 - DNA, 36 bp, Synthetic construct
  source 1..36: mol_type = other DNA
gtgctcgtgaccaccctgagccacggcgtgcagtgc

SEQ ID NO: 238 - DNA, 24 bp, Synthetic construct
  source 1..24: mol_type = other DNA
aaactaggtggcatcgccctcgcc

SEQ ID NO: 239 - DNA, 24 bp, Synthetic construct
  source 1..24: mol_type = other DNA
caccggcgagggcgatgccaccta

SEQ ID NO: 240 - DNA, 29 bp, Synthetic construct
  source 1..29: mol_type = other DNA
ctctaaaaccgattattatcagcctcagc

SEQ ID NO: 241 - DNA, 29 bp, Synthetic construct
  source 1..29: mol_type = other DNA
caccgctgaggctgataataatcggtttt

SEQ ID NO: 242 - DNA, 50 bp, Synthetic construct
  source 1..50: mol_type = other DNA
aaaatgaggctgataataagcttgctagccggcgatcagacagccccggt

SEQ ID NO: 243 - DNA, 50 bp, Synthetic construct
  source 1..50: mol_type = other DNA
gtgcaccggggctgtctgatcgccggctagcaagcttattatcagcctca

SEQ ID NO: 244 - DNA, 36 bp, Synthetic construct
  source 1..36: mol_type = other DNA
aaaaaggctgataataatctaggcggcgatcagaca

SEQ ID NO: 245 - DNA, 36 bp, Synthetic construct
  source 1..36: mol_type = other DNA
gtgctgtctgatcgccgcctagattattatcagcct

SEQ ID NO: 246 - DNA, 24 bp, Synthetic construct
  source 1..24: mol_type = other DNA
aaaccggtgtgggaaatcgtccgc

SEQ ID NO: 247 - DNA, 24 bp, Synthetic construct
  source 1..24: mol_type = other DNA
caccgcggacgatttcccacaccg

SEQ ID NO: 248 - DNA, 29 bp, Synthetic construct
  source 1..29: mol_type = other DNA
ctctaaaacgcgtcgggagagtcgcgtcc

SEQ ID NO: 249 - DNA, 29 bp, Synthetic construct
  source 1..29: mol_type = other DNA
caccggacgcgactctcccgacgcgtttt

SEQ ID NO: 250 - DNA, 39 bp, Synthetic construct
  source 1..39: mol_type = other DNA
aaaaacgcgactctcccgacagcggggaggctattctgc

SEQ ID NO: 251 - DNA, 39 bp, Synthetic construct
  source 1..39: mol_type = other DNA
gtgcgcagaatagcctccccgctgtcgggagagtcgcgt

SEQ ID NO: 252 - DNA, 24 bp, Synthetic construct
  source 1..24: mol_type = other DNA
aaacgcttctctgaaaggctctcc

SEQ ID NO: 253 - DNA, 24 bp, Synthetic construct
  source 1..24: mol_type = other DNA
caccggagagcctttcagagaagc

SEQ ID NO: 254 - DNA, 29 bp, Synthetic construct
  source 1..29: mol_type = other DNA
ctctaaaacccagctgcaagcatgtcatc

SEQ ID NO: 255 - DNA, 29 bp, Synthetic construct
  source 1..29: mol_type = other DNA
caccgatgacatgcttgcagctgggtttt

SEQ ID NO: 256 - DNA, 39 bp, Synthetic construct
  source 1..39: mol_type = other DNA
aaaatgacatgcttgcagctgtacgaaacaggttgtcct

SEQ ID NO: 257 - DNA, 39 bp, Synthetic construct
  source 1..39: mol_type = other DNA
gtgcaggacaacctgtttcgtacagctgcaagcatgtca

SEQ ID NO: 258 - DNA, 24 bp, Synthetic construct
  source 1..24: mol_type = other DNA
aaacatctttgctcctggctgtcc

SEQ ID NO: 259 - DNA, 24 bp, Synthetic construct
  source 1..24: mol_type = other DNA
caccggacagccaggagcaaagat

SEQ ID NO: 260 - DNA, 29 bp, Synthetic construct
  source 1..29: mol_type = other DNA
ctctaaaaccttggtggaagcgcaggctc

SEQ ID NO: 261 - DNA, 29 bp, Synthetic construct
  source 1..29: mol_type = other DNA
caccgagcctgcgcttccaccaaggtttt

SEQ ID NO: 262 - DNA, 38 bp, Synthetic construct
  source 1..38: mol_type = other DNA
aaaagcctgcgcttccaccaagggaggaggaccccgct

SEQ ID NO: 263 - DNA, 38 bp, Synthetic construct
  source 1..38: mol_type = other DNA
gtgcagcggggtcctcctcccttggtggaagcgcaggc

SEQ ID NO: 264 - DNA, 25 bp, Synthetic construct
  source 1..25: mol_type = other DNA
aaacctgcgctaaacgctggcagtc

SEQ ID NO: 265 - DNA, 25 bp, Synthetic construct
  source 1..25: mol_type = other DNA
caccgactgccagcgtttagcgcag

SEQ ID NO: 266 - DNA, 79 bp, Synthetic construct
  source 1..79: mol_type = other DNA
tagaactgcgcacacaaaaaaccaacacacagatctaatgaaaataaagatcttttattgtgtggaaggcgctgccccg

SEQ ID NO: 267 - DNA, 79 bp, Synthetic construct
  source 1..79: mol_type = other DNA
ccttccacacaataaaagatctttattttcattagatctgtgtgttggttttttgtgtgcgcagttctaggcaagcact

SEQ ID NO: 268 - DNA, 79 bp, Synthetic construct
  source 1..79: mol_type = other DNA
tttcccccttcacacaaaaaaccaacacacagatctaatgaaaataaagatcttttatttgttcacatcatctcacaaa

SEQ ID NO: 269 - DNA, 79 bp, Synthetic construct
  source 1..79: mol_type = other DNA
gatgtgaacaaataaaagatctttattttcattagatctgtgtgttggttttttgtgtgaagggggaaaagtcaatcca

SEQ ID NO: 270 - DNA, 59 bp, Synthetic construct
  source 1..59: mol_type = other DNA
acactctttccctacacgacgctcttccgatctnatgcgtaaacggccacaagttcagc

SEQ ID NO: 271 - DNA, 59 bp, Synthetic construct
  source 1..59: mol_type = other DNA
acactctttccctacacgacgctcttccgatctngatcgtaaacggccacaagttcagc

SEQ ID NO: 272 - DNA, 59 bp, Synthetic construct
  source 1..59: mol_type = other DNA
acactctttccctacacgacgctcttccgatctntgccgtaaacggccacaagttcagc

SEQ ID NO: 273 - DNA, 59 bp, Synthetic construct
  source 1..59: mol_type = other DNA
acactctttccctacacgacgctcttccgatctngaccagtgaaatctggcgagagcag

SEQ ID NO: 274 - DNA, 59 bp, Synthetic construct
  source 1..59: mol_type = other DNA
acactctttccctacacgacgctcttccgatctntgacagtgaaatctggcgagagcag

SEQ ID NO: 275 - DNA, 59 bp, Synthetic construct
  source 1..59: mol_type = other DNA
acactctttccctacacgacgctcttccgatctnactcagtgaaatctggcgagagcag

SEQ ID NO: 276 - DNA, 54 bp, Synthetic construct
  source 1..54: mol_type = other DNA
gactggagttcagacgtgtgctcttccgatcttcaaactatgtgttcgcgggtc

SEQ ID NO: 277 - DNA, 60 bp, Synthetic construct
  source 1..60: mol_type = other DNA
acactctttccctacacgacgctcttccgatctncgttctccggagcagaaatagaacag

SEQ ID NO: 278 - DNA, 60 bp, Synthetic construct
  source 1..60: mol_type = other DNA
acactctttccctacacgacgctcttccgatctnacgtctccggagcagaaatagaacag

SEQ ID NO: 279 - DNA, 60 bp, Synthetic construct
  source 1..60: mol_type = other DNA
acactctttccctacacgacgctcttccgatctngtatctccggagcagaaatagaacag

SEQ ID NO: 280 - DNA, 54 bp, Synthetic construct
  source 1..54: mol_type = other DNA
gactggagttcagacgtgtgctcttccgatctttcatgccgtcaagtctcattt

SEQ ID NO: 281 - DNA, 60 bp, Synthetic construct
  source 1..60: mol_type = other DNA
acactctttccctacacgacgctcttccgatctnatgattccatgggactgactttctgc

SEQ ID NO: 282 - DNA, 60 bp, Synthetic construct
  source 1..60: mol_type = other DNA
acactctttccctacacgacgctcttccgatctngatattccatgggactgactttctgc

SEQ ID NO: 283 - DNA, 60 bp, Synthetic construct
  source 1..60: mol_type = other DNA
acactctttccctacacgacgctcttccgatctntgcattccatgggactgactttctgc

SEQ ID NO: 284 - DNA, 54 bp, Synthetic construct
  source 1..54: mol_type = other DNA
gactggagttcagacgtgtgctcttccgatctcatctggacctgggtcttcagt

SEQ ID NO: 285 - DNA, 4104 bp, Synthetic construct
  source 1..4104: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgaccggggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtggccgagatcaacaactaccaccacgcccacgacgcctacctgaacgccgtcgtgggaaccgccctgatcaaaaagtaccctgccctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttcggctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 286 - DNA, 6393 bp, Synthetic construct
  source 1..6393: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggaagctctggtggcagcaagcggaccgccgacggctctgaattcgagagccctaagaagaaaagaaaggtgagcggaggctctagcggcggaagcaccctgaacattgaagacgagtatagactgcatgaaacaagcaaggaacccgacgtgtccctgggctccacctggctgtccgactttccccaggcctgggccgagacaggaggaatgggcctggccgtgcggcaggcacccctgatcatccctctgaaggccacctctacacccgtgagcatcaagcagtaccctatgtctcaggaggccagactgggcatcaagcctcacatccagaggctgctggaccagggcatcctggtgccatgccagagcccctggaacacaccactgctgcccgtgaagaagccaggcaccaatgactatagacccgtgcaggatctgagagaggtgaacaagagggtggaggatatccaccccaccgtgcccaacccttacaatctgctgtccggcctgcccccttctcaccagtggtatacagtgctggacctgaaggatgccttcttttgtctgagactgcaccctaccagccagccactgttcgcctttgagtggagggaccctgagatgggcatctctggccagctgacctggacacgcctgcctcagggcttcaagaatagcccaacactgtttaacgaggccctgcaccgcgacctggcagatttccggatccagcacccagatctgatcctgctgcagtacgtggacgatctgctgctggccgccaccagcgagctggattgccagcagggaacacgcgccctgctgcagaccctgggaaacctgggatatagggcatccgccaagaaggcccagatctgtcagaagcaggtgaagtacctgggctatctgctgaaggagggccagagatggctgacagaggccaggaaggagacagtgatgggccagccaacacccaagaccccaagacagctgagggagttcctgggcaaagcaggattttgcaggctgttcatcccaggattcgcagagatggcagcacctctgtacccactgaccaagccgggcaccctgtttaattggggccctgaccagcagaaggcctatcaggagatcaagcaggccctgctgacagcaccagccctgggcctgccagacctgaccaagcctttcgagctgtttgtggatgagaagcagggctacgccaagggcgtgctgacccagaagctgggaccatggagacggcccgtggcctatctgtccaagaagctggacccagtggcagcaggatggccaccatgcctgaggatggtggcagcaatcgccgtgctgacaaaggatgccggcaagctgaccatgggacagccactggtcatcctggcaccacacgcagtggaggccctggtgaagcagcctccagatcgctggctgtctaacgcccggatgacacactaccaggccctgctgctggacaccgatcgcgtgcagtttggccctgtggtggccctgaatccagccaccctgctgcctctgccagaggagggcctgcagcacaactgtctggacatcctggcagaggcacacggaacaaggccagacctgaccgatcagcccctgcctgacgccgatcacacatggtataccgatggaagctccctgctgcaggagggccagaggaaggcaggagcagcagtgaccacagagacagaagtgatctgggccaaggccctgccagcaggcacatccgcccagcgggccgagctgatcgccctgacccaggccctgaagatggccgagggcaagaagctgaacgtgtacacagactccagatatgccttcgccaccgcacacatccacggagagatctacaggcgccggggctggctgacctctgagggcaaggagatcaagaacaaggatgagatcctggccctgctgaaggccctgtttctgcccaagcggctgagcatcatccactgtcctggacaccagaagggacactccgccgaggcaaggggcaatcggatggccgaccaggccgccagaaaggctgctattactgaaactcccgacacttccactctgctgattgaaaactcctccccttctggcggctcaaaaagaaccgccgacggcagcgaattcgagtctcccaagaagaagaggaaagtcggctctggccctgccgctaagagagtgaagctggac

SEQ ID NO: 287 - DNA, 6393 bp, Synthetic construct
  source 1..6393: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctggccgacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactacgcccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggaagctctggtggcagcaagcggaccgccgacggctctgaattcgagagccctaagaagaaaagaaaggtgagcggaggctctagcggcggaagcaccctgaacattgaagacgagtatagactgcatgaaacaagcaaggaacccgacgtgtccctgggctccacctggctgtccgactttccccaggcctgggccgagacaggaggaatgggcctggccgtgcggcaggcacccctgatcatccctctgaaggccacctctacacccgtgagcatcaagcagtaccctatgtctcaggaggccagactgggcatcaagcctcacatccagaggctgctggaccagggcatcctggtgccatgccagagcccctggaacacaccactgctgcccgtgaagaagccaggcaccaatgactatagacccgtgcaggatctgagagaggtgaacaagagggtggaggatatccaccccaccgtgcccaacccttacaatctgctgtccggcctgcccccttctcaccagtggtatacagtgctggacctgaaggatgccttcttttgtctgagactgcaccctaccagccagccactgttcgcctttgagtggagggaccctgagatgggcatctctggccagctgacctggacacgcctgcctcagggcttcaagaatagcccaacactgtttaacgaggccctgcaccgcgacctggcagatttccggatccagcacccagatctgatcctgctgcagtacgtggacgatctgctgctggccgccaccagcgagctggattgccagcagggaacacgcgccctgctgcagaccctgggaaacctgggatatagggcatccgccaagaaggcccagatctgtcagaagcaggtgaagtacctgggctatctgctgaaggagggccagagatggctgacagaggccaggaaggagacagtgatgggccagccaacacccaagaccccaagacagctgagggagttcctgggcaaagcaggattttgcaggctgttcatcccaggattcgcagagatggcagcacctctgtacccactgaccaagccgggcaccctgtttaattggggccctgaccagcagaaggcctatcaggagatcaagcaggccctgctgacagcaccagccctgggcctgccagacctgaccaagcctttcgagctgtttgtggatgagaagcagggctacgccaagggcgtgctgacccagaagctgggaccatggagacggcccgtggcctatctgtccaagaagctggacccagtggcagcaggatggccaccatgcctgaggatggtggcagcaatcgccgtgctgacaaaggatgccggcaagctgaccatgggacagccactggtcatcctggcaccacacgcagtggaggccctggtgaagcagcctccagatcgctggctgtctaacgcccggatgacacactaccaggccctgctgctggacaccgatcgcgtgcagtttggccctgtggtggccctgaatccagccaccctgctgcctctgccagaggagggcctgcagcacaactgtctggacatcctggcagaggcacacggaacaaggccagacctgaccgatcagcccctgcctgacgccgatcacacatggtataccgatggaagctccctgctgcaggagggccagaggaaggcaggagcagcagtgaccacagagacagaagtgatctgggccaaggccctgccagcaggcacatccgcccagcgggccgagctgatcgccctgacccaggccctgaagatggccgagggcaagaagctgaacgtgtacacagactccagatatgccttcgccaccgcacacatccacggagagatctacaggcgccggggctggctgacctctgagggcaaggagatcaagaacaaggatgagatcctggccctgctgaaggccctgtttctgcccaagcggctgagcatcatccactgtcctggacaccagaagggacactccgccgaggcaaggggcaatcggatggccgaccaggccgccagaaaggctgctattactgaaactcccgacacttccactctgctgattgaaaactcctccccttctggcggctcaaaaagaaccgccgacggcagcgaattcgagtctcccaagaagaagaggaaagtcggctctggccctgccgctaagagagtgaagctggac

SEQ ID NO: 288 - DNA, 350 bp, Synthetic construct
  source 1..350: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttt

SEQ ID NO: 289 - DNA, 58 bp, Synthetic construct
  source 1..58: mol_type = other DNA
acactctttccctacacgacgctcttccgatctngacttacaggagccaggtcggtat

SEQ ID NO: 290 - DNA, 58 bp, Synthetic construct
  source 1..58: mol_type = other DNA
acactctttccctacacgacgctcttccgatctntgattacaggagccaggtcggtat

SEQ ID NO: 291 - DNA, 58 bp, Synthetic construct
  source 1..58: mol_type = other DNA
acactctttccctacacgacgctcttccgatctnactttacaggagccaggtcggtat

SEQ ID NO: 292 - DNA, 52 bp, Synthetic construct
  source 1..52: mol_type = other DNA
gactggagttcagacgtgtgctcttccgatctgtcaggtgcgtaacaatcgc

SEQ ID NO: 293 - protein, 2131 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD

SEQ ID NO: 294 - protein, 2131 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYAHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD